(19) United States
(12) Patent Application Publication
(10) Pub. No.: US 2012/0107888 A1
(43) Pub. Date: May 3, 2012

(54) MODULATION OF FERMENTATION PRODUCTS THROUGH VITAMIN SUPPLEMENTATION

(75) Inventors: Matthias Schmalisch, Marlborough, MA (US); William G. Latouf, Ashland, MA (US); Brandon Wolner, Auburn, MA (US); Patrick O'Mullan, Grafton, MA (US)

(73) Assignee: Qteros, Inc., Marlborough, MA (US)

(21) Appl. No.: 13/289,568

(22) Filed: Nov. 4, 2011

Related U.S. Application Data
(60) Provisional application No. 61/444,116, filed on Feb. 17, 2011.
(60) Provisional application No. 61/524,668, filed on Aug. 17, 2011.

(51) Int. Cl. C12P 7/02; C12M 1/00; C12N 1/21

(52) U.S. Cl. 435/155; 435/252.3; 435/289.1

(57) ABSTRACT

Improved yields of biofuels and other chemicals are obtained by culturing a cellulolytic microorganism, such as Clostridium phytofermentans, Clostridium sp. Q.D, or Clostridium biocatalysts thereof in modulated amounts of vitamins, such as thiamine or nicotinic acid. Provided are methods to increase yields of ethanol and other fermentation products through vitamin supplementation. Recombinant microorganisms with altered metabolic pathways that obviate the need to modulate media components during hydrolysis and fermentation of biomass are also described.

### CROSS-REFERENCE

This application claims the benefit of U.S. Provisional Application No. 61/444,116, filed Feb. 17, 2011 and U.S. Provisional Application No. 61/524,668, filed Aug. 17, 2011, each of which is incorporated herein by reference in its entirety.

### BACKGROUND

Microbial biocatalyst fermentation from biomass containing polymers such as cellulose, lignocellulose, pectin, polyglucose and/or polyfructose can provide much needed solutions for the world energy problem. Species of yeast, fungi and bacteria have been reported to be able to convert carbonaceous biomass to monomeric sugars to ethanol and other chemical products. However, many of these microorganisms grow slowly in fermentation and/or produce desired chemicals only at low concentrations. Such product production issues, in addition to affecting the chemical product makeup, can also affect overall efficiency and productivity.

Media components are an important part of fermentation. Biomass provides carbon sources and some essential growth factors for fungal and bacterial biocatalysts, but not all. Little is known regarding the effects of media composition, especially in anaerobic fermentation. In addition to affecting the total cost of the ethanol produced from biomass, growth media components control the effectiveness of biocatalysts. Vitamins, for example, are essential in supplying or enabling synthesis of specific co-factors for facilitating enzymatic reactions in most bacterial organisms. The effects of such components can be exploited to maximize the yields of fermentation products.

### SUMMARY

In one aspect, disclosed herein are genetically modified microorganisms adapted for decreased vitamin dependency that ferment a biomass to produce one or more fermentation end-products, wherein said microorganism comprises a genetic modification that decreases vitamin dependency. In one embodiment, the genetic modification comprises one or more heterologous polynucleotides that encode for enzymes in one or more metabolic pathways, wherein the metabolic pathways comprise a thiamine metabolic pathway, a nicotinate and nicotinamide metabolic pathway, a vitamin B₆ metabolic pathway, a one carbon pool by folate pathway, or a combination thereof. In one embodiment, at least one of the polynucleotides has at least about 60% identity to SEQ ID NO:20, SEQ ID NO:21, SEQ ID NO:22, SEQ ID NO:23, SEQ ID NO:24, SEQ ID NO:31, SEQ ID NO:32, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or a combination thereof. In one embodiment, at least one of the polynucleotides encodes for a polypeptide with at least about 60% identity to SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, or a combination thereof. In one embodiment, the microorganism hydrolyzes and ferments hemicellulosic or lignocellulosic material. In one embodiment, the microorganism is a genetically modified Clostridium species. In one embodiment, the microorganism is a genetically modified Clostridium phytofermentans or Clostridium sp Q.D. In one embodiment, the one or more fermentation end-products comprise one or more alcohols. In one embodiment, the one or more alcohols comprise ethanol.

In another aspect, disclosed herein are methods of producing one or more fermentation end-products comprising: (a) providing a biomass in a medium; (b) contacting the medium with a genetically modified microorganism adapted for decreased vitamin dependency, wherein said microorganism comprises a genetic modification that decreases vitamin dependency; and; (c) allowing sufficient time for the microorganism to produce the fermentation end-products from the biomass. In one embodiment, the genetic modification comprises one or more heterologous polynucleotides that encode for enzymes in one or more metabolic pathways, wherein the metabolic pathways comprise a thiamine metabolic pathway, a nicotinate and nicotinamide metabolic pathway, a vitamin B₆ metabolic pathway, a one carbon pool by folate pathway, or a combination thereof. In one embodiment, at least one of the polynucleotides has at least about 60% identity to SEQ ID NO:20, SEQ ID NO:21, SEQ ID NO:22, SEQ ID NO:23, SEQ ID NO:24, SEQ ID NO:31, SEQ ID NO:32, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or a combination thereof. In one embodiment, at least one of the polynucleotides encodes for a polypeptide with at least about 60% identity to SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, or a combination thereof. In one embodiment, the microorganism hydrolyzes and ferments hemicellulosic or lignocellulosic material. In one embodiment, the microorganism is a genetically modified Clostridium species. In one embodiment, the microorganism is a genetically modified Clostridium phytofermentans or Clostridium sp Q.D. In one embodiment, the one or more fermentation end-products comprise one or more alcohols. In one embodiment, the one or more alcohols comprise ethanol. In one embodiment, the media is supplemented with one or more vitamins, wherein the vitamins comprise thiamine, an NAD+ precursor, a vitamin B₆, a vitamin B₉, or a combination thereof. In one embodiment, the medium is supplemented with one or more vitamins, wherein at least one of the vitamins is at a concentration below the minimal nutritional requirements for growth of an unmodified microorganism of the same species. In one embodiment, the method further comprises a second microorganism, wherein the second microorganism produces at least one vitamin that is used by the genetically modified microorganism. In one embodiment, a yield of at least one of the fermentation end-products is between about 1% and about 100% higher than a yield of the fermentation end-product produced by an unmodified microorganism of the same species. In one embodiment, the microorganism produces a greater yield of one or more fermentation end-products than an unmodified microorganism of the same species. In one embodiment, the media is deficient in one or more vitamins. In one embodiment, the media does not comprise any of at least one of the one or more vitamins. In one embodiment, the media comprises less than the minimal nutritional requirements of at least one of the vitamins for growth of an unmodified microorganism of the same species. In one embodiment, the one or more vitamins comprise thiamine, an NAD+ precursor, a vitamin B₆, a vitamin B₉, or a combination thereof. In one embodiment, the NAD+ precursor comprises nicotinic acid, nicotinamide, nicotinamide riboside, or a combination thereof. In one embodiment, the vitamin B₆ comprises pyridoxine, pyridoxine 5′-phosphate, pyridoxal, pyridoxal 5′-phosphate, pyridoxamine, pyridoxamine 5′-phosphate, or a combination thereof. In one embodiment, the vitamin B9 comprises folic acid, folate, folinic acid, or a combination thereof.

Also disclosed herein are systems for the production of one or more fermentation end-products comprising: (a) a media comprising a biomass; (b) a genetically modified microorganism adapted for decreased vitamin dependency, wherein said microorganism comprises a genetic modification that decreases vitamin dependency; and, (c) a fermentor configured to house the media and the microorganism. In one embodiment, the genetic modification comprises one or more heterologous polynucleotides that encode for enzymes in one or more metabolic pathways, wherein the metabolic pathways comprise a thiamine metabolic pathway, a nicotinate and nicotinamide metabolic pathway, a vitamin B₆ metabolic pathway, a one carbon pool by folate pathway, or a combination thereof. In one embodiment, at least one of the polynucleotides has at least about 60% identity to SEQ ID NO:20, SEQ ID NO:21, SEQ ID NO:22, SEQ ID NO:23, SEQ ID NO:24, SEQ ID NO:31, SEQ ID NO:32, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or a combination thereof. In one embodiment, at least one of the polynucleotides encodes for a polypeptide with at least about 60% identity to SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, or a combination thereof. In one embodiment, the microorganism hydrolyzes and ferments hemicellulosic or lignocellulosic material. In one embodiment, the microorganism is a genetically modified Clostridium species. In one embodiment, the microorganism is a genetically modified Clostridium phytofermentans or Clostridium sp Q.D. In one embodiment, the one or more fermentation end-products comprise one or more alcohols. In one embodiment, the one or more alcohols comprise ethanol. In one embodiment, the media is supplemented with one or more vitamins, wherein the vitamins comprise thiamine, an NAD+ precursor, a vitamin B₆, a vitamin B₉, or a combination thereof. In one embodiment, the medium is supplemented with one or more vitamins, wherein at least one of the vitamins is at a concentration below the minimal nutritional requirements for growth of an unmodified microorganism of the same species. In one embodiment, the method further comprises a second microorganism, wherein the second microorganism produces at least one vitamin that is used by the genetically modified microorganism. In one embodiment, the microorganism can produce a greater yield one or more fermentation end-products than an unmodified microorganism of the same species. In one embodiment, a yield of at least one of the fermentation end-products is between about 1% and about 100% higher than a yield of the fermentation end-product produced by an unmodified microorganism of the same species. In one embodiment, the media is deficient in one or more vitamins. In one embodiment, the media does not comprise any of at least one of the one or more vitamins. In one embodiment, the media comprises less than the minimal nutritional requirements of at least one of the vitamins for growth of an unmodified microorganism of the same species. In one embodiment, the one or more vitamins comprise thiamine, an NAD+ precursor, a vitamin B₆, a vitamin B₉, or a combination thereof. In one embodiment, the NAD+ precursor comprises nicotinic acid, nicotinamide, nicotinamide riboside, or a combination thereof. In one embodiment, the vitamin B₆ comprises pyridoxine, pyridoxine 5′-phosphate, pyridoxal, pyridoxal 5′-phosphate, pyridoxamine, pyridoxamine 5′-phosphate, or a combination thereof. In one embodiment, the vitamin B9 comprises folic acid, folate, folinic acid, or a combination thereof.

Also disclosed herein are genetically modified microorganisms adapted for decreased vitamin dependency that ferment a biomass to produce one or more fermentation end-products, wherein said microorganism comprises a genetic modification that decreases vitamin dependency. In one embodiment, the microorganism comprises one or more genetic modifications that enables the microorganism to grow in a medium deficient in one or more vitamins required for growth of an unmodified microorganism of the same species. In one embodiment, the vitamins comprise thiamine, a nicotinamide adenine dinucleotide (NAD+) precursor (e.g., nicotinic acid, nicotinamide, or nicotinamide riboside), a vitamin B₆ (e.g., pyridoxine, pyridoxine 5′-phosphate, pyridoxal, pyridoxal 5′-phosphate, pyridoxamine, or pyridoxamine 5′-phosphate), a vitamin B₉ (e.g., folic acid, folate, or folinic acid), or a combination thereof. In one embodiment, the genetic modifications comprise a heterologous copy of one or more polynucleotides that encode for enzymes in one or more metabolic pathways, wherein the metabolic pathways comprises a thiamine metabolic pathway, a nicotinate and nicotinamide metabolic pathway, a vitamin B₆ metabolic pathway, a one carbon pool by folate pathway, or a combination thereof. In one embodiment, the polynucleotides comprise Ccel_{—}1989, Ccel_{—}1990, Ccel_{—}1991, Ccel_{—}1992, thiC, thiD, thiE, thiF, thiG, thiH, thiL, thiM, Ccel_{—}3480, Ccel_{—}3479, Ccel_{—}3478, Ccel_{—}1858, Ccel_{—}1859, Ccel_{—}1310, or a combination thereof. In one embodiment, at least one of the polynucleotides has at least about 60% identity to SEQ ID NO:20, SEQ ID NO:21, SEQ ID NO:22, SEQ ID NO:23, SEQ ID NO:24, SEQ ID NO:31, SEQ ID NO:32, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or a combination thereof. In one embodiment, at least one of the polynucleotides encodes for a polypeptide with at least about 60% identity to SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, or a combination thereof. In one embodiment, the vitamins comprise thiamine. In one embodiment, the one or more metabolic pathways comprise the thiamine metabolic pathway. In one embodiment, at least one of the polynucleotides are from Clostridium cellulolyticum. In one embodiment, the polynucleotides comprise Ccel_{—}1989, Ccel_{—}1990, Ccel_{—}1991, Ccel_{—}1992, or a combination thereof. In one embodiment, the polynucleotides comprise Ccel_{—}1989, Ccel_{—}1990, Ccel_{—}1991, and Ccel_{—}1992. In one embodiment, at least one of the polynucleotides has at least about 60% identity to SEQ ID NO: 21. In one embodiment, at least one of the polynucleotides has at least about 60% identity to SEQ ID NO:33, SEQ ID NO:35, SEQ ID NO:37, SEQ ID NO:39, or a combination thereof. In one embodiment, at least one of the polynucleotides encodes for a polypeptide with at least about 60% identity to SEQ ID NO:34, SEQ ID NO:36, SEQ ID NO:38, SEQ ID NO:40, or a combination thereof. In one embodiment, at least one of the polynucleotides are from Escherichia coli. In one embodiment, the polynucleotides comprise thiC, thiD, thiE, thiF, thiG, thiH, thiL, thiM, or a combination thereof. In one embodiment, the polynucleotides comprise thiC, thiD, thiE, thiF, thiG, thiH, thiL, and thiM. In one embodiment, at least one of the polynucleotides has at least about 60% identity to SEQ ID NO:22, SEQ ID NO: 23, SEQ ID NO:24, or a combination thereof. In one embodiment, at least one of the polynucleotides has at least about 60% identity to SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57 or a combination thereof. In one embodiment, at least one of the polynucleotides encodes for a polypeptide with at least about 60% identity to SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, or a combination thereof. In one embodiment, the microorganism synthesizes more thiamine than an unmodified microorganism of the same species. In one embodiment, the vitamins comprise an NAD+ precursor. In one embodiment, the NAD+ precursor is nicotinic acid, nicotinamide, nicotinamide riboside, or a combination thereof. In one embodiment, the metabolic pathways comprise the nicotinate and nicotinamide metabolic pathway. In one embodiment, at least one of the polynucleotides encodes an enzyme, wherein the enzyme has an activity corresponding to EC numbers 1.4.3.16, 2.5.1.72, or 2.4.2.19. In one embodiment, at least one of the polynucleotides is from Clostridium cellulolyticum. In one embodiment, the polynucleotides comprise Ccel_{—}3480, Ccel_{—}3479, Ccel_{—}3478, or a combination thereof. In one embodiment, the polynucleotides comprise Ccel_{—}3480, Ccel_{—}3479, and Ccel_{—}3478. In one embodiment, at least one of the polynucleotides has at least about 60% identity to SEQ ID NO: 20. In one embodiment, at least one of the polynucleotides has at least about 60% identity to SEQ ID NO:59, SEQ ID NO:61, SEQ ID NO:63, or a combination thereof. In one embodiment, at least one of the polynucleotides encodes for a polypeptide with at least about 60% identity to SEQ ID NO:60, SEQ ID NO:62, SEQ ID NO:64, or a combination thereof. In one embodiment, the microorganism produces more NAD+ than an unmodified microorganism of the same species. In one embodiment, the vitamins comprise a vitamin B₆. In one embodiment, the vitamin B₆ comprises pyridoxine, pyridoxine 5′-phosphate, pyridoxal, pyridoxal 5′-phosphate, pyridoxamine, pyridoxamine 5′-phosphate, or a combination thereof. In one embodiment, the metabolic pathways comprise the vitamin B₆ metabolic pathway. In one embodiment, at least one of the polynucleotides is from Clostridium cellulolyticum. In one embodiment, the polynucleotides comprise Ccel_{—}1858, Ccel_{—}1859, or a combination thereof. In one embodiment, the polynucleotides comprise Ccel_{—}1858 and Ccel_{—}1859. In one embodiment, at least one of the polynucleotides has at least about 60% identity to SEQ ID NO: 31. In one embodiment, at least one of the polynucleotides has at least about 60% identity to SEQ ID NO:65, SEQ ID NO:67, or a combination thereof. In one embodiment, at least one of the polynucleotides encodes for a polypeptide with at least about 60% identity to SEQ ID NO:66, SEQ ID NO:68, or a combination thereof. In one embodiment, the microorganism synthesizes more pyridoxal 5′-phosphate (PLP) than an unmodified microorganism of the same species. In one embodiment, the vitamins comprise the vitamin B₉. In one embodiment, the vitamin B₉ comprises folic acid, folate, folinic acid, or a combination thereof. In one embodiment, the metabolic pathways comprise the one carbon pool by folate metabolic pathway. In one embodiment, at least one of the polynucleotides is from Clostridium cellulolyticum. In one embodiment, at least one of the polynucleotides encodes for a dihydrofolate reductase. In one embodiment, the polynucleotides comprise Ccel_{—}1310. In one embodiment, at least one of the polynucleotides has at least about 60% identity to SEQ ID NO: 32. In one embodiment, at least one of the polynucleotides has at least about 60% identity to SEQ ID NO:69. In one embodiment, at least one of the polynucleotides encodes for a polypeptide with at least about 60% identity to SEQ ID NO:70. In one embodiment, the microorganism can produce more tetrahydrofolate (THF) than an unmodified microorganism of the same species. In one embodiment, the microorganism can produce a greater yield one or more fermentation end-products than an unmodified microorganism of the same species. In one embodiment, the one or more fermentation end-products comprise one or more alcohols. In one embodiment, the one or more alcohols comprise methanol, ethanol, propanol, butanol, or a combination thereof. In one embodiment, the one or more alcohols comprise ethanol. In one embodiment, the microorganism further produces a lower yield of one or more other fermentation end-product than an unmodified microorganism of the same species. In one embodiment, the one or more other fermentation end-products comprise one or more acids. In one embodiment, the one or more acids comprise lactic acid. In one embodiment, the microorganism can ferment C5 sugars. In one embodiment, the microorganism can ferment C6 sugars. In one embodiment, the microorganism can ferment C5 and C6 sugars. In one embodiment, the microorganism can hydrolyze cellulose. In one embodiment, the microorganism can hydrolyze hemicellulose. In one embodiment, the microorganism can hydrolyze lignocellulose. In one embodiment, the microorganism can hydrolyze and ferment cellulose. In one embodiment, the microorganism can hydrolyze and ferment hemicellulose. In one embodiment, the microorganism can hydrolyze and ferment lignocellulose. In one embodiment, the microorganism can hydrolyze and ferment cellulosic, hemicellulosic and lignocellulosic material. In one embodiment, the microorganism is a genetically modified Thermoanaerobacter species. In one embodiment, the microorganism is a genetically modified Thermoanaerobacter pseudethanolicus, Thermoanaerobacter mathranii, Thermoanaerobacter italicus, Thermoanaerobacter brockii, T. acetoethylicus, Thermoanaerobacter ethanolicus, Thermoanaerobacter kivui, Thermoanaerobacter siderophilus, Thermoanaerobacter sulfuragignens, Thermoanaerobacter sulfurophilus, Thermoanaerobacter thermocopriae, Thermoanaerobacter thermohydrosulfuricus, Thermoanaerobacter uzonensis, or Thermoanaerobacter wiegelii. In one embodiment, the microorganism is a genetically modified Clostridium species. In one embodiment, the microorganism is a genetically modified Clostridium phytofermentans, Clostridium Q.D or a variant thereof.

Also disclosed herein are methods of producing one or more fermentation end-products comprising: (a) providing a biomass in a media; (b) contacting the media with a genetically modified microorganism adapted for decreased vitamin dependency, wherein said microorganism comprises a genetic modification that decreases vitamin dependency; and, (c) allowing sufficient time for the microorganism to produce the fermentation end-products from the biomass. In one embodiment, the microorganism comprises one or more genetic modifications that enables the microorganism to grow in a medium deficient in one or more vitamins required for growth of an unmodified microorganism of the same species. In one embodiment, the vitamins comprise thiamine, a nicotinamide adenine dinucleotide (NAD+) precursor (e.g., nicotinic acid, nicotinamide, or nicotinamide riboside), a vitamin B₆ (e.g., pyridoxine, pyridoxine 5′-phosphate, pyridoxal, pyridoxal 5′-phosphate, pyridoxamine, or pyridoxamine 5′-phosphate), a vitamin B₉ (e.g., folic acid, folate, or folinic acid), or a combination thereof. In one embodiment, the genetic modifications comprise a heterologous copy of one or more polynucleotides that encode for enzymes in one or more metabolic pathways, wherein the metabolic pathways comprises a thiamine metabolic pathway, a nicotinate and nicotinamide metabolic pathway, a vitamin B₆ metabolic pathway, a one carbon pool by folate pathway, or a combination thereof. In one embodiment, the polynucleotides comprise Ccel_{—}1989, Ccel_{—}1990, Ccel_{—}1991, Ccel_{—}1992, thiC, thiD, thiE, thiF, thiG, thiH, thiL, thiM, Ccel_{—}3480, Ccel_{—}3479, Ccel_{—}3478, Ccel_{—}1858, Ccel_{—}1859, Ccel_{—}1310, or a combination thereof. In one embodiment, at least one of the polynucleotides has at least about 60% identity. to SEQ ID NO:20, SEQ ID NO:21, SEQ ID NO:22, SEQ ID NO:23, SEQ ID NO:24, SEQ ID NO:31, SEQ ID NO:32, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or a combination thereof. In one embodiment, at least one of the polynucleotides encodes for a polypeptide with at least about 60% identity to SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, or a combination thereof. In one embodiment, the vitamins comprise thiamine. In one embodiment, the metabolic pathways comprise the thiamine metabolic pathway. In one embodiment, at least one of the polynucleotides is from Clostridium cellulolyticum. In one embodiment, the polynucleotides comprise Ccel_{—}1989, Ccel_{—}1990, Ccel_{—}1991, Ccel_{—}1992, or a combination thereof. In one embodiment, the polynucleotides comprise Ccel_{—}1989, Ccel_{—}1990, Ccel_{—}1991, and Ccel_{—}1992. In one embodiment, at least one of the polynucleotides has at least about 60% identity to SEQ ID NO: 21. In one embodiment, at least one of the polynucleotides has at least about 60% identity to SEQ ID NO:33, SEQ ID NO:35, SEQ ID NO:37, SEQ ID NO:39, or a combination thereof. In one embodiment, at least one of the polynucleotides encodes for a polypeptide with at least about 60% identity to SEQ ID NO:34, SEQ ID NO:36, SEQ ID NO:38, SEQ ID NO:40, or a combination thereof. In one embodiment, at least one of the polynucleotides are from Escherichia coli. In one embodiment, the polynucleotides comprise thiC, thiD, thiE, thiF, thiG, thiH, thiL, thiM, or a combination thereof. In one embodiment, the polynucleotides comprise thiC, thiD, thiE, thiF, thiG, thiH, thiL, and thiM. In one embodiment, at least one of the polynucleotides has at least about 60% identity to SEQ ID NO:22, SEQ ID NO: 23, SEQ ID NO:24, or a combination thereof. In one embodiment, at least one of the polynucleotides has at least about 60% identity to SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57 or a combination thereof. In one embodiment, at least one of the polynucleotides encodes for a polypeptide with at least about 60% identity to SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, or a combination thereof. In one embodiment, the microorganism synthesizes more thiamine than an unmodified microorganism of the same species. In one embodiment, the vitamins comprise an NAD+ precursor. In one embodiment, the NAD+ precursor is nicotinic acid, nicotinamide, nicotinamide riboside, or a combination thereof. In one embodiment, the metabolic pathways comprise the nicotinate and nicotinamide metabolic pathway. In one embodiment, at least one of the polynucleotides encodes an enzyme, wherein the enzyme has an activity corresponding to EC numbers 1.4.3.16, 2.5.1.72, or 2.4.2.19. In one embodiment, at least one of the polynucleotides is from Clostridium cellulolyticum. In one embodiment, the polynucleotides comprise Ccel_{—}3480, Ccel_{—}3479, Ccel_{—}3478, or a combination thereof. In one embodiment, the polynucleotides comprise Ccel_{—}3480, Ccel_{—}3479, and Ccel_{—}3478. In one embodiment, at least one of the polynucleotides has at least about 60% identity to SEQ ID NO: 20. In one embodiment, at least one of the polynucleotides has at least about 60% identity to SEQ ID NO:59, SEQ ID NO:61, SEQ ID NO:63, or a combination thereof. In one embodiment, at least one of the polynucleotides encodes for a polypeptide with at least about 60% identity to SEQ ID NO:60, SEQ ID NO:62, SEQ ID NO:64, or a combination thereof. In one embodiment, the microorganism produces more NAD+ than an unmodified microorganism of the same species. In one embodiment, the vitamins comprise a vitamin B₆. In one embodiment, the vitamin B₆ comprises pyridoxine, pyridoxine 5′-phosphate, pyridoxal, pyridoxal 5′-phosphate, pyridoxamine, pyridoxamine 5′-phosphate, or a combination thereof. In one embodiment, the metabolic pathways comprise the vitamin B₆ metabolic pathway. In one embodiment, at least one of the polynucleotides is from Clostridium cellulolyticum. In one embodiment, the polynucleotides comprise Ccel_{—}1858, Ccel_{—}1859, or a combination thereof. In one embodiment, the polynucleotides comprise Ccel_{—}1858 and Cecil 859. In one embodiment, at least one of the polynucleotides has at least about 60% identity to SEQ ID NO: 31. In one embodiment, at least one of the polynucleotides has at least about 60% identity to SEQ ID NO:65, SEQ ID NO:67, or a combination thereof. In one embodiment, at least one of the polynucleotides encodes for a polypeptide with at least about 60% identity to SEQ ID NO:66, SEQ ID NO:68, or a combination thereof. In one embodiment, the microorganism synthesizes more pyridoxal 5′-phosphate (PLP) than an unmodified microorganism of the same species. In one embodiment, the vitamins comprise the vitamin B₉. In one embodiment, the vitamin B₉ comprises folic acid, folate, folinic acid, or a combination thereof. In one embodiment, the metabolic pathways comprise the one carbon pool by folate metabolic pathway. In one embodiment, at least one of the polynucleotides is from Clostridium cellulolyticum. In one embodiment, at least one of the polynucleotides encodes for a dihydrofolate reductase. In one embodiment, the polynucleotides comprise Ccel_{—}1310. In one embodiment, at least one of the polynucleotides has at least about 60% identity to SEQ ID NO: 32. In one embodiment, at least one of the polynucleotides has at least about 60% identity to SEQ ID NO:69. In one embodiment, at least one of the polynucleotides encodes for a polypeptide with at least about 60% identity to SEQ ID NO:70. In one embodiment, the microorganism can produce more tetrahydrofolate (THF) than an unmodified microorganism of the same species. In one embodiment, the microorganism produces a greater yield of one or more fermentation end-products than an unmodified microorganism of the same species. In one embodiment, the one or more fermentation end-products comprise one or more alcohols. In one embodiment, the one or more alcohols comprise methanol, ethanol, propanol, butanol, or a combination thereof. In one embodiment, the one or more alcohols comprise ethanol. In one embodiment, the microorganism further produces a lower yield of one or more other fermentation end-products than an unmodified microorganism of the same species. In one embodiment, the one or more other fermentation end-products comprise one or more acids. In one embodiment, the one or more acids comprise lactic acid. In one embodiment, the microorganism can ferment C6 sugars. In one embodiment, the microorganism can ferment C5 and C6 sugars. In one embodiment, the microorganism can hydrolyze cellulose. In one embodiment, the microorganism can hydrolyze hemicellulose. In one embodiment, the microorganism can hydrolyze lignocellulose. In one embodiment, the microorganism can hydrolyze and ferment cellulose. In one embodiment, the microorganism can hydrolyze and ferment hemicellulose. In one embodiment, the microorganism can hydrolyze and ferment lignocellulose. In one embodiment, the microorganism can hydrolyze and ferment cellulosic, hemicellulosic and lignocellulosic material. In one embodiment, the microorganism is a genetically modified Thermoanaerobacter species. In one embodiment, the microorganism is a genetically modified Thermoanaerobacter pseudethanolicus, Thermoanaerobacter mathranii, Thermoanaerobacter italicus, Thermoanaerobacter brockii, T. acetoethylicus, Thermoanaerobacter ethanolicus, Thermoanaerobacter kivui, Thermoanaerobacter siderophilus, Thermoanaerobacter sulfuragignens, Thermoanaerobacter sulfurophilus, Thermoanaerobacter thermocopriae, Thermoanaerobacter thermohydrosulfuricus, Thermoanaerobacter uzonensis, or Thermoanaerobacter wiegelii. In one embodiment, the microorganism is a genetically modified Clostridium species. In one embodiment, the microorganism is a genetically modified Clostridium phytofermentans, Clostridium Q.D or a variant thereof. In one embodiment, the biomass comprises C5 sugars, C6 sugars, or a combination thereof. In one embodiment, the biomass comprises cellulose. In one embodiment, the biomass comprises hemicellulosic or lignocellulosic material. In one embodiment, the biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In one embodiment, the biomass is pretreated to make polysaccharides more available to the microorganism. In one embodiment, the biomass is pretreated by acid, steam explosion, hot water treatment, alkali, catalase, or a detoxifying or chelating agent. In one embodiment, the media comprises one or more vitamins. In one embodiment, the vitamins comprise thiamine, an NAD+ precursor molecule, a vitamin B₆, a vitamin B₉, or a combination thereof. In one embodiment, the media is deficient in one or more vitamins. In one embodiment, the media does not comprise any of at least one of the one or more vitamins. In one embodiment, the media comprises less than the minimal nutritional requirements of at least one of the vitamins for growth of an unmodified microorganism of the same species. In one embodiment, the medium is supplemented with one or more vitamins, wherein at least one of the vitamins is at a concentration below the minimal nutritional requirements for growth of an unmodified microorganism of the same species. In one embodiment, the method further comprises a second microorganism, wherein the second microorganism produces at least one vitamin that is used by the genetically modified microorganism. In one embodiment, the one or more vitamins comprise thiamine, an NAD+ precursor, a vitamin B₆, a vitamin B₉, or a combination thereof. In one embodiment, the NAD+ precursor comprises nicotinic acid, nicotinamide, nicotinamide riboside, or a combination thereof. In one embodiment, the vitamin B₆ comprises pyridoxine, pyridoxine 5′-phosphate, pyridoxal, pyridoxal 5′-phosphate, pyridoxamine, pyridoxamine 5′-phosphate, or a combination thereof. In one embodiment, the vitamin B9 comprises folic acid, folate, folinic acid, or a combination thereof. In one embodiment, the genetically modified microorganism produces a first vitamin. In one embodiment, the first vitamin is thiamine, an NAD+ precursor (e.g., nicotinic acid, nicotinamide, nicotinamide riboside, or a combination thereof) a vitamin B₆ (e.g., pyridoxine, pyridoxine 5′-phosphate, pyridoxal, pyridoxal 5′-phosphate, pyridoxamine, pyridoxamine 5′-phosphate, or a combination thereof), a vitamin B₉ (e.g., folic acid, folate, or folinic acid, or a combination thereof), or a combination thereof. In one embodiment, the medium is supplemented with the first vitamin. In one embodiment, the medium is supplemented with the first vitamin at a level that is below the minimal nutritional requirements for growth of an unmodified microorganism of the same species. In one embodiment, the method further comprises contacting the medium with a second microorganism. In one embodiment, the second microorganism is a yeast, a bacteria, of a non-yeast fungi. In one embodiment, the second microorganism is Eremothecium ashbyii, Ashbya gossypii, Candida flaeri, Candida famata, Candida ammoniagenes, Corynebacterium sp., Serratia marcescens, Fusarium oxysporum, Brevibacterium ammoniagenes, Rhodococcus rhodochrous, Brevibacterium sp., Arthrobacter sp., Candida boidinii, Bacillus sp., Gluconobacter sp., Arthrobacter sp., Saccharomyces sake, Alcaligenes faecalis, Agrobacterium sp., Sporoblomyces salmonicolor, Pseudomonas sp., Propionibacterium shermanii, Pseudomonas denitrificans, Geotrichum candidum, Flavobacterium sp., or Mortierella alpina. In another embodiment, the second microorganism is Saccharomyces cerevisiae, C. thermocellum, C. acetobutylicum, C. cellovorans, or Zymomonas mobilis. In another embodiment, the second microorganism is Thermoanaerobacter pseudethanolicus, Thermoanaerobacter mathranii, Thermoanaerobacter italicus, Thermoanaerobacter brockii, T. acetoethylicus, Thermoanaerobacter ethanolicus, Thermoanaerobacter kivui, Thermoanaerobacter siderophilus, Thermoanaerobacter sulfuragignens, Thermoanaerobacter sulfurophilus, Thermoanaerobacter thermocopriae, Thermoanaerobacter thermohydrosulfuricus, Thermoanaerobacter uzonensis, or Thermoanaerobacter wiegelii. In one embodiment, the second microorganism is not genetically modified. In one embodiment, the second microorganism is genetically modified. In one embodiment, the second microorganism produces the first vitamin, wherein the first vitamin is used by the genetically modified microorganism.

Disclosed herein are systems for the production of one or more fermentation end-products comprising: (a) a media comprising a biomass; (b) a genetically modified microorganism adapted for decreased vitamin dependency, wherein said microorganism comprises a genetic modification that decreases vitamin dependency; and, (c) a fermentor configured to house the media and the microorganism. In one embodiment, the microorganism comprises one or more genetic modifications that enables the microorganism to grow in a medium deficient in one or more vitamins required for growth of an unmodified microorganism of the same species. In one embodiment, the vitamins comprise thiamine, a nicotinamide adenine dinucleotide (NAD+) precursor (e.g., nicotinic acid, nicotinamide, or nicotinamide riboside), a vitamin B₆ (e.g., pyridoxine, pyridoxine 5′-phosphate, pyridoxal, pyridoxal 5′-phosphate, pyridoxamine, or pyridoxamine 5′-phosphate), a vitamin B₉ (e.g., folic acid, folate, or folinic acid), or a combination thereof. In one embodiment, the genetic modifications comprise a heterologous copy of one or more polynucleotides that encode for enzymes in one or more metabolic pathways, wherein the metabolic pathways comprises a thiamine metabolic pathway, a nicotinate and nicotinamide metabolic pathway, a vitamin B₆ metabolic pathway, a one carbon pool by folate pathway, or a combination thereof. In one embodiment, the polynucleotides comprise Ccel_{—}1989, Ccel_{—}1990, Ccel_{—}1991, Ccel_{—}1992, thiC, thiD, thiE, thiF, thiG, thiH, thiL, thiM, Ccel_{—}3480, Ccel_{—}3479, Ccel_{—}3478, Ccel_{—}1858, Ccel_{—}1859, Ccel_{—}1310, or a combination thereof. In one embodiment, at least one of the polynucleotides has at least about 60% identity to SEQ ID NO:20, SEQ ID NO:21, SEQ ID NO:22, SEQ ID NO:23, SEQ ID NO:24, SEQ ID NO:31, SEQ ID NO:32, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or a combination thereof. In one embodiment, at least one of the polynucleotides encodes for a polypeptide with at least about 60% identity to SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, or a combination thereof. In one embodiment, the vitamins comprise thiamine. In one embodiment, the metabolic pathways comprise the thiamine metabolic pathway. In one embodiment, at least one of the polynucleotides are from Clostridium cellulolyticum. In one embodiment, the polynucleotides comprise Ccel_{—}1989, Ccel_{—}1990, Ccel_{—}1991, Ccel_{—}1992, or a combination thereof. In one embodiment, the polynucleotides comprise Ccel_{—}1989, Ccel_{—}1990, Ccel_{—}1991, and Ccel_{—}1992. In one embodiment, at least one of the polynucleotides has at least about 60% identity to SEQ ID NO: 21. In one embodiment, at least one of the polynucleotides has at least about 60% identity to SEQ ID NO:33, SEQ ID NO:35, SEQ ID NO:37, SEQ ID NO:39, or a combination thereof. In one embodiment, at least one of the polynucleotides encodes for a polypeptide with at least about 60% identity to SEQ ID NO:34, SEQ ID NO:36, SEQ ID NO:38, SEQ ID NO:40, or a combination thereof. In one embodiment, at least one of the polynucleotides is from Escherichia coli. In one embodiment, the polynucleotides comprise thiC, thiD, thiE, thiF, thiG, thiH, thiL, thiM, or a combination thereof. In one embodiment, the polynucleotides comprise thiC, thiD, thiE, thiF, thiG, thiH, thiL, and thiM. In one embodiment, at least one of the polynucleotides has at least about 60% identity to SEQ ID NO:22, SEQ ID NO: 23, SEQ ID NO:24, or a combination thereof. In one embodiment, at least one of the polynucleotides has at least about 60% identity to SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57 or a combination thereof. In one embodiment, at least one of the polynucleotides encodes for a polypeptide with at least about 60% identity to SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, or a combination thereof. In one embodiment, the microorganism synthesizes more thiamine than an unmodified microorganism of the same species. In one embodiment, the vitamins comprise an NAD+ precursor. In one embodiment, the NAD+ precursor is nicotinic acid, nicotinamide, nicotinamide riboside, or a combination thereof. In one embodiment, the metabolic pathways comprise the nicotinate and nicotinamide metabolic pathway. In one embodiment, at least one of the polynucleotides encodes an enzyme, wherein the enzyme has an activity corresponding to EC numbers 1.4.3.16, 2.5.1.72, or 2.4.2.19. In one embodiment, at least one of the polynucleotides is from Clostridium cellulolyticum. In one embodiment, the polynucleotides comprise Ccel_{—}3480, Ccel_{—}3479, Ccel_{—}3478, or a combination thereof. In one embodiment, the polynucleotides comprise Ccel_{—}3480, Ccel_{—}3479, and Ccel_{—}3478. In one embodiment, at least one of the polynucleotides has at least about 60% identity to SEQ ID NO: 20. In one embodiment, at least one of the polynucleotides has at least about 60% identity to SEQ ID NO:59, SEQ ID NO:61, SEQ ID NO:63, or a combination thereof. In one embodiment, at least one of the polynucleotides encodes for a polypeptide with at least about 60% identity to SEQ ID NO:60, SEQ ID NO:62, SEQ ID NO:64, or a combination thereof. In one embodiment, the microorganism produces more NAD+ than an unmodified microorganism of the same species. In one embodiment, the vitamins comprise a vitamin B₆. In one embodiment, the vitamin B₆ comprises pyridoxine, pyridoxine 5′-phosphate, pyridoxal, pyridoxal 5′-phosphate, pyridoxamine, pyridoxamine 5′-phosphate, or a combination thereof. In one embodiment, the metabolic pathways comprise the vitamin B₆ metabolic pathway. In one embodiment, at least one of the polynucleotides is from Clostridium cellulolyticum. In one embodiment, the polynucleotides comprise Ccel_{—}1858, Ccel_{—}1859, or a combination thereof. In one embodiment, the polynucleotides comprise Ccel_{—}1858 and Ccel_{—}1859. In one embodiment, at least one of the polynucleotides has at least about 60% identity to SEQ ID NO: 31. In one embodiment, at least one of the polynucleotides has at least about 60% identity to SEQ ID NO:65, SEQ ID NO:67, or a combination thereof. In one embodiment, at least one of the polynucleotides encodes for a polypeptide with at least about 60% identity to SEQ ID NO:66, SEQ ID NO:68, or a combination thereof. In one embodiment, the microorganism synthesizes more pyridoxal 5′-phosphate (PLP) than an unmodified microorganism of the same species. In one embodiment, the vitamins comprise the vitamin B₉. In one embodiment, the vitamin B₉ comprises folic acid, folate, folinic acid, or a combination thereof. In one embodiment, the metabolic pathways comprise the one carbon pool by folate metabolic pathway. In one embodiment, at least one of the polynucleotides is from Clostridium cellulolyticum. In one embodiment, at least one of the polynucleotides encodes for a dihydrofolate reductase. In one embodiment, the polynucleotides comprise Ccel_{—}1310. In one embodiment, at least one of the polynucleotides has at least about 60% identity to SEQ ID NO: 32. In one embodiment, at least one of the polynucleotides has at least about 60% identity to SEQ ID NO:69. In one embodiment, at least one of the polynucleotides encodes for a polypeptide with at least about 60% identity to SEQ ID NO:70. In one embodiment, the microorganism can produce more tetrahydrofolate (THF) than an unmodified microorganism of the same species. In one embodiment, the microorganism produces a greater yield of one or more fermentation end-products than an unmodified microorganism of the same species. In one embodiment, the one or more fermentation end-products comprise one or more alcohols. In one embodiment, the one or more alcohols comprise methanol, ethanol, propanol, butanol, or a combination thereof. In one embodiment, the one or more alcohols comprise ethanol. In one embodiment, the microorganism further produces a lower yield of one or more other fermentation end-products than an unmodified microorganism of the same species. In one embodiment, the one or more other fermentation end-products comprise one or more acids. In one embodiment, the one or more acids comprise lactic acid. In one embodiment, the microorganism can ferment C6 sugars. In one embodiment, the microorganism can ferment C5 and C6 sugars. In one embodiment, the microorganism can hydrolyze cellulose. In one embodiment, the microorganism can hydrolyze hemicellulose. In one embodiment, the microorganism can hydrolyze lignocellulose. In one embodiment, the microorganism can hydrolyze and ferment cellulose. In one embodiment, the microorganism can hydrolyze and ferment hemicellulose. In one embodiment, the microorganism can hydrolyze and ferment lignocellulose. In one embodiment, the microorganism can hydrolyze and ferment cellulosic, hemicellulosic and lignocellulosic material. In one embodiment, the microorganism is a genetically modified Thermoanaerobacter species. In one embodiment, the microorganism is a genetically modified Thermoanaerobacter pseudethanolicus, Thermoanaerobacter mathranii, Thermoanaerobacter italicus, Thermoanaerobacter brockii, T. acetoethylicus, Thermoanaerobacter ethanolicus, Thermoanaerobacter kivui, Thermoanaerobacter siderophilus, Thermoanaerobacter sulfuragignens, Thermoanaerobacter sulfurophilus, Thermoanaerobacter thermocopriae, Thermoanaerobacter thermohydrosulfuricus, Thermoanaerobacter uzonensis, or Thermoanaerobacter wiegelii. In one embodiment, the microorganism is a genetically modified Clostridium species. In one embodiment, the microorganism is a genetically modified Clostridium phytofermentans, Clostridium Q.D or a variant thereof. In one embodiment, the biomass comprises C5 sugars, C6 sugars, or a combination thereof. In one embodiment, the biomass comprises cellulose. In one embodiment, the biomass comprises hemicellulosic or lignocellulosic material. In one embodiment, the biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In one embodiment, the biomass is pretreated to make polysaccharides more available to the microorganism. In one embodiment, the biomass is pretreated by acid, steam explosion, hot water treatment, alkali, catalase, or a detoxifying or chelating agent. In one embodiment, the media comprises one or more vitamins. In one embodiment, the vitamins comprise thiamine, an NAD+ precursor molecule, a vitamin B₆, a vitamin B₉, or a combination thereof. In one embodiment, the media is deficient in one or more vitamins. In one embodiment, the media does not comprise any of at least one of the one or more vitamins. In one embodiment, the media comprises less than the minimal nutritional requirements of at least one of the vitamins for growth of an unmodified microorganism of the same species. In one embodiment, the medium is supplemented with one or more vitamins, wherein at least one of the vitamins is at a concentration below the minimal nutritional requirements for growth of an unmodified microorganism of the same species. In one embodiment, the method further comprises a second microorganism, wherein the second microorganism produces at least one vitamin that is used by the genetically modified microorganism. In one embodiment, the one or more vitamins comprise thiamine, an NAD+ precursor, a vitamin B₆, a vitamin B₉, or a combination thereof. In one embodiment, the NAD+ precursor comprises nicotinic acid, nicotinamide, nicotinamide riboside, or a combination thereof. In one embodiment, the vitamin B₆ comprises pyridoxine, pyridoxine 5′-phosphate, pyridoxal, pyridoxal 5′-phosphate, pyridoxamine, pyridoxamine 5′-phosphate, or a combination thereof. In one embodiment, the vitamin B9 comprises folic acid, folate, folinic acid, or a combination thereof. In one embodiment, the genetically modified microorganism produces a first vitamin. In one embodiment, the first vitamin is thiamine, an NAD+ precursor (e.g., nicotinic acid, nicotinamide, nicotinamide riboside, or a combination thereof) a vitamin B₆ (e.g., pyridoxine, pyridoxine 5′-phosphate, pyridoxal, pyridoxal 5′-phosphate, pyridoxamine, pyridoxamine 5′-phosphate, or a combination thereof), a vitamin B₉ (e.g., folic acid, folate, or folinic acid, or a combination thereof), or a combination thereof. In one embodiment, the medium is supplemented with the first vitamin. In one embodiment, the medium is supplemented with the first vitamin at a level that is below the minimal nutritional requirements for growth of an unmodified microorganism of the same species. In one embodiment, the method further comprises contacting the medium with a second microorganism. In one embodiment, the second microorganism is a yeast, a bacteria, of a non-yeast fungi. In one embodiment, the second microorganism is Eremothecium ashbyii, Ashbya gossypii, Candida flaeri, Candida famata, Candida ammoniagenes, Corynebacterium sp., Serratia marcescens, Fusarium oxysporum, Brevibacterium ammoniagenes, Rhodococcus rhodochrous, Brevibacterium sp., Arthrobacter sp., Candida boidinii, Bacillus sp., Gluconobacter sp., Arthrobacter sp., Saccharomyces sake, Alcaligenes faecalis, Agrobacterium sp., Sporoblomyces salmonicolor, Pseudomonas sp., Propionibacterium shermanii, Pseudomonas denitrificans, Geotrichum candidum, Flavobacterium sp., or Mortierella alpina. In another embodiment, the second microorganism is Saccharomyces cerevisiae, C. thermocellum, C. acetobutylicum, C. cellovorans, or Zymomonas mobilis. In another embodiment, the second microorganism is Thermoanaerobacter pseudethanolicus, Thermoanaerobacter mathranii, Thermoanaerobacter italicus, Thermoanaerobacter brockii, T. acetoethylicus, Thermoanaerobacter ethanolicus, Thermoanaerobacter kivui, Thermoanaerobacter siderophilus, Thermoanaerobacter sulfuragignens, Thermoanaerobacter sulfurophilus, Thermoanaerobacter thermocopriae, Thermoanaerobacter thermohydrosulfuricus, Thermoanaerobacter uzonensis, or Thermoanaerobacter wiegelii. In one embodiment, the second microorganism is not genetically modified. In one embodiment, the second microorganism is genetically modified. In one embodiment, the second microorganism produces the first vitamin, wherein the first vitamin is used by the genetically modified microorganism.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features disclosed herein are set forth with particularity in the appended claims. A better understanding of the features and advantages will be obtained by reference to the following detailed description that sets forth illustrative embodiments, in which the principles of the disclosure are utilized, and the accompanying drawings of which:

FIG. 1 illustrates ethanol and acid production with thiamine and without thiamine.

FIG. 2 illustrates ethanol and lactic acid production with and without thiamine.

FIG. 3 illustrates the structure of thiamine pyrophosphate (TPP).

FIG. 4 illustrates the anaerobic fermentation pathway for synthesis of several products.

FIG. 5 illustrates the use of TPP as a coenzyme of pyruvate ferredoxin oxidoreductase in the synthesis of Acetyl-CoA.

FIG. 6 illustrates pathways for thiamine biosynthesis with points where C. phytofermentans can be lacking enzymes.

FIG. 7 illustrates pathways for thiamine biosynthesis in C. phytofermentans completed with heterologous C. cellulolyticum enzyme expression.

FIG. 8 illustrates the structure of an exogenous operon for C. phytofermentans.

FIG. 9 discloses primer polynucleotide sequences for cloning of C. cellulolyticum Thiamine biosynthesis operon (SEQ ID NOs:2&3).

FIG. 10 illustrates pathways for thiamine biosynthesis in C. phytofermentans completed with heterologous E. coli enzyme expression.

FIG. 11 illustrates three E. coli operons containing thiamine biosynthesis pathway genes.

FIG. 12 illustrates a cloning strategy and discloses primer polynucleotide sequences for cloning of E. coli genes (SEQ ID NOs: 4-9) and construction of exogenous operon.

FIG. 13 illustrates the plasmid pUniExp-thiamine E. coli.

FIG. 14 illustrates ethanol production with and without additional nicotinic acid.

FIG. 15 illustrates ethanol production with increasing amounts of additional nicotinic acid.

FIG. 16 illustrates pathways for nicotinate and nicotinamide metabolism; highlighted are genes involved in Nicotinate D-ribonucleotide synthesis that are present or absent in C. phytofermentans.

FIG. 17 illustrates a portion of the C. cellulolyticum NAD biosynthesis operon involved in Nicotinate D-ribonucleotide synthesis: Ccel_{—}3478: nicotinate-nucleotide pyrophosphorylase, Ccel_{—}3479: L-aspartate oxidase, Ccel_{—}3480: quinolinate synthetase complex, subunit alpha.

FIG. 18 discloses primer polynucleotide sequences (SEQ ID NOs:10-19) used in cloning plasmid pMTL-NAD; underlined sequence optimizes ribosome binding site of Ccel_{—}3480; double underlined sequence is restriction enzyme recognition site.

FIG. 19 discloses the polynucleotide sequence of the Ccel_{—}3478-3480 operon (SEQ ID NO: 20); start and stop codons are double underlined; putative ribosome binding sites are underlined; the predicted terminator is boxed; coding regions are capitalized (in order: Ccel_{—}3480, Ccel_{—}3479, and Ccel_{—}3478.

FIG. 20 illustrates the plasmid pMTL-NAD.

FIG. 21 illustrates the plasmid pMTL82351UniExp.

FIG. 22 depicts the plasmid pQInt.

FIG. 23 (FIGS. 23A and 23B) depicts the plasmids pQInt1 and pQInt2.

FIG. 24 depicts a method for producing fermentation end-products from biomass by first treating biomass with an acid at elevated temperature and pressure in a hydrolysis unit.

FIG. 25 depicts a method for producing fermentation end-products from biomass by charging biomass to a fermentation vessel.

FIG. 26 discloses pretreatments that produce hexose or pentose saccharides or oligomers that are then unprocessed or processed further and either fermented separately or together.

FIG. 27A-C discloses the polynucleotide sequence of plasmid pMTL82351-P3558-3202 (SEQ ID NO: 1).

FIG. 28 discloses the polynucleotide sequence of the Clostridium cellulolyticum Thiamine operon containing genes Ccel_{—}1992, Cel_{—}1991, Ccel_{—}1990, and Ccel_{—}1989 (SEQ ID NO: 21).

FIG. 29 A-C discloses the polynucleotide sequence of three Escherichia coli Thiamine operons; operon I contains genes thiC, thiE, thiF, this, thiG, and thiH (SEQ ID NO:22); operon II contains genes thiD and thiM (SEQ ID NO:23); and operon III contains the thiL gene (SEQ ID NO:24).

FIG. 30 discloses the polynucleotide sequence of a Clostridium cellulolyticum Pyridoxal-5-Phosphate operon containing genes Ccel_{—}1858 and Ccel_{—}1859 (SEQ ID NO:31).

FIG. 31 discloses the polynucleotide sequence of Clostridium cellulolyticum Ccel_{—}1310 (SEQ ID NO: 32) which encodes a Dihydrofolate reductase.

FIG. 32 illustrates the plasmid pMTL-Pyridoxal.

FIG. 33 illustrates the plasmid pMTL-DHF.

FIG. 34 discloses the polynucleotide sequence of the chromosomal region between genes Cphy_{—}1606 and Cphy_{—}1607 in Clostridium phytofermentans (SEQ ID NO: 33).

FIG. 35 illustrates plasmid pMTL82351uniExp-int1606-1607.

FIG. 36 illustrates plasmid pMTL-NAD-int1606-1607.

FIG. 37 illustrates plasmid pMTL-Pyridoxal-int1606-1607.

FIG. 38 illustrates plasmid pMTL-DHF-int1606-1607.

FIG. 39 illustrates ethanol production by C. phytofermentans strains QX45 and Q.8 in minimal media with or without supplementation with pyridoxine; x-axis is time in hours; y-axis is ethanol yield in g/L.

FIG. 40 illustrates ethanol production by C. phytofermentans strains QX45 and Q.8 in complex media with or without supplementation with pyridoxine; x-axis is time in hours; y-axis is ethanol yield in g/L.

FIG. 41 illustrates a portion of the vitamin B6 (pyridoxal-5′-phosphate) metabolic pathways; solid round box and arrow highlight existing pathways for the conversion of pyridoxal to pyridoxal-5′-phosphate in Clostridium phytofermentans; dashed round box and arrow indicate missing pathways for synthesis of pyridoxal-5′-phosphate from pentose phosphate pathway and glycolysis products in Clostridium phytofermentans.

FIG. 42 illustrates a portion of the vitamin B6 (pyridoxal-5′-phosphate) metabolic pathways; solid round box and arrow highlight existing pathways for synthesis of pyridoxal-5′-phosphate from pentose phosphate pathway and glycolysis products in Clostridium cellulolyticum.

FIG. 43 illustrates a one carbon pool by folate metabolic pathway; dashed circle highlights dihydrofolate reductase, which is missing in Clostridium phytofermentans.

### DETAILED DESCRIPTION

The present disclosure can be understood more readily by reference to the following detailed description, the Examples included therein and to the Figures and their previous and following description. The following description and examples illustrate some exemplary embodiments of the disclosure in detail. Those of skill in the art will recognize that there are numerous variations and modifications of this disclosure that are encompassed by its scope. Accordingly, the description of a certain exemplary embodiment should not be deemed to limit the scope of the present disclosure. Before the present compounds, compositions, articles, devices, and/or methods are disclosed and described, it is to be understood that this disclosure is not limited to specific synthetic methods, specific purified proteins, or to particular nucleic acids, as such can, of course, vary. It is also to be understood that the terminology used herein is for the purpose of describing particular embodiments only and is not intended to be limiting.

Genetically Modified Metabolic Pathways

Vitamins are used to supply specific co-factors that facilitate enzymatic reactions in most bacterial organisms. In some cases, the vitamin itself is a cofactor. In some cases, the vitamin is used as a substrate in a metabolic pathway to synthesize a vitamin metabolite, wherein the vitamin metabolite is a co-factor. In some cases, a vitamin metabolite can by synthesized by a microorganism without requiring the vitamin. If the microorganism environment is deficient in a vitamin, and the microorganism is unable to synthesize sufficient quantities of the vitamin or vitamin metabolite, the microorganism can fail to grow. In other words, a microorganism's inability to synthesize a vitamin or vitamin metabolite without an external source of the vitamins can limit the range of environments in which the microorganism can grow.

As used herein, the term “vitamin” can encompass a vitamin, a vitamin precursor, a vitamin substitute, or a vitamin metabolite. Exemplary vitamins can include vitamin A (e.g., retinol), vitamin Bₚ (e.g., choline), vitamin B₁ (e.g., thiamin), vitamin B₂ (e.g., riboflavin) vitamin B₃ (e.g., niacin, nicotinic acid, nicotinamide, nicotinamide riboside), vitamin B₅ (e.g., pantothenic acid), vitamin B₆ (e.g., pyridoxine, pyridoxamine, pyridoxal), vitamin B₇ (e.g., biotin), vitamin B₉ (e.g., folic acid, folate, folinic acid), vitamin B₁₂ (e.g., cobalamin), vitamin C (e.g., ascorbic acid), vitamin D (e.g., ergocalciferol, cholecalciferol), vitamin E (e.g., tocopherol), vitamin K (e.g., naphthoquinoids), or a combination thereof.

Disclosed herein are genetically modified microorganisms adapted for decreased vitamin dependency. In one embodiment, the genetically modified microorganism is capable of growth in a media that is deficient in one or more vitamins required for growth of an unmodified microorganism of the same species. As used herein, the term “deficient” can mean inadequate in amount; for example, a media that is deficient in a vitamin does not contain a sufficient amount of the vitamin. In one embodiment, the vitamins comprise vitamin B₁ (e.g., thiamin). In another embodiment, the vitamins comprise vitamin B₃ (e.g., niacin, nicotinic acid, nicotinamide, nicotinamide riboside). In another embodiment, the vitamins comprise vitamin B₆ (e.g., pyridoxine, pyridoxamine, pyridoxal). In another embodiment, the vitamins comprise vitamin B₉ (e.g., folic acid, folate, folinic acid). In one embodiment the genetically modified microorganism produces one or more fermentation end-products from a biomass. In another embodiment the genetically modified microorganism can hydrolyze and/or ferment hemicelluloses or lignocelluloses. In another embodiment the genetically modified microorganism can ferment C5 and/or C6 sugars. In another embodiment the genetically modified microorganism can hydrolyze and/or ferment oligosaccharides. In another embodiment the genetically modified microorganism is a Clostridium strain. In another embodiment the genetically modified microorganism is a genetically modified Clostridium phytofermentans or Clostridium Q.D. strain. In another embodiment, the genetically modified microorganism is a genetically modified Thermoanaerobacter species. In another embodiment, the microorganism is a genetically modified Thermoanaerobacter pseudethanolicus, Thermoanaerobacter mathranii, Thermoanaerobacter italicus, Thermoanaerobacter brockii, T. acetoethylicus, Thermoanaerobacter ethanolicus, Thermoanaerobacter kivui, Thermoanaerobacter siderophilus, Thermoanaerobacter sulfuragignens, Thermoanaerobacter sulfurophilus, Thermoanaerobacter thermocopriae, Thermoanaerobacter thermohydrosulfuricus, Thermoanaerobacter uzonensis, or Thermoanaerobacter wiegelii.

Also disclosed herein are methods of producing one or more fermentation end-products comprising: contacting a genetically modified microorganism adapted for decreased vitamin dependency with a biomass in a medium. In one embodiment, the genetically modified microorganism is capable of growth in a medium that is deficient in one or more vitamins required for growth of an unmodified microorganism of the same species. In one embodiment, the vitamins comprise vitamin B₁ (e.g., thiamin). In another embodiment, the vitamins comprise vitamin B₃ (e.g., niacin, nicotinic acid, nicotinamide, nicotinamide riboside). In another embodiment, the vitamins comprise vitamin B₆ (e.g., pyridoxine, pyridoxamine, pyridoxal). In another embodiment, the vitamins comprise vitamin B₉ (e.g., folic acid, folate, folinic acid). In one embodiment the genetically modified microorganism produces one or more fermentation end-products from a biomass. In another embodiment the genetically modified microorganism can hydrolyze and/or ferment hemicelluloses or lignocelluloses. In another embodiment the genetically modified microorganism can ferment C5 and/or C6 sugars. In another embodiment the genetically modified microorganism can hydrolyze and/or ferment oligosaccharides. In another embodiment the genetically modified microorganism is a Clostridium strain. In another embodiment the genetically modified microorganism is a genetically modified Clostridium Phytofermentans or Clostridium Q.D. strain. In another embodiment, the genetically modified microorganism is a genetically modified Thermoanaerobacter species. In another embodiment, the microorganism is a genetically modified Thermoanaerobacter pseudethanolicus, Thermoanaerobacter mathranii, Thermoanaerobacter italicus, Thermoanaerobacter brockii, T. acetoethylicus, Thermoanaerobacter ethanolicus, Thermoanaerobacter kivui, Thermoanaerobacter siderophilus, Thermoanaerobacter sulfuragignens, Thermoanaerobacter sulfurophilus, Thermoanaerobacter thermocopriae, Thermoanaerobacter thermohydrosulfuricus, Thermoanaerobacter uzonensis, or Thermoanaerobacter wiegelii. In one embodiment, the biomass comprises hemicelluloses or lignocelluloses. In another embodiment, the biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, hulls, bagasse, poplar, or algae. In one embodiment, the fermentation end-products comprise an alcohol. In another embodiment, the alcohol is methanol, ethanol, propanol, butanol, or a combination thereof. In another embodiment, the alcohol is ethanol.

Also disclosed herein are methods of genetically modifying a microorganism such that the resulting genetically modified microorganism is capable of growth in a medium that lacks a sufficient quantity of one or more vitamins sufficient for growth of an unmodified microorganism of the same species. In one embodiment, the vitamins comprise vitamin B₁ (e.g., thiamin). In another embodiment, the vitamins comprise vitamin B₃ (e.g., niacin, nicotinic acid, nicotinamide, nicotinamide riboside). In another embodiment, the vitamins comprise vitamin B₆ (e.g., pyridoxine, pyridoxamine, pyridoxal). In another embodiment, the vitamins comprise vitamin B₉ (e.g., folic acid, folate, folinic acid). In one embodiment the genetically modified microorganism produces one or more fermentation end-products from a biomass. In another embodiment the genetically modified microorganism can hydrolyze and/or ferment hemicelluloses or lignocelluloses. In another embodiment the genetically modified microorganism can ferment C5 and/or C6 sugars. In another embodiment the genetically modified microorganism can hydrolyze and/or ferment oligosaccharides. In another embodiment the genetically modified microorganism is a Clostridium strain. In another embodiment the genetically modified microorganism is a genetically modified Clostridium Phytofermentans or Clostridium Q.D. strain. In another embodiment, the genetically modified microorganism is a genetically modified Thermoanaerobacter species. In another embodiment, the microorganism is a genetically modified Thermoanaerobacter pseudethanolicus, Thermoanaerobacter mathranii, Thermoanaerobacter italicus, Thermoanaerobacter brockii, T. acetoethylicus, Thermoanaerobacter ethanolicus, Thermoanaerobacter kivui, Thermoanaerobacter siderophilus, Thermoanaerobacter sulfuragignens, Thermoanaerobacter sulfurophilus, Thermoanaerobacter thermocopriae, Thermoanaerobacter thermohydrosulfuricus, Thermoanaerobacter uzonensis, or Thermoanaerobacter wiegelii.

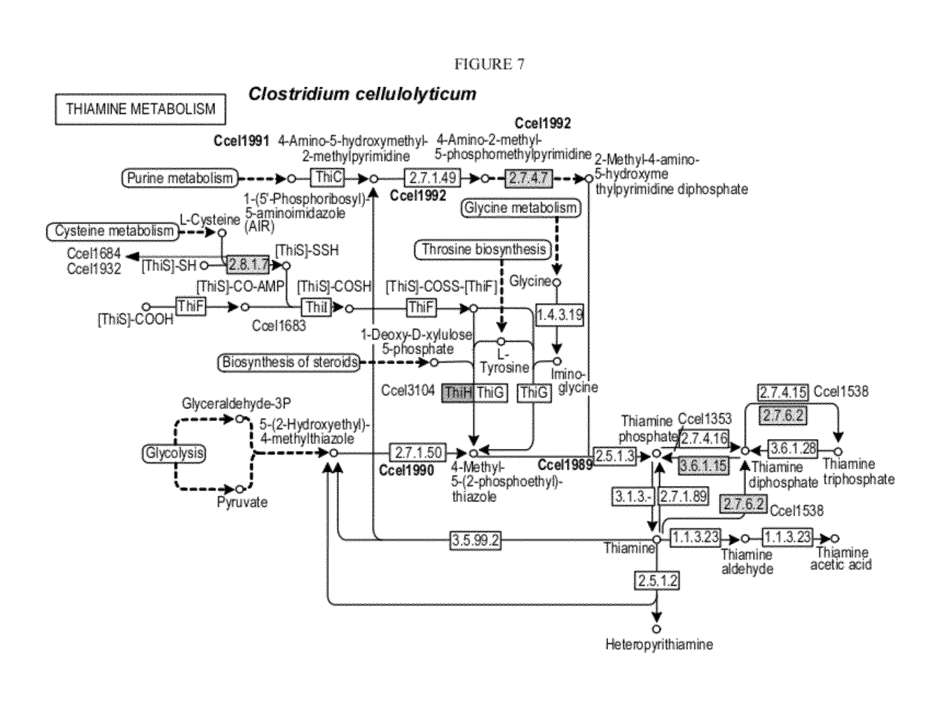

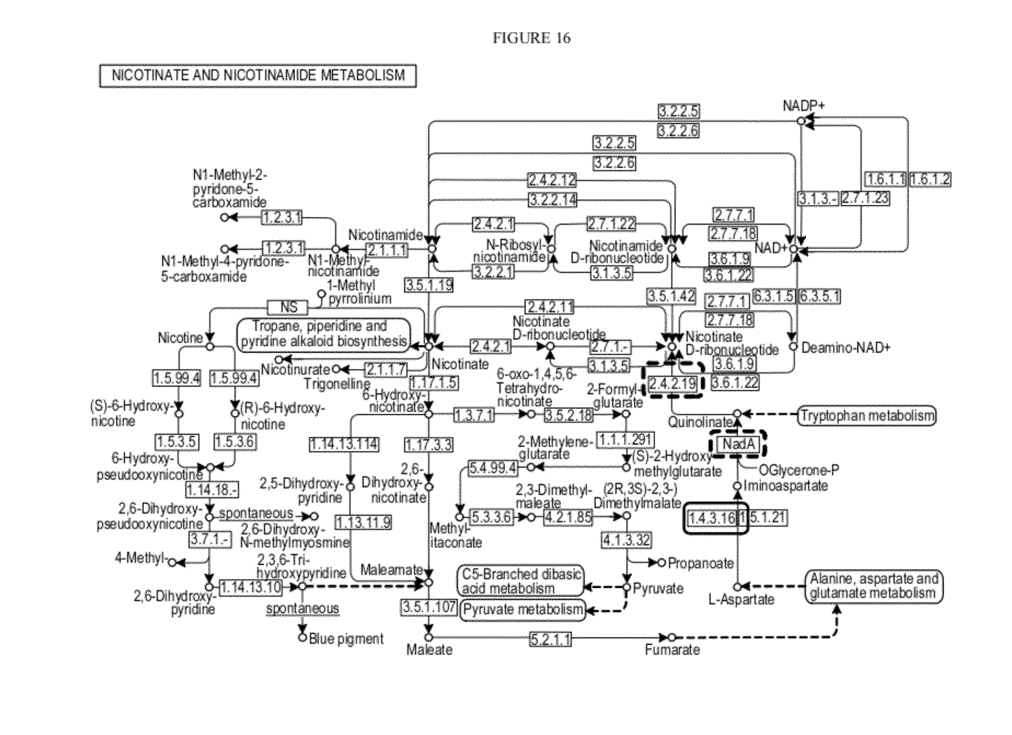

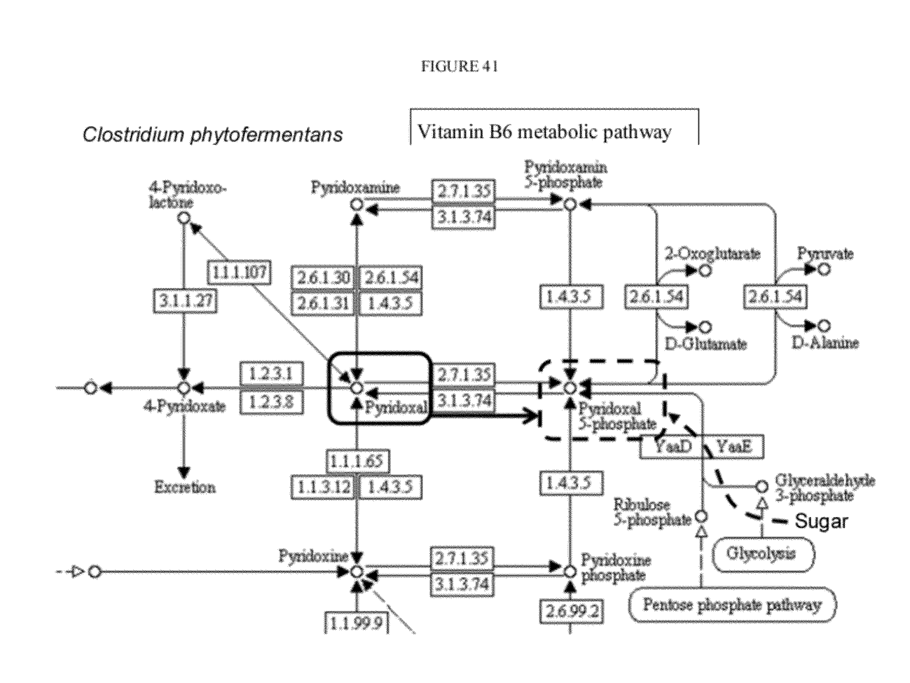

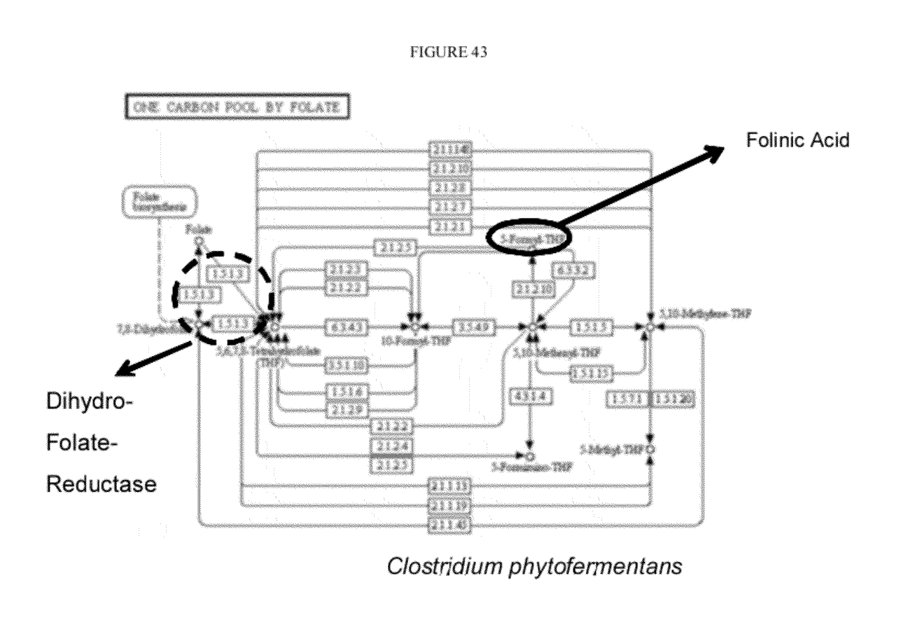

In one embodiment, a genetically modified microorganism capable of growth in a medium that is deficient in one or more vitamins required for growth of an unmodified microorganism of the same species can comprise one or more genetic modifications in one or more metabolic pathways. In one embodiment, the metabolic pathways are metabolic pathways that synthesize the vitamins. In another embodiment, the metabolic pathways are metabolic pathways that synthesize vitamin metabolites without requiring the vitamins. The metabolic pathways can comprise a thiamine metabolic pathway (e.g., FIG. 7), a nicotinate and nicotinamide metabolic pathway (e.g., FIG. 16), a vitamin B₆ metabolic pathway (e.g., FIG. 41), a one carbon pool by folate metabolic pathway (e.g., FIG. 43), or a combination thereof. In one embodiment, the genetic modification restores function to the metabolic pathway. In another embodiment, the unmodified microorganism lacks one or more genes that express enzymes in the metabolic pathway. In another embodiment, the unmodified microorganism contains one or more genes in the metabolic pathway that encode for enzymes with sub-optimal activity. In another embodiment, the unmodified microorganism does not produce the vitamins. In another embodiment the unmodified microorganism produces the vitamins at a level that is not sufficient to support growth of the unmodified microorganism without an external source of the vitamins. The genetic modification can be the result of a directed evolution process. In one embodiment, the directed evolution process comprises treatment with one or more mutagenic agents. In another embodiment, the directed evolution process comprises site-directed mutagenesis. In another embodiment, the directed evolution process comprises selection for microorganism growth in media lacking the vitamins. The genetic modification can be a heterologous copy of one or more polynucleotides that encode for enzymes in the metabolic pathways; for example, metabolic pathways that produces the vitamins or vitamin metabolites. In one embodiment, the polynucleotides have at least about 50%, 55%, 60%, 65%, 70%, 75%, 80%, 85%, 90%, 95%, 96%, 97%, 98%, 99%, or 100% identity to at least one of SEQ ID NOs: 20, 21, 22, 23, 24, 31, 32, 33, 35, 37, 39, 41, 43, 45, 47, 49, 51, 53, 55, 57, 59, 61, 63, 65, 67, or 69. In another embodiment, the polynucleotides encode a polypeptide that has at least about 50%, 55%, 60%, 65%, 70%, 75%, 80%, 85%, 90%, 95%, 96%, 97%, 98%, 99%, or 100% identity to at least one of SEQ ID NOs: 34, 36, 38, 40, 42, 44, 46, 48, 50, 52, 54, 56, 58, 60, 62, 64, 66, 68, or 70. In one embodiment, the polynucleotides are located on a vector, such as a self replicating vector. In another embodiment, the polynucleotides are integrated into the genome of the microorganism.

In another embodiment, a genetically modified microorganism capable of growth in a medium that is deficient in one or more vitamins required for growth of an unmodified microorganism of the same species can produce greater yields of one or more fermentation end-products than the unmodified microorganism. In one embodiment, the greater yields are between about 1 and 300% more of the fermentation end-products; for example, the genetically modified microorganism can produce about 1-300%, 1-250%, 1-200%, 1-150%, 1-100%, 1-90%, 1-80%, 1-70%, 1-60%, 1-50%, 1-40%, 1-30%, 1-20%, 1-10%, 10-300%, 10-250%, 10-200%, 10-150%, 10-100%, 10-90%, 10-80%, 10-70%, 10-60%, 10-50%, 10-40%, 10-30%, 10-20%, 20-300%, 20-250%, 20-200%, 20-150%, 20-100%, 20-90%, 20-80%, 20-70%, 20-60%, 20-50%, 20-40%, 20-30%, 30-300%, 30-250%, 30-200%, 30-150%, 30-100%, 30-90%, 30-80%, 30-70%, 30-60%, 30-50%, 30-40%, 40-300%, 40-250%, 40-200%, 40-150%, 40-100%, 40-90%, 40-80%, 40-70%, 40-60%, 40-50%, 50-300%, 50-250%, 50-200%, 50-150%, 50-100%, 50-90%, 50-80%, 50-70%, 50-60%, 60-300%, 60-250%, 60-200%, 60-150%, 60-100%, 60-90%, 60-80%, 60-70%, 70-300%, 70-250%, 70-200%, 70-150%, 70-100%, 70-90%, 70-80%, 80-300%, 80-250%, 80-200%, 80-150%, 80-100%, 80-90%, 90-300%, 90-250%, 90-200%, 90-150%, 90-100%, 100-300%, 100-250%, 100-200%, 100-150%, 150-300%, 150-250%, 150-200%, 200-300%, 200-250%, 250-300%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 220%, 240%, 260%, 280%, or 300%, or more, of the fermentation end-products in comparison to the unmodified microorganism of the same species. In one embodiment, the greater yields are produced in a medium that is supplemented with the vitamins. In another embodiment, the greater yields are produced in a medium that is not supplemented with the vitamins.

In another embodiment, a genetically modified microorganism capable of growth in a medium that is deficient in one or more vitamins required for growth of an unmodified microorganism of the same species can produce one or more fermentation end-products at greater rates than the unmodified microorganism of the same species. In one embodiment, the increased rates are between about 1 and 300% faster; for example, the genetically modified microorganism produces the fermentation end-products at rates that are about 1-300%, 1-250%, 1-200%, 1-150%, 1-100%, 1-90%, 1-80%, 1-70%, 1-60%, 1-50%, 1-40%, 1-30%, 1-20%, 1-10%, 10-300%, 10-250%, 10-200%, 10-150%, 10-100%, 10-90%, 10-80%, 10-70%, 10-60%, 10-50%, 10-40%, 10-30%, 10-20%, 20-300%, 20-250%, 20-200%, 20-150%, 20-100%, 20-90%, 20-80%, 20-70%, 20-60%, 20-50%, 20-40%, 20-30%, 30-300%, 30-250%, 30-200%, 30-150%, 30-100%, 30-90%, 30-80%, 30-70%, 30-60%, 30-50%, 30-40%, 40-300%, 40-250%, 40-200%, 40-150%, 40-100%, 40-90%, 40-80%, 40-70%, 40-60%, 40-50%, 50-300%, 50-250%, 50-200%, 50-150%, 50-100%, 50-90%, 50-80%, 50-70%, 50-60%, 60-300%, 60-250%, 60-200%, 60-150%, 60-100%, 60-90%, 60-80%, 60-70%, 70-300%, 70-250%, 70-200%, 70-150%, 70-100%, 70-90%, 70-80%, 80-300%, 80-250%, 80-200%, 80-150%, 80-100%, 80-90%, 90-300%, 90-250%, 90-200%, 90-150%, 90-100%, 100-300%, 100-250%, 100-200%, 100-150%, 150-300%, 150-250%, 150-200%, 200-300%, 200-250%, 250-300%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 220%, 240%, 260%, 280%, or 300% faster than the unmodified microorganism of the same species. In one embodiment, the faster rates of production are obtained in a medium that is supplemented with the vitamins. In another embodiment, the faster rates of production are obtained in a medium that is not supplemented with the vitamins.

In another embodiment, a genetically modified microorganism capable of growth in a medium that is deficient in one or more vitamins required for growth of an unmodified microorganism of the same species can produce lower yields of one or more fermentation end-products than the unmodified microorganism. For example, a genetically modified microorganism can produce yields of at least one fermentation end-product that is about 1-100% lower than the amount produced by the unmodified microorganism, such as about 1-100%, 1-90%, 1-80%, 1-70%, 1-60%, 1-50%, 1-40%, 1-30%, 1-20%, 1-10%, 10-100%, 10-90%, 10-80%, 10-70%, 10-60%, 10-50%, 10-40%, 10-30%, 10-20%, 20-100%, 20-90%, 20-80%, 20-70%, 20-60%, 20-50%, 20-40%, 20-30%, 30-100%, 30-90%, 30-80%, 30-70%, 30-60%, 30-50%, 30-40%, 40-100%, 40-90%, 40-80%, 40-70%, 40-60%, 40-50%, 50-100%, 50-90%, 50-80%, 50-70%, 50-60%, 60-100%, 60-90%, 60-80%, 60-70%, 70-100%, 70-90%, 70-80%, 80-100%, 80-90%, 90-100%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% lower. In one embodiment, the fermentation end-products comprise acids. In one embodiment, the acids comprise lactic acid.

In another embodiment, a genetically modified microorganism capable of growth in a medium that is deficient in one or more vitamins required for growth of an unmodified microorganism of the same species can comprise genetic modifications in more than one metabolic pathways; for example, 2, 3, 4, 5, 6, 7, 8, 9, 10, or more metabolic pathways. In one embodiment, the genetically modified microorganism comprises genetic modifications in a thiamine metabolic pathway and a nicotinamide and nicotinate metabolic pathway. In one embodiment, the genetically modified microorganism comprises genetic modifications in a thiamine metabolic pathway and a vitamin B₆ metabolic pathway. In one embodiment, the genetically modified microorganism comprises genetic modifications in a thiamine metabolic pathway and a one carbon pool by folate metabolic pathway. In one embodiment, the genetically modified microorganism comprises genetic modifications in a nicotinamide and nicotinate metabolic pathway and a vitamin B₆ metabolic pathway. In one embodiment, the genetically modified microorganism comprises genetic modifications in a nicotinamide and nicotinate metabolic pathway and a one carbon pool by folate metabolic pathway. In one embodiment, the genetically modified microorganism comprises genetic modifications in a vitamin B₆ metabolic pathway and a one carbon pool by folate metabolic pathway. In one embodiment, the genetically modified microorganism comprises genetic modifications in a thiamine metabolic pathway, a nicotinamide and nicotinate metabolic pathway, and a vitamin B₆ metabolic pathway. In one embodiment, the genetically modified microorganism comprises genetic modifications in a thiamine metabolic pathway, a nicotinamide and nicotinate metabolic pathway, and a one carbon pool by folate metabolic pathway. In one embodiment, the genetically modified microorganism comprises genetic modifications in a thiamine metabolic pathway, a vitamin B₆ metabolic pathway, and a one carbon pool by folate metabolic pathway. In one embodiment, the genetically modified microorganism comprises genetic modifications in a nicotinamide and nicotinate metabolic pathway, a vitamin B₆ metabolic pathway, and a one carbon pool by folate metabolic pathway. In one embodiment, the genetically modified microorganism comprises genetic modifications in a thiamine metabolic pathway, a nicotinamide and nicotinate metabolic pathway, a vitamin B₆ metabolic pathway, and a one carbon pool by folate metabolic pathway.

In one embodiment, a synergistic effect in production of one or more fermentation end-products can be obtained when utilizing a genetically modified microorganism that comprises genetic modifications in two or more metabolic pathways. As used herein, the terms “synergy” or “synergistic effect” means that two or more components function together to produce a result not independently obtainable or a result that is greater than would be expected based upon the addition of independent results. In one embodiment, the synergistic effect is obtained in a yield of one or more fermentation end-products. For example, if a microorganism comprising a genetic modification in a first metabolic pathway produces A % more of the fermentation end-products than an unmodified microorganism and a microorganism comprising a genetic modification in a second metabolic pathway produces B % more fermentation end-products than the unmodified microorganism, then a genetically modified microorganism comprising genetic modifications in both pathways produces C % more fermentation end-products, wherein C is between about 1% and 300% greater than A, B, or A+B; for example, about 1-300%, 1-250%, 1-200%, 1-150%, 1-100%, 1-90%, 1-80%, 1-70%, 1-60%, 1-50%, 1-40%, 1-30%, 1-20%, 1-10%, 10-300%, 10-250%, 10-200%, 10-150%, 10-100%, 10-90%, 10-80%, 10-70%, 10-60%, 10-50%, 10-40%, 10-30%, 10-20%, 20-300%, 20-250%, 20-200%, 20-150%, 20-100%, 20-90%, 20-80%, 20-70%, 20-60%, 20-50%, 20-40%, 20-30%, 30-300%, 30-250%, 30-200%, 30-150%, 30-100%, 30-90%, 30-80%, 30-70%, 30-60%, 30-50%, 30-40%, 40-300%, 40-250%, 40-200%, 40-150%, 40-100%, 40-90%, 40-80%, 40-70%, 40-60%, 40-50%, 50-300%, 50-250%, 50-200%, 50-150%, 50-100%, 50-90%, 50-80%, 50-70%, 50-60%, 60-300%, 60-250%, 60-200%, 60-150%, 60-100%, 60-90%, 60-80%, 60-70%, 70-300%, 70-250%, 70-200%, 70-150%, 70-100%, 70-90%, 70-80%, 80-300%, 80-250%, 80-200%, 80-150%, 80-100%, 80-90%, 90-300%, 90-250%, 90-200%, 90-150%, 90-100%, 100-300%, 100-250%, 100-200%, 100-150%, 150-300%, 150-250%, 150-200%, 200-300%, 200-250%, 250-300%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, or 300% greater. In one embodiment, the synergistic effect on yield is obtained in a medium that is supplemented with the vitamins. In another embodiment, the synergistic effect on yield is obtained in a medium that is not supplemented with the vitamins.

In another embodiment, a synergistic effect is obtained in a rate of production of one or more fermentation end-products. For example, if a microorganism comprising a genetic modification in a first metabolic pathway produces the fermentation end-products at a rate that is A % faster than an unmodified microorganism and a microorganism comprising a genetic modification in a second metabolic pathway produces the fermentation end-products at a rate that is B % faster than the unmodified microorganism, then a genetically modified microorganism comprising genetic modifications in both pathways produces the fermentation end-products at a rate that is C % faster than the unmodified microorganism, wherein C is between about 1% and 300% greater than A, B, or A+B; for example, about 1-300%, 1-250%, 1-200%, 1-150%, 1-100%, 1-90%, 1-80%, 1-70%, 1-60%, 1-50%, 1-40%, 1-30%, 1-20%, 1-10%, 10-300%, 10-250%, 10-200%, 10-150%, 10-100%, 10-90%, 10-80%, 10-70%, 10-60%, 10-50%, 10-40%, 10-30%, 10-20%, 20-300%, 20-250%, 20-200%, 20-150%, 20-100%, 20-90%, 20-80%, 20-70%, 20-60%, 20-50%, 20-40%, 20-30%, 30-300%, 30-250%, 30-200%, 30-150%, 30-100%, 30-90%, 30-80%, 30-70%, 30-60%, 30-50%, 30-40%, 40-300%, 40-250%, 40-200%, 40-150%, 40-100%, 40-90%, 40-80%, 40-70%, 40-60%, 40-50%, 50-300%, 50-250%, 50-200%, 50-150%, 50-100%, 50-90%, 50-80%, 50-70%, 50-60%, 60-300%, 60-250%, 60-200%, 60-150%, 60-100%, 60-90%, 60-80%, 60-70%, 70-300%, 70-250%, 70-200%, 70-150%, 70-100%, 70-90%, 70-80%, 80-300%, 80-250%, 80-200%, 80-150%, 80-100%, 80-90%, 90-300%, 90-250%, 90-200%, 90-150%, 90-100%, 100-300%, 100-250%, 100-200%, 100-150%, 150-300%, 150-250%, 150-200%, 200-300%, 200-250%, 250-300%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, or 300% faster. In one embodiment, the synergistic effect on rate of production is obtained in a medium that is supplemented with the vitamins. In another embodiment, the synergistic effect on rate of production is obtained in a medium that is not supplemented with the vitamins.

Thiamine Metabolic Pathway

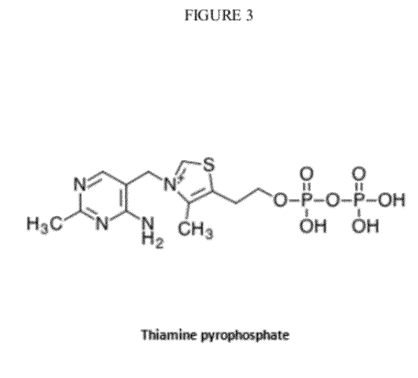

Vitamins can be used to supply or enable synthesis of specific co-factors that facilitate enzymatic reactions in microorganisms. Thiamine (or thiamin, vitamin B₁) is a sulfur-containing, water-soluble vitamin that can be essential to the survival of living organisms. Thiamine can be synthesized by some bacteria, some protozoans, fungi and plants. The active forms of thiamine are phosphorylated thiamine derivatives. There are four recognized derivatives: thiamine monophosphate (ThMP), thiamine pyrophosphate (TPP) which is also known as thiamine diphosphate (ThDP), thiamine triphosphate (ThTP), and adenosine thiamine triphosphate (AThTP)/adenosine thiamine diphosphate (AThDP). TPP can play an important role as a cofactor (coenzyme) in enzymatic reactions. Its molecular structure is shown in FIG. 3.

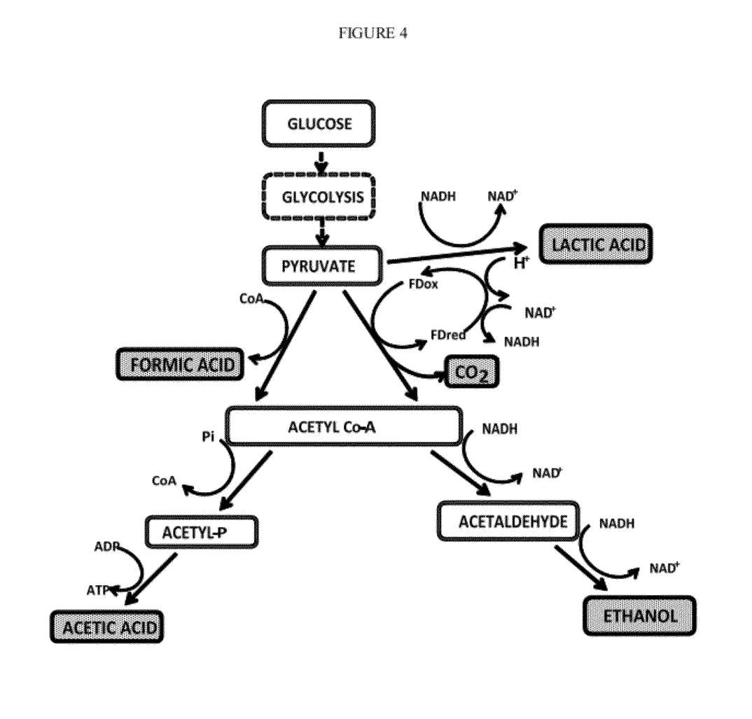

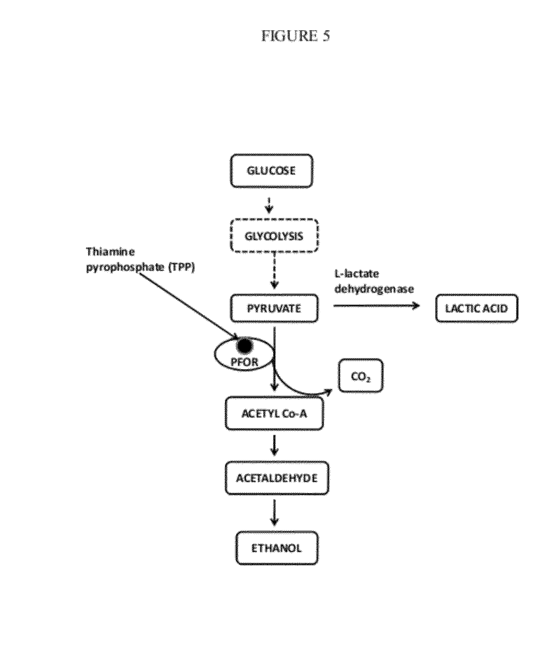

Pyruvate can be an end-product of glycolysis. Under anaerobic conditions, pyruvate can be fermented into products such as lactate (lactic acid), CO₂, ethanol, acetate (acetic acid), formic acid, L-proprionate, butanoate, and other compounds depending on the enzymes present and energy requirements (FIG. 4). For example, lactate dehydrogenase regenerates NAD+ by reducing pyruvate (FIGS. 4 & 5) to lactic acid (L-1-lactate). Pyruvate decarboxylase in aerobic bacteria (using TPP as a cofactor) decarboxylates pyruvate to produce acetyl-CoA, which can be further reduced to ethanol or used to regenerate ATP through conversion to acetic acid. In some anaerobic bacteria, pyruvate decarboxylase is not synthesized and pyruvate ferredoxin oxidoreductase decarboxylates pyruvate to acetyl-CoA. This includes Clostridial microorganisms, such as C. phytofermentans (C. phy), wherein pyruvate ferredoxin oxidoreductase (PFOR) can be used to catalyze the ferredoxin-dependent oxidative decarboxylation of pyruvate using the reduction of ferredoxin, to produce acetyl-CoA and CO₂ (FIG. 5).

Pyruvate+CoA+ferredoxinₒₓ=acetyl-CoA+CO₂+ferrodoxin_{red} See, e.g., Uyeda, K and J. C. Rabinowitz. 1971 J. Biol. Chem. 246:3111-3119.

PFOR is a multi-enzyme complex that can play a role in anaerobic energy production from pyruvate. TPP is a coenzyme that can be an essential part of the enzyme for activity as it can catalyze the transfer of two-carbon units, in particular the dehydrogenation (decarboxylation and subsequent conjugation with coenzyme A) of alpha-keto acids. Uyeda, K and J. C. Rabinowitz. 1971 J. Biol. Chem. 246:3120-3125, which is hereby incorporated by reference in its entirety.

In one embodiment, a genetically modified microorganism that comprises one or more genetic modifications in a thiamine metabolic pathway is capable of growth in a medium that lacks a quantity of thiamine that is sufficient for growth of a corresponding unmodified microorganism of the same species. In one embodiment, the medium completely lacks thiamine. In another embodiment, the medium comprises less thiamine that the minimum amount needed for growth of the corresponding unmodified microorganism of the same species. In one embodiment, the genetic modification increases the synthesis of thiamine by the genetically microorganism in comparison to the unmodified microorganism. In another embodiment, the genetic modification restores function to the thiamine metabolic pathway. In another embodiment, the genetic modification is a gain of function modification. In one embodiment, the unmodified microorganism lacks one or more genes that express enzymes in the thiamine metabolic pathway. In another embodiment, the unmodified microorganism comprises one or more genes in the thiamine metabolic pathway that encode for enzymes with sub-optimal activity. In one embodiment, the unmodified microorganism does not produce any thiamine. In another embodiment the unmodified microorganism produces thiamine at a level that is insufficient to support growth of the unmodified microorganism without an external source of thiamine. In one embodiment, the genetic modification can be the result of a directed evolution process. In one embodiment, the directed evolution process involves treatment with a mutagenic agent. In another embodiment, the directed evolution process involves site-directed mutagenesis. In another embodiment, the directed evolution process involves selection for microorganism growth in media deficient in thiamine. In one embodiment, the genetic modification can be a heterologous copy of one or more polynucleotides that encode for enzymes in the thiamine metabolic pathway. In one embodiment, the polynucleotides are from Clostridium cellulolyticum. In another embodiment, the polynucleotides comprise Ccel_{—}1989, Ccel_{—}1990, Ccel_{—}1991, Ccel_{—}1992, or a combination thereof. In one embodiment, the polynucleotides have at least about 50%, 55%, 60%, 65%, 70%, 75%, 80%, 85%, 90%, 95%, 96%, 97%, 98%, 99%, or 100% identity to at least one of SEQ ID NOs: 21, 33, 35, 37, or 39. In another embodiment, the polynucleotides encode a polypeptide that has at least about 50%, 55%, 60%, 65%, 70%, 75%, 80%, 85%, 90%, 95%, 96%, 97%, 98%, 99%, or 100% identity to at least one of SEQ ID NOs: 34, 36, 38, or 40. In another embodiment, the polynucleotides are Escherichia coli. In one embodiment, the polynucleotides comprise thiC, thiD, thiE, thiF, thiG, thiH, thiL, thiM, or a combination thereof. In one embodiment, the polynucleotides have at least about 50%, 55%, 60%, 65%, 70%, 75%, 80%, 85%, 90%, 95%, 96%, 97%, 98%, 99%, or 100% identity to at least one of SEQ ID NOs: 22, 23, 24, 41, 43, 45, 47, 49, 51, 53, 55, or 57. In another embodiment, the polynucleotides encode a polypeptide that has at least about 50%, 55%, 60%, 65%, 70%, 75%, 80%, 85%, 90%, 95%, 96%, 97%, 98%, 99%, or 100% identity to at least one of SEQ ID NOs: 42, 44, 46, 48, 50, 52, 54, 56, or 58. In one embodiment, the polynucleotides are located on a vector, such as a self replicating vector. In another embodiment, the polynucleotides are integrated into the genome of the microorganism.

In one embodiment, a genetically modified microorganism capable of growth in a medium that lacks a quantity of thiamine sufficient for growth of an unmodified microorganism of the same species can produce greater yields of one or more fermentation end-products than the unmodified microorganism. In one embodiment, the unmodified microorganism produced thiamine at a level that favored production of a secondary fermentation end-product over a first fermentation end-product. In one embodiment, the secondary fermentation end-product is an acid. In one embodiment, the acid is lactic acid. In another embodiment, the unmodified microorganism produced thiamine at a level which caused lower levels of the first fermentation end-product to be produced. In one embodiment, the first fermentation end-product is an alcohol. In one embodiment, the alcohol is methanol, ethanol, propanol, butanol, or a combination thereof. In one embodiment, the alcohol is ethanol.

In another embodiment, a genetically modified microorganism capable of growth in a medium that lacks a quantity of thiamine sufficient for growth of an unmodified microorganism of the same species can produce an amount of a first fermentation end-product that is about 1-300% or higher than the amount produced by the unmodified microorganism, such as about 1-300%, 1-250%, 1-200%, 1-150%, 1-100%, 1-90%, 1-80%, 1-70%, 1-60%, 1-50%, 1-40%, 1-30%, 1-20%, 1-10%, 10-300%, 10-250%, 10-200%, 10-150%, 10-100%, 10-90%, 10-80%, 10-70%, 10-60%, 10-50%, 10-40%, 10-30%, 10-20%, 20-300%, 20-250%, 20-200%, 20-150%, 20-100%, 20-90%, 20-80%, 20-70%, 20-60%, 20-50%, 20-40%, 20-30%, 30-300%, 30-250%, 30-200%, 30-150%, 30-100%, 30-90%, 30-80%, 30-70%, 30-60%, 30-50%, 30-40%, 40-300%, 40-250%, 40-200%, 40-150%, 40-100%, 40-90%, 40-80%, 40-70%, 40-60%, 40-50%, 50-300%, 50-250%, 50-200%, 50-150%, 50-100%, 50-90%, 50-80%, 50-70%, 50-60%, 60-300%, 60-250%, 60-200%, 60-150%, 60-100%, 60-90%, 60-80%, 60-70%, 70-300%, 70-250%, 70-200%, 70-150%, 70-100%, 70-90%, 70-80%, 80-300%, 80-250%, 80-200%, 80-150%, 80-100%, 80-90%, 90-300%, 90-250%, 90-200%, 90-150%, 90-100%, 100-300%, 100-250%, 100-200%, 100-150%, 150-300%, 150-250%, 150-200%, 200-300%, 200-250%, 250-300%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 220%, 240%, 260%, 280%, 300%, or higher. In one embodiment, the first fermentation end-product is an alcohol. In one embodiment, the alcohol is methanol, ethanol, propanol, butanol, or a combination thereof. In one embodiment, the alcohol is ethanol.

In another embodiment, a genetically modified microorganism capable of growth in a medium that lacks a quantity of thiamine sufficient for growth of an unmodified microorganism of the same species can produce an amount of a second fermentation end-product that is about 1-100% lower than the amount produced by the unmodified microorganism, such as about 1-100%, 1-90%, 1-80%, 1-70%, 1-60%, 1-50%, 1-40%, 1-30%, 1-20%, 1-10%, 10-100%, 10-90%, 10-80%, 10-70%, 10-60%, 10-50%, 10-40%, 10-30%, 10-20%, 20-100%, 20-90%, 20-80%, 20-70%, 20-60%, 20-50%, 20-40%, 20-30%, 30-100%, 30-90%, 30-80%, 30-70%, 30-60%, 30-50%, 30-40%, 40-100%, 40-90%, 40-80%, 40-70%, 40-60%, 40-50%, 50-100%, 50-90%, 50-80%, 50-70%, 50-60%, 60-100%, 60-90%, 60-80%, 60-70%, 70-100%, 70-90%, 70-80%, 80-100%, 80-90%, 90-100%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% lower. In one embodiment, the secondary fermentation end-product is an acid. In one embodiment, the acid is lactic acid.

In one embodiment, production of one or more fermentation end-products with a genetically modified microorganism capable of growth in a medium that lacks a quantity of thiamine sufficient for growth of an unmodified microorganism of the same species can occur in a media supplemented with one or more vitamins. In one embodiment, the media is supplemented with thiamine. In another embodiment, the media is not supplemented with thiamine. In another embodiment, the media is supplemented with an NAD+ precursor molecule (e.g., nicotinic acid, nicotinamide, nicotinamide riboside, or a combination thereof), pyridoxine, folinic acid, or a combination thereof. In one embodiment, the genetically modified microorganism produces greater amounts of the fermentation end-products with the same levels, or lower levels, of supplementation than the unmodified microorganism. In one embodiment, the genetically modified microorganism produces the same amount of the fermentation end-products with lower levels of supplementation than the unmodified microorganism. In one embodiment, the genetically modified microorganism produces the same amount of the fermentation end-products without supplementation as the unmodified microorganism with supplementation.

Nicotinate and Nicotinamide Metabolic Pathway

Nicotinic acid (also known as niacin, nicotinate, vitamin B₃, and vitamin PP) is another vitamin. The corresponding amide is called nicotinamide or niacinamide. These vitamins are not directly interconvertable; however, both nicotinate and nicotinamide are precursors in the synthesis of redox pairs NAD+/NADH (nicotinamide adenine dinucleotide) and NADP+/NADPH (nicotinamide adenine dinucleotide phosphate). The nicotinate and nicotinamide metabolic pathway can be referred to herein as the NAD+ synthesis pathway. NAD+/NADH can be an important component of glycolysis, which is the process whereby glucose is broken down into pyruvate. As discussed supra, under anaerobic conditions, pyruvate can be fermented into products such as lactate (lactic acid), CO₂, ethanol, acetate (acetic acid), formic acid, L-proprionate, butanoate, and other compounds depending on the enzymes present and energy requirements (FIG. 4).

NAD+/NADH can be synthesized through two metabolic pathways: a salvage pathway and a de novo pathway. In the salvage pathway, NAD+/NADH can be synthesized from external sources of precursor compounds (e.g., nicotinic acid, nicotinamide, nicotinamide riboside, etc.). In the de novo pathway, NAD+/NADH can be synthesized from quinolinate produced during the metabolism of amino acids (e.g., tryptophan, aspartate, etc.). Many microorganisms do not naturally express all of the enzymes used for de novo synthesis of NAD+/NADH from amino acids. For example, Clostridium phytofermentans is missing two key enzymes (dashed boxes, FIG. 16) and can therefore considered an NAD+auxotroph.

In one embodiment, a genetically modified microorganism that comprises one or more genetic modifications in a nicotinate and nicotinamide metabolic pathway is capable of growth in a medium that lacks a quantity of an NAD+ precursor (e.g., nicotinic acid, nicotinamide, nicotinamide riboside, etc.) that is sufficient for growth of an unmodified microorganism of the same species. In one embodiment, the medium completely lacks NAD precursors. In one embodiment, the medium comprises less NAD+ precursor than the minimum amount needed for growth of the corresponding unmodified microorganism of the same species. In one embodiment, the genetic modification is in an NAD+ salvage pathway. In another embodiment, the genetic modification is in a de novo NAD+ synthesis pathway. In one embodiment, the genetic modification increases the synthesis of NAD+ by the genetically modified microorganisms in comparison to the unmodified microorganism. In another embodiment, the genetic modification restores function to the nicotinate and nicotinamide metabolic pathway. In another embodiment, the genetic modification is a gain of function modification. In one embodiment, the unmodified microorganism lacks one or more genes that express enzymes in the nicotinate and nicotinamide metabolic pathway. In one embodiment, the unmodified microorganism contains one or more genes in the nicotinate and nicotinamide metabolic pathway that encode for enzymes with sub-optimal activity. In one embodiment, the unmodified microorganism did not produce any NAD+ through the de novo NAD+ synthesis pathway before it was genetically modified. In another embodiment, the unmodified microorganism produced NAD+ at a level that is insufficient to support growth of the unmodified microorganism without an external source of the NAD+ precursor. In one embodiment, the genetic modification can be the result of a directed evolution process. In one embodiment, the directed evolution process involves treatment with a mutagenic agent. In another embodiment, the directed evolution process involves site-directed mutagenesis. In another embodiment, the directed evolution process involves selection for microorganism growth in media lacking any NAD+ precursor molecules (e.g., nicotinic acid, nicotinamide, nicotinamide riboside, etc.). In one embodiment, the genetic modification can be a heterologous copy of one or more polynucleotides that encode for enzymes in the nicotinate and nicotinamide metabolic pathway. In one embodiment, the polynucleotides encode enzymes with an activity that is classified by EC numbers 1.4.3.16, 2.5.1.72, 2.4.2.19, or a combination thereof. In another embodiment, the heterologous polynucleotides are from Clostridium cellulolyticum. In another embodiment, the heterologous genes are Ccel_{—}3480, Ccel_{—}3479, Ccel_{—}3478, or a combination thereof. In one embodiment, the polynucleotides have at least about 50%, 55%, 60%, 65%, 70%, 75%, 80%, 85%, 90%, 95%, 96%, 97%, 98%, 99%, or 100% identity to at least one of SEQ ID NOs: 20, 59, 61, or 63. In another embodiment, the polynucleotides encode a polypeptide that has at least about 50%, 55%, 60%, 65%, 70%, 75%, 80%, 85%, 90%, 95%, 96%, 97%, 98%, 99%, or 100% identity to at least one of SEQ ID NOs: 60, 62, or 64. In one embodiment, the polynucleotides are located on a vector, such as a self replicating vector. In another embodiment, the polynucleotides are integrated into the genome of the microorganism.

In one embodiment, a genetically modified microorganism capable of growth in a medium that lacks a quantity of an NAD+ precursor (e.g., nicotinic acid, nicotinamide, nicotinamide riboside, etc.) sufficient for growth of an unmodified microorganism of the same species can produce greater yields of one or more fermentation end-products than the unmodified microorganism. In one embodiment, the yield of at least one of the fermentation end-product is between about 1% and 300% higher; for example, about 1-300%, 1-250%, 1-200%, 1-150%, 1-100%, 1-90%, 1-80%, 1-70%, 1-60%, 1-50%, 1-40%, 1-30%, 1-20%, 1-10%, 10-300%, 10-250%, 10-200%, 10-150%, 10-100%, 10-90%, 10-80%, 10-70%, 10-60%, 10-50%, 10-40%, 10-30%, 10-20%, 20-300%, 20-250%, 20-200%, 20-150%, 20-100%, 20-90%, 20-80%, 20-70%, 20-60%, 20-50%, 20-40%, 20-30%, 30-300%, 30-250%, 30-200%, 30-150%, 30-100%, 30-90%, 30-80%, 30-70%, 30-60%, 30-50%, 30-40%, 40-300%, 40-250%, 40-200%, 40-150%, 40-100%, 40-90%, 40-80%, 40-70%, 40-60%, 40-50%, 50-300%, 50-250%, 50-200%, 50-150%, 50-100%, 50-90%, 50-80%, 50-70%, 50-60%, 60-300%, 60-250%, 60-200%, 60-150%, 60-100%, 60-90%, 60-80%, 60-70%, 70-300%, 70-250%, 70-200%, 70-150%, 70-100%, 70-90%, 70-80%, 80-300%, 80-250%, 80-200%, 80-150%, 80-100%, 80-90%, 90-300%, 90-250%, 90-200%, 90-150%, 90-100%, 100-300%, 100-250%, 100-200%, 100-150%, 150-300%, 150-250%, 150-200%, 200-300%, 200-250%, 250-300%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 220%, 240%, 260%, 280%, 300%, or higher. In one embodiment, the fermentation end-product is an alcohol. In one embodiment, the alcohol is methanol, ethanol, propanol, butanol, or a combination thereof. In one embodiment, the alcohol is ethanol.

In one embodiment, production of one or more fermentation end-products with a genetically modified microorganism capable of growth in a medium that lacks a quantity of an NAD+ precursor (e.g., nicotinic acid, nicotinamide, nicotinamide riboside) can occur in a media supplemented with one or more vitamins. In one embodiment, the vitamin is an NAD+ precursor (e.g., nicotinic acid, nicotinamide, nicotinamide riboside, or a combination thereof). In one embodiment, the vitamin is nicotinic acid. In one embodiment, the media is not supplemented with an NAD+ precursor molecule. In one embodiment, the media is supplemented with thiamine, pyridoxine, folinic acid, or a combination thereof. In one embodiment, the genetically modified microorganism produces greater amounts of the fermentation end-products with the same levels, or lower levels, of supplementation than the unmodified microorganism. In one embodiment, the genetically modified microorganism produces the same amount of the fermentation end-products with lower levels of supplementation than the unmodified microorganism. In one embodiment, the genetically modified microorganism produces the same amount of the fermentation end-products without supplementation as the unmodified microorganism with supplementation.

Vitamin B₆ Metabolic Pathway/PLP Synthesis Pathway

There are six interconvertable forms of vitamin B₆: pyridoxine, pyridoxine 5′-phosphate, pyridoxal, pyridoxal 5′-phosphate, pyridoxamine, and pyridoxamine 5′-phosphate. Of these, pyridoxal 5′-phosphate (PLP) is the metabolically active form. PLP can be a coenzyme involved in numerous cellular processes such as amino acid metabolism (e.g., amino acid catabolism, amino acid interconversion, etc.); gluconeogenesis, both through amino acid catabolism and as a coenzyme for glycogen phosphorylase; lipid metabolism (e.g., biosynthesis of sphingolipids); and gene expression, both through conversion of homocysteine into cysteine and through interactions with transcription factors.

As discussed supra, PLP can be produced from the conversion of other forms of vitamin B₆. PLP can also be synthesized from ribulose 5-phosphate, which is a product of the pentose phosphate pathway, and glyceraldehydes 3-phosphate, which is a product of the glycolysis pathway (see FIG. 41). Some microorganisms lack the enzymes to synthesize PLP from ribulose 5-phosphate and glyceraldehydes 3-phosphate, and therefore require external sources of vitamin B₆.

In one embodiment, a genetically modified microorganism that comprises one or more genetic modifications in a vitamin B₆ metabolic pathway is capable of growth in a medium that lacks a quantity of vitamin B₆ that is sufficient for growth of an unmodified microorganism of the same species. As used herein, the term vitamin B₆ encompasses all the interconvertable forms of vitamin B6 (e.g., pyridoxine, pyridoxine 5′-phosphate, pyridoxal, pyridoxal 5′-phosphate, pyridoxamine, and pyridoxamine 5′-phosphate). In one embodiment, the genetic modification increases the synthesis of PLP by the microorganisms. In one embodiment, the genetic modification restores function to the vitamin B₆ metabolic pathway. In one embodiment, the genetic modification is a gain of function modification. In one embodiment, the unmodified microorganism lacks one or more genes that express enzymes to produce PLP from ribulose 5-phosphate and glyceraldehydes 3-phosphate. In one embodiment, the unmodified microorganism contains one or more genes in the vitamin B₆ metabolic pathway that encode for enzymes with sub-optimal activity; for example, enzymes involved in the interconversion of vitamin B6 forms or enzymes that convert ribulose 5-phosphate and glyceraldehydes 3-phosphate into PLP. In one embodiment, the unmodified microorganism does not produce any PLP from ribulose 5-phosphate and glyceraldehydes 3-phosphate before it was genetically modified. The genetic modification can be the result of a directed evolution process. In one embodiment, the directed evolution process involves treatment with a mutagenic agent. In another embodiment, the directed evolution process involves site-directed mutagenesis. In another embodiment, the directed evolution process involves selection for microorganism growth in media lacking any vitamin B₆. The genetic modification can be a heterologous copy of one or more polynucleotides that encode for enzymes in the vitamin B₆ metabolic pathway. In one embodiment, the polynucleotides comprise YaaD (pdxS), YaaE (pdxT), or a combination thereof. In another embodiment, the polynucleotides are from Clostridium cellulolyticum. In another embodiment, the polynucleotides comprise Ccel_{—}1858, Ccel_{—}1859, or a combination thereof. In one embodiment, the polynucleotides have at least about 50%, 55%, 60%, 65%, 70%, 75%, 80%, 85%, 90%, 95%, 96%, 97%, 98%, 99%, or 100% identity to at least one of SEQ ID NOs: 31, 65 or 67. In another embodiment, the polynucleotides encode a polypeptide that has at least about 50%, 55%, 60%, 65%, 70%, 75%, 80%, 85%, 90%, 95%, 96%, 97%, 98%, 99%, or 100% identity to at least one of SEQ ID NOs: 66 or 68. In one embodiment, the polynucleotides are located on a vector, such as a self replicating vector. In another embodiment, the polynucleotides are integrated into the genome of the microorganism.

In one embodiment, a genetically modified microorganism capable of growth in a medium that lacks a quantity of a vitamin B₆ sufficient for growth of an unmodified microorganism of the same species can produce greater yields of one or more fermentation end-products than the unmodified microorganism. In one embodiment, the yield of at least one fermentation end-product is between 1% and 300% higher; for example, about 1-300%, 1-250%, 1-200%, 1-150%, 1-100%, 1-90%, 1-80%, 1-70%, 1-60%, 1-50%, 1-40%, 1-30%, 1-20%, 1-10%, 10-300%, 10-250%, 10-200%, 10-150%, 10-100%, 10-90%, 10-80%, 10-70%, 10-60%, 10-50%, 10-40%, 10-30%, 10-20%, 20-300%, 20-250%, 20-200%, 20-150%, 20-100%, 20-90%, 20-80%, 20-70%, 20-60%, 20-50%, 20-40%, 20-30%, 30-300%, 30-250%, 30-200%, 30-150%, 30-100%, 30-90%, 30-80%, 30-70%, 30-60%, 30-50%, 30-40%, 40-300%, 40-250%, 40-200%, 40-150%, 40-100%, 40-90%, 40-80%, 40-70%, 40-60%, 40-50%, 50-300%, 50-250%, 50-200%, 50-150%, 50-100%, 50-90%, 50-80%, 50-70%, 50-60%, 60-300%, 60-250%, 60-200%, 60-150%, 60-100%, 60-90%, 60-80%, 60-70%, 70-300%, 70-250%, 70-200%, 70-150%, 70-100%, 70-90%, 70-80%, 80-300%, 80-250%, 80-200%, 80-150%, 80-100%, 80-90%, 90-300%, 90-250%, 90-200%, 90-150%, 90-100%, 100-300%, 100-250%, 100-200%, 100-150%, 150-300%, 150-250%, 150-200%, 200-300%, 200-250%, 250-300%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 220%, 240%, 260%, 280%, 300%, or higher. In one embodiment, the fermentation end-product is an alcohol. In one embodiment, the alcohol is methanol, ethanol, propanol, butanol, or a combination thereof. In one embodiment, the alcohol is ethanol.

In one embodiment, production of one or more fermentation end-products with a genetically modified microorganism capable of growth in a medium that lacks a quantity of a vitamin B₆ can occur in a media supplemented with one or more vitamins. In one embodiment, the vitamin is vitamin B₆ (e.g., pyridoxine, pyridoxine 5′-phosphate, pyridoxal, pyridoxal 5′-phosphate, pyridoxamine, or pyridoxamine 5′-phosphate). In one embodiment, the media is supplemented with pyridoxine. In one embodiment, the media is not supplemented with pyridoxine. In one embodiment, the media is supplemented with thiamine, an NAD+precursor, folinic acid, or a combination thereof. In one embodiment, the genetically modified microorganism produces greater amounts of the fermentation end-products with the same levels, or lower levels, of supplementation than the unmodified microorganism. In one embodiment, the genetically modified microorganism produces the same amount of the fermentation end-products with lower levels of supplementation than the unmodified microorganism. In one embodiment, the genetically modified microorganism produces the same amount of the fermentation end-products without supplementation as the unmodified microorganism with supplementation.

One Carbon Pool by Folate Metabolic Pathway

Vitamin B₉, also known as folic acid, and folate are non-biologically active vitamins that can be converted into tetrahydrofolate (THF) and other derivatives. THF can act as coenzymes in many cellular processes such as the metabolism of amino acids and nucleic acids. THF can be considered to be particularly important for rapidly dividing cells (e.g., bacteria, yeast, etc.).

THF can be synthesized from folate: folate can be first converted to 7,8-dihydrofolate (DHF), which can be converted to THF through the action of an enzyme dihydrofolate reductase. THF can also be synthesized from 5-formyl-tetrahydrofolate, also called folinic acid. Folinic acid can be considered a vitamin B₉ substitute. As used herein, the term vitamin B₉ encompasses both vitamin B₉ and vitamin B₉ substitutes; for example, the term vitamin B₉ encompasses folic acid, folate, and folinic acid. Some microorganisms lack the enzyme dihydrofolate reductase and therefore require an external source of folinic acid for growth.

In one embodiment, a genetically modified microorganism that comprises one or more genetic modifications in a one carbon pool by folate metabolic pathway is capable of growth in a medium that lacks a quantity of vitamin B₉ that is sufficient for growth of an unmodified microorganism of the same species. In one embodiment, the genetic modification increases the synthesis of THF by the genetically modified microorganism in comparison to the unmodified microorganism. In one embodiment, the genetic modification restores a function in the one carbon pool by folate metabolic pathway. In another embodiment, the genetic modification is a gain of function modification. In another embodiment, the unmodified microorganism lacks one or more genes that express enzymes to produce THF from folic acid, folate, or folinic acid. In one embodiment, the unmodified microorganism contains one or more genes in the one carbon pool by folate metabolic pathway that encode for enzymes with sub-optimal activity; for example, enzymes involved in the conversion of folic acid, folate, dihydrofolate or folinic acid into THF. In one embodiment the unmodified microorganism does not produce any THF from dihydrofolate before it was genetically modified. The genetic modification can be the result of a directed evolution process. In one embodiment, the directed evolution process involves treatment with a mutagenic agent. In another embodiment, the directed evolution process involves site-directed mutagenesis. In another embodiment, the directed evolution process involves selection for microorganism growth in media lacking vitamin B₉. The genetic modification can be a heterologous copy of one or more polynucleotides that encode for enzymes in the one carbon pool by folate metabolic pathway. In one embodiment, at least one of the polynucleotides encodes a dihydrofolate reductase. In another embodiment, the polynucleotides are from Clostridium cellulolyticum. In another embodiment, the polynucleotides comprise Ccel_{—}1310. In one embodiment, the polynucleotides have at least about 50%, 55%, 60%, 65%, 70%, 75%, 80%, 85%, 90%, 95%, 96%, 97%, 98%, 99%, or 100% identity to SEQ ID NO: 32 or 69. In another embodiment, the polynucleotides encode a polypeptide that has at least about 50%, 55%, 60%, 65%, 70%, 75%, 80%, 85%, 90%, 95%, 96%, 97%, 98%, 99%, or 100% identity to SEQ ID NOs: 70. In one embodiment, the polynucleotides are located on a vector, such as a self replicating vector. In another embodiment, the polynucleotides are integrated into the genome of the microorganism.

In one embodiment, a genetically modified microorganism capable of growth in a medium that lacks a quantity of vitamin B₉ sufficient for growth of an unmodified microorganism of the same species can produce greater yields of one or more fermentation end-products than the unmodified microorganism. In one embodiment, the yield of at least one of the fermentation end-product is between about 1% and 300% higher; for example, about 1-300%, 1-250%, 1-200%, 1-150%, 1-100%, 1-90%, 1-80%, 1-70%, 1-60%, 1-50%, 1-40%, 1-30%, 1-20%, 1-10%, 10-300%, 10-250%, 10-200%, 10-150%, 10-100%, 10-90%, 10-80%, 10-70%, 10-60%, 10-50%, 10-40%, 10-30%, 10-20%, 20-300%, 20-250%, 20-200%, 20-150%, 20-100%, 20-90%, 20-80%, 20-70%, 20-60%, 20-50%, 20-40%, 20-30%, 30-300%, 30-250%, 30-200%, 30-150%, 30-100%, 30-90%, 30-80%, 30-70%, 30-60%, 30-50%, 30-40%, 40-300%, 40-250%, 40-200%, 40-150%, 40-100%, 40-90%, 40-80%, 40-70%, 40-60%, 40-50%, 50-300%, 50-250%, 50-200%, 50-150%, 50-100%, 50-90%, 50-80%, 50-70%, 50-60%, 60-300%, 60-250%, 60-200%, 60-150%, 60-100%, 60-90%, 60-80%, 60-70%, 70-300%, 70-250%, 70-200%, 70-150%, 70-100%, 70-90%, 70-80%, 80-300%, 80-250%, 80-200%, 80-150%, 80-100%, 80-90%, 90-300%, 90-250%, 90-200%, 90-150%, 90-100%, 100-300%, 100-250%, 100-200%, 100-150%, 150-300%, 150-250%, 150-200%, 200-300%, 200-250%, 250-300%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 220%, 240%, 260%, 280%, 300%, or higher. In one embodiment, the fermentation end-products comprise one or more alcohols. In one embodiment, the alcohols are methanol, ethanol, propanol, butanol, or a combination thereof. In one embodiment, the alcohol is ethanol.

In one embodiment, production of one or more fermentation end-products with a genetically modified microorganism capable of growth in a medium that lacks a quantity of vitamin B₉ can occur in a media supplemented with one or more vitamins. In one embodiment, the vitamins comprise vitamin B₉ (e.g., folic acid, folate) or a vitamin B₉ substitute (e.g., folinic acid). In one embodiment, the vitamin is folinic acid. In another embodiment, the media is not supplemented with folinic acid. In another embodiment, the media is supplemented with thiamine, an NAD+ precursor molecule, pyridoxine, or a combination thereof. In one embodiment, the genetically modified microorganism produces greater amounts of the fermentation end-products with the same levels, or lower levels, of supplementation than the unmodified microorganism. In one embodiment, the genetically modified microorganism produces the same amount of the fermentation end-products with lower levels of supplementation than the unmodified microorganism. In one embodiment, the genetically modified microorganism produces the same amount of the fermentation end-products without supplementation as the unmodified microorganism with supplementation.

Isolated Microorganisms

Disclosed herein are genetically modified microorganisms, wherein the genetically modified microorganisms are capable of growth in a medium that is deficient in one or more vitamins required for growth of an unmodified microorganism of the same species. The genetically modified microorganisms described herein can be used to produce one or more fermentation end-products from a biomass. A genetically modified microorganism can be a bacteria, a yeast, or another fungus. Examples of yeast include, but are not limited to, species found in the genus Ascoidea, Brettanomyces, Candida, Cephaloascus, Coccidiascus, Dipodascus, Eremothecium, Galactomyces, Kluyveromyces, Pichia, Saccharomyces, Schizosaccharomyces, Sporopachydermia, Torulaspora, Yarrowia, or Zygosaccharomyces; for example, Ascoidea rebescens, Brettanomyces anomalus, Brettanomyces bruxellensis, Brettanomyces claussenii, Brettanomyces custersianus, Brettanomyces lambicus, Brettanomyces naardenensis, Brettanomyces nanus, Candida albicans, Candida ascalaphidarum, Candida amphixiae, Candida antarctica, Candida argentea, Candida atlantica, Candida atmosphaerica, Candida blattae, Candida carpophila, Candida cerambycidarum, Candida chauliodes, Candida corydali, Candida dosseyi, Candida dubliniensis, Candida ergatensis, Candida fructus, Candida glabrata, Candida fermentati, Candida guilliermondii, Candida haemulonii, Candida insectamens, Candida insectorum, Candida intermedia, Candida jeffresii, Candida kefyr, Candida krusei, Candida lusitaniae, Candida lyxosophila, Candida maltosa, Candida marina, Candida membranifaciens, Candida milleri, Candida oleophila, Candida oregonensis, Candida parapsilosis, Candida quercitrusa, Candida rugosa, Candida sake, Candida shehatea, Candida temnochilae, Candida tenuis, Candida tropicalis, Candida tsuchiyae, Candida sinolaborantium, Candida sojae, Candida subhashii, Candida viswanathii, Candida utilis, Cephaloascus fragrans, Coccidiascus legeri, Dypodascus albidus, Eremothecium cymbalariae, Galactomyces candidum, Galactomyces geotrichum, Kluyveromyces aestuarii, Kluyveromyces africanus, Kluyveromyces bacillisporus, Kluyveromyces blattae, Kluyveromyces dobzhanskii, Kluyveromyces hubeiensis, Kluyveromyces lactis, Kluyveromyces lodderae, Kluyveromyces marxianus, Kluyveromyces nonfermentans, Kluyveromyces piceae, Kluyveromyces sinensis, Kluyveromyces thermotolerans, Kluyveromyces waltii, Kluyveromyces wickerhamii, Kluyveromyces yarrowii, Pichia anomola, Pichia heedii, Pichia guilliermondii, Pichia kluyveri, Pichia membranifaciens, Pichia norvegensis, Pichia ohmeri, Pichia pastoris, Pichia subpelliculosa, Saccharomyces bayanus, Saccharomyces boulardii, Saccharomyces bulderi, Saccharomyces cariocanus, Saccharomyces cariocus, Saccharomyces cerevisiae, Saccharomyces chevalieri, Saccharomyces dairenensis, Saccharomyces ellipsoideus, Saccharomyces eubayanus, Saccharomyces exiguus, Saccharomyces florentinus, Saccharomyces kluyveri, Saccharomyces martiniae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, Saccharomyces zonatus, Schizosaccharomyces cryophilus, Schizosaccharomyces japonicus, Schizosaccharomyces octosporus, Schizosaccharomyces pombe, Sporopachydermia cereana, Sporopachydermia lactativora, Sporopachydermia quercuum, Torulaspora delbrueckii, Torulaspora franciscae, Torulaspora globosa, Torulaspora pretoriensis, Yarrowia lipolytica, Zygosaccharomyces bailii, Zygosaccharomyces bisporus, Zygosaccharomyces cidri, Zygosaccharomyces fermentati, Zygosaccharomyces florentinus, Zygosaccharomyces kombuchaensis, Zygosaccharomyces lentus, Zygosaccharomyces mellis, Zygosaccharomyces microellipsoides, Zygosaccharomyces mrakii, Zygosaccharomyces pseudorouxii, or Zygosaccharomyces rouxii. Examples of bacteria include, but are not limited to, any bacterium found in the genus of Butyrivibrio, Ruminococcus, Eubacterium, Bacteroides, Acetivibrio, Caldibacillus, Acidothermus, Cellulomonas, Curtobacterium, Micromonospora, Actinoplanes, Streptomyces, Thermobifida, Thermomonospora, Microbispora, Fibrobacter, Sporocytophaga, Cytophaga, Flavobacterium, Achromobacter, Xanthomonas, Cellvibrio, Pseudomonas, Myxobacter, Escherichia, Klebsiella, Thermoanaerobacterium, Thermoanaerobacter, Geobacillus, Saccharococcus, Paenibacillus, Bacillus, Caldicellulosiruptor, Anaerocellum, Anoxybacillus, Zymomonas, Clostridium; for example, Butyrivibrio fibrisolvens, Ruminococcus flavefaciens, Ruminococcus succinogenes, Ruminococcus albus, Eubacterium cellulolyticum, Bacteroides cellulosolvens, Acetivibrio cellulolyticus, Acetivibrio cellulosolvens, Caldibacillus cellulovorans, Bacillus circulans, Acidothermus cellulolyticus, Cellulomonas cartae, Cellulomonas cellasea, Cellulomonas cellulans, Cellulomonas fimi, Cellulomonas flavigena, Cellulomonas gelida, Cellulomonas iranensis, Cellulomonas persica, Cellulomonas uda, Curtobacterium falcumfaciens, Micromonospora melonosporea, Actinoplanes aurantiaca, Streptomyces reticuli, Streptomyces alboguseolus, Streptomyces aureofaciens, Streptomyces cellulolyticus, Streptomyces flavogriseus, Streptomyces lividans, Streptomyces nitrosporeus, Streptomyces olivochromogenes, Streptomyces rochei, Streptomyces thermovulgaris, Streptomyces viridosporus, Thermobifida alba, Thermobifida fusca, Thermobifida cellulolytica, Thermomonospora curvata, Microbispora bispora, Fibrobacter succinogenes, Sporocytophaga myxococcoides, Cytophaga sp., Flavobacterium johnsoniae, Achromobacter piechaudii, Xanthomonas sp., Cellvibrio vulgaris, Cellvibrio fulvus, Cellvibrio gilvus, Cellvibrio mixtus, Pseudomonas fluorescens, Pseudomonas mendocina, Myxobacter sp. AL-1, Escherichia albertii, Escherichia blattae, Escherichia coli, Escherichia fergusonii, Escherichia hermannii, Escherichia vulneris, Klebsiella granulomatis, Klebsiella oxytoca, Klebsiella pneumonia, Klebsiella terrigena, Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brocki, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, Anaerocellum thermophilum, Clostridium thermocellum, Clostridium cellulolyticum, Clostridium straminosolvens, Clostridium acetobutylicum, Clostridium aerotolerans, Clostridium beijerinckii, Clostridium bifermentans, Clostridium botulinum, Clostridium butyricum, Clostridium cadaveris, Clostridium chauvoei, Clostridium clostridioforme, Clostridium colicanis, Clostridium difficile, Clostridium fallax, Clostridium formicaceticum, Clostridium histolyticum, Clostridium innocuum, Clostridium ljungdahlii, Clostridium laramie, Clostridium lavalense, Clostridium novyi, Clostridium oedematiens, Clostridium paraputrificum, Clostridium perfringens, Clostridium phytofermentans (including NRRL B-50364 or NRRL B-50351), Clostridium piliforme, Clostridium ramosum, Clostridium scatologenes, Clostridium septicum, Clostridium sordellii, Clostridium sporogenes, Clostridium sp. Q.D (such as NRRL B-50361, NRRL B-50362, or NRRL B-50363), Clostridium tertium, Clostridium tetani, Clostridium tyrobutyricum, Clostridium thermobutyricum, Zymomonas mobilis or variants thereof (e.g. C. phytofermentans Q.12 or C. phytofermentans Q.13). In one embodiment, the microorganism is a Thermoanaerobacter species. In one embodiment, the Thermoanaerobacter species is Thermoanaerobacter pseudethanolicus, Thermoanaerobacter mathranii, Thermoanaerobacter italicus, Thermoanaerobacter brockii. In one embodiment, the Thermoanaerobacter species is T. acetoethylicus, T. ethanolicus, T. Kivui, T. siderophilus, T. sulfuragignens, T. sulfurophilus, T. thermocopriae, T. thermohydrosulfuricus, T. uzonensis, or T. wiegelii. In one embodiment, the microorganism is a Clostridium species. In one embodiment, the Clostridium species is Clostridium thermocellum, Clostridium beijerinickii, Clostridium acetobutylicum, Clostridium cellulolyticum, Clostridium tyrobutyricum, or Clostridium thermobutyricum. In one embodiment, the Clostridium species is a Clostridium phytofermentans strain. In one embodiment, the Clostridium phytofermentans strain can be, for example, Clostridium phytofermentans Q.8, Clostridium phytofermentans Q.33, or Clostridium phytofermentans Q.32. In one embodiment, the Clostridium species is Clostridium Q.D. In one embodiment, the microorganism is Clostridium phytofermentans, Clostridium Q.D, or a variant thereof. In one embodiment, the microorganism can ferment C5 sugars. In one embodiment, the microorganism can ferment C6 sugars. In one embodiment, the microorganism can hydrolyze and ferment C5 and C6 sugars. In one embodiment, the microorganism can hydrolyze and ferment cellulosic materials. In one embodiment, the microorganism can hydrolyze and ferment hemicellulosic or lignocellulosic material.

In one embodiment a wild type or a genetically modified microorganism can be used for alcohol production by fermentation. For example Clostridium phytofermentans, Clostridium sp. Q.D, Thermoanaerobacter ethanolicus, Clostridium thermocellum, Clostridium beijerinickii, Clostridium acetobutylicum, Clostridium tyrobutyricum, Clostridium thermobutyricum, Thermoanaerobacterium saccharolyticum, Thermoanaerobacter thermohydrosulfuricus, and Saccharomyces cerevisiae, Clostridium acetobutylicum, Moorella ssp., Carboxydocella ssp., Zymomonas mobilis, recombinant E. Coli, Klebsiella oxytoca and Clostridium beijerickii as well as other microorganisms. In one embodiment a microorganism that hydrolyzes and ferments polysaccharides can be used as a biocatalyst in the biofuel or biochemical industry. In one embodiment the microorganism that both hydrolyzes and ferments biomass is Clostridium phytofermentans (ISDg^{T}, American Type Culture Collection 700394^{T}). See U.S. Pat. No. 7,682,811 B2, which is hereby incorporated by reference in its entirety. In another embodiment the microorganism that both hydrolyzes and ferments biomass is a recombinant strains or mutant of Clostridium phytofermentans (ISDg^{T}, American Type Culture Collection 700394¹), or a recombinant strains or mutants of Clostridium sp. Q.D. (collectively members of the “Clostridium biocatalysts”). Without being limiting, these include, e.g., C. phytofermentans strains Q.32 (NRRL B-50511), Q.33 (NRRL B-50512), Q.8 (NRRL B-50351), Q.8_I2_C10 (NRRL B-50447), Q.8_I2_C11 (NRRL B-50448), Q.8_I2_C12 (NRRL B-50449), and Q.8_I2_H9 (NRRL B-50450), Q.7D (NRRL B-50364), Clostridium sp. Q.D (NRRL B-50361), Clostridium sp. Q.D-5 (NRRL B-50362), Clostridium sp. Q.D-7 (NRRL B-50363). See, e.g., U.S. Provisional Patent application Ser. Nos. 61/425,787; 61,436,575; 61/327,051, and U.S. patent application Ser. No. 12/729,037, each of which is hereby incorporated by reference in its entirety.

In another embodiment, provided herein are isolated Gram-positive Clostridium phytofermentans bacterial strains, wherein the bacteria are obligate anaerobic, mesophilic, cellulolytic organisms that can use polysaccharides as a sole carbon source and can oxidize glucose into ethanol or one or more organic acids as its fermentation product. In another embodiment, provided herein are bacteria designated Clostridium phytofermentans Q.32 or Clostridium phytofermentans Q.33, having the NRRL patent deposit designations NRRL B-50511 or NRRL B-50512, respectively.

In another embodiment a genetically modified microorganism adapted for decreased vitamin dependency can, or is further modified to, express one or more proteins that modulate the activity of a metabolic pathway to produce energy-rich products from the conversion of carbohydrates. See, e.g., Lynd, et al. Curr. Opinion Biotechnol. 16:577-583 (2005), which is hereby incorporated by reference in its entirety. In another embodiment strain development provides processes by which new organisms can be derived and screened that possess the attributes for enhanced yields on industrial scales.

In another embodiment, processes that identify such organisms can be useful in screening of other organisms that show the same basic characteristics. Routine procedures for microbial species identification rely on examination of the colony (pigmentation of the surface and reverse sides, topography, texture, and rate of growth) and microscopic morphology (size and shape of cells and spores) and staining (gram-positive v. gram-negative). Further identification characteristics include nutritional requirements (vitamins and amino acids) and temperature tolerance, as well as product production, etc. Morphological and physiological characteristics can frequently vary; in fact, the phenotypic features can be easily influenced by outside factors such as temperature variation, medium, and chemotherapy and therefore strain identification is often difficult.

### DEFINITIONS

In this specification and in the claims which follow, reference will be made to a number of terms which can be defined to have the following meanings. Unless characterized otherwise, technical and scientific terms used herein have the same meaning as commonly understood by one of ordinary skill in the art.

As used in the specification and the appended claims, the singular forms “a”, “an” and “the” include plural referents unless the context clearly dictates otherwise. Thus, for example, reference to “a purified polypeptide” includes mixtures of two or more purified polypeptides.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5.

All numbers expressing quantities of ingredients, reaction conditions, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

“Optional” or “optionally” means that the subsequently described event or circumstance may or may not occur, and that the description includes instances where the event or circumstance occurs and instances where it does not. For example, the phrase “the medium can optionally contain glucose” means that the medium may or may not contain glucose as an ingredient and that the description includes both media containing glucose and media not containing glucose.

As used herein, “obligate” means required or compulsory. As used herein, a “mesophilic” is a bacterium that preferentially ferments a carbon source at about 30-40° C. Clostridium biocatalysts consist of motile rods that form terminal spores.

It is understood that as discussed herein, the terms “similar” or “similarity” mean the same thing as “homology” and “identity.” Thus, for example, if the use of the word homology is used to refer to two non-natural sequences, it is understood that this is not necessarily indicating an evolutionary relationship between these two sequences, but rather is looking at the similarity or relatedness between their nucleic acid or amino acid sequences. Many of the methods for determining similarity between two evolutionarily related molecules are routinely applied to any two or more nucleic acids or polypeptides for the purpose of measuring sequence similarity regardless of whether they are evolutionarily related.

The term “increased” or “increasing” as used herein, refers to the ability of one or more recombinant microorganisms to produce a greater amount of a given product or molecule (e.g., commodity chemical, biofuel, or intermediate product thereof) as compared to a control microorganism, such as an unmodified microorganism or a differently-modified microorganism. An “increased” amount is typically a “statistically significant” amount, and can include an increase that is 1, 2, 3, 4, 5, 6, 7, 8, 9, 10, 15, 20, 30 or more times (including all integers and decimal points in between, e.g., 1.5, 1.6, 1.7. 1.8, etc.) the amount produced by an unmodified microorganism or a differently modified microorganism.

In general, it is understood that one way to define any known variants and derivatives or those that might arise, of the disclosed nucleic acids and polypeptides herein, is through defining the variants and derivatives in terms of similarity, or homology, to specific known sequences. In general, variants of nucleic acids and polypeptides herein disclosed can typically have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% percent similarity, or homology, to the stated sequence or the native sequence. Those of skill in the art readily understand how to determine the similarity of two polypeptides or nucleic acids. For example, the similarity can be calculated after aligning the two sequences so that the similarity is at its highest level.

Another way of calculating similarity, or homology, can be performed by published algorithms. Optimal alignment of sequences for comparison can be conducted by the local homology algorithm of Smith and Waterman Adv. Appl. Math. 2: 482 (1981), by the homology alignment. algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (www.ncbi.nlm.nih.goviblast/b12seq/b12.html)), or by inspection.

The same types of similarity, or homology, can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if similarity is found with at least one of these methods, the sequences would be understood to have the stated similarity.

For example, as used herein, a sequence recited as having a particular percent similarity to another sequence refers to sequences that have the recited similarity as calculated by any one or more of the calculation methods described above. For example, a first sequence has 80 percent similarity, as defined herein, to a second sequence if the first sequence is calculated to have 80 percent similarity to the second sequence using the Zuker calculation method even if the first sequence does not have 80 percent similarity to the second sequence as calculated by any of the other calculation methods. As another example, a first sequence has 80 percent similarity, as defined herein, to a second sequence if the first sequence is calculated to have 80 percent similarity to the second sequence using both the Zuker calculation method and the Pearson and Lipman calculation method even if the first sequence does not have 80 percent similarity to the second sequence as calculated by the Smith and Waterman calculation method, the Needleman and Wunsch calculation method, the Jaeger calculation methods, or any of the other calculation methods. As yet another example, a first sequence has 80 percent similarity, as defined herein, to a second sequence if the first sequence is calculated to have 80 percent similarity to the second sequence using each of the calculation methods (although, in practice, the different calculation methods will often result in different calculated similarity percentages).

As used herein, the term “nucleic acid” refers to single or multiple stranded molecules which can be DNA or RNA, or any combination thereof, including modifications to those nucleic acids. The nucleic acid can represent a coding strand or its complement, or any combination thereof. Nucleic acids can be identical in sequence to the sequences which are naturally occurring for any of the moieties discussed herein or can include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides), a reduction in the AT content of AT rich regions, or replacement of non-preferred codon usage of the expression system to preferred codon usage of the expression system. The nucleic acid can be directly cloned into an appropriate vector, or if desired, can be modified to facilitate the subsequent cloning steps. Such modification steps are routine, an example of which is the addition of oligonucleotide linkers which contain restriction sites to the termini of the nucleic acid. General methods are set forth in Sambrook et al. (2001) Molecular Cloning—A Laboratory Manual (3rd ed.) Vol. 1-3, Cold Spring Harbor Laboratory, Cold Spring Harbor Press, NY, (Sambrook).

The nucleic acid can be detected with a probe capable of hybridizing to the nucleic acid of a cell or a sample. This probe can be a nucleic acid comprising the nucleotide sequence of a coding strand or its complementary strand or the nucleotide sequence of a sense strand or antisense strand, or a fragment thereof. The nucleic acid can comprise the nucleic acid of the bacterial genome, or fragments thereof. In one embodiment, the probe can be either DNA or RNA and can bind either DNA or RNA, or both, in the biological sample. In one embodiment, a polynucleotide probe or primer, e.g., such as those disclosed in U.S. Provisional application Serial No. 6/425,787, comprising at least 15 contiguous nucleotides can be utilized to detect a nucleic acid of the disclosed bacterial strains.

As used herein, the term “nucleic acid probe” refers to a nucleic acid fragment that selectively hybridizes under stringent conditions with a nucleic acid comprising a nucleic acid set forth in a sequence listed herein. This hybridization can be specific. The degree of complementarity between the hybridizing nucleic acid and the sequence to which it hybridizes should be at least enough to exclude hybridization with a nucleic acid encoding an unrelated protein.

As used herein, the term “primer” refers to a single-stranded oligonucleotide that is extended by covalent bonding of nucleotide monomers during amplification or polymerization of a nucleic acid molecule.

The terms “polynucleotide” or “nucleic acid” as used herein designates mRNA, RNA, cRNA, rRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

As will be understood by those skilled in the art, a polynucleotide sequence can include genomic sequences, extra-genomic and plasmid-encoded sequences and smaller engineered gene segments that express, or can be adapted to express, proteins, polypeptides, peptides and the like. Such segments can be naturally isolated, or modified synthetically by the hand of man.

The terms “polynucleotide variant” and “variant” and the like refer to polynucleotides that display substantial sequence identity with any of the reference polynucleotide sequences or genes described herein, and to polynucleotides that hybridize with any polynucleotide reference sequence described herein, or any polynucleotide coding sequence of any gene or protein referred to herein, under low stringency, medium stringency, high stringency, or very high stringency conditions that are defined hereinafter and known in the art. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide, or has increased activity in relation to the reference polynucleotide (i.e., optimized). Polynucleotide variants include, for example, polynucleotides having at least 50% (and at least 51% to at least 99% and all integer percentages in between) sequence identity with a reference polynucleotide described herein.

The terms “polynucleotide variant” and “variant” also include naturally-occurring allelic variants that encode these enzymes. Examples of naturally-occurring variants include allelic variants (same locus), homologs (different locus), and orthologs (different microorganism). Naturally occurring variants such as these can be identified and isolated using well-known molecular biology techniques including, for example, various polymerase chain reaction (PCR) and hybridization-based techniques as known in the art. Naturally-occurring variants can be isolated from any microorganism that encodes one or more genes having a suitable enzymatic activity described herein (e.g., C-C ligase, diol dehydrogenase, pectate lyase, alginate lyase, diol dehydratase, transporter, etc.).

Non-naturally occurring variants can be made by mutagenesis techniques, including those applied to polynucleotides, cells, or microorganisms. The variants can contain nucleotide substitutions, deletions, inversions and insertions. Variation can occur in either or both the coding and non-coding regions. In certain aspects, non-naturally occurring variants can have been optimized for use in a given microorganism (e.g., E. coli), such as by engineering and screening the enzymes for increased activity, stability, or any other desirable feature. The variations can produce both conservative and non-conservative amino acid substitutions (as compared to the originally encoded product). For polynucleotide sequences, conservative variants include those sequences that, because of the degeneracy of the genetic code, encode the amino acid sequence of a reference polypeptide. Variant polynucleotide sequences also include synthetically derived nucleotide sequences, such as those generated, for example, by using site-directed mutagenesis but which still encode a biologically active polypeptide. Generally, variants of a reference polynucleotide sequence will have at least about 30%, 40% 50%, 55%, 60%, 65%, 70%, generally at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity with the reference polynucleotide sequence as determined by sequence alignment programs described elsewhere herein using default parameters. In one embodiment a variant polynucleotide sequence encodes a protein with substantially similar activity compared to a protein encoded by the respective reference polynucleotide sequence. Substantially similar activity means variant protein activity that is within +/−15% of the activity of a protein encoded by the respective reference polynucleotide sequence. In another embodiment a variant polynucleotide sequence encodes a protein with greater activity compared to a protein encoded by the respective reference polynucleotide sequence.

In one embodiment a method is disclosed which uses variants of full-length polypeptides having any of the enzymatic activities described herein, truncated fragments of these full-length polypeptides, variants of truncated fragments, as well as their related biologically active fragments. Typically, biologically active fragments of a polypeptide can participate in an interaction, for example, an intra-molecular or an inter-molecular interaction. An inter-molecular interaction can be a specific binding interaction or an enzymatic interaction (e.g., the interaction can be transient and a covalent bond is formed or broken). Biologically active fragments of a polypeptide/enzyme an enzymatic activity described herein include peptides comprising amino acid sequences sufficiently similar to, or derived from, the amino acid sequences of a (putative) full-length reference polypeptide sequence. Typically, biologically active fragments comprise a domain or motif with at least one enzymatic activity, and can include one or more (and in some cases all) of the various active domains. A biologically active fragment of an enzyme can be a polypeptide fragment which is, for example, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 220, 240, 260, 280, 300, 320, 340, 360, 380, 400, 450, 500, 600 or more contiguous amino acids, including all integers in between, of a reference polypeptide sequence. In certain embodiments, a biologically active fragment comprises a conserved enzymatic sequence, domain, or motif, as described elsewhere herein and known in the art. Suitably, the biologically-active fragment has no less than about 1%, 10%, 25%, or 50% of an activity of the wild-type polypeptide from which it is derived. Additional methods for genetic modification can be found in U.S. Patent Publication US20100086981A1, which is herein incorporated by reference in its entirety.

The term “exogenous” as used herein, refers to a polynucleotide sequence or polypeptide that does not naturally occur in a given wild-type cell or microorganism, but is typically introduced into the cell by a molecular biological technique, i.e., engineering to produce a recombinant microorganism. Examples of “exogenous” polynucleotides include vectors, plasmids, and/or man-made nucleic acid constructs encoding a desired protein or enzyme.

The term “endogenous” as used herein, refers to naturally-occurring polynucleotide sequences or polypeptides that can be found in a given wild-type cell or microorganism. For example, certain naturally-occurring bacterial or yeast species do not typically contain a benzaldehyde lyase gene, and, therefore, do not comprise an “endogenous” polynucleotide sequence that encodes a benzaldehyde lyase. In this regard, it is also noted that even though a microorganism can comprise an endogenous copy of a given polynucleotide sequence or gene, the introduction of a plasmid or vector encoding that sequence, such as to over-express or otherwise regulate the expression of the encoded protein, represents an “exogenous” copy of that gene or polynucleotide sequence. Any of the pathways, genes, or enzymes described herein can utilize or rely on an “endogenous” sequence, or can be provided as one or more “exogenous” polynucleotide sequences, and/or can be used according to the endogenous sequences already contained within a given microorganism.

The term “heterologous” as used herein, refers to an exogenous polynucleotide sequence, an additional copy of an endogenous polynucleotide sequence, or a polypeptide encoded by either.

The term “sequence identity” for example, comprising a “sequence 50% identical to,” as used herein, refers to the extent that sequences are identical on a nucleotide-by-nucleotide basis or an amino acid-by-amino acid basis over a window of comparison. Thus, a “percentage of sequence identity” can be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, H is, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The term “transformation” as used herein, refers to the permanent, heritable alteration in a cell resulting from the uptake and incorporation of foreign DNA into the host-cell genome. This includes the transfer of an exogenous gene from one microorganism into the genome of another microorganism as well as the addition of additional copies of an endogenous gene into a microorganism.

The term “vector” as used herein, refers to a polynucleotide molecule, such as a DNA molecule. It can be derived, from a plasmid, bacteriophage, yeast or virus, into which a polynucleotide can be inserted or cloned. A vector can contain one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. Such a vector can comprise specific sequences that allow recombination into a particular, desired site of the host chromosome. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. A vector can be one which is operably functional in a bacterial cell, such as a cyanobacterial cell. The vector can include a reporter gene, such as a green fluorescent protein (GFP), which can be either fused in frame to one or more of the encoded polypeptides, or expressed separately. The vector can also include a selection marker such as an antibiotic resistance gene that can be used for selection of suitable transformants.

The terms “wild-type” and “naturally-occurring” as used herein are used interchangeably to refer to a gene or gene product that has the characteristics of that gene or gene product when isolated from a naturally occurring source. A wild type gene or gene product (e.g., a polypeptide) is that which is most frequently observed in a population and is thus arbitrarily designed the “normal” or “wild-type” form of the gene.

In one embodiment, provided is a method of fermenting a carbonaceous material, comprising contacting the carbonaceous material with an effective, fermenting amount of an isolated Gram-positive bacterium, wherein the bacterium is an anaerobic, obligate mesophile, wherein the bacterium can use polysaccharides as a sole carbon source and can reduce acetaldehyde into ethanol, whereby contacting the carbonaceous material with the bacterium ferments the carbonaceous material. As used herein, an “effective amount” is within the knowledge of one skilled in the art. Various methods are known by which a person of skill can determine the amount of bacteria useful to effectively ferment a carbonaceous material, e.g., biomass, of interest. The carbonaceous materials can be any one or more of the materials disclosed herein. In one aspect, the bacterial strain is C. phytofermentans. In another aspect, the bacterial strain is C. sp. Q.D.

In one embodiment, the contacting step of the disclosed method occurs at a pH of from about 5.0 to about 7.5. In one embodiment, the contacting step occurs at a pH of from about 6.0 to about 6.5. The contacting step can occur at a pH of about 5, 6, 7, or 8. In one embodiment, the method disclosed is carried out at a temperature from about 30° C. to about 40° C. In one embodiment, the disclosed method is carried out at a temperature from about 35° C. to about 37° C. Additional temperatures at which the disclosed method is carried out are 31° C., 32° C., 33° C., 34° C., 35° C., 36° C., 37° C., 38° C., and 39° C.

In one embodiment, the present disclosure provides a method of growing an isolated Gram-positive bacterium, such as a designated Clostridium biocatalyst. In one embodiment, the bacterium is an anaerobic, obligate mesophile, wherein the bacterium can use cellulose as a sole carbon source and can oxidize acetaldehyde into ethanol, comprising culturing the bacterium at a temperature and on a medium effective to promote growth of the bacterium. The bacterium can grow at a temperature from about 30° C. to about 40° C. In one aspect, the bacterium can grow at a temperature from about 35° C. to about 37° C. Further, the bacterium can grow on medium wherein the pH is from about 5.0 to about 7.5. In one aspect, the pH of the medium can be from about 6.0 to about 6.5. Media are currently known that are effective in promoting growth of the disclosed bacterium. Therefore, a person of skill would know which media would be effective in promoting the growth of the novel bacterium. Examples of media on which the bacterium can grow are shown below.

“Fermentation end-product” is used herein to include biofuels, chemicals, and compounds suitable as liquid fuels, gaseous fuels, reagents, chemical feedstocks, chemical additives, processing aids, food additives, and other products. Examples of fermentation end-products include but are not limited to 1,4 diacids (succinic, fumaric and malic), 2,5 furan dicarboxylic acid, 3 hydroxy propionic acid, aspartic acid, glucaric acid, glutamic acid, itaconic acid, levulinic acid, 3-hydroxybutyrolactone, glycerol, sorbitol, xylitol/arabinitol, butanediol, butanol, methane, methanol, ethane, ethene, ethanol, n-propane, 1-propene, 1-propanol, propanal, acetone, propionate, n-butane, 1-butene, 1-butanol, butanal, butanoate, isobutanal, isobutanol, 2-methylbutanal, 2-methylbutanol, 3-methylbutanal, 3-methylbutanol, 2-butene, 2-butanol, 2-butanone, 2,3-butanediol, 3-hydroxy-2-butanone, 2,3-butanedione, ethylbenzene, ethenylbenzene, 2-phenylethanol, phenylacetaldehyde, 1-phenylbutane, 4-phenyl-1-butene, 4-phenyl-2-butene, 1-phenyl-2-butene, 1-phenyl-2-butanol, 4-phenyl-2-butanol, 1-phenyl-2-butanone, 4-phenyl-2-butanone, 1-phenyl-2,3-butandiol, 1-phenyl-3-hydroxy-2-butanone, 4-phenyl-3-hydroxy-2-butanone, 1-phenyl-2,3-butanedione, n-pentane, ethylphenol, ethenylphenol, 2-(4-hydroxyphenyl)ethanol, 4-hydroxyphenylacetaldehyde, 1-(4-hydroxyphenyl) butane, 4-(4-hydroxyphenyl)-1-butene, 4-(4-hydroxyphenyl)-2-butene, 1-(4-hydroxyphenyl)-1-butene, 1-(4-hydroxyphenyl)-2-butanol, 4-(4-hydroxyphenyl)-2-butanol, 1-(4-hydroxyphenyl)-2-butanone, 4-(4-hydroxyphenyl)-2-butanone, 1-(4-hydroxyphenyl)-2,3-butandiol, 1-(4-hydroxyphenyl)-3-hydroxy-2-butanone, 4-(4-hydroxyphenyl)-3-hydroxy-2-butanone, 1-(4-hydroxyphenyl)-2,3-butanonedione, indolylethane, indolylethene, 2-(indole-3-)ethanol, n-pentane, 1-pentene, 1-pentanol, pentanal, pentanoate, 2-pentene, 2-pentanol, 3-pentanol, 2-pentanone, 3-pentanone, 4-methylpentanal, 4-methylpentanol, 2,3-pentanediol, 2-hydroxy-3-pentanone, 3-hydroxy-2-pentanone, 2,3-pentanedione, 2-methylpentane, 4-methyl-1-pentene, 4-methyl-2-pentene, 4-methyl-3-pentene, 4-methyl-2-pentanol, 2-methyl-3-pentanol, 4-methyl-2-pentanone, 2-methyl-3-pentanone, 4-methyl-2,3-pentanediol, 4-methyl-2-hydroxy-3-pentanone, 4-methyl-3-hydroxy-2-pentanone, 4-methyl-2,3-pentanedione, 1-phenylpentane, 1-phenyl-1-pentene, 1-phenyl-2-pentene, 1-phenyl-3-pentene, 1-phenyl-2-pentanol, 1-phenyl-3-pentanol, 1-phenyl-2-pentanone, 1-phenyl-3-pentanone, 1-phenyl-2,3-pentanediol, 1-phenyl-2-hydroxy-3-pentanone, 1-phenyl-3-hydroxy-2-pentanone, 1-phenyl-2,3-pentanedione, 4-methyl-1-phenylpentane, 4-methyl-1-phenyl-1-pentene, 4-methyl-1-phenyl-2-pentene, 4-methyl-1-phenyl-3-pentene, 4-methyl-1-phenyl-3-pentanol, 4-methyl-1-phenyl-2-pentanol, 4-methyl-1-phenyl-3-pentanone, 4-methyl-1-phenyl-2-pentanone, 4-methyl-1-phenyl-2,3-pentanediol, 4-methyl-1-phenyl-2,3-pentanedione, 4-methyl-1-phenyl-3-hydroxy-2-pentanone, 4-methyl-1-phenyl-2-hydroxy-3-pentanone, 1-(4-hydroxyphenyl) pentane, 1-(4-hydroxyphenyl)-1-pentene, 1-(4-hydroxyphenyl)-2-pentene, 1-(4-hydroxyphenyl)-3-pentene, 1-(4-hydroxyphenyl)-2-pentanol, 1-(4-hydroxyphenyl)-3-pentanol, 1-(4-hydroxyphenyl)-2-pentanone, 1-(4-hydroxyphenyl)-3-pentanone, 1-(4-hydroxyphenyl)-2,3-pentanediol, 1-(4-hydroxyphenyl)-2-hydroxy-3-pentanone, 1-(4-hydroxyphenyl)-3-hydroxy-2-pentanone, 1-(4-hydroxyphenyl)-2,3-pentanedione, 4-methyl-1-(4-hydroxyphenyl) pentane, 4-methyl-1-(4-hydroxyphenyl)-2-pentene, 4-methyl-1-(4-hydroxyphenyl)-3-pentene, 4-methyl-1-(4-hydroxyphenyl)-1-pentene, 4-methyl-1-(4-hydroxyphenyl)-3-pentanol, 4-methyl-1-(4-hydroxyphenyl)-2-pentanol, 4-methyl-1-(4-hydroxyphenyl)-3-pentanone, 4-methyl-1-(4-hydroxyphenyl)-2-pentanone, 4-methyl-1-(4-hydroxyphenyl)-2,3-pentanediol, 4-methyl-1-(4-hydroxyphenyl)-2,3-pentanedione, 4-methyl-1-(4-hydroxyphenyl)-3-hydroxy-2-pentanone, 4-methyl-1-(4-hydroxyphenyl)-2-hydroxy-3-pentanone, 1-indole-3-pentane, 1-(indole-3)-1-pentene, 1-(indole-3)-2-pentene, 1-(indole-3)-3-pentene, 1-(indole-3)-2-pentanol, 1-(indole-3)-3-pentanol, 1-(indole-3)-2-pentanone, 1-(indole-3)-3-pentanone, 1-(indole-3)-2,3-pentanediol, 1-(indole-3)-2-hydroxy-3-pentanone, 1-(indole-3)-3-hydroxy-2-pentanone, 1-(indole-3)-2,3-pentanedione, 4-methyl-1-(indole-3-)pentane, 4-methyl-1-(indole-3)-2-pentene, 4-methyl-1-(indole-3)-3-pentene, 4-methyl-1-(indole-3)-1-pentene, 4-methyl-2-(indole-3)-3-pentanol, 4-methyl-1-(indole-3)-2-pentanol, 4-methyl-1-(indole-3)-3-pentanone, 4-methyl-1-(indole-3)-2-pentanone, 4-methyl-1-(indole-3)-2,3-pentanediol, 4-methyl-1-(indole-3)-2,3-pentanedione, 4-methyl-1-(indole-3)-3-hydroxy-2-pentanone, 4-methyl-1-(indole-3)-2-hydroxy-3-pentanone, n-hexane, 1-hexene, 1-hexanol, hexanal, hexanoate, 2-hexene, 3-hexene, 2-hexanol, 3-hexanol, 2-hexanone, 3-hexanone, 2,3-hexanediol, 2,3-hexanedione, 3,4-hexanediol, 3,4-hexanedione, 2-hydroxy-3-hexanone, 3-hydroxy-2-hexanone, 3-hydroxy-4-hexanone, 4-hydroxy-3-hexanone, 2-methylhexane, 3-methylhexane, 2-methyl-2-hexene, 2-methyl-3-hexene, 5-methyl-1-hexene, 5-methyl-2-hexene, 4-methyl-1-hexene, 4-methyl-2-hexene, 3-methyl-3-hexene, 3-methyl-2-hexene, 3-methyl-1-hexene, 2-methyl-3-hexanol, 5-methyl-2-hexanol, 5-methyl-3-hexanol, 2-methyl-3-hexanone, 5-methyl-2-hexanone, 5-methyl-3-hexanone, 2-methyl-3,4-hexanediol, 2-methyl-3,4-hexanedione, 5-methyl-2,3-hexanediol, 5-methyl-2,3-hexanedione, 4-methyl-2,3-hexanediol, 4-methyl-2,3-hexanedione, 2-methyl-3-hydroxy-4-hexanone, 2-methyl-4-hydroxy-3-hexanone, 5-methyl-2-hydroxy-3-hexanone, 5-methyl-3-hydroxy-2-hexanone, 4-methyl-2-hydroxy-3-hexanone, 4-methyl-3-hydroxy-2-hexanone, 2,5-dimethylhexane, 2,5-dimethyl-2-hexene, 2,5-dimethyl-3-hexene, 2,5-dimethyl-3-hexanol, 2,5-dimethyl-3-hexanone, 2,5-dimethyl-3,4-hexanediol, 2,5-dimethyl-3,4-hexanedione, 2,5-dimethyl-3-hydroxy-4-hexanone, 5-methyl-1-phenylhexane, 4-methyl-1-phenylhexane, 5-methyl-1-phenyl-1-hexene, 5-methyl-1-phenyl-2-hexene, 5-methyl-1-phenyl-3-hexene, 4-methyl-1-phenyl-1-hexene, 4-methyl-1-phenyl-2-hexene, 4-methyl-1-phenyl-3-hexene, 5-methyl-1-phenyl-2-hexanol, 5-methyl-1-phenyl-3-hexanol, 4-methyl-1-phenyl-2-hexanol, 4-methyl-1-phenyl-3-hexanol, 5-methyl-1-phenyl-2-hexanone, 5-methyl-1-phenyl-3-hexanone, 4-methyl-1-phenyl-2-hexanone, 4-methyl-1-phenyl-3-hexanone, 5-methyl-1-phenyl-2,3-hexanediol, 4-methyl-1-phenyl-2,3-hexanediol, 5-methyl-1-phenyl-3-hydroxy-2-hexanone, 5-methyl-1-phenyl-2-hydroxy-3-hexanone, 4-methyl-1-phenyl-3-hydroxy-2-hexanone, 4-methyl-1-phenyl-2-hydroxy-3-hexanone, 5-methyl-1-phenyl-2,3-hexanedione, 4-methyl-1-phenyl-2,3-hexanedione, 4-methyl-1-(4-hydroxyphenyl)hexane, 5-methyl-1-(4-hydroxyphenyl)-1-hexene, 5-methyl-1-(4-hydroxyphenyl)-2-hexene, 5-methyl-1-(4-hydroxyphenyl)-3-hexene, 4-methyl-1-(4-hydroxyphenyl)-1-hexene, 4-methyl-1-(4-hydroxyphenyl)-2-hexene, 4-methyl-1-(4-hydroxyphenyl)-3-hexene, 5-methyl-1-(4-hydroxyphenyl)-2-hexanol, 5-methyl-1-(4-hydroxyphenyl)-3-hexanol, 4-methyl-1-(4-hydroxyphenyl)-2-hexanol, 4-methyl-1-(4-hydroxyphenyl)-3-hexanol, 5-methyl-1-(4-hydroxyphenyl)-2-hexanone, 5-methyl-1-(4-hydroxyphenyl)-3-hexanone, 4-methyl-1-(4-hydroxyphenyl)-2-hexanone, 4-methyl-1-(4-hydroxyphenyl)-3-hexanone, 5-methyl-1-(4-hydroxyphenyl)-2,3-hexanediol, 4-methyl-1-(4-hydroxyphenyl)-2,3-hexanediol, 5-methyl-1-(4-hydroxyphenyl)-3-hydroxy-2-hexanone, 5-methyl-1-(4-hydroxyphenyl)-2-hydroxy-3-hexanone, 4-methyl-1-(4-hydroxyphenyl)-3-hydroxy-2-hexanone, 4-methyl-1-(4-hydroxyphenyl)-2-hydroxy-3-hexanone, 5-methyl-1-(4-hydroxyphenyl)-2,3-hexanedione, 4-methyl-1-(4-hydroxyphenyl)-2,3-hexanedione, 4-methyl-1-(indole-3-)hexane, 5-methyl-1-(indole-3)-1-hexene, 5-methyl-1-(indole-3)-2-hexene, 5-methyl-1-(indole-3)-3-hexene, 4-methyl-1-(indole-3)-1-hexene, 4-methyl-1-(indole-3)-2-hexene, 4-methyl-1-(indole-3)-3-hexene, 5-methyl-1-(indole-3)-2-hexanol, 5-methyl-1-(indole-3)-3-hexanol, 4-methyl-1-(indole-3)-2-hexanol, 4-methyl-1-(indole-3)-3-hexanol, 5-methyl-1-(indole-3)-2-hexanone, 5-methyl-1-(indole-3)-3-hexanone, 4-methyl-1-(indole-3)-2-hexanone, 4-methyl-1-(indole-3)-3-hexanone, 5-methyl-1-(indole-3)-2,3-hexanediol, 4-methyl-1-(indole-3)-2,3-hexanediol, 5-methyl-1-(indole-3)-3-hydroxy-2-hexanone, 5-methyl-1-(indole-3)-2-hydroxy-3-hexanone, 4-methyl-1-(indole-3)-3-hydroxy-2-hexanone, 4-methyl-1-(indole-3)-2-hydroxy-3-hexanone, 5-methyl-1-(indole-3)-2,3-hexanedione, 4-methyl-1-(indole-3)-2,3-hexanedione, n-heptane, 1-heptene, 1-heptanol, heptanal, heptanoate, 2-heptene, 3-heptene, 2-heptanol, 3-heptanol, 4-heptanol, 2-heptanone, 3-heptanone, 4-heptanone, 2,3-heptanediol, 2,3-heptanedione, 3,4-heptanediol, 3,4-heptanedione, 2-hydroxy-3-heptanone, 3-hydroxy-2-heptanone, 3-hydroxy-4-heptanone, 4-hydroxy-3-heptanone, 2-methylheptane, 3-methylheptane, 6-methyl-2-heptene, 6-methyl-3-heptene, 2-methyl-3-heptene, 2-methyl-2-heptene, 5-methyl-2-heptene, 5-methyl-3-heptene, 3-methyl-3-heptene, 2-methyl-3-heptanol, 2-methyl-4-heptanol, 6-methyl-3-heptanol, 5-methyl-3-heptanol, 3-methyl-4-heptanol, 2-methyl-3-heptanone, 2-methyl-4-heptanone, 6-methyl-3-heptanone, 5-methyl-3-heptanone, 3-methyl-4-heptanone, 2-methyl-3,4-heptanediol, 2-methyl-3,4-heptanedione, 6-methyl-3,4-heptanediol, 6-methyl-3,4-heptanedione, 5-methyl-3,4-heptanediol, 5-methyl-3,4-heptanedione, 2-methyl-3-hydroxy-4-heptanone, 2-methyl-4-hydroxy-3-heptanone, 6-methyl-3-hydroxy-4-heptanone, 6-methyl-4-hydroxy-3-heptanone, 5-methyl-3-hydroxy-4-heptanone, 5-methyl-4-hydroxy-3-heptanone, 2,6-dimethylheptane, 2,5-dimethylheptane, 2,6-dimethyl-2-heptene, 2,6-dimethyl-3-heptene, 2,5-dimethyl-2-heptene, 2,5-dimethyl-3-heptene, 3,6-dimethyl-3-heptene, 2,6-dimethyl-3-heptanol, 2,6-dimethyl-4-heptanol, 2,5-dimethyl-3-heptanol, 2,5-dimethyl-4-heptanol, 2,6-dimethyl-3,4-heptanediol, 2,6-dimethyl-3,4-heptanedione, 2,5-dimethyl-3,4-heptanediol, 2,5-dimethyl-3,4-heptanedione, 2,6-dimethyl-3-hydroxy-4-heptanone, 2,6-dimethyl-4-hydroxy-3-heptanone, 2,5-dimethyl-3-hydroxy-4-heptanone, 2,5-dimethyl-4-hydroxy-3-heptanone, n-octane, 1-octene, 2-octene, 1-octanol, octanal, octanoate, 3-octene, 4-octene, 4-octanol, 4-octanone, 4,5-octanediol, 4,5-octanedione, 4-hydroxy-5-octanone, 2-methyloctane, 2-methyl-3-octene, 2-methyl-4-octene, 7-methyl-3-octene, 3-methyl-3-octene, 3-methyl-4-octene, 6-methyl-3-octene, 2-methyl-4-octanol, 7-methyl-4-octanol, 3-methyl-4-octanol, 6-methyl-4-octanol, 2-methyl-4-octanone, 7-methyl-4-octanone, 3-methyl-4-octanone, 6-methyl-4-octanone, 2-methyl-4,5-octanediol, 2-methyl-4,5-octanedione, 3-methyl-4,5-octanediol, 3-methyl-4,5-octanedione, 2-methyl-4-hydroxy-5-octanone, 2-methyl-5-hydroxy-4-octanone, 3-methyl-4-hydroxy-5-octanone, 3-methyl-5-hydroxy-4-octanone, 2,7-dimethyloctane, 2,7-dimethyl-3-octene, 2,7-dimethyl-4-octene, 2,7-dimethyl-4-octanol, 2,7-dimethyl-4-octanone, 2,7-dimethyl-4,5-octanediol, 2,7-dimethyl-4,5-octanedione, 2,7-dimethyl-4-hydroxy-5-octanone, 2,6-dimethyloctane, 2,6-dimethyl-3-octene, 2,6-dimethyl-4-octene, 3,7-dimethyl-3-octene, 2,6-dimethyl-4-octanol, 3,7-dimethyl-4-octanol, 2,6-dimethyl-4-octanone, 3,7-dimethyl-4-octanone, 2,6-dimethyl-4,5-octanediol, 2,6-dimethyl-4,5-octanedione, 2,6-dimethyl-4-hydroxy-5-octanone, 2,6-dimethyl-5-hydroxy-4-octanone, 3,6-dimethyloctane, 3,6-dimethyl-3-octene, 3,6-dimethyl-4-octene, 3,6-dimethyl-4-octanol, 3,6-dimethyl-4-octanone, 3,6-dimethyl-4,5-octanediol, 3,6-dimethyl-4,5-octanedione, 3,6-dimethyl-4-hydroxy-5-octanone, n-nonane, 1-nonene, 1-nonanol, nonanal, nonanoate, 2-methylnonane, 2-methyl-4-nonene, 2-methyl-5-nonene, 8-methyl-4-nonene, 2-methyl-5-nonanol, 8-methyl-4-nonanol, 2-methyl-5-nonanone, 8-methyl-4-nonanone, 8-methyl-4,5-nonanediol, 8-methyl-4,5-nonanedione, 8-methyl-4-hydroxy-5-nonanone, 8-methyl-5-hydroxy-4-nonanone, 2,8-dimethylnonane, 2,8-dimethyl-3-nonene, 2,8-dimethyl-4-nonene, 2,8-dimethyl-5-nonene, 2,8-dimethyl-4-nonanol, 2,8-dimethyl-5-nonanol, 2,8-dimethyl-4-nonanone, 2,8-dimethyl-5-nonanone, 2,8-dimethyl-4,5-nonanediol, 2,8-dimethyl-4,5-nonanedione, 2,8-dimethyl-4-hydroxy-5-nonanone, 2,8-dimethyl-5-hydroxy-4-nonanone, 2,7-dimethylnonane, 3,8-dimethyl-3-nonene, 3,8-dimethyl-4-nonene, 3,8-dimethyl-5-nonene, 3,8-dimethyl-4-nonanol, 3,8-dimethyl-5-nonanol, 3,8-dimethyl-4-nonanone, 3,8-dimethyl-5-nonanone, 3,8-dimethyl-4,5-nonanediol, 3,8-dimethyl-4,5-nonanedione, 3,8-dimethyl-4-hydroxy-5-nonanone, 3,8-dimethyl-5-hydroxy-4-nonanone, n-decane, 1-decene, 1-decanol, decanoate, 2,9-dimethyldecane, 2,9-dimethyl-3-decene, 2,9-dimethyl-4-decene, 2,9-dimethyl-5-decanol, 2,9-dimethyl-5-decanone, 2,9-dimethyl-5,6-decanediol, 2,9-dimethyl-6-hydroxy-5-decanone, 2,9-dimethyl-5,6-decanedionen-undecane, 1-undecene, 1-undecanol, undecanal. undecanoate, n-dodecane, 1-dodecene, 1-dodecanol, dodecanal, dodecanoate, n-dodecane, 1-decadecene, n-tridecane, 1-tridecene, 1-tridecanol, tridecanal, tridecanoate, n-tetradecane, 1-tetradecene, 1-tetradecanol, tetradecanal, tetradecanoate, n-pentadecane, 1-pentadecene, 1-pentadecanol, pentadecanal, pentadecanoate, n-hexadecane, 1-hexadecene, 1-hexadecanol, hexadecanal, hexadecanoate, n-heptadecane, 1-heptadecene, 1-heptadecanol, heptadecanal, heptadecanoate, n-octadecane, 1-octadecene, 1-octadecanol, octadecanal, octadecanoate, n-nonadecane, 1-nonadecene, 1-nonadecanol, nonadecanal, nonadecanoate, eicosane, 1-eicosene, 1-eicosanol, eicosanal, eicosanoate, 3-hydroxy propanal, 1,3-propanediol, 4-hydroxybutanal, 1,4-butanediol, 3-hydroxy-2-butanone, 2,3-butandiol, 1,5-pentane diol, homocitrate, homoisocitorate, b-hydroxy adipate, glutarate, glutarsemialdehyde, glutaraldehyde, 2-hydroxy-1-cyclopentanone, 1,2-cyclopentanediol, cyclopentanone, cyclopentanol, (S)-2-acetolactate, (R)-2,3-Dihydroxy-isovalerate, 2-oxoisovalerate, isobutyryl-CoA, isobutyrate, isobutyraldehyde, 5-amino pentaldehyde, 1,10-diaminodecane, 1,10-diamino-5-decene, 1,10-diamino-5-hydroxydecane, 1,10-diamino-5-decanone, 1,10-diamino-5,6-decanediol, 1,10-diamino-6-hydroxy-5-decanone, phenylacetoaldehyde, 1,4-diphenylbutane, 1,4-diphenyl-1-butene, 1,4-diphenyl-2-butene, 1,4-diphenyl-2-butanol, 1,4-diphenyl-2-butanone, 1,4-diphenyl-2,3-butanediol, 1,4-diphenyl-3-hydroxy-2-butanone, 1-(4-hydeoxyphenyl)-4-phenylbutane, 1-(4-hydeoxyphenyl)-4-phenyl-1-butene, 1-(4-hydeoxyphenyl)-4-phenyl-2-butene, 1-(4-hydeoxyphenyl)-4-phenyl-2-butanol, 1-(4-hydeoxyphenyl)-4-phenyl-2-butanone, 1-(4-hydeoxyphenyl)-4-phenyl-2,3-butanediol, 1-(4-hydeoxyphenyl)-4-phenyl-3-hydroxy-2-butanone, 1-(indole-3)-4-phenylbutane, 1-(indole-3)-4-phenyl-1-butene, 1-(indole-3)-4-phenyl-2-butene, 1-(indole-3)-4-phenyl-2-butanol, 1-(indole-3)-4-phenyl-2-butanone, 1-(indole-3)-4-phenyl-2,3-butanediol, 1-(indole-3)-4-phenyl-3-hydroxy-2-butanone, 4-hydroxyphenylacetoaldehyde, 1,4-di(4-hydroxyphenyl)butane, 1,4-di(4-hydroxyphenyl)-1-butene, 1,4-di(4-hydroxyphenyl)-2-butene, 1,4-di(4-hydroxyphenyl)-2-butanol, 1,4-di(4-hydroxyphenyl)-2-butanone, 1,4-di(4-hydroxyphenyl)-2,3-butanediol, 1,4-di(4-hydroxyphenyl)-3-hydroxy-2-butanone, 1-(4-hydroxyphenyl)-4-(indole-3-)butane, 1-(4-hydroxyphenyl)-4-(indole-3)-1-butene, 1-di(4-hydroxyphenyl)-4-(indole-3)-2-butene, 1-(4-hydroxyphenyl)-4-(indole-3)-2-butanol, 1-(4-hydroxyphenyl)-4-(indole-3)-2-butanone, 1-(4-hydroxyphenyl)-4-(indole-3)-2,3-butanediol, 1-(4-hydroxyphenyl-4-(indole-3)-3-hydroxy-2-butanone, indole-3-acetoaldehyde, 1,4-di(indole-3-) butane, 1,4-di(indole-3)-1-butene, 1,4-di(indole-3)-2-butene, 1,4-di(indole-3)-2-butanol, 1,4-di(indole-3)-2-butanone, 1,4-di(indole-3)-2,3-butanediol, 1,4-di(indole-3)-3-hydroxy-2-butanone, succinate semialdehyde, hexane-1,8-dicarboxylic acid, 3-hexene-1,8-dicarboxylic acid, 3-hydroxy-hexane-1,8-dicarboxylic acid, 3-hexanone-1,8-dicarboxylic acid, 3,4-hexanediol-1,8-dicarboxylic acid, 4-hydroxy-3-hexanone-1,8-dicarboxylic acid, fucoidan, iodine, chlorophyll, carotenoid, calcium, magnesium, iron, sodium, potassium, phosphate, lactic acid, acetic acid; formic acid, isoprenoids, and polyisoprenes, including rubber. Further, such products can include succinic acid, pyruvic acid, enzymes such as cellulases, polysaccharases, lipases, proteases, ligninases, and hemicellulases and can be present as a pure compound, a mixture, or an impure or diluted form.

The term “fatty acid comprising material” as used herein has its ordinary meaning as known to those skilled in the art and can comprise one or more chemical compounds that include one or more fatty acid moieties as well as derivatives of these compounds and materials that comprise one or more of these compounds. Common examples of compounds that include one or more fatty acid moieties include triacylglycerides, diacylglycerides, monoacylglycerides, phospholipids, lysophospholipids, free fatty acids, fatty acid salts, soaps, fatty acid comprising amides, esters of fatty acids and monohydric alcohols, esters of fatty acids and polyhydric alcohols including glycols (e.g. ethylene glycol, propylene glycol, etc.), esters of fatty acids and polyethylene glycol, esters of fatty acids and polyethers, esters of fatty acids and polyglycol, esters of fatty acids and saccharides, esters of fatty acids with other hydroxyl-containing compounds, etc. A fatty acid comprising material can be one or more of these compounds in an isolated or purified form. It can be a material that includes one or more of these compounds that is combined or blended with other similar or different materials. It can be a material where the fatty acid comprising material occurs with or is provided with other similar or different materials, such as vegetable and animal oils; mixtures of vegetable and animal oils; vegetable and animal oil byproducts; mixtures of vegetable and animal oil byproducts; vegetable and animal wax esters; mixtures, derivatives and byproducts of vegetable and animal wax esters; seeds; processed seeds; seed byproducts; nuts; processed nuts; nut byproducts; animal matter; processed animal matter; byproducts of animal matter; corn; processed corn; corn byproducts; distiller's grains; beans; processed beans; bean byproducts; soy products; lipid containing plant, fish or animal matter; processed lipid containing plant or animal matter; byproducts of lipid containing plant, fish or animal matter; lipid containing microbial material; processed lipid containing microbial material; and byproducts of lipid containing microbial matter. Such materials can be utilized in liquid or solid forms. Solid forms include whole forms, such as cells, beans, and seeds; ground, chopped, slurried, extracted, flaked, milled, etc. The fatty acid portion of the fatty acid comprising compound can be a simple fatty acid, such as one that includes a carboxyl group attached to a substituted or un-substituted alkyl group. The substituted or unsubstituted alkyl group can be straight or branched, saturated or unsaturated. Substitutions on the alkyl group can include hydroxyls, phosphates, halogens, alkoxy, or aryl groups. The substituted or unsubstituted alkyl group can have 7 to 29 carbons (e.g., 8 to 30 carbons counting the carboxyl group). The substituted or unsubstituted alkyl group can have 11 to 23 carbons (e.g., 12 to 24 carbons counting the carboxyl group). The carbons can be arranged in a linear chain with or without side chains and/or substitutions. Addition of the fatty acid comprising compound can be by way of adding a material comprising the fatty acid comprising compound.

The term “pH modifier” as used herein has its ordinary meaning as known to those skilled in the art and can include any material that will tend to increase, decrease or hold steady the pH of the broth or medium. A pH modifier can be an acid, a base, a buffer, or a material that reacts with other materials present to serve to raise, lower, or hold steady the pH. In one embodiment, more than one pH modifier can be used, such as more than one acid, more than one base, one or more acid with one or more bases, one or more acids with one or more buffers, one or more bases with one or more buffers, or one or more acids with one or more bases with one or more buffers. In one embodiment, a buffer can be produced in the broth or medium or separately and used as an ingredient by at least partially reacting in acid or base with a base or an acid, respectively. When more than one pH modifiers are utilized, they can be added at the same time or at different times. In one embodiment, one or more acids and one or more bases are combined, resulting in a buffer. In one embodiment, media components, such as a carbon source or a nitrogen source serve as a pH modifier; suitable media components include those with high or low pH or those with buffering capacity. Exemplary media components include acid- or base-hydrolyzed plant polysaccharides having residual acid or base, ammonia fiber explosion (AFEX) treated plant material with residual ammonia, lactic acid, corn steep solids or liquor.

The term “fermentation” as used herein has its ordinary meaning as known to those skilled in the art and can include culturing of a microorganism or group of microorganisms in or on a suitable medium for the microorganisms. The microorganisms can be aerobes, anaerobes, facultative anaerobes, heterotrophs, autotrophs, photoautotrophs, photoheterotrophs, chemoautotrophs, and/or chemoheterotrophs. The microorganisms can be growing aerobically or anaerobically. They can be in any phase of growth, including lag (or conduction), exponential, transition, stationary, death, dormant, vegetative, sporulating, etc.

“Growth phase” is used herein to describe the type of cellular growth that occurs after the “Initiation phase” and before the “Stationary phase” and the “Death phase.” The growth phase is sometimes referred to as the exponential phase or log phase or logarithmic phase.

The term “plant polysaccharide” as used herein has its ordinary meaning as known to those skilled in the art and can comprise one or more polymers of sugars and sugar derivatives as well as derivatives of sugar polymers and/or other polymeric materials that occur in plant matter. Exemplary plant polysaccharides include lignin, cellulose, starch, pectin, and hemicellulose. Others are chitin, sulfonated polysaccharides such as alginic acid, agarose, carrageenan, porphyran, furcelleran and funoran. Generally, the polysaccharide can have two or more sugar units or derivatives of sugar units. The sugar units and/or derivatives of sugar units can repeat in a regular pattern, or otherwise. The sugar units can be hexose units or pentose units, or combinations of these. The derivatives of sugar units can be sugar alcohols, sugar acids, amino sugars, etc. The polysaccharides can be linear, branched, cross-linked, or a mixture thereof. One type or class of polysaccharide can be cross-linked to another type or class of polysaccharide.

The term “fermentable sugars” as used herein has its ordinary meaning as known to those skilled in the art and can include one or more sugars and/or sugar derivatives that can be utilized as a carbon source by the microorganism, including monomers, dimers, and polymers of these compounds including two or more of these compounds. In some cases, the organism can break down these polymers, such as by hydrolysis, prior to incorporating the broken down material. Exemplary fermentable sugars include, but are not limited to glucose, xylose, arabinose, galactose, mannose, rhamnose, cellobiose, lactose, sucrose, maltose, and fructose.

The term “saccharification” as used herein has its ordinary meaning as known to those skilled in the art and can include conversion of plant polysaccharides to lower molecular weight species that can be utilized by the organism at hand. For some organisms, this would include conversion to monosaccharides, disaccharides, trisaccharides, and oligosaccharides of up to about seven monomer units, as well as similar sized chains of sugar derivatives and combinations of sugars and sugar derivatives. For some organisms, the allowable chain-length can be longer and for some organisms the allowable chain-length can be shorter.

The term “biomass” as used herein refers to organic material derived from living organisms, including any member from the kingdoms: Monera, Protista, Fungi, Plantae, or Animalia. Organic material that comprises oligosaccharides (e.g., pentose saccharides, hexose saccharides, or longer saccharides) is of particular use in the processes disclosed herein. Organic material includes organisms or material derived therefrom. Organic material includes cellulosic, hemicellulosic, and/or lignocellulosic material. In one embodiment biomass comprises genetically-modified organisms or parts of organisms, such as genetically-modified plant matter, algal matter, animal matter. In another embodiment biomass comprises non-genetically modified organisms or parts of organisms, such as non-genetically modified plant matter, algal matter, animal matter The term “feedstock” is also used to refer to biomass being used in a process, such as those described herein.

Plant matter comprises members of the kingdom Plantae, such as terrestrial plants and aquatic or marine plants. In one embodiment terrestrial plants comprise crop plants (such as fruit, vegetable or grain plants). In one embodiment aquatic or marine plants include, but are not limited to, sea grass, salt marsh grasses (such as Spartina sp. or Phragmites sp.) or the like. In one embodiment a crop plant comprises a plant that is cultivated or harvested for oral consumption, or for utilization in an industrial, pharmaceutical, or commercial process. In one embodiment, crop plants include but are not limited to corn, wheat, rice, barley, soybeans, bamboo, cotton, crambe, jute, sorghum, high biomass sorghum, oats, tobacco, grasses, (e.g., Miscanthus grass or switch grass), trees (softwoods and hardwoods) or tree leaves, beans rape/canola, alfalfa, flax, sunflowers, safflowers, millet, rye, sugarcane, sugar beets, cocoa, tea, Brassica sp., cotton, coffee, sweet potatoes, flax, peanuts, clover; lettuce, tomatoes, cucurbits, cassaya, potatoes, carrots, radishes, peas, lentils, cabbages, cauliflower, broccoli, Brussels sprouts, grapes, peppers, or pineapples; tree fruits or nuts such as citrus, apples, pears, peaches, apricots, walnuts, almonds, olives, avocadoes, bananas, or coconuts; flowers such as orchids, carnations and roses; nonvascular plants such as ferns; oil producing plants (such as castor beans, jatropha, or olives); or gymnosperms such as palms. Plant matter also comprises material derived from a member of the kingdom Plantae, such as woody plant matter, non-woody plant matter, cellulosic material, lignocellulosic material, or hemicellulosic material. Plant matter includes carbohydrates (such as pectin, starch, inulin, fructans, glucans, lignin, cellulose, or xylan). Plant matter also includes sugar alcohols, such as glycerol. In one embodiment plant matter comprises a corn product, (e.g. corn stover, corn cobs, corn grain, corn steep liquor, corn steep solids, or corn grind), stillage, bagasse, leaves, pomace, or material derived therefrom. In another embodiment plant matter comprises distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, pits, fermentation waste, skins, straw, seeds, shells, beancake, sawdust, wood flour, wood pulp, paper pulp, paper pulp waste streams, rice or oat hulls, bagasse, grass clippings, lumber, or food leftovers. These materials can come from farms, forestry, industrial sources, households, etc. In another embodiment plant matter comprises an agricultural waste byproduct or side stream. In another embodiment plant matter comprises a source of pectin such as citrus fruit (e.g., orange, grapefruit, lemon, or limes), potato, tomato, grape, mango, gooseberry, carrot, sugar-beet, and apple, among others. In another embodiment plant matter comprises plant peel (e.g., citrus peels) and/or pomace (e.g., grape pomace). In one embodiment plant matter is characterized by the chemical species present, such as proteins, polysaccharides or oils. In one embodiment plant matter is from a genetically modified plant. In one embodiment a genetically-modified plant produces hydrolytic enzymes (such as a cellulase, hemicellulase, or pectinase etc.) at or near the end of its life cycles. In another embodiment a genetically-modified plant encompasses a mutated species or a species that can initiate the breakdown of cell wall components. In another embodiment plant matter is from a non-genetically modified plant.

Animal matter comprises material derived from a member of the kingdom Animaliae (e.g., bone meal, hair, heads, tails, beaks, eyes, feathers, entrails, skin, shells, scales, meat trimmings, hooves or feet) or animal excrement (e.g., manure). In one embodiment animal matter comprises animal carcasses, milk, meat, fat, animal processing waste, or animal waste (manure from cattle, poultry, and hogs).

Algal matter comprises material derived from a member of the kingdoms Monera (e.g. Cyanobacteria) or Protista (e.g. algae (such as green algae, red algae, glaucophytes, cyanobacteria,) or fungus-like members of Protista (such as slime molds, water molds, etc). Algal matter includes seaweed (such as kelp or red macroalgae), or marine microflora, including plankton.

Organic material comprises waste from farms, forestry, industrial sources, households or municipalities. In one embodiment organic material comprises sewage, garbage, food waste (e.g., restaurant waste), waste paper, toilet paper, yard clippings, or cardboard.

The term “carbonaceous biomass” as used herein has its ordinary meaning as known to those skilled in the art and can include one or more biological materials that can be converted into a biofuel, chemical or other product. Carbonaceous biomass can comprise municipal waste (waste paper, recycled toilet papers, yard clippings, etc.), wood, plant material, plant matter, plant extract, bacterial matter (e.g. bacterial cellulose), distillers' grains, a natural or synthetic polymer, or a combination thereof.

In one embodiment, biomass does not include fossilized sources of carbon, such as hydrocarbons that are typically found within the top layer of the Earth's crust (e.g., natural gas, nonvolatile materials composed of almost pure carbon, like anthracite coal, etc.).

“Broth” is used herein to refer to inoculated medium at any stage of growth, including the point immediately after inoculation and the period after any or all cellular activity has ceased and can include the material after post-fermentation processing. It includes the entire contents of the combination of soluble and insoluble matter, suspended matter, cells and medium, as appropriate.

The term “productivity” as used herein has its ordinary meaning as known to those skilled in the art and can include the mass of a material of interest produced in a given time in a given volume. Units can be, for example, grams per liter-hour, or some other combination of mass, volume, and time. In fermentation, productivity is frequently used to characterize how fast a product can be made within a given fermentation volume. The volume can be referenced to the total volume of the fermentation vessel, the working volume of the fermentation vessel, or the actual volume of broth being fermented. The context of the phrase will indicate the meaning intended to one of skill in the art. Productivity is different from “titer” in that productivity includes a time term, and titer is analogous to concentration. Titer and Productivity can generally be measured at any time during the fermentation, such as at the beginning, the end, or at some intermediate time, with titer relating the amount of a particular material present or produced at the point in time of interest and the productivity relating the amount of a particular material produced per liter in a given amount of time. The amount of time used in the productivity determination can be from the beginning of the fermentation or from some other time, and go to the end of the fermentation, such as when no additional material is produced or when harvest occurs, or some other time as indicated by the context of the use of the term. “Overall productivity” refers to the productivity determined by utilizing the final titer and the overall fermentation time. “Productivity to maximum titer” refers to the productivity determined utilizing the maximum titer and the time to achieve the maximum titer. “Instantaneous productivity” refers to the productivity at a moment in time and can be determined from the slope of the titer v. time curve for the compound of interest, or by other appropriate means as determined by the circumstances of the operation and the context of the language. “Incremental productivity” refers to productivity over a portion of the fermentation time, such as several minutes, an hour, or several hours. Frequently, an incremental productivity is used to imply or approximate instantaneous productivity. Other types of productivity can be used as well, with the context indicating how the value should be determined.

“Titer” refers to the amount of a particular material present in a fermentation broth. It is similar to concentration and can refer to the amount of material made by the organism in the broth from all fermentation cycles, or the amount of material made in the current fermentation cycle or over a given period of time, or the amount of material present from whatever source, such as produced by the organism or added to the broth. Frequently, the titer of soluble species will be referenced to the liquid portion of the broth, with insolubles removed, and the titer of insoluble species will be referenced to the total amount of broth with insoluble species being present, however, the titer of soluble species can be referenced to the total broth volume and the titer of insoluble species can be referenced to the liquid portion, with the context indicating the which system is used with both reference systems intended in some cases. Frequently, the value determined referenced to one system will be the same or a sufficient approximation of the value referenced to the other. “Concentration” when referring to material in the broth generally refers to the amount of a material present from all sources, whether made by the organism or added to the broth. Concentration can refer to soluble species or insoluble species, and is referenced to either the liquid portion of the broth or the total volume of the broth, as for “titer.”

The term “biocatalyst” as used herein has its ordinary meaning as known to those skilled in the art and can include one or more enzymes and microorganisms, including solutions, suspensions, and mixtures of enzymes and microorganisms. In some contexts this word will refer to the possible use of either enzymes or microorganisms to serve a particular function, in other contexts the word will refer to the combined use of the two, and in other contexts the word will refer to only one of the two. The context of the phrase will indicate the meaning intended to one of skill in the art.

The terms “conversion efficiency” or “yield” as used herein have their ordinary meaning as known to those skilled in the art and can include the mass of product made from a mass of substrate. The term can be expressed as a percentage yield of the product from a starting mass of substrate. For the production of ethanol from glucose, the net reaction is generally accepted as:

C₆H₁₂O₆→2C₂H₅OH+2CO₂

and the theoretical maximum conversion efficiency, or yield, is 51% (wt.). Frequently, the conversion efficiency will be referenced to the theoretical maximum, for example, “80% of the theoretical maximum.” In the case of conversion of glucose to ethanol, this statement would indicate a conversion efficiency of 41% (wt.). The context of the phrase will indicate the substrate and product intended to one of skill in the art. For substrates comprising a mixture of different carbon sources such as found in biomass (xylan, xylose, glucose, cellobiose, arabinose cellulose, hemicellulose etc.), the theoretical maximum conversion efficiency of the biomass to ethanol is an average of the maximum conversion efficiencies of the individual carbon source constituents weighted by the relative concentration of each carbon source. In some cases, the theoretical maximum conversion efficiency is calculated based on an assumed saccharification yield. In one embodiment, given carbon source comprising 10 g of cellulose, the theoretical maximum conversion efficiency can be calculated by assuming saccharification of the cellulose to the assimilable carbon source glucose of about 75% by weight. In this embodiment, 10 g of cellulose can provide 7.5 g of glucose which can provide a maximum theoretical conversion efficiency of about 7.5 g*51% or 3.8 g of ethanol. In other cases, the efficiency of the saccharification step can be calculated or determined, i.e., saccharification yield.

Saccharification yields can include between about 10-100%, about 20-90%, about 30-80%, about 40-70% or about 50-60%, such as about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% for any carbohydrate carbon sources larger than a single monosaccharide subunit.

The saccharification yield takes into account the amount of ethanol, and acidic products produced plus the amount of residual monomeric sugars detected in the media. The ethanol figures resulting from media components are not adjusted in this experiment. These can account for up to 3 g/l ethanol production or equivalent of up to 6 g/l sugar as much as +/−10%-15% saccharification yield (or saccharification efficiency). For this reason the saccharification yield % can be greater than 100% for some plots. The terms “fed-batch” or “fed-batch fermentation” as used herein has its ordinary meaning as known to those skilled in the art and can include a method of culturing microorganisms where nutrients, other medium components, or biocatalysts (including, for example, enzymes, fresh microorganisms, extracellular broth, etc.) are supplied to the fermentor during cultivation, but culture broth is not harvested from the fermentor until the end of the fermentation, although it can also include “self seeding” or “partial harvest” techniques where a portion of the fermentor volume is harvested and then fresh medium is added to the remaining broth in the fermentor, with at least a portion of the inoculum being the broth that was left in the fermentor. In some embodiments, a fed-batch process might be referred to with a phrase such as, “fed-batch with cell augmentation.” This phrase can include an operation where nutrients and microbial cells are added or one where microbial cells with no substantial amount of nutrients are added. The more general phrase “fed-batch” encompasses these operations as well. The context where any of these phrases is used will indicate to one of skill in the art the techniques being considered.

“Pretreatment” or “pretreated” is used herein to refer to any mechanical, chemical, thermal, biochemical process or combination of these processes whether in a combined step or performed sequentially, that achieves disruption or expansion of the biomass so as to render the biomass more susceptible to attack by enzymes and/or microorganisms. In one embodiment, pretreatment includes removal or disruption of lignin so as to make the cellulose and hemicellulose polymers in the plant biomass more available to cellulolytic enzymes and/or microorganisms, for example, by treatment with acid or base. In one embodiment, pretreatment includes the use of a microorganism of one type to render plant polysaccharides more accessible to microorganisms of another type, for example, by treatment with acid or base. In one embodiment, pretreatment includes disruption or expansion of cellulosic and/or hemicellulosic material. Steam explosion, and ammonia fiber expansion (or explosion) (AFEX) are well known thermal/chemical techniques. Hydrolysis, including methods that utilize acids, bases, and/or enzymes can be used. Other thermal, chemical, biochemical, enzymatic techniques can also be used.

“Fed-batch” or “fed-batch fermentation” can be used herein to include methods of culturing microorganisms where nutrients, other medium components, or biocatalysts (including, for example, enzymes, fresh organisms, extracellular broth, genetically modified plants and/or organisms, etc.) are supplied to the fermentor during cultivation, but culture broth is not harvested from the fermentor until the end of the fermentation, although it can also include “self seeding” or “partial harvest” techniques where a portion of the fermentor volume is harvested and then fresh medium is added to the remaining broth in the fermentor, with at least a portion of the inoculum being the broth that was left in the fermentor. During a fed-batch fermentation, the broth volume can increase, at least for a period, by adding medium or nutrients to the broth while fermentation organisms are present. In some fed-batch fermentations, the broth volume can be insensitive to the addition of nutrients and in some cases not change from the addition of nutrients. Suitable nutrients which can be utilized include those that are soluble, insoluble, and partially soluble, including gasses, liquids and solids. In one embodiment, a fed-batch process is referred to with a phrase such as, “fed-batch with cell augmentation.” This phrase can include an operation where nutrients and cells are added or one where cells with no substantial amount of nutrients are added. The more general phrase “fed-batch” encompasses these operations as well. The context where any of these phrases is used will indicate to one of skill in the art the techniques being considered.

A term “phytate” as used herein has its ordinary meaning as known to those skilled in the art can be include phytic acid, its salts, and its combined forms as well as combinations of these.

“Sugar compounds” is used herein to include monosaccharide sugars, including but not limited to hexoses and pentoses; sugar alcohols; sugar acids; sugar amines; compounds containing two or more of these linked together directly or indirectly through covalent or ionic bonds; and mixtures thereof. Included within this description are disaccharides; trisaccharides; oligosaccharides; polysaccharides; and sugar chains, branched and/or linear, of any length.

“Dry cell weight” is used herein to refer to a method of determining the cell content of a broth or inoculum, and the value so determined. Generally, the method includes rinsing or washing a volume of broth followed by drying and weighing the residue, but is not necessary. In some cases, a sample of broth is simply centrifuged with the layer containing cells collected, dried, and weighed. Frequently, the broth is centrifuged, then resuspended in water or a mixture of water and other ingredients, such as a buffer, ingredients to create an isotonic condition, ingredients to control any change in osmotic pressure, etc. The centrifuge-resuspend steps can be repeated, if desired, and different resuspending solutions can be used prior to the final centrifuging and drying. When an insoluble medium component is present, the presence of the insoluble component can be ignored, with the value determined as above. Methods when insoluble medium components are present can include those where the insoluble component is reacted to a soluble form, dissolved or extracted into a different solvent that can include water, or separated by an appropriate method, such as by centrifugation, gradient centrifugation, flotation, filtration, or other suitable technique or combination of techniques.

### Clostridium Biocatalysts

Clostridium biocatalysts are fast-growing, high yielding strains of Clostridium phytofermentans or Clostridium sp. Q.D, and can in some embodiments be defined based on the phenotypic and genotypic characteristics of the cultured strain as described infra. Aspects described herein generally include systems, methods, and compositions for producing fuels, such as ethanol, and/or other useful organic products involving, for example, Clostridium biocatalysts and/or any other strain of the species, including those which can be derived from Clostridium phytofermentans or Clostridium sp. Q.D, including genetically modified strains, or strains separately isolated. Some exemplary species can be defined using standard taxonomic considerations (Stackebrandt and Goebel, International Journal of Systematic Bacteriology, 44:846-9, 1994): Strains with 16S rRNA sequence homology values of 98% and higher as compared to the type Clostridium biocatalysts, and strains with DNA re-association values of at least about 70% can be considered Clostridium biocatalysts. For example, strains with 16S rRNA sequence homology values of at least 97.1, 97.2, 97.3, 97.4, 97.5, 97.6, 97.7, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9% can be considered Clostridium biocatalysts. In one embodiment, strains with DNA re-association values of at least about 71, 72, 73, 74, 75, 76, 77, 78, 79, 80, 81, 82, 83, 84, 85, 86, 87, 88, 89, 90, 91, 92, 93, 94, 95, 96, 97, 98, 99% can be considered Clostridium biocatalysts. Considerable evidence exists to indicate that many microorganisms which have 70% or greater DNA re-association values also have at least 98% DNA sequence identity and share phenotypic traits defining a species. Analyses of the genome sequence of Clostridium biocatalysts indicate the presence of large numbers of genes and genetic loci that are likely to be involved in mechanisms and pathways for plant polysaccharide fermentation, giving rise to the unusual fermentation properties of these biocatalysts which can be found in all or nearly all strains of the species Clostridium biocatalysts and can be natural isolates, or genetically modified strains.

### Attributes of Clostridium Biocatalysts

In one embodiment, the microorganisms, Clostridium biocatalysts, provide useful advantages for the conversion of biomass to fermentation end-products, such as biofuels or chemicals. In a further embodiment, the biofuel is an alcohol, such as ethanol. One advantage of this microorganism is its ability to produce enzymes capable of hydrolyzing polysaccharides and higher molecular weight saccharides to lower molecular weight saccharides, such as oligosaccharides, disaccharides, and monosaccharides. In one embodiment, Clostridium biocatalysts produce a hydrolytic enzyme which facilitates fermenting of a biomass material. Examples of biomass material that can be fermented include, but are not limited to, cellulosic, hemicellulosic, lignocellulosic materials; pectins; starches; wood; paper; agricultural products; forest waste; tree waste; tree bark; leaves; switchgrasses; sawgrass; woody plant matter; non-woody plant matter; carbohydrates; pectin; starch; inulin; fructans; glucans; corn; sugar cane; other grasses; bamboo, algae, seeds, hulls, distillers' grains, and material derived from these materials. The organisms can usually produce these enzymes as needed, frequently without excessive production of unnecessary hydrolytic enzymes, or in one embodiment, one or more enzymes is added to further improve the organism's production capability. This ability to produce a very wide range of hydrolytic enzymes gives Clostridium biocatalysts and the associated technology distinct advantages in biomass fermentation, especially those fermentations not utilizing simple sugars as the feedstock. Various fermentation conditions can enhance the activities of the organism, resulting in higher yields, higher productivity, greater product selectivity, and/or greater conversion efficiency. In one embodiment, fermentation conditions include fed batch operation and fed batch operation with cell augmentation; addition of complex nitrogen sources such as corn steep powder or yeast extract; addition of specific amino acids including proline, glycine, isoleucine, and/or histidine; addition of a complex material containing one or more of these amino acids; addition of other nutrients or other compounds such as phytate, proteases enzymes, or polysaccharase enzymes. In one embodiment, fermentation conditions can include supplementation of a medium with an organic nitrogen source. In another embodiment, fermentation conditions can include supplementation of a medium with an inorganic nitrogen source. In one embodiment, the addition of one material provides supplements that fit into more than one category, such as providing amino acids and phytate.

In one embodiment, a Clostridium biocatalyst is used to hydrolyze various higher saccharides (higher molecular weight) present in biomass to lower saccharides (lower molecular weight), such as in preparation for fermentation to produce ethanol, hydrogen, or other chemicals such as organic acids including formic acid, acetic acid, and lactic acid. In one embodiment an advantage of Clostridium biocatalysts are their ability to hydrolyze polysaccharides and higher saccharides that contain hexose sugar units or that contain pentose sugar units, and that contain both, into lower saccharides and in some cases monosaccharides. These enzymes and/or the hydrolysate can be used in fermentations to produce various products including fuels, and other chemicals. In another embodiment an advantage of Clostridium biocatalysts are their ability to produce ethanol, hydrogen, and other fuels or compounds such as organic acids including acetic acid, formic acid, and lactic acid from lower sugars (lower molecular weight) such as monosaccharides. In another embodiment an advantage of Clostridium biocatalysts are their ability to perform the combined steps of hydrolyzing a higher molecular weight biomass containing sugars and/or higher saccharides or polysaccharides to lower sugars and fermenting these lower sugars into desirable products including ethanol, hydrogen, and other compounds such as organic acids including formic acid, acetic acid, and lactic acid.

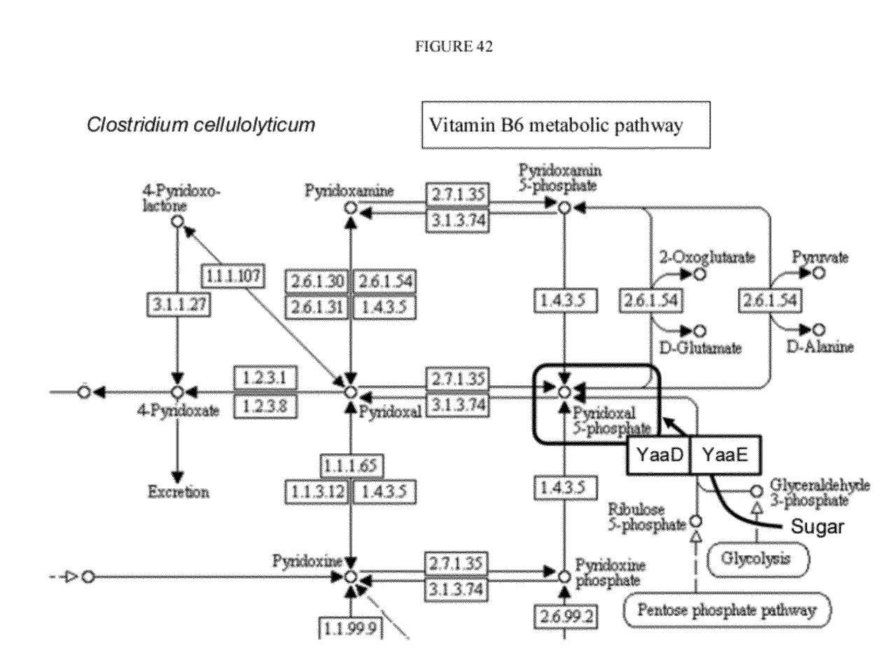

In one embodiment, a Clostridium biocatalyst is used to hydrolyze various higher saccharides (higher molecular weight) present in biomass to lower saccharides (lower molecular weight), such as in preparation for enhanced fermentation end-products, such as biofuels and chemicals, wherein the Clostridium biocatalyst has modified activity of enzymes regulating any of the metabolic pathways described in FIGS. 4-7 and 10, thereby reducing lactic acid formation. In another embodiment, a Clostridium biocatalyst has modified activity of any of the metabolic pathways described in FIG. 16, thereby enhancing glycolysis and fermentation. In another embodiment, a Clostridium biocatalyst has modified activity in any of the metabolic pathways described in FIGS. 41-43. Modified activity of the Clostridium biocatalyst enzymes can be through genetic modification of the Clostridium biocatalyst or through the addition of exogenous agents.

In another embodiment an advantage of Clostridium biocatalysts are their ability to grow under conditions that include elevated ethanol concentration, high sugar concentration, low sugar concentration, utilize insoluble carbon sources, and/or operate under anaerobic conditions. These characteristics, in various combinations, can be used to achieve operation with long fermentation cycles and can be used in combination with batch fermentations, fed batch fermentations, self-seeding/partial harvest fermentations, and recycle of cells from the final fermentation as inoculum.

In one example, the process for converting biomass material into ethanol includes pretreating the biomass material (e.g., “feedstock”), hydrolyzing the pretreated biomass to convert polysaccharides to oligosaccharides, further hydrolyzing the oligosaccharides to monosaccharides, and converting the monosaccharides to ethanol. In one example, the biomass can be hydrolyzed directly to monosaccharides or other saccharides that are utilized by the fermentation organism to produce ethanol or other products. If a different final product is desired, such as hydrocarbons, hydrogen, methane, hydroxy compounds such as alcohols (e.g. butanol, propanol, methanol, etc.), carbonyl compounds such as aldehydes and ketones (e.g. acetone, formaldehyde, 1-propanal, etc.), organic acids, derivatives of organic acids such as esters (e.g. wax esters, glycerides, etc.) and other functional compounds including, but not limited to, 1,2-propanediol, 1,3-propanediol, lactic acid, formic acid, acetic acid, succinic acid, pyruvic acid, enzymes such as cellulases, polysaccharases, lipases, proteases, ligninases, and hemicellulases, the monosaccharides can be used in the biosynthesis of that particular compound. Biomass material that can be utilized includes woody plant matter, non-woody plant matter, cellulosic material, lignocellulosic material, hemicellulosic material, carbohydrates, pectin, starch, inulin, fructans, glucans, corn, algae, sugar cane, grasses, switchgrass, bamboo, citrus peels, sorghum, high biomass sorghum, oat hulls, and material derived from these. The final product can then be separated and/or purified, as indicated by the properties for the desired final product. In some instances, compounds related to sugars such as sugar alcohols or sugar acids can be utilized as well.

In one embodiment, more than one of these steps can occur at any given time. For example, hydrolysis of the pretreated feedstock and hydrolysis of the oligosaccharides can occur simultaneously, and one or more of these can occur simultaneously to the conversion of monosaccharides to ethanol.

In another embodiment, an enzyme can directly convert the polysaccharide to monosaccharides. In some instances, an enzyme can hydrolyze the polysaccharide to oligosaccharides and the enzyme or another enzyme can hydrolyze the oligosaccharides to monosaccharides.

In another embodiment, the enzymes present in the fermentation can be produced separately and then added to the fermentation or they can be produced by microorganisms present in the fermentation. In one embodiment, the microorganisms present in the fermentation produces some enzymes. In another embodiment, enzymes are produced separately and added to the fermentation.

For the overall conversion of pretreated biomass to final product to occur at high rates, it is generally necessary for each of the necessary enzymes for each conversion step to be present with sufficiently high activity. If one of these enzymes is missing or is present in insufficient quantities, the production rate of ethanol, or other desired product will be reduced. The production rate can also be reduced if the microorganisms responsible for the conversion, of monosaccharides to product only slowly take up monosaccharides and/or have only limited capability for translocation of the monosaccharides and intermediates produced during the conversion to ethanol.

In another embodiment, the enzymes of the method are produced by Clostridium biocatalysts, including a range of hydrolytic enzymes suitable for the biomass materials used in the fermentation methods. In one embodiment, Clostridium biocatalysts are grown under conditions appropriate to induce and/or promote production of the enzymes needed for the saccharification of the polysaccharide present. The production of these enzymes can occur in a separate vessel, such as a seed fermentation vessel or other fermentation vessel, or in the production fermentation vessel where ethanol production occurs. When the enzymes are produced in a separate vessel, they can, for example, be transferred to the production fermentation vessel along with the cells, or as a relatively cell free solution liquid containing the intercellular medium with the enzymes. When the enzymes are produced in a separate vessel, they can also be dried and/or purified prior to adding them to the production fermentation vessel. The conditions appropriate for production of the enzymes are frequently managed by growing the cells in a medium that includes the biomass that the cells will be expected to hydrolyze in subsequent fermentation steps. Additional medium components, such as salt supplements, growth factors, and cofactors including, but not limited to phytate, amino acids, and peptides can also assist in the production of the enzymes utilized by the microorganism in the production of the desired products. In one embodiment, additional medium components can include thiamine. In another embodiment, additional medium components can include an NAD+ precursor molecule or vitamin (e.g., nicotinic acid, nicotinamide, nicotinamide riboside, etc.).

### Feedstock and Pretreatment of Feedstock

In one embodiment, the feedstock contains cellulosic, hemicellulosic, and/or lignocellulosic material. The feedstock can be derived from agricultural crops, crop residues, trees, woodchips, sawdust, paper, cardboard, grasses, algae and other sources.

Cellulose is a linear polymer of glucose where the glucose units are connected via β(1→4) linkages. Hemicellulose is a branched polymer of a number of sugar monomers including glucose, xylose, mannose, galactose, rhamnose and arabinose, and can have sugar acids such as mannuronic acid and galacturonic acid present as well. Lignin is a cross-linked, racemic macromolecule of mostly p-coumaryl alcohol, conferyl alcohol and sinapyl alcohol. These three polymers occur together in lignocellulosic materials in plant biomass. The different characteristics of the three polymers can make hydrolysis of the combination difficult as each polymer tends to shield the others from enzymatic attack.

In one embodiment, methods are provided for the pretreatment of feedstock used in the fermentation and production of the biofuels and ethanol. The pretreatment steps can include mechanical, thermal, pressure, chemical, thermochemical, and/or biochemical tests pretreatment prior to being used in a bioprocess for the production of fuels and chemicals, but untreated biomass material can be used in the process as well. Mechanical processes can reduce the particle size of the biomass material so that it can be more conveniently handled in the bioprocess and can increase the surface area of the feedstock to facilitate contact with chemicals/biochemicals/biocatalysts. Mechanical processes can also separate one type of biomass material from another. The biomass material can also be subjected to thermal and/or chemical pretreatments to render plant polymers more accessible. Multiple steps of treatment can also be used.

Mechanical processes include, but are not limited to, washing, soaking, milling, size reduction, screening, shearing, size classification and density classification processes. Chemical processes include, but are not limited to, bleaching, oxidation, reduction, acid treatment, base treatment, sulfite treatment, acid sulfite treatment, basic sulfite treatment, ammonia treatment, and hydrolysis. Thermal processes include, but are not limited to, sterilization, ammonia fiber expansion or explosion (“AFEX”), steam explosion, holding at elevated temperatures, pressurized or unpressurized, in the presence or absence of water, and freezing. Biochemical processes include, but are not limited to, treatment with enzymes, including enzymes produced by genetically-modified plants, and treatment with microorganisms. Various enzymes that can be utilized include cellulase, amylase, β-glucosidase, xylanase, gluconase, and other polysaccharases; lysozyme; laccase, and other lignin-modifying enzymes; lipoxygenase, peroxidase, and other oxidative enzymes; proteases; and lipases. One or more of the mechanical, chemical, thermal, thermochemical, and biochemical processes can be combined or used separately. Such combined processes can also include those used in the production of paper, cellulose products, microcrystalline cellulose, and cellulosics and can include pulping, kraft pulping, acidic sulfite processing. The feedstock can be a side stream or waste stream from a facility that utilizes one or more of these processes on a biomass material, such as cellulosic, hemicellulosic or lignocellulosic material. Examples include paper plants, cellulosics plants cotton processing plants, and microcrystalline cellulose plants. The feedstock can also include cellulose-containing or cellulosic containing waste materials. The feedstock can also be biomass materials, such as wood, grasses, corn, starch, or sugar, produced or harvested as an intended feedstock for production of ethanol or other products such as by Clostridium biocatalysts.

In another embodiment, a method can utilize a pretreatment process disclosed in U.S. Patents and Patent Applications US20040152881, US20040171136, US20040168960, US20080121359, US20060069244, US20060188980, US20080176301, 5693296, 6262313, US20060024801, 5969189, 6043392, US20020038058, U.S. Pat. No. 5,865,898, U.S. Pat. No. 5,865,898, U.S. Pat. No. 6,478,965, 5,986,133, or US20080280338, each of which is incorporated by reference herein in its entirety

An AFEX process can be used for pretreatment of biomass. In one embodiment, the AFEX process is used in the preparation of cellulosic, hemicellulosic or lignocellulosic materials for fermentation to ethanol or other products. The process can generally include combining the feedstock with ammonia, heating under pressure, and suddenly releasing the pressure. Water can be present in various amounts. The AFEX process has been the subject of numerous patents and publications.

In another embodiment, the pretreatment of biomass comprises the addition of calcium hydroxide to a biomass to render the biomass susceptible to degradation. Pretreatment comprises the addition of calcium hydroxide and water to the biomass to form a mixture, and maintaining the mixture at a relatively high temperature. Alternatively, an oxidizing agent, selected from the group consisting of oxygen and oxygen-containing gasses, can be added under pressure to the mixture. Examples of carbon hydroxide treatments are disclosed in U.S. Pat. No. 5,865,898 to Holtzapple and S. Kim and M. T. Holzapple, Bioresource Technology, 96, (2005) 1994, incorporated by reference herein in its entirety.

In one embodiment, pretreatment of biomass comprises dilute acid hydrolysis. Example of dilute acid hydrolysis treatment are disclosed in T. A. Lloyd and C. E Wyman, Bioresource Technology, (2005) 96, 1967), incorporated by reference herein in its entirety. Dilute acid for hydrolysis can be derived from inorganic acids, such as sulfuric acid or hydrochloric acid, or from organic acids, such as acetic acid, malic acid, lactic acid, carboxylic acid, or citric acid.

In another embodiment, pretreatment of biomass comprises pH controlled liquid hot water treatment. Examples of pH controlled liquid hot water treatments are disclosed in N. Mosier et al., Bioresource Technology, (2005) 96, 1986, incorporated by reference herein in its entirety.

In one embodiment, pretreatment of biomass comprises aqueous ammonia recycle process (ARP). Examples of aqueous ammonia recycle process are described in T. H. Kim and Y. Y. Lee, Bioresource Technology, (2005)₉₆, 2007, incorporated by reference herein in its entirety.

In one embodiment, the above mentioned methods have two steps: a pretreatment step that leads to a wash stream, and an enzymatic hydrolysis step of pretreated-biomass that produces a hydrolysate stream. In the above methods, the pH at which the pretreatment step is carried out includes acid hydrolysis, hot water pretreatment, steam explosion or alkaline reagent based methods (AFEX, ARP, and lime pretreatments). Dilute acid and hot water treatment methods solubilize mostly hemicellulose, whereas methods employing alkaline reagents remove most lignin during the pretreatment step. As a result, the wash stream from the pretreatment step in the former methods contains mostly hemicellulose-based sugars, whereas this stream has mostly lignin for the high-pH methods. The subsequent enzymatic hydrolysis of the residual biomass leads to mixed sugars (C5 and C6) in the alkali based pretreatment methods, while glucose is the major product in the hydrolyzate from the low and neutral pH methods. In one embodiment, the treated material is additionally treated with catalase or another similar chemical, chelating agents, surfactants, and other compounds to remove impurities or toxic chemicals or further release polysaccharides.

In one embodiment, pretreatment of biomass comprises ionic liquid pretreatment. Biomass can be pretreated by incubation with an ionic liquid, followed by ionic liquid extraction with a wash solvent such as alcohol or water. The treated biomass can then be separated from the ionic liquid/wash-solvent solution by centrifugation or filtration, and sent to the saccharification reactor or vessel. Examples of ionic liquid pretreatment are disclosed in US publication No. 2008/0227162, incorporated herein by reference in its entirety.

In another embodiment, a method can utilize a pretreatment process disclosed in U.S. Pat. No. 4,600,590 to Dale, U.S. Pat. No. 4,644,060 to Chou, U.S. Pat. No. 5,037,663 to Dale. U.S. Pat. No. 5,171,592 to Holtzapple, et al., U.S. Pat. No. 5,939,544 to Karstens, et al., U.S. Pat. No. 5,473,061 to Bredereck, et al., U.S. Pat. No. 6,416,621 to Karstens., U.S. Pat. No. 6,106,888 to Dale, et al., U.S. Pat. No. 6,176,176 to Dale, et al., PCT publication WO2008/020901 to Dale, et al., Felix, A., et al., Anim. Prod. 51, 47-61 (1990)., Wais, A. C., Jr., et al., Journal of Animal Science, 35, No. 1, 109-112 (1972), which are incorporated herein by reference in their entireties.

Alteration of the pH of a pretreated feedstock can be accomplished by washing the feedstock (e.g., with water) one or more times to remove an alkaline or acidic substance, or other substance used or produced during pretreatment. Washing can comprise exposing the pretreated feedstock to an equal volume of water 2, 3, 4, 5, 6, 7, 8, 9, 10, 11, 12, 13, 14, 15, 16, 17, 18, 19, 20, 21, 22, 23, 24, 25 or more times. In another embodiment, a pH modifier can be added. For example, an acid, a buffer, or a material that reacts with other materials present can be added to modulate the pH of the feedstock. In one embodiment, more than one pH modifier can be used, such as one or more bases, one or more bases with one or more buffers, one or more acids, one or more acids with one or more buffers, or one or more buffers. When more than one pH modifiers are utilized, they can be added at the same time or at different times. Other non-limiting exemplary methods for neutralizing feedstocks treated with alkaline substances have been described, for example in U.S. Pat. Nos. 4,048,341; 4,182,780; and 5,693,296.

In one embodiment, one or more acids can be combined, resulting in a buffer. Suitable acids and buffers that can be used as pH modifiers include any liquid or gaseous acid that is compatible with the microorganism. Non-limiting examples include peroxyacetic acid, sulfuric acid, lactic acid, citric acid, phosphoric acid, and hydrochloric acid. In some instances, the pH can be lowered to neutral pH or acidic pH, for example a pH of 7.0, 6.5, 6.0, 5.5, 5.0, 4.5, 4.0, or lower. In some embodiments, the pH is lowered and/or maintained within a range of about pH 4.5 to about 7.1, or about 4.5 to about 6.9, or about pH 5.0 to about 6.3, or about pH 5.5 to about 6.3, or about pH 6.0 to about 6.5, or about pH 5.5 to about 6.9 or about pH 6.2 to about 6.7.

In another embodiment, biomass can be pre-treated at an elevated temperature and/or pressure. In one embodiment biomass is pre treated at a temperature range of 20° C. to 400° C. In another embodiment biomass is pretreated at a temperature of about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C., 350° C., 400° C. or higher. In another embodiment, elevated temperatures are provided by the use of steam, hot water, or hot gases. In one embodiment steam can be injected into a biomass containing vessel. In another embodiment the steam, hot water, or hot gas can be injected into a vessel jacket such that it heats, but does not directly contact the biomass.

In another embodiment, a biomass can be treated at an elevated pressure. In one embodiment biomass is pre treated at a pressure range of about 1 psi to about 30 psi. In another embodiment biomass is pre treated at a pressure or about 1 psi, 2 psi, 3 psi, 4 psi, 5 psi, 6 psi, 7 psi, 8 psi, 9 psi, 10 psi, 12 psi, 15 psi, 18 psi, 20 psi, 22 psi, 24 psi, 26 psi, 28 psi, 30 psi or more. In some embodiments, biomass can be treated with elevated pressures by the injection of steam into a biomass containing vessel. In one embodiment, the biomass can be treated to vacuum conditions prior or subsequent to alkaline or acid treatment or any other treatment methods provided herein.

In one embodiment alkaline or acid pretreated biomass is washed (e.g. with water (hot or cold) or other solvent such as alcohol (e.g. ethanol)), pH neutralized with an acid, base, or buffering agent (e.g. phosphate, citrate, borate, or carbonate salt) or dried prior to fermentation. In one embodiment, the drying step can be performed under vacuum to increase the rate of evaporation of water or other solvents. Alternatively, or additionally, the drying step can be performed at elevated temperatures such as about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C. or more.

In one embodiment, the pretreatment step includes a step of solids recovery. The solids recovery step can be during or after pretreatment (e.g., acid or alkali pretreatment), or before the drying step. In one embodiment, the solids recovery step provided by the methods disclosed herein includes the use of a sieve, filter, screen, or a membrane for separating the liquid and solids fractions. In one embodiment a suitable sieve pore diameter size ranges from about 0.001 microns to 8 mm, such as about 0.005 microns to 3 mm or about 0.01 microns to 1 mm. In one embodiment a sieve pore size has a pore diameter of about 0.01 microns, 0.02 microns, 0.05 microns, 0.1 microns, 0.5 microns, 1 micron, 2 microns, 4 microns, 5 microns, 10 microns, 20 microns, 25 microns, 50 microns, 75 microns, 100 microns, 125 microns, 150 microns, 200 microns, 250 microns, 300 microns, 400 microns, 500 microns, 750 microns, 1 mm or more.

In one embodiment, biomass (e.g. corn stover) is processed or pretreated prior to fermentation. In one embodiment a method of pre-treatment includes but is not limited to, biomass particle size reduction, such as for example shredding, milling, chipping, crushing, grinding, or pulverizing. In one embodiment, biomass particle size reduction can include size separation methods such as sieving, or other suitable methods known in the art to separate materials based on size. In one embodiment size separation can provide for enhanced yields. In one embodiment, separation of finely shredded biomass (e.g. particles smaller than about 8 mm in diameter, such as, 8, 7.9, 7.7, 7.5, 7.3, 7, 6.9, 6.7, 6.5, 6.3, 6, 5.9, 5.7, 5.5, 5.3, 5, 4.9, 4.7, 4.5, 4.3, 4, 3.9, 3.7, 3.5, 3.3, 3, 2.9, 2.7, 2.5, 2.3, 2, 1.9, 1.7, 1.5, 1.3, 1, 0.9, 0.8, 0.7, 0.6, 0.5, 0.4, 0.3, 0.2, or 0.1 mm) from larger particles allows the recycling of the larger particles back into the size reduction process, thereby increasing the final yield of processed biomass. In one embodiment, a fermentative mixture is provided which comprises a pretreated lignocellulosic feedstock comprising less than about 50% of a lignin component present in the feedstock prior to pretreatment and comprising more than about 60% of a hemicellulose component present in the feedstock prior to pretreatment; and a microorganism capable of fermenting a five-carbon sugar, such as xylose, arabinose or a combination thereof, and a six-carbon sugar, such as glucose, galactose, mannose or a combination thereof. In some instances, pretreatment of the lignocellulosic feedstock comprises adding an alkaline substance which raises the pH to an alkaline level, for example NaOH. In one embodiment, NaOH is added at a concentration of about 0.5% to about 2% by weight of the feedstock. In one embodiment, pretreatment also comprises addition of a chelating agent. In one embodiment, the microorganism is a bacterium, such as a member of the genus Clostridium, for example, Clostridium biocatalysts.

The present disclosure also provides a fermentative mixture comprising: a cellulosic feedstock pre-treated with an alkaline substance which maintains an alkaline pH, and at a temperature of from about 80° C. to about 120° C.; and a microorganism capable of fermenting a five-carbon sugar and a six-carbon sugar. In one embodiment, the five-carbon sugar is xylose, arabinose, or a combination thereof. In one embodiment, the six-carbon sugar is glucose, galactose, mannose, or a combination thereof. In one embodiment, the alkaline substance is NaOH. In some embodiments, NaOH is added at a concentration of about 0.5% to about 2% by weight of the feedstock. In one embodiment, the microorganism is a bacterium, such as a member of the genus Clostridium, for example, Clostridium biocatalysts. In still another embodiment, the microorganism is genetically modified to enhance activity of one or more hydrolytic enzymes. In another embodiment, the microorganism is genetically modified to enable to enhance production of thiamine. In another embodiment, the microorganism is genetically modified to enable or enhance the synthesis of nicotinate D-ribonucleotide from amino acid metabolism. In another embodiment, the microorganism is genetically modified to enable or enhance the synthesis of NAD+ from amino acid metabolism.

Further provided herein is a fermentative mixture comprising a cellulosic feedstock pre-treated with an alkaline substance which increases the pH to an alkaline level, at a temperature of from about 80° C. to about 120° C.; and a microorganism capable of uptake and fermentation of an oligosaccharide. In one embodiment the alkaline substance is NaOH. In some embodiments, NaOH is added at a concentration of about 0.5% to about 2% by weight of the feedstock. In one embodiment, the microorganism is a bacterium, such as a member of the genus Clostridium, for example, Clostridium biocatalysts. In one embodiment, the microorganism is genetically modified to express or increase expression of an enzyme capable of hydrolyzing said oligosaccharide, a transporter capable of transporting the oligosaccharide, or a combination thereof.

In one embodiment, pretreatment of biomass comprises enzyme hydrolysis. In one embodiment a biomass is pretreated with an enzyme or a mixture of enzymes, e.g., endonucleases, exonucleases, cellobiohydrolases, cellulase, beta-glucosidases, glycoside hydrolases, glycosyltransferases, lyases, esterases and proteins containing carbohydrate-binding modules. In one embodiment, the enzyme or mixture of enzymes is one or more individual enzymes with distinct activities. In another embodiment, the enzyme or mixture of enzymes can be enzyme domains with a particular catalytic activity. For example, an enzyme with multiple activities can have multiple enzyme domains, including for example glycoside hydrolases, glycosyltransferases, lyases and/or esterases catalytic domains.

In one embodiment, pretreatment of biomass comprises enzyme hydrolysis with one or more enzymes from a Clostridium biocatalyst. In one embodiment, pretreatment of biomass comprises enzyme hydrolysis with one or more enzymes from Clostridium biocatalysts, wherein the one or more enzyme is selected from the group consisting of endonucleases, exonucleases, cellobiohydrolases, beta-glucosidases, glycoside hydrolases, glycosyltransferases, lyases, esterases and proteins containing carbohydrate-binding modules. In one embodiment, biomass can be pretreated with a hydrolase identified in C. phytofermentans.

In one embodiment, pretreatment of biomass comprises enzyme hydrolysis with one or more of enzymes listed in Table 1. Table 1 show examples of known activities of some of the glycoside hydrolases, lyases, esterases, and proteins containing carbohydrate-binding modules family members predicted to be present in Clostridia, for example, C. phytofermentans. Known activities are listed by activity and corresponding PC number as determined by the International Union of Biochemistry and Molecular Biology.

TABLE 1
Known activities of glycoside hydrolase family members

| Glycoside | | Number of domains |
|---|---|---|
| Hydrolase | | predicted in |
| Family | Known activities | C. phytofermentans |
| 1 | beta-glucosidase (EC 3.2.1.21); beta-galactosidase (EC 3.2.1.23); beta- | 1 |
| | mannosidase (EC 3.2.1.25); beta-glucuronidase (EC 3.2.1.31); beta-D- | |
| | fucosidase (EC 3.2.1.38); phlorizin hydrolase (EC 3.2.1.62); 6- | |
| | phospho--galactosidase (EC 3.2.1.85); 6-phospho-beta-glucosidase | |
| | (EC 3.2.1.86); strictosidinebeta-glucosidase (EC 3.2.1.105); lactase (EC | |
| | 3.2.1.108); amygdalinbeta-glucosidase (EC 1 3.2.1.117); prunasin beta- | |
| | glucosidase (EC 3.2.1.118); raucaifricine beta-glucosidase (EC | |
| | 3.2.1.125); thioglucosidase (EC 3.2.1.147); beta-primeverosidase (EC | |
| | 3.2.1.149); isoflavonod 7-0-beta-apiosyl--glucosidase (EC 3.2.1.161); | |
| | hydroxyisourate hydrolase (EC_3.—.—.—);_beta-glycosidase_(EC_3.2.1.—) | |
| 2 | beta-galactosidase (EC 3.2.1.23); beta-mannosidase (EC 3.2.1.25); | 5 |
| | beta-glucuronidase (EC 3.2.1.31); mannosylglycoprotein 5 endo-beta- | |
| | mannosidase (EC 3.2.1.152); exo-beta glucosaminidase_(E 3.2.1.—) | |
| 3 | beta-glucosidase (EC 3.2.1.21); xylan 1,4-beta-xylosidase (EC | 8 |
| | 3.2.1.37); beta-N-acetylhexosaminidase (EC 3.2.1.52); glucan 1,3- | |
| | beta-glucosiclase (EC 3.2.1.58); glucan 1,4-beta-glucosidase (EC 3.2.1.74); | |
| | exo-1,3-1,4-glucanase (EC 3.2.1.—); alpha-L arabinofuranosidase | |
| | (EC 3.2.1.55). | |
| 4 | maltose-6-phosphate glucosidase (EC 3.2.1.122); alpha glucosidase | 3 |
| | (EC 3.2.1.20); alpha-galactosidase (EC 3.2.1.22); 6-phospho-beta- | |
| | glucosidase (EC 3.2.1.86); alpha-glucuronidase (EC 3.2.1.139). | |
| 5 | chitosanase (EC 3.2.1.132); beta-mannosidase (EC 3.2.1.25); Cellulase | 3 |
| | (EC 3.2.1.4); glucan 1,3-beta-glucosidase (EC 3.2.1.58); licheninase | |
| | (EC 3.2.1.73); glucan endo-1,6-beta-glucosidase (EC 3.2.1.75); mannan | |
| | endo-1,4-beta-mannosidase (EC 3.2.1.78); 3 Endo-1,4-beta-xylanase | |
| | (EC 3.2.1.8); cellulose 1,4-beta-cellobiosidase (EC 3.2.1.91); endo-1,6- | |
| | beta-galactanase (EC 3.2.1.—); beta-1,3-mannanase (EC 3.2.1.—); | |
| | xyloglucan-specific endo-beta-1,4-glucanase (EC 3.2.1.151) | |
| 8 | chitosanase (EC 3.2.1.132); cellulase (EC 3.2.1.4); licheninase (EC | 1 |
| | 3.2.1.73); endo-1,4-beta-xylanase (EC 3.2.1.8); reducing-end-xylose | |
| | releasing exo-oligoxylanase (EC 3.2.1.156) | |
| 9 | endoglucanase (EC 3.2.1.4); cellobiohydrolase (EC 3.2.1.91); beta- | 1 |
| | glucosidase (EC 3.2.1.21) | |
| 10 | xylanase (EC 3.2.1.8); endo-1,3-beta-xylanase (EC 3.2.1.32) | 6 |
| 11 | xylanase (EC 3.2.1.8). | 1 |
| 12 | endoglucanase (EC 3.2.1.4); xyloglucan hydrolase (EC 3.2.1.151); | 1 |
| | beta-1,3-1,4-glucanase (EC 3.2.1.73); xyloglucan endotransglycosylase | |
| | (EC 2.4.1.207) | |
| 13 | apha-amylase (EC 3.2.1.1); pullulanase (EC 3.2.1.41); | 7 |
| | cyclomaltodextrin glucanotransferase (EC 2.4.1.19); | |
| | cyclornaltodextrinase (EC 3.2.1.54); trehalose-6-phosphate hydrolase | |
| | (EC 3.2.1.93); oligo-alpha-glucosiclase (EC 3.2.1.10); maltogenic | |
| | amylase (EC 3.2.1.133); neopullulanase (EC 3.2.1.135); alpha- | |
| | glucosidase (EC 3.2.1.20); maltotetraose-forming 3 alpha-amylase (EC | |
| | 3.2.1.60); isoamylase (EC 3.2.1.68); glucodextranase (EC 12.170); | |
| | maltohexaose-forming alphaamylase (EC 3.2.1.98); branching enzyme | |
| | (EC 2.4.1.18); trehalose synthase (EC 5.4.99.16); 4--glucanotransferase | |
| | (EC 2.4.1.25); maltopentaose-forming-amylase (EC 3.2.1.—); | |
| | amylosucrase (EC 2.4.1.4): sucrose phosphorylase (EC 2.4.1.7); malto- | |
| | oligosyltrehalose trehalohydrolase (EC 3.2.1.141); isomaltulose | |
| | synthase (EC 5.4.99.11). | |
| 16 | xyloglucan: xyloglucosyltransferase (EC 2.4.1.207); keratan-sulfate | 1 |
| | endo-1,4-beta-galactosidase (EC 3.2.1.103); Glucan endo-1,3-beta-D- | |
| | glucosidase (EC 3.2.1.39); endo-1,3(4)-beta-glucanase (EC 3.21.6); | |
| | Licheninase (EC 3.2.1.73): agarase (EC 3.2.1.81); betacarrageenase | |
| | (EC 3.2.1.83); xyioglucanase (EC 3.2.1.151) | |
| 18 | chitinase (EC 3.2.1.14); endo-beta-N-acetylglucosaminidase (EC | 6 |
| | 3.2.1.96); non-catalytic proteins: xylanase inhibitors; concanavalin B; | |
| | narbonin | |
| 19 | chitinase(EC 3.2.1.14). | 2 |
| 20 | beta-hexosaminidase (EC 3.2.1.52); lacto-N-biosidase (EC 3.2.1.140); - | 3 |
| | 1,6-N-acetylglucosaminidase) (EC 3.2.1.—) | |
| 25 | lysozyme(EC 3.2.1.17) | 1 |
| 26 | beta-mannanase (EC 3.2.1.78); beta-1,3-xylanase (EC 3.2.1.32) | 3 |
| 28 | polygalacturonase (EC 3.2.1.15); exo-polygalacturonase (EC 3.2.1.67); | 5 |
| | exo-polygalacturonosidase (EC 3.2.1.82); rhamnogalacturonase (EC | |
| | 3.2.1.—); endo-xylogalacturonan hydrolase (EC 3.2.1.—; | |
| | rhamnogalacturonan alpha-L-rhamnopyranohydrolase (EC 3.2.1.40) | |
| 29 | alpha-L-fucosidase (EC 3.2.1.51) | 3 |
| 30 | glucosylceramidase (EC 3.2.1.45); beta-1,6-glucanase (EC 3.2.1.75); | 2 |
| | beta-xylosidase (EC 3.2.1.37) | |
| 31 | alpha-glucosidase (EC 3.2.1.20): alpha-1,3-glucosidase (EC 3.2.1.84); | 3 |
| | sucrase-isomaltase (EC 3.2.1.48) (EC 3.2.1.10); alpha-xylosidase (EC | |
| | 3.2.1.—); alpha-glucan lyase (EC 4.2.2.13); isomaltosyltransferase_(EC | |
| | 2.4.1.—). | |
| 36 | alpha-galactosidase (EC 3.2.1.22); alpha-N-acetylgalactosaminidase | 2 |
| | (EC 3.2.1.49); stachyose synthase (EC 2.4.1.67); raffinose synthase | |
| | (EC 2.4.1.82) | |
| 38 | alpha-mannosidase (EC 3.2.1.24); alpha-mannosidase (EC 3.2.1.114) | 1 |
| 43 | beta-xylosidase (EC 3.2.1.37); beta-1,3-xylosidase (EC 3.2.1.—); alpha- | 8 |
| | L-arabinofuranosidase (EC 3.2.1.55); arabinanase (EC 3.2.1.99); | |
| | xylanase (EC 3.2.1.8); galactan 1,3-beta-galactosidase (EC 3.2.1.145) | |
| 48 | endoglucanase (EC 3.2.1.4); chitinase (EC 3.2.1.14); | 1 |
| | cellobiohydrolases some cellobiohydrolases of this family have been | |
| | reported to act from the reducing ends of cellulose (EC 3.2.1.—), while | |
| | others have been reported to operate from the non-reducing ends to | |
| | liberate cellobiose or cellotriose or cellotetraose (EC 3.2.1.—). This | |
| | family also contains endo-processive celtulases (EC 3.2.1.—), whose | |
| | activity is hard to distinguish from that of cellobiohydrolases. | |
| 51 | alpha-L-arabinofuranosidase (EC 3.2.1.55); endoglucanase (EC 3.2.1.4) | 1 |
| 65 | trehalase (EC 3.2.1.28); maltose phosphorylase (EC 2.4.1.8); trehalose | 4 |
| | phosphorylase (EC 2.4.1.64); kojibiose phosphorylase (EC 2.4.1.230) | |
| 67 | alpha-glucuronidase (EC 3.2.1.139); xylan alpha-I,2-glucuronosidase | 1 |
| | (EC_3.2.1.131) | |
| 73 | peptidoglycan hydrolases with endo-beta-N-acetylglucosam inidase | 1 |
| | (EC 3.2.1.—) specificity; there is only one, unconfirmed, report of beta- | |
| | i,4-N-acetylmuramoylhydrolase (EC 3.2.1.17) activity | |
| 77 | amylomaltase or 4-aipha-glucanotransferase (EC 2.4.1.25) | 1 |
| 85 | endo-beta-N-acetylglucosaminidase (EC 3.2.1.96) | 1 |
| 87 | mycodextranase (EC 3.2.1.61); alpha-1,3-glucanase (EC 3.2.1.59) | 3 |
| 88 | d-4,5 unsaturated beta-glucuronyl hydrolase (EC 3.2.1.—) | 4 |
| 94 | cellobiose phosphorylase (EC 2.4.1.20); cellodextrin phosphorylase | 5 |
| | (EC 2.4.1.49); chitobiose phosphorylase (EC 2.4.1.—); cyclic beta-1,2- | |
| | glucan synthase (EC 2.4.1.—) | |
| 95 | alpha-1,2-L-fucosidase (EC 3.2.1.63); alpha-L-fucosidase (EC 3.2.1.51) | 2 |
| 105 | unsaturated rhamnogalacturonyl hydrolase (EC 3.2.1.—) | 3 |
| 106 | alpha-L-rhamnosidase (EC 3.2.1.40) | 1 |
| 112 | lacto-N-biose phosphorylase or galacto-N-biose phosphorylase (EC | 3 |
| | 2.4.1.211) | |

In one embodiment, enzymes that degrade polysaccharides are used for the pretreatment of biomass and can include enzymes that degrade cellulose, namely, cellulases. Examples of some cellulases include endocellulases (EC 3.2.1.4) and exo-cellulases (EC 3.2.1.91), that hydrolyze beta-1,4-glucosidic bonds. Members of the GH5, GH9 and GH48 families can have both exo- and endo-cellulase activity.

In one embodiment, enzymes that degrade polysaccharides are used for the pretreatment of biomass and can include enzymes that have the ability to degrade hemicellulose, namely, hemicellulases. Hemicellulose can be a major component of plant biomass and can contain a mixture of pentoses and hexoses, for example, D-xylopyranose, L-arabinofuranose, D-mannopyranose, D-glucopyranose, D-galactopyranose, D-glucopyranosyluronic acid and other sugars. In one embodiment, predicted hemicellulases identified in C. phytofermentans that can be used in the pretreatment of biomass include enzymes active on the linear backbone of hemicellulose, for example, endo-beta-1,4-D-xylanase (EC 3.2.1.8), such as GH5, GH10, GH11, and GH43 family members; 1,4-beta-D-xyloside xylohydrolase (EC 3.2.1.37), such as GH30, GH43, and GH3 family members; and beta-mannanase (EC 3.2.1.78), such as GH26 family members.

In one embodiment, enzymes that degrade polysaccharides are used for the pretreatment of biomass and can include enzymes that have the ability to degrade pectin, namely, pectinases. In plant cell walls, the cross-linked cellulose network can be embedded in a matrix of pectins that can be covalently cross-linked to xyloglucans and certain structural proteins. Pectin can comprise homogalacturonan (HG) or rhamnogalacturonan (RH).

In one embodiment, pretreatment of biomass includes enzymes that can hydrolyze starch. C. phytofermentans can degrade starch and chitin (Warnick, T. A., Methe, B. A. & Leschine, S. B. Clostridium phytofermentans sp. nov., a cellulolytic mesophile from forest soil. Int. J. Syst. Evol. Microbiol. 52, 1155-1160 (2002); Leschine, S. B. in Handbook on Clostridia (ed Dürre, P.) (CRC Press, Boca Raton, 2005); Reguera, G. & Leschine, S. B. Chitin degradation by cellulolytic anaerobes and facultative aerobes from soils and sediments. FEMS Micro biol. Lett. 204, 367-374 (2001)). Enzymes that hydrolyze starch include alpha-amylase, glucoamylase, beta-amylase, exo-alpha-1,4-glucanase, and pullulanase.

In one embodiment, pretreatment of biomass comprises hydrolases that can include enzymes that hydrolyze chitin. Examples of enzymes that can hydrolyze chitin include GH18 and GH19 family members. In another embodiment, hydrolases can include enzymes that hydrolyze lichen, namely, lichenase, for example, GH16 family members.

In one embodiment, after pretreatment by any of the above methods the feedstock contains cellulose, hemicellulose, soluble oligomers, simple sugars, lignin, volatiles and ash. The parameters of the pretreatment can be changed to vary the concentration of the components of the pretreated feedstock. For example, in one embodiment a pretreatment is chosen so that the concentration of soluble oligomers is high and the concentration of lignin is low after pretreatment. Examples of parameters of the pretreatment include temperature, pressure, time, and pH.

In one embodiment, the parameters of the pretreatment are changed to vary the concentration of the components of the pretreated feedstock such that concentration of the components in the pretreated stock is optimal for fermentation with a microorganism such as a Clostridium biocatalyst. In one embodiment, the parameters of the pretreatment are changed to encourage the release of the components of a genetically modified feedstock such as enzymes stored within a vacuole to increase or complement the enzymes synthesized by Clostridium biocatalysts to produce optimal release of the fermentable components during hydrolysis and fermentation.

In one embodiment, the parameters of the pretreatment are changed such that concentration of accessible cellulose in the pretreated feedstock is 1%, 5%, 10%, 12%, 13%, 14%, 15%, 16%, 17%, 19%, 20%, 30%, 40% or 50%. In one embodiment, the parameters of the pretreatment are changed such that concentration of accessible cellulose in the pretreated feedstock is 5% to 30%. In one embodiment, the parameters of the pretreatment are changed such that concentration of accessible cellulose in the pretreated feedstock is 10% to 20%.

In one embodiment, the parameters of the pretreatment are changed such that concentration of hemicellulose in the pretreated feedstock is 1%, 5%, 10%, 12%, 13%, 14%, 15%, 16%, 17%, 19%, 20%, 21%, 22%, 23%, 24%, 25%, 26%, 27%, 28%, 29%, 30%, 40% or 50%. In one embodiment, the parameters of the pretreatment are changed such that concentration of hemicellulose in the pretreated feedstock is 5% to 40%. In one embodiment, the parameters of the pretreatment are changed such that concentration of hemicellulose in the pretreated feedstock is 10% to 30%.

In one embodiment, the parameters of the pretreatment are changed such that concentration of soluble oligomers in the pretreated feedstock is 1%, 10%, 15%, 20%, 25%, 30%, 35%, 40%, 45%, 50%, 55%, 60%, 65%, 70%, 75%, 80%, 85%, 90%, 95%, or 99%. Examples of soluble oligomers include, but are not limited to, cellobiose and xylobiose. In one embodiment, the parameters of the pretreatment are changed such that concentration of soluble oligomers in the pretreated feedstock is 30% to 90%. In one embodiment, the parameters of the pretreatment are changed such that concentration of soluble oligomers in the pretreated feedstock is 45% to 80%. In one embodiment, the parameters of the pretreatment are changed such that concentration of soluble oligomers in the pretreated feedstock is 45% to 80% and the soluble oligomers are primarily cellobiose and xylobiose.

In one embodiment, the parameters of the pretreatment are changed such that concentration of simple sugars in the pretreated feedstock is 1%, 5%, 10%, 12%, 13%, 14%, 15%, 16%, 17%, 19%, 20%, 30%, 40% or 50%. In one embodiment, the parameters of the pretreatment are changed such that concentration of simple sugars in the pretreated feedstock is 0% to 20%. In one embodiment, the parameters of the pretreatment are changed such that concentration of simple sugars in the pretreated feedstock is 0% to 5%. Examples of simple sugars include, but are not limited to, C5 and C6 monomers and dimers.

In one embodiment, the parameters of the pretreatment are changed such that concentration of lignin in the pretreated feedstock is 1%, 5%, 10%, 12%, 13%, 14%, 15%, 16%, 17%, 19%, 20%, 30%, 40% or 50%. In one embodiment, the parameters of the pretreatment are changed such that concentration of lignin in the pretreated feedstock is 0% to 20%. In one embodiment, the parameters of the pretreatment are changed such that concentration of lignin in the pretreated feedstock is 0% to 5%. In one embodiment, the parameters of the pretreatment are changed such that concentration of lignin in the pretreated feedstock is less than 1% to 2%. In one embodiment, the parameters of the pretreatment are changed such that the concentration of phenolics is minimized.

In one embodiment, the parameters of the pretreatment are changed such that concentration of furfural and low molecular weight lignin in the pretreated feedstock is less than 10%, 9%, 8%, 7%, 6%, 5%, 4%, 3%, 2%, or 1%. In one embodiment, the parameters of the pretreatment are changed such that concentration of furfural and low molecular weight lignin in the pretreated feedstock is less than 1% to 2%.

In one embodiment, the parameters of the pretreatment are changed such that concentration of accessible cellulose is 10% to 20%, the concentration of hemicellulose is 10% to 30%, the concentration of soluble oligomers is 45% to 80%, the concentration of simple sugars is 0% to 5%, and the concentration of lignin is 0% to 5% and the concentration of furfural and low molecular weight lignin in the pretreated feedstock is less than 1% to 2%.

In one embodiment, the parameters of the pretreatment are changed to obtain a high concentration of hemicellulose and a low concentration of lignin. In one embodiment, the parameters of the pretreatment are changed to obtain a high concentration of hemicellulose and a low concentration of lignin such that concentration of the components in the pretreated stock is optimal for fermentation with a microorganism such as a Clostridium biocatalyst.

### Co-Culture Fermentations

In one embodiment, a method of producing one or more fermentation end-products with a genetically modified microorganism adapted for decreased vitamin dependency, wherein the microorganism comprises a genetic modification that decreases vitamin dependency, further comprises a second microorganism. In one embodiment, the second microorganism is a yeast, a bacteria, or a non-yeast fungus, wherein the second microorganism is a different species than the genetically modified microorganism. In one embodiment, the second microorganism is genetically modified. In another embodiment, the second microorganism is not genetically modified. Examples of yeast that can be the second microorganism include, but are not limited to, species found in the genus Ascoidea, Brettanomyces, Candida, Cephaloascus, Coccidiascus, Dipodascus, Eremothecium, Galactomyces, Kluyveromyces, Pichia, Saccharomyces, Schizosaccharomyces, Sporopachydermia, Torulaspora, Yarrowia, or Zygosaccharomyces; for example, Ascoidea rebescens, Brettanomyces anomalus, Brettanomyces bruxellensis, Brettanomyces claussenii, Brettanomyces custersianus, Brettanomyces lambicus, Brettanomyces naardenensis, Brettanomyces nanus, Candida albicans, Candida ascalaphidarum, Candida amphixiae, Candida antarctica, Candida argentea, Candida atlantica, Candida atmosphaerica, Candida blattae, Candida carpophila, Candida cerambycidarum, Candida chauliodes, Candida corydali, Candida dosseyi, Candida dubliniensis, Candida ergatensis, Candida fructus, Candida glabrata, Candida fermentati, Candida guilliermondii, Candida haemulonii, Candida insectamens, Candida insectorum, Candida intermedia, Candida jeffresii, Candida kefyr, Candida krusei, Candida lusitaniae, Candida lyxosophila, Candida maltosa, Candida marina, Candida membranifaciens, Candida milleri, Candida oleophila, Candida oregonensis, Candida parapsilosis, Candida quercitrusa, Candida rugosa, Candida sake, Candida shehatea, Candida temnochilae, Candida tenuis, Candida tropicalis, Candida tsuchiyae, Candida sinolaborantium, Candida sojae, Candida subhashii, Candida viswanathii, Candida utilis, Cephaloascus fragrans, Coccidiascus legeri, Dypodascus albidus, Eremothecium cymbalariae, Galactomyces candidum, Galactomyces geotrichum, Kluyveromyces aestuarii, Kluyveromyces africanus, Kluyveromyces bacillisporus, Kluyveromyces blattae, Kluyveromyces dobzhanskii, Kluyveromyces hubeiensis, Kluyveromyces lactis, Kluyveromyces lodderae, Kluyveromyces marxianus, Kluyveromyces nonfermentans, Kluyveromyces piceae, Kluyveromyces sinensis, Kluyveromyces thermotolerans, Kluyveromyces waltii, Kluyveromyces wickerhamii, Kluyveromyces yarrowii, Pichia anomola, Pichia heedii, Pichia guilliermondii, Pichia kluyveri, Pichia membranifaciens, Pichia norvegensis, Pichia ohmeri, Pichia pastoris, Pichia subpelliculosa, Saccharomyces bayanus, Saccharomyces boulardii, Saccharomyces bulderi, Saccharomyces cariocanus, Saccharomyces cariocus, Saccharomyces cerevisiae, Saccharomyces chevalieri, Saccharomyces dairenensis, Saccharomyces ellipsoideus, Saccharomyces eubayanus, Saccharomyces exiguus, Saccharomyces florentinus, Saccharomyces kluyveri, Saccharomyces martiniae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, Saccharomyces zonatus, Schizosaccharomyces cryophilus, Schizosaccharomyces japonicus, Schizosaccharomyces octosporus, Schizosaccharomyces pombe, Sporopachydermia cereana, Sporopachydermia lactativora, Sporopachydermia quercuum,Torulaspora delbrueckii, Torulaspora franciscae, Torulaspora globosa, Torulaspora pretoriensis, Yarrowia lipolytica, Zygosaccharomyces bailii, Zygosaccharomyces bisporus, Zygosaccharomyces cidri, Zygosaccharomyces fermentati, Zygosaccharomyces florentinus, Zygosaccharomyces kombuchaensis, Zygosaccharomyces lentus, Zygosaccharomyces mellis, Zygosaccharomyces microellipsoides, Zygosaccharomyces mrakii, Zygosaccharomyces pseudorouxii, or Zygosaccharomyces rouxii. Examples of bacteria that can be the second microorganism include, but are not limited to, any bacterium found in the genus of Butyrivibrio, Ruminococcus, Eubacterium, Bacteroides, Acetivibrio, Caldibacillus, Acidothermus, Cellulomonas, Curtobacterium, Micromonospora, Actinoplanes, Streptomyces, Thermobifida, Thermomonospora, Microbispora, Fibrobacter, Sporocytophaga, Cytophaga, Flavobacterium, Achromobacter, Xanthomonas, Cellvibrio, Pseudomonas, Myxobacter, Escherichia, Klebsiella, Thermoanaerobacterium, Thermoanaerobacter, Geobacillus, Saccharococcus, Paenibacillus, Bacillus, Caldicellulosiruptor, Anaerocellum, Anoxybacillus, Zymomonas, Clostridium; for example, Butyrivibrio fibrisolvens, Ruminococcus flavefaciens, Ruminococcus succinogenes, Ruminococcus albus, Eubacterium cellulolyticum, Bacteroides cellulosolvens, Acetivibrio cellulolyticus, Acetivibrio cellulosolvens, Caldibacillus cellulovorans, Bacillus circulans, Acidothermus cellulolyticus, Cellulomonas cartae, Cellulomonas cellasea, Cellulomonas cellulans, Cellulomonas fimi, Cellulomonas flavigena, Cellulomonas gelida, Cellulomonas iranensis, Cellulomonas persica, Cellulomonas uda, Curtobacterium falcumfaciens, Micromonospora melonosporea, Actinoplanes aurantiaca, Streptomyces reticuli, Streptomyces alboguseolus, Streptomyces aureofaciens, Streptomyces cellulolyticus, Streptomyces flavogriseus, Streptomyces lividans, Streptomyces nitrosporeus, Streptomyces olivochromogenes, Streptomyces rochei, Streptomyces thermovulgaris, Streptomyces viridosporus, Thermobifida alba, Thermobifida fusca, Thermobifida cellulolytica, Thermomonospora curvata, Microbispora bispora, Fibrobacter succinogenes, Sporocytophaga myxococcoides, Cytophaga sp., Flavobacterium johnsoniae, Achromobacter piechaudii, Xanthomonas sp., Cellvibrio vulgaris, Cellvibrio fulvus, Cellvibrio gilvus, Cellvibrio mixtus, Pseudomonas fluorescens, Pseudomonas mendocina, Myxobacter sp. AL-1, Escherichia albertii, Escherichia blattae, Escherichia coli, Escherichia fergusonii, Escherichia hermannii, Escherichia vulneris, Klebsiella granulomatis, Klebsiella oxytoca, Klebsiella pneumonia, Klebsiella terrigena, Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brocki, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Caldicellulosiruptor acetigenus, Caldicellulosiruptor saccharolyticus, Caldicellulosiruptor kristjanssonii, Caldicellulosiruptor ow ensensis, Caldicellulosiruptor lactoaceticus, Anaerocellum thermophilum, Clostridium thermocellum, Clostridium cellulolyticum, Clostridium straminosolvens, Clostridium acetobutylicum, Clostridium aerotolerans, Clostridium beijerinckii, Clostridium bifermentans, Clostridium botulinum, Clostridium butyricum, Clostridium cadaveric, Clostridium chauvoei, Clostridium clostridioforme, Clostridium colicanis, Clostridium difficile, Clostridium fallax, Clostridium formicaceticum, Clostridium histolyticum, Clostridium innocuum, Clostridium ljungdahlii, Clostridium laramie, Clostridium lavalense, Clostridium novyi, Clostridium oedematiens, Clostridium paraputrificum, Clostridium perfringens, Clostridium phytofermentans (including NRRL B-50364 or NRRL B-50351), Clostridium piliforme, Clostridium ramosum, Clostridium scatologenes, Clostridium septicum, Clostridium sordellii, Clostridium sporogenes, Clostridium sp. Q.D (such as NRRL B-50361, NRRL B-50362, or NRRL B-50363), Clostridium tertium, Clostridium tetani, Clostridium tyrobutyricum, Clostridium thermobutyricum, Zymomonas mobilis or variants thereof (e.g. C. phytofermentans Q.12 or C. phytofermentans Q.13). In one embodiment, the second microorganism is Saccharomyces cerevisiae, C. thermocellum, C. acetobutylicum, C. cellovorans, or Zymomonas mobilis. In another embodiment, the second microorganism is Thermoanaerobacter pseudethanolicus, Thermoanaerobacter mathranii, Thermoanaerobacter italicus, Thermoanaerobacter brockii, T. acetoethylicus, Thermoanaerobacter ethanolicus, Thermoanaerobacter kivui, Thermoanaerobacter siderophilus, Thermoanaerobacter sulfuragignens, Thermoanaerobacter sulfurophilus, Thermoanaerobacter thermocopriae, Thermoanaerobacter thermohydrosulfuricus, Thermoanaerobacter uzonensis, or Thermoanaerobacter wiegelii. In another embodiment, the second microorganism is Eremothecium ashbyii, Ashbya gossypii, Candida flaeri, Candida famata, Candida ammoniagenes, Corynebacterium sp., Serratia marcescens, Fusarium oxysporum, Brevibacterium ammoniagenes, Rhodococcus rhodochrous, Brevibacterium sp., Arthrobacter sp., Candida boidinii, Bacillus sp., Gluconobacter sp., Arthrobacter sp., Saccharomyces sake, Alcaligenes faecalis, Agrobacterium sp., Sporoblomyces salmonicolor, Pseudomonas sp., Propionibacterium shermanii, Pseudomonas denitrificans, Geotrichum candidum, Flavobacterium sp., or Mortierella alpina.

### Recovery of Ethanol or Other Fermentation End-Products

In another aspect, methods are provided for the recovery of the fermentation end-products, such as an alcohol (e.g., ethanol, propanol, methanol, butanol, etc.) another biofuel or chemical product. In one embodiment, broth will be harvested at some point during of the fermentation, and fermentation end-product or products will be recovered. The broth with ethanol to be recovered will include both ethanol and impurities. The impurities include materials such as water, cell bodies, cellular debris, excess carbon substrate, excess nitrogen substrate, other remaining nutrients, non-ethanol metabolites, and other medium components or digested medium components. During the course of processing the broth, the broth can be heated and/or reacted with various reagents, resulting in additional impurities in the broth.

In one embodiment, the processing steps to recover ethanol frequently includes several separation steps, including, for example, distillation of a high concentration ethanol material from a less pure ethanol-containing material. In one embodiment, the high concentration ethanol material can be further concentrated to achieve very high concentration ethanol, such as 98% or 99% or 99.5% (wt.) or even higher. Other separation steps, such as filtration, centrifugation, extraction, adsorption, etc. can also be a part of some recovery processes for ethanol as a product or biofuel, or other biofuels or chemical products.

In one embodiment a process can be scaled to produce commercially useful biofuels. In another embodiment Clostridium biocatalysts are used to produce an alcohol, e.g., ethanol, butanol, propanol, methanol, or a fuel such as hydrocarbons hydrogen, methane, and hydroxy compounds. In another embodiment Clostridium biocatalysts are used to produce a carbonyl compound such as an aldehyde or ketone (e.g. acetone, formaldehyde, 1-propanal, etc.), an organic acid, a derivative of an organic acid such as an ester (e.g. wax ester, glyceride, etc.), 1,2-propanediol, 1,3-propanediol, lactic acid, formic acid, acetic acid, succinic acid, pyruvic acid, or an enzyme such as a cellulase, polysaccharase, lipases, protease, ligninase, and hemicellulase.

In one embodiment, a fed-batch fermentation for production of fermentation end-product is described. In another embodiment, a fed-batch fermentation for production of ethanol is described. Fed-batch culture is a kind of microbial process in which medium components, such as carbon substrate, nitrogen substrate, vitamins, minerals, growth factors, cofactors, etc. or biocatalysts (including, for example, fresh organisms, enzymes prepared by a Clostridium biocatalyst in a separate fermentation, enzymes prepared by other organisms, or a combination of these) are supplied to the fermentor during cultivation, but culture broth is not harvested at the same time and volume. To improve bioconversion from soluble and insoluble substrates, such as those that can be used in biofuels production, various feeding strategies can be utilized to improve yields and/or productivity. This technique can be used to achieve a high cell density within a given time. It can also be used to maintain a good supply of nutrients and substrates for the bioconversion process. It can also be used to achieve higher titer and productivity of desirable products that might otherwise be achieved more slowly or not at all.

In another embodiment, the feeding strategy balances the cell production rate and the rate of hydrolysis of the biomass feedstock with the production of ethanol. Sufficient medium components are added in quantities to achieved sustained cell production and hydrolysis of the biomass feedstock with production of ethanol. In one embodiment, sufficient carbon and nitrogen substrate are added in quantities to achieve sustained production of fresh cells and hydrolytic enzymes for conversion of polysaccharides into lower sugars as well as sustained conversion of the lower sugars into fresh cells and ethanol.

In another embodiment, the level of a medium component is maintained at a desired level by adding additional medium component as the component is consumed or taken up by the organism. Examples of medium components included, but are not limited to, carbon substrate, nitrogen substrate, vitamins (e.g., thiamine and nicotinic acid), minerals, growth factors, cofactors, and biocatalysts. The medium component can be added continuously or at regular or irregular intervals. In one embodiment, additional medium component is added prior to the complete depletion of the medium component in the medium. In one embodiment, complete depletion can effectively be used, for example to initiate different metabolic pathways, to simplify downstream operations, or for other reasons as well. In one embodiment, the medium component level is allowed to vary by about 10% around a midpoint, in one embodiment, it is allowed to vary by about 30% around a midpoint, and in one embodiment, it is allowed to vary by 60% or more around a midpoint. In one embodiment, the medium component level is maintained by allowing the medium component to be depleted to an appropriate level, followed by increasing the medium component level to another appropriate level. In one embodiment, a medium component, such as vitamin, is added at two different time points during fermentation process. For example, one-half of a total amount of vitamin is added at the beginning of fermentation and the other half is added at midpoint of fermentation.

Disclosed herein are media compositions that are supplemented with one or more additional media additives. The media additives can comprise, for example, carbon substrates, nitrogen substrates, vitamins, minerals, growth factors, cofactors, and biocatalysts. The media composition can be supplemented with one or more vitamins. The vitamins can comprise thiamine, an NAD+ precursor, vitamin B₆, vitamin B₉, or a combination thereof. In one embodiment, the vitamin is thiamine. In another embodiment the vitamin is a NAD+ precursor. An NAD+ precursor molecule can be nicotinic acid, nicotinamide or nicotinamide riboside.

In another embodiment, the vitamin is vitamin B₆ (e.g., pyridoxine, pyridoxine 5′-phosphate, pyridoxal, pyridoxal 5′-phosphate, pyridoxamine, and pyridoxamine 5′-phosphate). In another embodiment, the vitamin is pyridoxine. In another embodiment, the vitamin is vitamin B₉ (e.g., folic acid, folate, folinic acid). In one embodiment, the vitamin is folinic acid.

In another embodiment, the nitrogen level is maintained at a desired level by adding additional nitrogen-containing material as nitrogen is consumed or taken up by the organism. The nitrogen-containing material can be added continuously or at regular or irregular intervals. In one embodiment, additional nitrogen-containing material is added prior to the complete depletion of the nitrogen available in the medium. In one embodiment, complete depletion can effectively be used, for example to initiate different metabolic pathways, to simplify downstream operations, or for other reasons as well. In one embodiment, the nitrogen level (as measured by the grams of actual nitrogen in the nitrogen-containing material per liter of broth) is allowed to vary by about 10% around a midpoint, in some embodiments, it is allowed to vary by about 30% around a midpoint, and in some embodiments, it is allowed to vary by 60% or more around a midpoint. In one embodiment, the nitrogen level is maintained by allowing the nitrogen to be depleted to an appropriate level, followed by increasing the nitrogen level to another appropriate level. Useful nitrogen levels include levels of about 5 to about 10 g/L. In one embodiment levels of about 1 to about 12 g/L can also be usefully employed. In another embodiment levels, such as about 0.5, 0.1 g/L or even lower, and higher levels, such as about 20, 30 g/L or even higher are used. In another embodiment a useful nitrogen level is about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22 23, 24, 25, 26, 27, 28, 29 or 30 g/L. Such nitrogen levels can facilitate the production of fresh cells and of hydrolytic enzymes. Increasing the level of nitrogen can lead to higher levels of enzymes and/or greater production of cells, and result in higher productivity of desired products. Nitrogen can be supplied as a simple nitrogen-containing material, such as an ammonium compounds (e.g. ammonium sulfate, ammonium hydroxide, ammonia, ammonium nitrate, or any other compound or mixture containing an ammonium moiety), nitrate or nitrite compounds (e.g. potassium, sodium, ammonium, calcium, or other compound or mixture containing a nitrate or nitrite moiety), or as a more complex nitrogen-containing material, such as amino acids, proteins, hydrolyzed protein, hydrolyzed yeast, yeast extract, dried brewer's yeast, yeast hydrolysates, distillers' grains, soy protein, hydrolyzed soy protein, fermentation products, and processed or corn steep powder or unprocessed protein-rich vegetable or animal matter, including those derived from bean, seeds, soy, legumes, nuts, milk, pig, cattle, mammal, fish, as well as other parts of plants and other types of animals. Nitrogen-containing materials useful in various embodiments also include materials that contain a nitrogen-containing material, including, but not limited to mixtures of a simple or more complex nitrogen-containing material mixed with a carbon source, another nitrogen-containing material, or other nutrients or non-nutrients, and AFEX treated plant matter.

In another embodiment, the carbon level is maintained at a desired level by adding sugar compounds or material containing sugar compounds (“Sugar-Containing Material”) as sugar is consumed or taken up by the organism. The sugar-containing material can be added continuously or at regular or irregular intervals. In one embodiment, additional sugar-containing material is added prior to the complete depletion of the sugar compounds available in the medium. In one embodiment, complete depletion can effectively be used, for example to initiate different metabolic pathways, to simplify downstream operations, or for other reasons as well. In one embodiment, the carbon level (as measured by the grams of sugar present in the sugar-containing material per liter of broth) is allowed to vary by about 10% around a midpoint, in one embodiment, it is allowed to vary by about 30% around a midpoint, and in one embodiment, it is allowed to vary by 60% or more around a midpoint. In one embodiment, the carbon level is maintained by allowing the carbon to be depleted to an appropriate level, followed by increasing the carbon level to another appropriate level. In some embodiments, the carbon level can be maintained at a level of about 5 to about 120 g/L. However, levels of about 30 to about 100 g/L can also be usefully employed as well as levels of about 60 to about 80 g/L. In one embodiment, the carbon level is maintained at greater than 25 g/L for a portion of the culturing. In another embodiment, the carbon level is maintained at about 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L, 101 g/L, 102 g/L, 103 g/L, 104 g/L, 105 g/L, 106 g/L, 107 g/L, 108 g/L, 109 g/L, 110 g/L, 111 g/L, 112 g/L, 113 g/L, 114 g/L, 115 g/L, 116 g/L, 117 g/L, 118 g/L, 119 g/L, 120 g/L, 121 g/L, 122 g/L, 123 g/L, 124 g/L, 125 g/L, 126 g/L, 127 g/L, 128 g/L, 129 g/L, 130 g/L, 131 g/L, 132 g/L, 133 g/L, 134 g/L, 135 g/L, 136 g/L, 137 g/L, 138 g/L, 139 g/L, 140 g/L, 141 g/L, 142 g/L, 143 g/L, 144 g/L, 145 g/L, 146 g/L, 147 g/L, 148 g/L, 149 g/L, or 150 g/L.

The carbon substrate, like the nitrogen substrate, can be used for cell production and enzyme production, but unlike the nitrogen substrate, it serves as the raw material for ethanol. Frequently, more carbon substrate can lead to greater production of ethanol. In another embodiment, it can be advantageous to operate with the carbon level and nitrogen level related to each other for at least a portion of the fermentation time. In one embodiment, the ratio of carbon to nitrogen is maintained within a range of about 30:1 to about 10:1. In another embodiment, the ratio of carbon nitrogen is maintained from about 20:1 to about 10:1 or from about 15:1 to about 10:1. In another embodiment the ratio of carbon nitrogen is about 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or 1:1.

Maintaining the ratio of carbon and nitrogen ratio within particular ranges can result in benefits to the operation such as the rate of hydrolysis of carbon substrate, which depends on the amount of carbon substrate and the amount and activity of enzymes present, being balanced to the rate of ethanol production. Such balancing can be important, for example, due to the possibility of inhibition of cellular activity due to the presence of a high concentration of low molecular weight saccharides, and the need to maintain enzymatic hydrolytic activity throughout the period where longer chain saccharides are present and available for hydrolysis. Balancing the carbon to nitrogen ratio can, for example, facilitate the sustained production of these enzymes such as to replace those which have lost activity.

In another embodiment, the amount and/or timing of carbon, nitrogen, or other medium component addition can be related to measurements taken during the fermentation. For example, the amount of monosaccharides present, the amount of insoluble polysaccharide present, the polysaccharase activity, the amount of ethanol present, the amount of cellular material (for example, packed cell volume, dry cell weight, etc.) and/or the amount of nitrogen (for example, nitrate, nitrite, ammonia, urea, proteins, amino acids, etc.) present can be measured. The concentration of the particular species, the total amount of the species present in the fermentor, the number of hours the fermentation has been running, and the volume of the fermentor can be considered. In various embodiments, these measurements can be compared to each other and/or they can be compared to previous measurements of the same parameter previously taken from the same fermentation or another fermentation. Adjustments to the amount of a medium component can be accomplished such as by changing the flow rate of a stream containing that component or by changing the frequency of the additions for that component. In one embodiment, the amount of polysaccharide can be reduced when the monosaccharides level increases faster than the ethanol level increases. In another embodiment, the amount of polysaccharide can be increased when the amount or level of monosaccharides decreases while the ethanol production approximately remains steady. In another embodiment, the amount of nitrogen can be increased when the monosaccharides level increases faster than the viable cell level. The amount of polysaccharide can also be increased when the cell production increases faster than the ethanol production. In another embodiment the amount of nitrogen can be increased when the enzyme activity level decreases.

In another embodiment, different levels or complete depletion of a medium component can effectively be used, for example to initiate different metabolic pathways or to change the yield of the different products of the fermentation process. For instance, different levels or complete depletion of a medium component can effectively be used to increase the ethanol yield and productivity, to improve carbon utilization (e.g., g ethanol/g sugar fermented) and reduced acid production (e.g., g acid/g ethanol and g acid/g sugar fermented). In some embodiments, different levels or complete depletion of nitrogen can effectively be used to increase the ethanol yield and productivity, to improve carbon utilization (e.g., g ethanol/g sugar fermented) and reduced acid production (e.g., g acid/g ethanol and g acid/g sugar fermented). In some embodiments, different levels or complete depletion of carbon can effectively be used to increase the ethanol yield and productivity, to improve carbon utilization (e.g., g ethanol/g sugar fermented) and reduced acid production (e.g., g acid/g ethanol and g acid/g sugar fermented). In some embodiments, the ratio of carbon level to nitrogen level for at least a portion of the fermentation time can effectively be used to increase the ethanol yield and productivity, to improve carbon utilization (e.g., g ethanol/g sugar fermented) and reduced acid production (e.g., g acid/g ethanol and g acid/g sugar fermented).

In another embodiment, a fed batch operation can be employed, wherein medium components and/or fresh cells are added during the fermentation without removal of a portion of the broth for harvest prior to the end of the fermentation. In one embodiment a fed-batch process is based on feeding a growth limiting nutrient medium to a culture of microorganisms. In one embodiment the feed medium is highly concentrated to avoid dilution of the bioreactor. In another embodiment the controlled addition of the nutrient directly affects the growth rate of the culture and avoids overflow metabolism such as the formation of side metabolites. In one embodiment the growth limiting nutrient is a nitrogen source or a saccharide source.

In another embodiment, a modified fed batch operation can be employed wherein a portion of the broth is harvested at discrete times. Such a modified fed batch operation can be advantageously employed when, for example, very long fermentation cycles are employed. Under very long fermentation conditions, the volume of liquid inside the fermentor increases. In order to operate for very long periods, it can be advantageous to partially empty the fermentor, for example, when the volume is nearly full. A partial harvest of broth followed by supplementation with fresh medium ingredients, such as with a fed batch operation, can improve fermentor utilization and can facilitate higher plant throughputs due to a reduction in the time for tasks such as cleaning and sterilization of equipment. When the “partial harvest” type of operation is employed, the fermentation can be seeded with the broth that remains in the fermentor, or with fresh inoculum, or with a mixture of the two. In addition, broth can be recycled for use as fresh inoculum either alone or in combination with other fresh inoculum.

In one embodiment, a fed batch operation can be employed, wherein medium components and/or fresh cells are added during the fermentation when the hydrolytic activity of the broth has decreased. In one embodiment, medium components and/or fresh cells are added during the fermentation when the hydrolytic activity of the broth has decreased about 5%, 10%, 15%, 20%, 25%, 30%, 35%, 40%, 45%, 50%, 55%, 60%, 65%, 75%, 80%, 85%, 90%, 95%, or 100%.

While Clostridium biocatalysts can be used in long or short fermentation cycles, it can be particularly well-suited for long fermentation cycles and for use in fermentations with partial harvest, self-seeding, and broth recycle operations due to the anaerobic conditions of the fermentation, the presence of alcohol, the very fast growth rate of the strains compared to other Clostridia, and, in one embodiment, the use of a solid carbon substrate, whether or not resulting in low sugar concentrations in the broth.

In another embodiment, a fermentation to produce ethanol is performed by culturing a strain of a Clostridium biocatalyst in a medium having a high concentration of one or more carbon sources, and/or augmenting the culture with addition of fresh cells of Clostridium biocatalysts during the course of the fermentation. The resulting production of ethanol can be up to 1-fold, 2-fold, 3-fold, 4-fold, 5-fold, 6-fold, 7-fold, 8-fold, 9-fold, and in some cases up to 10-fold and higher in volumetric productivity than a batch process and achieve a carbon conversion efficiency approaching the theoretical maximum. The theoretical maximum can vary with the substrate and product. For example, the generally accepted maximum efficiency for conversion of glucose to ethanol is 0.51 g ethanol/g glucose. In one embodiment Clostridium biocatalysts can produce about 40-100% of a theoretical maximum yield of ethanol. In another embodiment, Clostridium biocatalysts can produce up to about 40% of the theoretical maximum yield of ethanol. In another embodiment, Clostridium biocatalysts can produce up to about 50% of the theoretical maximum yield of ethanol. In another embodiment, Clostridium biocatalysts can produce about 70% of the theoretical maximum yield of ethanol. In another embodiment, Clostridium biocatalysts can produce about 90% of the theoretical maximum yield of ethanol. In another embodiment, Clostridium biocatalysts can produce about 95% of the theoretical maximum yield of ethanol. In another embodiment, Clostridium biocatalysts can produce about 95% of the theoretical maximum yield of ethanol. In another embodiment, Clostridium biocatalysts can produce about 99% of the theoretical maximum yield of ethanol. In another embodiment, Clostridium biocatalysts can produce about 100% of the theoretical maximum yield of ethanol. In one embodiment Clostridium biocatalysts can produce up to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.99%, or 100% of a theoretical maximum yield of ethanol.

Clostridium biocatalyst cells used for the seed inoculum or for cell augmentation can be prepared or treated in ways that relate to their ability to produce enzymes useful for hydrolyzing the components of the production medium. For example, in one embodiment, the cells can produce useful enzymes after they are transferred to the production medium or production fermentor. In another embodiment, Clostridium biocatalyst cells can have already produced useful enzymes prior to transfer to the production medium or the production fermentor. In another embodiment, Clostridium biocatalyst cells can be ready to produce useful enzymes once transferred to the production medium or the production fermentor, or Clostridium biocatalyst cells can have some combination of these enzyme production characteristics. In one embodiment, the seed can be grown initially in a medium containing a simple sugar source, such as corn syrup or dried brewer's yeast, and then transitioned to the production medium carbon source prior to transfer to the production medium. In another embodiment, the seed is grown on a combination of simple sugars and production medium carbon source prior to transfer to the production medium. In another embodiment, the seed is grown on the production medium carbon source from the start. In another embodiment, the seed is grown on one production medium carbon source and then transitioned to another production medium carbon source prior to transfer to the production medium. In another embodiment, the seed is grown on a combination of production medium carbon sources prior to transfer to the production medium. In another embodiment, the seed is grown on a carbon source that favors production of hydrolytic enzymes with activity toward the components of the production medium.

In another embodiment, a fermentation to produce ethanol is performed by culturing a strain of a Clostridium biocatalyst microorganism and adding fresh medium components and fresh Clostridium biocatalyst cells while the cells in the fermentor are growing. Medium components, such as carbon, nitrogen, and combinations of these, can be added as disclosed herein, as well as other nutrients, including vitamins, factors, cofactors, enzymes, minerals, salts, and such, sufficient to maintain an effective level of these nutrients in the medium. The medium and Clostridium biocatalyst can be added simultaneously, or one at a time. In another embodiment, fresh Clostridium biocatalyst cells can be added when hydrolytic enzyme activity decreases, especially when the activity of those hydrolytic enzymes that are more sensitive to the presence of alcohol decreases. After the addition of fresh Clostridium biocatalyst cells, a nitrogen feed or a combination of nitrogen and carbon feed and/or other medium components can be fed, prolonging the enzymatic production or other activity of the cells. In another embodiment, the cells can be added with sufficient carbon and nitrogen to prolong the enzymatic production or other activity of the cells sufficiently until the next addition of fresh cells. In another embodiment, fresh Clostridium biocatalyst cells can be added when both the nitrogen level and carbon level present in the fermentor increase. In another embodiment, Clostridium biocatalyst cells can be added when the viable cell count decreases, especially when the nitrogen level is relatively stable or increasing. In another embodiment, fresh cells can be added when a significant portion of the viable cells are in the process of sporulation, or have sporulated. Appropriate times for adding fresh cells can be when the portion of cells in the process of sporulation or have sporulated is about 2% to about 100%, about 10% to about 75%, about 20% to about 50%, or about 25% to about 30% of the cells are in the process of sporulation or have sporulated.

### Medium Compositions

In various embodiments, particular medium components can have beneficial effects on the performance of the fermentation, such as increasing the titer of desired products, or increasing the rate that the desired products are produced. Specific compounds can be supplied as a specific, pure ingredient, such as a particular amino acid, or it can be supplied as a component of a more complex ingredient, such as using a microbial, plant or animal product as a medium ingredient to provide a particular amino acid, promoter, cofactor, or other beneficial compound. In some cases, the particular compound supplied in the medium ingredient can be combined with other compounds by the organism resulting in a fermentation-beneficial compound. One example of this situation would be where a medium ingredient provides a specific amino acid which the organism uses to make an enzyme beneficial to the fermentation. Other examples can include medium components that are used to generate growth or product promoters, etc. In such cases, it can be possible to obtain a fermentation-beneficial result by supplementing the enzyme, promoter, growth factor, etc. or by adding the precursor. In some situations, the specific mechanism whereby the medium component benefits the fermentation is not known, only that a beneficial result is achieved.

In one embodiment, beneficial fermentation results can be achieved by adding yeast extract. The addition of the yeast extract can result in increased ethanol titer in batch fermentation, improved productivity and reduced production of side products such as organic acids. In one embodiment beneficial results with yeast extract can be achieved at usage levels of about 0.5 to about 50 g/L, about 5 to about 30 g/L, or about 10 to about 30 g/L.

In one embodiment, beneficial fermentation results can be achieved by adding corn steep powder to the fermentation. The addition of the corn steep powder can result in increased ethanol titer in batch fermentation, improved productivity and reduced production of side products such as organic acids. In another embodiment beneficial results with corn steep powder can be achieved at usage levels of about 3 to about 20 g/L, about 5 to about 15 g/L, or about 8 to about 12 g/L. In another embodiment beneficial results with steep powder can be achieved at a level of about 3 g/L, 3.1 g/L, 3.2 g/L, 3.3 g/L, 3.4 g/L, 3.5 g/L, 3.6 g/L, 3.7 g/L, 3.8 g/L, 3.9 g/L, 4 g/L, 4.1 g/L, 4.2 g/L, 4.3 g/L, 4.4 g/L, 4.5 g/L, 4.6 g/L, 4.7 g/L, 4.8 g/L, 4.9 g/L, 5 g/L, 5.1 g/L, 5.2 g/L, 5.3 g/L, 5.4 g/L, 5.5 g/L, 5.6 g/L, 5.7 g/L, 5.8 g/L, 5.9 g/L, 6 g/L, 6.1 g/L, 6.2 g/L, 6.3 g/L, 6.4 g/L, 6.5 g/L, 6.6 g/L, 6.7 g/L, 6.8 g/L, 6.9 g/L, 7 g/L, 7.1 g/L, 7.2 g/L, 7.3 g/L, 7.4 g/L, 7.5 g/L, 7.6 g/L, 7.7 g/L, 7.8 g/L, 7.9 g/L, 8 g/L, 8.1 g/L, 8.2 g/L, 8.3 g/L, 8.4 g/L, 8.5 g/L, 8.6 g/L, 8.7 g/L, 8.8 g/L, 8.9 g/L, 9 g/L, 9.1 g/L, 9.2 g/L, 9.3 g/L, 9.4 g/L, 9.5 g/L, 9.6 g/L, 9.7 g/L, 9.8 g/L, 9.9 g/L, 10 g/L, 10.1 g/L, 10.2 g/L, 10.3 g/L, 10.4 g/L, 10.5 g/L, 10.6 g/L, 10.7 g/L, 10.8 g/L, 10.9 g/L, 11 g/L, 11.1 g/L, 11.2 g/L, 11.3 g/L, 11.4 g/L, 11.5 g/L, 11.6 g/L, 11.7 g/L, 11.8 g/L, 11.9 g/L, 12 g/L, 12.1 g/L, 12.2 g/L, 12.3 g/L, 12.4 g/L, 12.5 g/L, 12.6 g/L, 12.7 g/L, 12.8 g/L, 12.9 g/L, 13 g/L, 13.1 g/L, 13.2 g/L, 13.3 g/L, 13.4 g/L, 13.5 g/L, 13.6 g/L, 13.7 g/L, 13.8 g/L, 13.9 g/L, 14 g/L, 14.1 g/L, 14.2 g/L, 14.3 g/L, 14.4 g/L, 14.5 g/L, 14.6 g/L, 14.7 g/L, 14.8 g/L, 14.9 g/L, 15 g/L, 15.1 g/L, 15.2 g/L, 15.3 g/L, 15.4 g/L, 15.5 g/L, 15.6 g/L, 15.7 g/L, 15.8 g/L, 15.9 g/L, 16 g/L, 16.1 g/L, 16.2 g/L, 16.3 g/L, 16.4 g/L, 16.5 g/L, 16.6 g/L, 16.7 g/L, 16.8 g/L, 16.9 g/L, 17 g/L, 17.1 g/L, 17.2 g/L, 17.3 g/L, 17.4 g/L, 17.5 g/L, 17.6 g/L, 17.7 g/L, 17.8 g/L, 17.9 g/L, 18 g/L, 18.1 g/L, 18.2 g/L, 18.3 g/L, 18.4 g/L, 18.5 g/L, 18.6 g/L, 18.7 g/L, 18.8 g/L, 18.9 g/L, 19 g/L, 19.1 g/L, 19.2 g/L, 19.3 g/L, 19.4 g/L, 19.5 g/L, 19.6 g/L, 19.7 g/L, 19.8 g/L, 19.9 g/L, or 20 g/L.

In one embodiment corn steep powder can also be fed throughout the course of the entire fermentation or a portion of the fermentation, continuously or delivered at intervals. In another embodiment usage levels include maintaining a nitrogen concentration of about 0.05 g/L to about 3 g/L (as nitrogen), where at least a portion of the nitrogen is supplied from corn steep powder; about 0.3 g/L to 1.3 g/L; or about 0.4 g/L to about 0.9 g/L. In another embodiment the nitrogen level is about 0.05 g/L, 0.06 g/L, 0.07 g/L, 0.08 g/L, 0.09 g/L, 0.1 g/L, 0.11 g/L, 0.12 g/L, 0.13 g/L, 0.14 g/L, 0.15 g/L, 0.16 g/L, 0.17 g/L, 0.18 g/L, 0.19 g/L, 0.2 g/L, 0.21 g/L, 0.22 g/L, 0.23 g/L, 0.24 g/L, 0.25 g/L, 0.26 g/L, 0.27 g/L, 0.28 g/L, 0.29 g/L, 0.3 g/L, 0.31 g/L, 0.32 g/L, 0.33 g/L, 0.34 g/L, 0.35 g/L, 0.36 g/L, 0.37 g/L, 0.38 g/L, 0.39 g/L, 0.4 g/L, 0.41 g/L, 0.42 g/L, 0.43 g/L, 0.44 g/L, 0.45 g/L, 0.46 g/L, 0.47 g/L, 0.48 g/L, 0.49 g/L, 0.5 g/L, 0.51 g/L, 0.52 g/L, 0.53 g/L, 0.54 g/L, 0.55 g/L, 0.56 g/L, 0.57 g/L, 0.58 g/L, 0.59 g/L, 0.6 g/L, 0.61 g/L, 0.62 g/L, 0.63 g/L, 0.64 g/L, 0.65 g/L, 0.66 g/L, 0.67 g/L, 0.68 g/L, 0.69 g/L, 0.7 g/L, 0.71 g/L, 0.72 g/L, 0.73 g/L, 0.74 g/L, 0.75 g/L, 0.76 g/L, 0.77 g/L, 0.78 g/L, 0.79 g/L, 0.8 g/L, 0.81 g/L, 0.82 g/L, 0.83 g/L, 0.84 g/L, 0.85 g/L, 0.86 g/L, 0.87 g/L, 0.88 g/L, 0.89 g/L, 0.9 g/L, 0.91 g/L, 0.92 g/L, 0.93 g/L, 0.94 g/L, 0.95 g/L, 0.96 g/L, 0.97 g/L, 0.98 g/L, 0.99 g/L, 1 g/L, 1.01 g/L, 1.02 g/L, 1.03 g/L, 1.04 g/L, 1.05 g/L, 1.06 g/L, 1.07 g/L, 1.08 g/L, 1.09 g/L, 1.1 g/L, 1.11 g/L, 1.12 g/L, 1.13 g/L, 1.14 g/L, 1.15 g/L, 1.16 g/L, 1.17 g/L, 1.18 g/L, 1.19 g/L, 1.2 g/L, 1.21 g/L, 1.22 g/L, 1.23 g/L, 1.24 g/L, 1.25 g/L, 1.26 g/L, 1.27 g/L, 1.28 g/L, 1.29 g/L, 1.3 g/L, 1.31 g/L, 1.32 g/L, 1.33 g/L, 1.34 g/L, 1.35 g/L, 1.36 g/L, 1.37 g/L, 1.38 g/L, 1.39 g/L, 1.4 g/L, 1.41 g/L, 1.42 g/L, 1.43 g/L, 1.44 g/L, 1.45 g/L, 1.46 g/L, 1.47 g/L, 1.48 g/L, 1.49 g/L, 1.5 g/L, 1.51 g/L, 1.52 g/L, 1.53 g/L, 1.54 g/L, 1.55 g/L, 1.56 g/L, 1.57 g/L, 1.58 g/L, 1.59 g/L, 1.6 g/L, 1.61 g/L, 1.62 g/L, 1.63 g/L, 1.64 g/L, 1.65 g/L, 1.66 g/L, 1.67 g/L, 1.68 g/L, 1.69 g/L, 1.7 g/L, 1.71 g/L, 1.72 g/L, 1.73 g/L, 1.74 g/L, 1.75 g/L, 1.76 g/L, 1.77 g/L, 1.78 g/L, 1.79 g/L, 1.8 g/L, 1.81 g/L, 1.82 g/L, 1.83 g/L, 1.84 g/L, 1.85 g/L, 1.86 g/L, 1.87 g/L, 1.88 g/L, 1.89 g/L, 1.9 g/L, 1.91 g/L, 1.92 g/L, 1.93 g/L, 1.94 g/L, 1.95 g/L, 1.96 g/L, 1.97 g/L, 1.98 g/L, 1.99 g/L, 2 g/L, 2.01 g/L, 2.02 g/L, 2.03 g/L, 2.04 g/L, 2.05 g/L, 2.06 g/L, 2.07 g/L, 2.08 g/L, 2.09 g/L, 2.1 g/L, 2.11 g/L, 2.12 g/L, 2.13 g/L, 2.14 g/L, 2.15 g/L, 2.16 g/L, 2.17 g/L, 2.18 g/L, 2.19 g/L, 2.2 g/L, 2.21 g/L, 2.22 g/L, 2.23 g/L, 2.24 g/L, 2.25 g/L, 2.26 g/L, 2.27 g/L, 2.28 g/L, 2.29 g/L, 2.3 g/L, 2.31 g/L, 2.32 g/L, 2.33 g/L, 2.34 g/L, 2.35 g/L, 2.36 g/L, 2.37 g/L, 2.38 g/L, 2.39 g/L, 2.4 g/L, 2.41 g/L, 2.42 g/L, 2.43 g/L, 2.44 g/L, 2.45 g/L, 2.46 g/L, 2.47 g/L, 2.48 g/L, 2.49 g/L, 2.5 g/L, 2.51 g/L, 2.52 g/L, 2.53 g/L, 2.54 g/L, 2.55 g/L, 2.56 g/L, 2.57 g/L, 2.58 g/L, 2.59 g/L, 2.6 g/L, 2.61 g/L, 2.62 g/L, 2.63 g/L, 2.64 g/L, 2.65 g/L, 2.66 g/L, 2.67 g/L, 2.68 g/L, 2.69 g/L, 2.7 g/L, 2.71 g/L, 2.72 g/L, 2.73-85—WSGR No. 37836-739.201 g/L, 2.74 g/L, 2.75 g/L, 2.76 g/L, 2.77 g/L, 2.78 g/L, 2.79 g/L, 2.8 g/L, 2.81 g/L, 2.82 g/L, 2.83 g/L, 2.84 g/L, 2.85 g/L, 2.86 g/L, 2.87 g/L, 2.88 g/L, 2.89 g/L, 2.9 g/L, 2.91 g/L, 2.92 g/L, 2.93 g/L, 2.94 g/L, 2.95 g/L, 2.96 g/L, 2.97 g/L, 2.98 g/L, 2.99 g/L, or 3 g/L.

In another embodiment, other related products can be used, such as dried brewer's yeast (DBY) or spent brewers yeast, corn steep liquor or corn steep powder. When corn steep liquor is used, the usage rate would be approximately the same as for corn steep powder on a solids basis. In another embodiment, the corn steep powder (or solids or liquor) is added in relation to the amount of carbon substrate that is present or that will be added. When added in this way, beneficial amounts of corn steep powder (or liquor or solids) can include about 1:1 to about 1:6 g/g carbon, about 1:1 to about 1:5 g/g carbon, or about 1:2 to about 1:4 g/g carbon. In another embodiment ratios as high as about 1.5:1 g/g carbon or about 3:1 g/g carbon or as low as about 1:8 g/g carbon or about 1:10 g/g carbon are used. In another embodiment the ratio is 2:1 g/g carbon, 1.9:1 g/g carbon, 1.8:1 g/g carbon, 1.7:1 g/g carbon, 1.6:1 g/g carbon, 1.5:1 g/g carbon, 1.4:1 g/g carbon, 1.3:1 g/g carbon, 1.2:1 g/g carbon, 1.1:1 g/g carbon, 1:1 g/g carbon, 1:1.1 g/g carbon, 1:1.2 g/g carbon, 1:1.3 g/g carbon, 1:1.4 g/g carbon, 1:1.5 g/g carbon, 1:1.6 g/g carbon, 1:1.7 g/g carbon, 1:1.8 g/g carbon, 1:1.9 g/g carbon, 1:2 g/g carbon, 1:2.1 g/g carbon, 1:2.2 g/g carbon, 1:2.3 g/g carbon, 1:2.4 g/g carbon, 1:2.5 g/g carbon, 1:2.6 g/g carbon, 1:2.7 g/g carbon, 1:2.8 g/g carbon, 1:2.9 g/g carbon, 1:3 g/g carbon, 1:3.1 g/g carbon, 1:3.2 g/g carbon, 1:3.3 g/g carbon, 1:3.4 g/g carbon, 1:3.5 g/g carbon, 1:3.6 g/g carbon, 1:3.7 g/g carbon, 1:3.8 g/g carbon, 1:3.9 g/g carbon, 1:4 g/g carbon, 1:4.1 g/g carbon, 1:4.2 g/g carbon, 1:4.3 g/g carbon, 1:4.4 g/g carbon, 1:4.5 g/g carbon, 1:4.6 g/g carbon, 1:4.7 g/g carbon, 1:4.8 g/g carbon, 1:4.9 g/g carbon, 1:5 g/g carbon, 1:5.1 g/g carbon, 1:5.2 g/g carbon, 1:5.3 g/g carbon, 1:5.4 g/g carbon, 1:5.5 g/g carbon, 1:5.6 g/g carbon, 1:5.7 g/g carbon, 1:5.8 g/g carbon, 1:5.9 g/g carbon, 1:6 g/g carbon, 1:6.1 g/g carbon, 1:6.2 g/g carbon, 1:6.3 g/g carbon, 1:6.4 g/g carbon, 1:6.5 g/g carbon, 1:6.6 g/g carbon, 1:6.7 g/g carbon, 1:6.8 g/g carbon, 1:6.9 g/g carbon, 1:7 g/g carbon, 1:7.1 g/g carbon, 1:7.2 g/g carbon, 1:7.3 g/g carbon, 1:7.4 g/g carbon, 1:7.5 g/g carbon, 1:7.6 g/g carbon, 1:7.7 g/g carbon, 1:7.8 g/g carbon, 1:7.9 g/g carbon, 1:8 g/g carbon, 1:8.1 g/g carbon, 1:8.2 g/g carbon, 1:8.3 g/g carbon, 1:8.4 g/g carbon, 1:8.5 g/g carbon, 1:8.6 g/g carbon, 1:8.7 g/g carbon, 1:8.8 g/g carbon, 1:8.9 g/g carbon, 1:9 g/g carbon, 1:9.1 g/g carbon, 1:9.2 g/g carbon, 1:9.3 g/g carbon, 1:9.4 g/g carbon, 1:9.5 g/g carbon, 1:9.6 g/g carbon, 1:9.7 g/g carbon, 1:9.8 g/g carbon, 1:9.9 g/g carbon, or 1:10 g/g carbon.

In one embodiment, beneficial fermentation results can be achieved by adding corn steep powder in combination with yeast extract to the fermentation. Beneficial results with corn steep powder in combination with yeast extract can be achieved at corn steep powder usage levels of about 3 to about 20 g/L, about 5 to about 15 g/L, or about 8 to about 12 g/L and yeast extract usage levels of about 3 to 50 g/L, about 5 to about 30 g/L, or about 10 to about 30 g/L. The corn steep powder and yeast extract can also be fed throughout the course of the entire fermentation or a portion of the fermentation, continuously or delivered at intervals. Both compounds provide a source of thiamine.

In one embodiment, the beneficial compounds from corn steep powder and/or yeast extract, such as glycine, histidine, isoleucine, proline, or phytate as well as combinations of these compounds can be added to the medium or broth to obtain a beneficial effect.

In one embodiment, Clostridium biocatalysts are fermented with a substrate at about pH 5-8.5 In one embodiment a Clostridium biocatalysts are fermented at pH of about 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5.

Vitamin Supplementation of Media

Thiamin

Vitamins are essential in supplying or enabling synthesis of specific co-factors that facilitate enzymatic reactions in most bacterial organisms. Thiamine (or thiamin, vitamin B₁) is a sulfur-containing, water-soluble vitamin that is essential to the survival of living organisms. It can be synthesized by some bacteria, some protozoans, fungi and plants. Because it is used for enzyme function in many different metabolic pathways, deficiencies can lead to severe mental and physiological symptoms, such as beriberi. While thiamine deficiencies have been extensively studied in organisms, little is known about the effect of thiamine supplementation above minimal levels for growth, especially in bacteria. The few studies that have been performed have not focused on one vitamin alone but the supplementation of a combination of media components. See, e.g., Mironova, R. et al. 2005 Mol. Microbiol. 55(6):1801-1811; Ghosh, A. C. et al. 2005 Engin. Life Sci. 5(4): 378-382; and Takeno, K. et al. 2001 Appl. Microbiol. Biotech. 56(1-2):280-285.

C. phytofermentans is a microorganism capable of hydrolyzing and fermenting both hexose (C6) and pentose (C5) polysaccharides to ethanol as its primary product. In a typical fermentation reaction, C. phytofermentans produces a mixture of ethanol and small amounts of lactic, formic and acetic acid from one or more of these polysaccharides. Because it does not express pyruvate dehydrogenase, almost all pyruvate from glycolysis is converted to acetyl-CoA through PFOR.

In an effort to determine if conversion of pyruvate to acetyl-CoA could be increased, C. phytofermentans fermentation medium was supplemented with B₁ vitamin, thiamine, as a means to improve ethanol yield and to decrease lactic acid production. The mechanism of improvement is thought to be associated with Cphy gene 3558 which encodes pyruvate-ferredoxin oxidoreductase (PFOR), one of the most highly expressed genes in the C. phytofermentans genome. In C. phytofermentans, thiamine, as TPP, plays a role in the conversion of pyruvate to Acetyl Co-A using enzymatic reaction catalyzed by PFOR as part of its active site. PFOR contains thiamine diphosphate and [4Fe-4S] clusters. This enzyme is one of four 2-oxoacid oxidoreductases that are differentiated by their abilities to oxidatively decarboxylate different 2-oxoacids and form their CoA derivatives.) Thus, a deficiency of TPP reduces or effectively blocks the production of Acetyl-CoA by pyruvate-ferredoxin oxidoreductase (PFOR). The glycolytic flux then results in an accumulation of pyruvate because acetyl-CoA cannot be synthesized quickly enough, driving the dehydrogenation of pyruvate through L-lactate dehydrogenase to lactic acid, a process that does not require TPP as a coenzyme (FIG. 5).

To improve the efficiency of Acetyl-CoA synthesis and further reduce lactic acid production, thiamine was supplemented to fermentation media in an effort to determine if increased levels of TPP would result in increased pyruvate ferredoxin oxidoreductase activity. In these experiments, the addition of thiamine at a final concentration of 5 mg/L results in a significant increase in ethanol titer and overall yield corresponding to a significant decrease in the production of lactic acid.

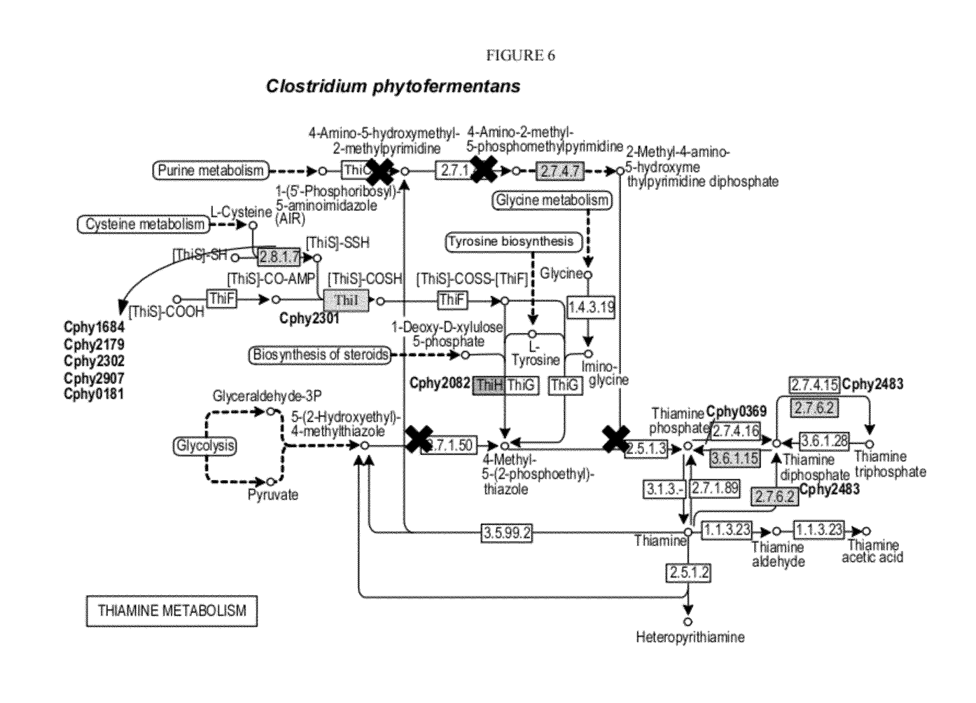

The increase in ethanol titer with thiamine supplementation indicates that C. phytofermentans is dependent on exogenous sources of thiamine. A study of the annotated genome sequence of C. phytofermentans suggests that it lacks one or more genes necessary to synthesize thiamine. The Kegg diagram for the synthesis of thiamine and thiamine derivatives (FIG. 6) has been annotated to show where these genes (FIG. 7) can be supplemented or substituted from other Clostridium species, such as C. cellulolyticum to complete one or more thiamine metabolic pathways in C. phytofermentans. C. phytofermentans does synthesize and express pyrophosphate, and when supplemented with thiamine, produces TPP.

Unlike a number of Clostridium species, C. phytofermentans degrades plant material which, in a natural environment, would have adequate thiamine for its existence in a natural environment. However, to reach high yields of ethanol and reduce lactic acid (lactate) synthesis during fermentation, increased levels of this cofactor can be useful. FIG. 4 illustrates several of the pathways that can occur depending on the conversion of pyruvate in the presence of certain enzymes and their cofactors.

In one embodiment a microorganism, genetically modified microorganism (e.g., a genetically modified microorganism adapted for decreaced vitamin dependency), or mutant thereof ferments and hydrolyzes a biomass material in the presence of thiamine to produce a first fermentation end-product and a second fermentation end-product. The microorganism can be a Clostridium biocatalyst. In another embodiment, the Clostridium biocatalyst is a Clostridium phytofermentans strain. In another embodiment, the Clostridium biocatalyst is Clostridium Q.D. A Clostridium phytofermentans strain can be, for example, Clostridium phytofermentans Q.8, Clostridium phytofermentans Q.33, or Clostridium phytofermentans Q.32. In one embodiment the amount of the first fermentation end-product produced in the presence of thiamine is higher than the amount of the first fermentation end-product produced in the absence of thiamine. In another embodiment the amount of the first fermentation end-product produced in the presence of thiamine is about 1-300% or higher than the amount of the first fermentation end-product produced in the absence of thiamine, such as about 1-300%, 1-250%, 1-200%, 1-150%, 1-100%, 1-90%, 1-80%, 1-70%, 1-60%, 1-50%, 1-40%, 1-30%, 1-20%, 1-10%, 10-300%, 10-250%, 10-200%, 10-150%, 10-100%, 10-90%, 10-80%, 10-70%, 10-60%, 10-50%, 10-40%, 10-30%, 10-20%, 20-300%, 20-250%, 20-200%, 20-150%, 20-100%, 20-90%, 20-80%, 20-70%, 20-60%, 20-50%, 20-40%, 20-30%, 30-300%, 30-250%, 30-200%, 30-150%, 30-100%, 30-90%, 30-80%, 30-70%, 30-60%, 30-50%, 30-40%, 40-300%, 40-250%, 40-200%, 40-150%, 40-100%, 40-90%, 40-80%, 40-70%, 40-60%, 40-50%, 50-300%, 50-250%, 50-200%, 50-150%, 50-100%, 50-90%, 50-80%, 50-70%, 50-60%, 60-300%, 60-250%, 60-200%, 60-150%, 60-100%, 60-90%, 60-80%, 60-70%, 70-300%, 70-250%, 70-200%, 70-150%, 70-100%, 70-90%, 70-80%, 80-300%, 80-250%, 80-200%, 80-150%, 80-100%, 80-90%, 90-300%, 90-250%, 90-200%, 90-150%, 90-100%, 100-300%, 100-250%, 100-200%, 100-150%, 150-300%, 150-250%, 150-200%, 200-300%, 200-250%, 250-300%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 220%, 240%, 260%, 280%, 300%, or higher. In one embodiment, the first fermentation end-product is an alcohol. In one embodiment, the alcohol is ethanol.

In one embodiment a microorganism, a genetically modified microorganism (e.g., a genetically modified microorganism adapted for decreaced vitamin dependency), or mutant thereof ferments and hydrolyzes a biomass material in the presence of thiamine to produce a first fermentation end-product and a second fermentation end-product. In one embodiment, the amount of the second fermentation end-product produced in the presence of thiamine is about 1-100% lower than the amount of second fermentation end-product produced in the absence of thiamine, such as about 1-100%, 1-90%, 1-80%, 1-70%, 1-60%, 1-50%, 1-40%, 1-30%, 1-20%, 1-10%, 10-100%, 10-90%, 10-80%, 10-70%, 10-60%, 10-50%, 10-40%, 10-30%, 10-20%, 20-100%, 20-90%, 20-80%, 20-70%, 20-60%, 20-50%, 20-40%, 20-30%, 30-100%, 30-90%, 30-80%, 30-70%, 30-60%, 30-50%, 30-40%, 40-100%, 40-90%, 40-80%, 40-70%, 40-60%, 40-50%, 50-100%, 50-90%, 50-80%, 50-70%, 50-60%, 60-100%, 60-90%, 60-80%, 60-70%, 70-100%, 70-90%, 70-80%, 80-100%, 80-90%, 90-100%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% lower. In one embodiment the second fermentation end-product is lactate or lactic acid.

A biomass can be a source of thiamine. In one embodiment the biomass is selected for fermentation and hydrolysis based in part on its thiamine content. In another embodiment the thiamine is from exogenous source used to supplement a media in which the microorganism (e.g., a Clostridium biocatalyst; e.g., Clostridium Q.D. or a Clostridium phytofermentans strain; e.g., Clostridium phytofermentans Q.8, Clostridium phytofermentans Q.33, or Clostridium phytofermentans Q.32), a genetically modified microorganism (e.g., a genetically modified microorganism adapted for decreaced vitamin dependency), or mutant thereof ferments and hydrolyzes a biomass material. In one embodiment the thiamine is added to an initial concentration of about 1-90 mg/L, 1-80 mg/L, 1-70 mg/L, 1-60 mg/L, 1-50 mg/L, 1-40 mg/L, 1-30 mg/L, 1-20 mg/L, 1-10 mg/L, 10-90 mg/L, 10-80 mg/L, 10-70 mg/L, 10-60 mg/L, 10-50 mg/L, 10-40 mg/L, 10-30 mg/L, 10-20 mg/L, 20-90 mg/L, 20-80 mg/L, 20-70 mg/L, 20-60 mg/L, 20-50 mg/L, 20-40 mg/L, 20-30 mg/L, 30-90 mg/L, 30-80 mg/L, 30-70 mg/L, 30-60 mg/L, 30-50 mg/L, 30-40 mg/L, 40-90 mg/L, 40-80 mg/L, 40-70 mg/L, 40-60 mg/L, 40-50 mg/L, 50-90 mg/L, 50-80 mg/L, 50-70 mg/L, 50-60 mg/L, 60-90 mg/L, 60-80 mg/L, 60-70 mg/L, 70-90 mg/L, 70-80 mg/L, 80-90 mg/L. In one embodiment, thiamine is added to about 3 mg to 8 mg/liter, about 1 mg to 10 mg/liter, about 2 mg to 20 mg/liter, or about 3 mg to 30 mg/liter, about 4 mg to 40 mg/liter, about 5 mg to 50 mg/liter, about 10 mg to 100 mg/liter, about 20 mg to 80 mg/liter, about 50 mg to 150 mg/liter, about 100 mg to 200 mg/liter, about 150 to 300 mg/liter, about 200 mg to about 400 mg/liter, about 300 to 600 mg/liter, about 400 to 800 mg/liter, or 500 to 1 g/liter, (such as about 1-1000 mg/L, 1 mg/L, 2 mg/L, 3 mg/L, 4 mg/L, 5 mg/L, 6 mg/L, 7 mg/L, 8 mg/L, 9 mg/L, 10 mg/L, 11 mg/L, 12 mg/L, 13 mg/L, 14 mg/L, 15 mg/L, 16 mg/L, 17 mg/L, 18 mg/L, 19 mg/L, 20 mg/L, 21 mg/L, 22 mg/L, 23 mg/L, 24 mg/L, 25 mg/L, 26 mg/L, 27 mg/L, 28 mg/L, 29 mg/L, 30 mg/L, 31 mg/L, 32 mg/L, 33 mg/L, 34 mg/L, 35 mg/L, 36 mg/L, 37 mg/L, 38 mg/L, 39 mg/L, 40 mg/L, 41 mg/L, 42 mg/L, 43 mg/L, 44 mg/L, 45 mg/L, 46 mg/L, 47 mg/L, 48 mg/L, 49 mg/L, 50 mg/L, 51 mg/L, 52 mg/L, 53 mg/L, 54 mg/L, 55 mg/L, 56 mg/L, 57 mg/L, 58 mg/L, 59 mg/L, 60 mg/L, 61 mg/L, 62 mg/L, 63 mg/L, 64 mg/L, 65 mg/L, 66 mg/L, 67 mg/L, 68 mg/L, 69 mg/L, 70 mg/L, 71 mg/L, 72 mg/L, 73 mg/L, 74 mg/L, 75 mg/L, 76 mg/L, 77 mg/L, 78 mg/L, 79 mg/L, 80 mg/L, 81 mg/L, 82 mg/L, 83 mg/L, 84 mg/L, 85 mg/L, 86 mg/L, 87 mg/L, 88 mg/L, 89 mg/L, 90 mg/L, 91 mg/L, 92 mg/L, 93 mg/L, 94 mg/L, 95 mg/L, 96 mg/L, 97 mg/L, 98 mg/L, 99 mg/L, 100 mg/L, 101 mg/L, 102 mg/L, 103 mg/L, 104 mg/L, 105 mg/L, 106 mg/L, 107 mg/L, 108 mg/L, 109 mg/L, 110 mg/L, 111 mg/L, 112 mg/L, 113 mg/L, 114 mg/L, 115 mg/L, 116 mg/L, 117 mg/L, 118 mg/L, 119 mg/L, 120 mg/L, 121 mg/L, 122 mg/L, 123 mg/L, 124 mg/L, 125 mg/L, 126 mg/L, 127 mg/L, 128 mg/L, 129 mg/L, 130 mg/L, 131 mg/L, 132 mg/L, 133 mg/L, 134 mg/L, 135 mg/L, 136 mg/L, 137 mg/L, 138 mg/L, 139 mg/L, 140 mg/L, 141 mg/L, 142 mg/L, 143 mg/L, 144 mg/L, 145 mg/L, 146 mg/L, 147 mg/L, 148 mg/L, 149 mg/L, 150 mg/L, 151 mg/L, 152 mg/L, 153 mg/L, 154 mg/L, 155 mg/L, 156 mg/L, 157 mg/L, 158 mg/L, 159 mg/L, 160 mg/L, 161 mg/L, 162 mg/L, 163 mg/L, 164 mg/L, 165 mg/L, 166 mg/L, 167 mg/L, 168 mg/L, 169 mg/L, 170 mg/L, 171 mg/L, 172 mg/L, 173 mg/L, 174 mg/L, 175 mg/L, 176 mg/L, 177 mg/L, 178 mg/L, 179 mg/L, 180 mg/L, 181 mg/L, 182 mg/L, 183 mg/L, 184 mg/L, 185 mg/L, 186 mg/L, 187 mg/L, 188 mg/L, 189 mg/L, 190 mg/L, 191 mg/L, 192 mg/L, 193 mg/L, 194 mg/L, 195 mg/L, 196 mg/L, 197 mg/L, 198 mg/L, 199 mg/L, 200 mg/L, 210 mg/L, 220 mg/L, 230 mg/L, 240 mg/L, 250 mg/L, 260 mg/L, 270 mg/L, 280 mg/L, 290 mg/L, 300 mg/L, 310 mg/L, 320 mg/L, 330 mg/L, 340 mg/L, 350 mg/L, 360 mg/L, 370 mg/L, 380 mg/L, 390 mg/L, 400 mg/L, 410 mg/L, 420 mg/L, 430 mg/L, 440 mg/L, 450 mg/L, 460 mg/L, 470 mg/L, 480 mg/L, 490 mg/L, 500 mg/L, 510 mg/L, 520 mg/L, 530 mg/L, 540 mg/L, 550 mg/L, 560 mg/L, 570 mg/L, 580 mg/L, 590 mg/L, 500 mg/L, 610 mg/L, 620 mg/L, 630 mg/L, 640 mg/L, 650 mg/L, 660 mg/L, 670 mg/L, 680 mg/L, 690 mg/L, 700 mg/L, 710 mg/L, 720 mg/L, 730 mg/L, 740 mg/L, 750 mg/L, 760 mg/L, 770 mg/L, 780 mg/L, 790 mg/L, 800 mg/L, 810 mg/L, 820 mg/L, 830 mg/L, 840 mg/L, 850 mg/L, 860 mg/L, 870 mg/L, 880 mg/L, 890 mg/L, 900 mg/L, 910 mg/L, 920 mg/L, 930 mg/L, 940 mg/L, 950 mg/L, 960 mg/L, 970 mg/L, 980 mg/L, 990 mg/L, or 1000 mg/L).

Nicotinic Acid, Vitamin B₆, Vitamin B₉

Nicotinic acid (also known as niacin, nicotinate, vitamin B3, and vitamin PP) is another essential vitamin. The corresponding amide is called nicotinamide or niacinamide. These vitamins are not directly interconvertable; however, both nicotinate and nicotinamide are precursors in the synthesis of redox pairs NAD+/NADH (nicotinamide adenine dinucleotide) and NADP+/NADPH (nicotinamide adenine dinucleotide phosphate). NAD+/NADH is an essential component of glycolysis whereby glucose is broken down into pyruvate. As discussed supra, under anaerobic conditions, pyruvate can be fermented into products such as lactate (lactic acid), CO₂, ethanol, acetate (acetic acid), formic acid, L-proprionate, butanoate, and other compounds depending on the enzymes present and energy requirements (FIG. 4). NAD+/NADH can be synthesized through two metabolic pathways: a salvage pathway and a de novo pathway. In the salvage pathway, NAD+/NADH can be synthesized from external sources of precursor compounds (e.g., nicotinic acid, nicotinamide, nicotinamide riboside, etc.). In the de novo pathway, NAD+NADH is synthesized from quinolinate produced during the metabolism of amino acids (e.g., tryptophan, aspartate, etc.). Many microorganisms do not naturally express all of the enzymes used for de novo synthesis of NAD+/NADH from amino acids. For example, Clostridium phytofermentans is missing two key enzymes (dashed boxes, FIG. 16) and is therefore considered an NAD+auxotroph.

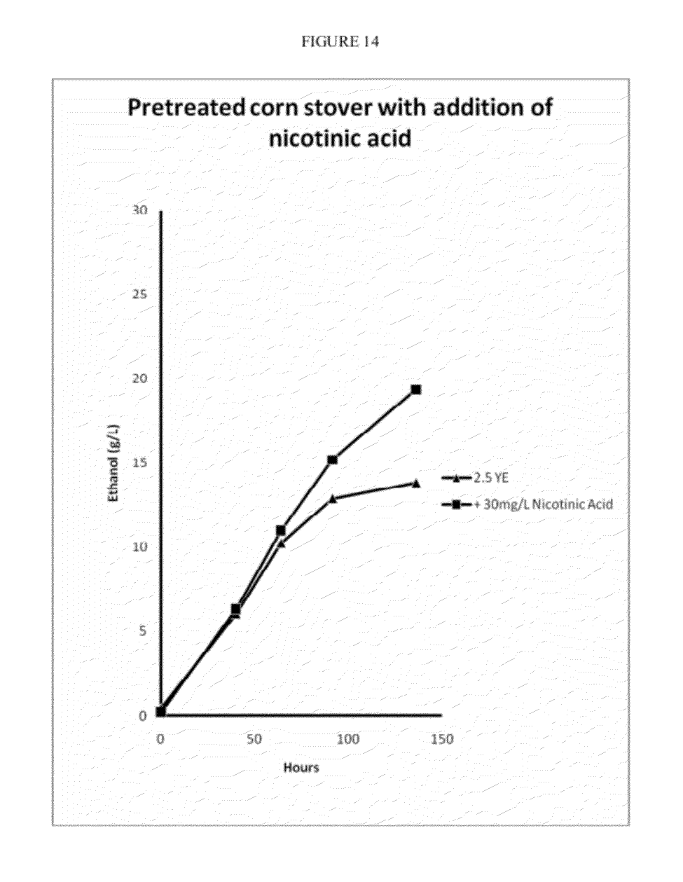

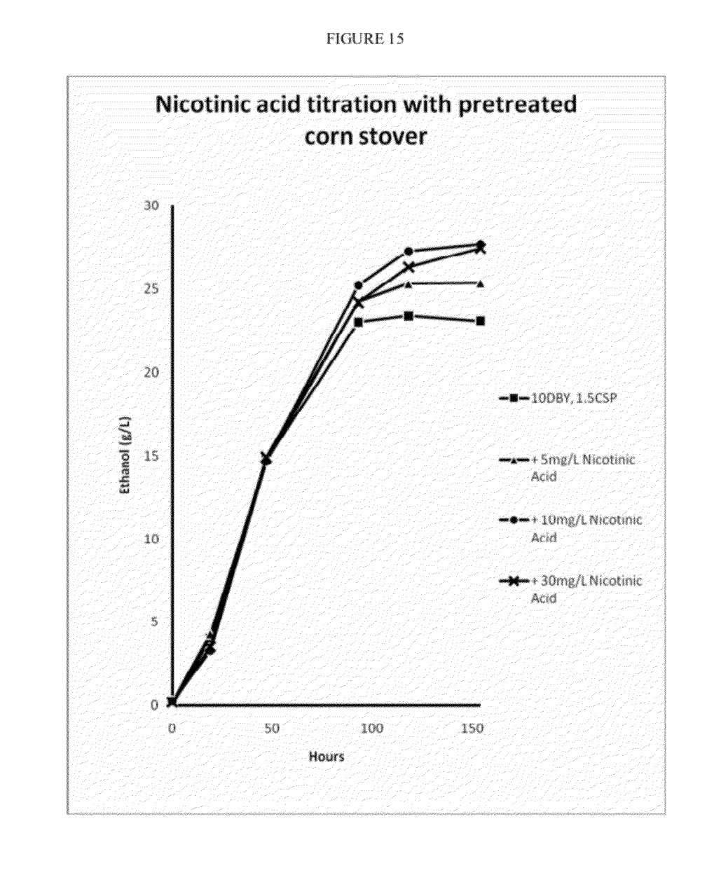

In order to determine whether increased levels of NAD+/NADH precursor molecules would increase ethanol yields, fermentation media was supplemented with nicotinic acid. In this experiment, fermentation of pretreated corn stover by Clostridium phytofermentans in media supplemented to 30 mg/L nicotinic acid produced a higher yields of ethanol compared to fermentation without nicotinic acid supplementation (FIG. 14). This experiment was performed using fermentation media containing yeast extract. Titration experiments were also performed whereby increasing amounts of nicotinic acid were added to a basal medium. Fermentation of pretreated corn stover by Clostridium phytofermentans produced greater amounts of ethanol with increased levels of nicotinic acid (FIG. 15) with a saturation effect seen above supplementation to 10 mg/L nicotinic acid.

There are six interconvertable forms of vitamin B₆: pyridoxine, pyridoxine 5′-phosphate, pyridoxal, pyridoxal 5′-phosphate, pyridoxamine, and pyridoxamine 5′-phosphate. Of these, pyridoxal 5′-phosphate (PLP) is the metabolically active form. PLP can be a coenzyme involved in numerous cellular processes such as amino acid metabolism (e.g., amino acid catabolism, amino acid interconversion, etc.); gluconeogenesis, both through amino acid catabolism and as a coenzyme for glycogen phosphorylase; lipid metabolism (e.g., biosynthesis of sphingolipids); and gene expression, both through conversion of homocysteine into cysteine and through interactions with transcription factors.

As discussed supra, PLP can be produced from the conversion of other forms of vitamin B₆. PLP can also be synthesized from ribulose 5-phosphate, which is a product of the pentose phosphate pathway, and glyceraldehydes 3-phosphate, which is a product of the glycolysis pathway (see FIG. 41). Some microorganisms lack the enzymes to synthesize PLP from ribulose 5-phosphate and glyceraldehydes 3-phosphate, and therefore require external sources of vitamin B₆.

Vitamin B₉, also known as folic acid, and folate are non-biologically active vitamins that can be converted into tetrahydrofolate (THF) and other derivatives. THF can act as coenzymes in many cellular processes such as the metabolism of amino acids and nucleic acids. THF can be considered to be particularly important for rapidly dividing cells (e.g., bacteria, yeast, etc.).

THF can be synthesized from folate: folate can be first converted to 7,8-dihydrofolate (DHF), which can be converted to THF through the action of an enzyme dihydrofolate reductase. THF can also be synthesized from 5-formyl-tetrahydrofolate, also called folinic acid. Folinic acid can be considered a vitamin B₉ substitute. As used herein, the term vitamin B₉ encompasses both vitamin B₉ and vitamin B₉ substitutes; for example, the term vitamin B₉ encompasses folic acid, folate, and folinic acid. Some microorganisms lack the enzyme dihydrofolate reductase and therefore require an external source of folinic acid for growth.

In one embodiment, provided herein are media compositions and methods of producing one or more fermentation end-product using the media compositions with a microorganism, a genetically modified microorganism (e.g., a genetically modified microorganism adapted for decreaced vitamin dependency), or a variant thereof, wherein the media compositions are supplemented with an NAD+ precursor, a vitamin B₆, a vitamin B₉, or a combination thereof. The NAD+ precursor molecule can be, for example, nicotinic acid, nicotinamide, nicotinamide riboside, or a combination thereof. In some embodiments, the media is supplemented with nicotinic acid. The vitamin B₆ can be pyridoxine, pyridoxine 5′-phosphate, pyridoxal, pyridoxal 5′-phosphate, pyridoxamine, pyridoxamine 5′-phosphate, or a combination thereof. In one embodiment, the vitamin B₆ is pyridoxine. The vitamin B₉ can be folic acid, folate, folinic acid, or a combination thereof. In one embodiment, the vitamin B₉ is folinic acid. In one embodiment, the media composition comprises thiamine. Media compositions disclosed herein can be supplemented to, for example, between about 1 mg/L and 90 mg/L of the NAD+ precursor, the vitamin B₆, the vitamin B₉, and/or thiamine; for example about 1-90 mg/L, 1-80 mg/L, 1-70 mg/L, 1-60 mg/L, 1-50 mg/L, 1-40 mg/L, 1-30 mg/L, 1-20 mg/L, 1-10 mg/L, 10-90 mg/L, 10-80 mg/L, 10-70 mg/L, 10-60 mg/L, 10-50 mg/L, 10-40 mg/L, 10-30 mg/L, 10-20 mg/L, 20-90 mg/L, 20-80 mg/L, 20-70 mg/L, 20-60 mg/L, 20-50 mg/L, 20-40 mg/L, 20-30 mg/L, 30-90 mg/L, 30-80 mg/L, 30-70 mg/L, 30-60 mg/L, 30-50 mg/L, 30-40 mg/L, 40-90 mg/L, 40-80 mg/L, 40-70 mg/L, 40-60 mg/L, 40-50 mg/L, 50-90 mg/L, 50-80 mg/L, 50-70 mg/L, 50-60 mg/L, 60-90 mg/L, 60-80 mg/L, 60-70 mg/L, 70-90 mg/L, 70-80 mg/L, or 80-90 mg/L. Media compositions can be supplemented to about 1 mg/L, 2 mg/L, 3 mg/L, 4 mg/L, 5 mg/L, 6 mg/L, 7 mg/L, 8 mg/L, 9 mg/L, 10 mg/L, 11 mg/L, 12 mg/L, 13 mg/L, 14 mg/L, 15 mg/L, 16 mg/L, 17 mg/L, 18 mg/L, 19 mg/L, 20 mg/L, 21 mg/L, 22 mg/L, 23 mg/L, 24 mg/L, 25 mg/L, 26 mg/L, 27 mg/L, 28 mg/L, 29 mg/L, 30 mg/L, 31 mg/L, 32 mg/L, 33 mg/L, 34 mg/L, 35 mg/L, 36 mg/L, 37 mg/L, 38 mg/L, 39 mg/L, 40 mg/L, 41 mg/L, 42 mg/L, 43 mg/L, 44 mg/L, 45 mg/L, 46 mg/L, 47 mg/L, 48 mg/L, 49 mg/L, 50 mg/L, 51 mg/L, 52 mg/L, 53 mg/L, 54 mg/L, 55 mg/L, 56 mg/L, 57 mg/L, 58 mg/L, 59 mg/L, 60 mg/L, 61 mg/L, 62 mg/L, 63 mg/L, 64 mg/L, 65 mg/L, 66 mg/L, 67 mg/L, 68 mg/L, 69 mg/L, 70 mg/L, 71 mg/L, 72 mg/L, 73 mg/L, 74 mg/L, 75 mg/L, 76 mg/L, 77 mg/L, 78 mg/L, 79 mg/L, 80 mg/L, 81 mg/L, 82 mg/L, 83 mg/L, 84 mg/L, 85 mg/L, 86 mg/L, 87 mg/L, 88 mg/L, 89 mg/L, 90 mg/L, or more of NAD+ precursor, the vitamin B₆, the vitamin B₉, and/or thiamine.

In some embodiments, a yield of one or more fermentation end-products can be increased by between about 1% and 300% or more when using a media composition supplemented with an NAD+ precursor, a vitamin B₆, ae vitamin B₉, and/or thiamine. For example, the yield of at least one fermentation end-product can be increased by about 1-300%, 1-250%, 1-200%, 1-150%, 1-100%, 1-90%, 1-80%, 1-70%, 1-60%, 1-50%, 1-40%, 1-30%, 1-20%, 1-10%, 10-300%, 10-250%, 10-200%, 10-150%, 10-100%, 10-90%, 10-80%, 10-70%, 10-60%, 10-50%, 10-40%, 10-30%, 10-20%, 20-300%, 20-250%, 20-200%, 20-150%, 20-100%, 20-90%, 20-80%, 20-70%, 20-60%, 20-50%, 20-40%, 20-30%, 30-300%, 30-250%, 30-200%, 30-150%, 30-100%, 30-90%, 30-80%, 30-70%, 30-60%, 30-50%, 30-40%, 40-300%, 40-250%, 40-200%, 40-150%, 40-100%, 40-90%, 40-80%, 40-70%, 40-60%, 40-50%, 50-300%, 50-250%, 50-200%, 50-150%, 50-100%, 50-90%, 50-80%, 50-70%, 50-60%, 60-300%, 60-250%, 60-200%, 60-150%, 60-100%, 60-90%, 60-80%, 60-70%, 70-300%, 70-250%, 70-200%, 70-150%, 70-100%, 70-90%, 70-80%, 80-300%, 80-250%, 80-200%, 80-150%, 80-100%, 80-90%, 90-300%, 90-250%, 90-200%, 90-150%, 90-100%, 100-300%, 100-250%, 100-200%, 100-150%, 150-300%, 150-250%, 150-200%, 200-300%, 200-250%, 250-300%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 220%, 240%, 260%, 280%, 300%, or higher in comparison to a media composition not supplemented with NAD+ precursor, the vitamin B₆, the vitamin B₉, and/or thiamine.

Vitamin Secretion by Microorganisms

In one embodiment, a fermentation media comprising a first microorganism can be supplemented with one or more vitamins, wherein the vitamins are secreted by a second microorganism. In one embodiment, the one or more vitamins comprise vitamin A (e.g., retinol), vitamin Bₚ (e.g., choline), vitamin B₁ (e.g., thiamin), vitamin B₂ (e.g., riboflavin) vitamin B₃ (e.g., niacin, nicotinic acid, nicotinamide, nicotinamide riboside), vitamin B₅ (e.g., pantothenic acid), vitamin B₆ (e.g., pyridoxine, pyridoxamine, pyridoxal), vitamin B₇ (e.g., biotin), vitamin B₉ (e.g., folic acid, folate, folinic acid), vitamin B₁₂ (e.g., cobalamin), vitamin C (e.g., ascorbic acid), vitamin D (e.g., ergocalciferol, cholecalciferol), vitamin E (e.g., tocopherol), vitamin K (e.g., naphthoquinoids), or a combination thereof. In one embodiment, the vitamins comprise thiamine, an NAD+precursor (e.g., nicotinic acid, nicotinamide, nicotinamide riboside, or a combination thereof), a vitamin B₆ (e.g., pyridoxine, pyridoxine 5′-phosphate, pyridoxal, pyridoxal 5′-phosphate, pyridoxamine, and pyridoxamine 5′-phosphate, or a combination thereof), a vitamin B₉ (e.g., folate, folic acid, folinic acid, or a combination thereof), or a combination thereof. In one embodiment, the first microorganism is a Clostridium strain. In another embodiment, the first microorganism is Clostridium phytofermentans, Clostridium sp Q.D, or a variant thereof. In one embodiment, the first microorganism is not genetically modified. In one embodiment, the first microorganism is genetically modified. In one embodiment, the first microorganism is a genetically modified microorganism adapted for decreased vitamin dependency. In one embodiment, at least one of the vitamins is at a concentration that is less than the minimum nutritional requirements for growth of an unmodified microorganism of the same species as the genetically modified microorganism adapted for decreased vitamin dependency. In another embodiment, at least one of the vitamins is at a concentration that is greater than the minimum nutritional requirements for growth of the first microorganism. In one embodiment, the second microorganism is genetically modified. In another embodiment, the second microorganism is not genetically modified. In one embodiment, the second microorganism is a yeast, a bacteria, or a non-yeast fungus, wherein the second microorganism is a different species from the first microorganism. In another embodiment, the second microorganism is Eremothecium ashbyii, Ashbya gossypii, Candida flaeri, Candida famata, Candida ammoniagenes, Corynebacterium sp., Serratia marcescens, Fusarium oxysporum, Brevibacterium ammoniagenes, Rhodococcus rhodochrous, Brevibacterium sp., Arthrobacter sp., Candida boidinii, Bacillus sp., Gluconobacter sp., Arthrobacter sp., Saccharomyces sake, Alcaligenes faecalis, Agrobacterium sp., Sporoblomyces salmonicolor, Pseudomonas sp., Propionibacterium shermanii, Pseudomonas denitrificans, Geotrichum candidum, Flavobacterium sp., or Mortierella alpina.

In one embodiment, a fermentation media comprising a first microorganism can be supplemented with one or more vitamins, wherein the vitamins are secreted by a second microorganism, wherein the first microorganism produces a greater yield of one or more fermentation end-products than can be produced in a fermentation media that is not so supplemented. In one embodiment, the yield is between about 1 and about 300% greater; for example, about 1-300%, 1-250%, 1-200%, 1-150%, 1-100%, 1-90%, 1-80%, 1-70%, 1-60%, 1-50%, 1-40%, 1-30%, 1-20%, 1-10%, 10-300%, 10-250%, 10-200%, 10-150%, 10-100%, 10-90%, 10-80%, 10-70%, 10-60%, 10-50%, 10-40%, 10-30%, 10-20%, 20-300%, 20-250%, 20-200%, 20-150%, 20-100%, 20-90%, 20-80%, 20-70%, 20-60%, 20-50%, 20-40%, 20-30%, 30-300%, 30-250%, 30-200%, 30-150%, 30-100%, 30-90%, 30-80%, 30-70%, 30-60%, 30-50%, 30-40%, 40-300%, 40-250%, 40-200%, 40-150%, 40-100%, 40-90%, 40-80%, 40-70%, 40-60%, 40-50%, 50-300%, 50-250%, 50-200%, 50-150%, 50-100%, 50-90%, 50-80%, 50-70%, 50-60%, 60-300%, 60-250%, 60-200%, 60-150%, 60-100%, 60-90%, 60-80%, 60-70%, 70-300%, 70-250%, 70-200%, 70-150%, 70-100%, 70-90%, 70-80%, 80-300%, 80-250%, 80-200%, 80-150%, 80-100%, 80-90%, 90-300%, 90-250%, 90-200%, 90-150%, 90-100%, 100-300%, 100-250%, 100-200%, 100-150%, 150-300%, 150-250%, 150-200%, 200-300%, 200-250%, 250-300%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 220%, 240%, 260%, 280%, 300%, or greater in comparison to a yield in a fermentation media that is not so supplemented.

Synergy

In one aspect, a synergistic effect on fermentation yields or results can be obtained with media compositions comprising two or more media additives or co-factors (e.g., thiamine; an NAD+ precursor molecule such as nicotinic acid, nicotinamide, or nicotinamide riboside; a vitamin B₆ such as pyridoxine, pyridoxine 5′-phosphate, pyridoxal, pyridoxal 5′-phosphate, pyridoxamine, or pyridoxamine 5′-phosphate; or a vitamin B₉ such as folic acid, folate, or folinic acid). In one embodiment, synergy between two or more media additives enables lower levels of the two or more media additives to be used in order to achieve the same fermentation yield or result. In another embodiment, synergy between two or more media additives results in fermentation yield increases that are greater than can be achieved when utilizing the two or more media components separately. In another embodiment, synergy between two or more media components results in increases in fermentation yield that are greater than would be expected based upon addition of the fermentation yield increases obtained when utilizing the two or more media components separately. For example, if addition of a first co-factor alone results in yields of a fermentation end-product that are A % higher than are achieved without addition of the first co-factor and addition of a second co-factor alone results in yields of a fermentation end-product that are B % higher than are achieved without addition of the second cofactor, then addition of both co-factors to the media can synergistically produce C % more fermentation end-product than is yielded without co-factor supplementation of the media wherein C is between about 1% and 300% greater than A+B (e.g., about 1-300%, 1-250%, 1-200%, 1-150%, 1-100%, 1-90%, 1-80%, 1-70%, 1-60%, 1-50%, 1-40%, 1-30%, 1-20%, 1-10%, 10-300%, 10-250%, 10-200%, 10-150%, 10-100%, 10-90%, 10-80%, 10-70%, 10-60%, 10-50%, 10-40%, 10-30%, 10-20%, 20-300%, 20-250%, 20-200%, 20-150%, 20-100%, 20-90%, 20-80%, 20-70%, 20-60%, 20-50%, 20-40%, 20-30%, 30-300%, 30-250%, 30-200%, 30-150%, 30-100%, 30-90%, 30-80%, 30-70%, 30-60%, 30-50%, 30-40%, 40-300%, 40-250%, 40-200%, 40-150%, 40-100%, 40-90%, 40-80%, 40-70%, 40-60%, 40-50%, 50-300%, 50-250%, 50-200%, 50-150%, 50-100%, 50-90%, 50-80%, 50-70%, 50-60%, 60-300%, 60-250%, 60-200%, 60-150%, 60-100%, 60-90%, 60-80%, 60-70%, 70-300%, 70-250%, 70-200%, 70-150%, 70-100%, 70-90%, 70-80%, 80-300%, 80-250%, 80-200%, 80-150%, 80-100%, 80-90%, 90-300%, 90-250%, 90-200%, 90-150%, 90-100%, 100-300%, 100-250%, 100-200%, 100-150%, 150-300%, 150-250%, 150-200%, 200-300%, 200-250%, 250-300%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, or 300% greater).

### Acidic Culture Conditions

In another aspect, methods of producing one or more fermentation end-products (e.g., alcohol, e.g., ethanol) comprising culturing a genetically modified microorganism adapted for reduced vitamin dependency (e.g., a genetically modified Clostridium biocatalyst) in a medium under conditions of controlled pH. In one embodiment, a culture of the genetically modified microorganism can be grown at an acidic pH are provided herein. The medium that the culture is grown in can include a carbon source such as agricultural crops, algae, crop residues, modified crop plants, trees, wood chips, sawdust, paper, cardboard, or other materials containing cellulose, hemicellulosic, lignocellulose, pectin, polyglucose, polyfructose, and/or hydrolyzed forms of these (collectively, “Feedstock”). Additional nutrients can be present including sulfur- and nitrogen-containing compounds such as amino acids, proteins, hydrolyzed proteins, ammonia, urea, nitrate, nitrite, soy, soy derivatives, casein, casein derivatives, milk powder, milk derivatives, whey, yeast extract, hydrolyzed yeast, autolyzed yeast, dried brewer's yeast, corn steep liquor, corn steep solids, monosodium glutamate, and/or other fermentation nitrogen sources, vitamins, cofactors and/or mineral supplements. The Feedstock can be pretreated or not, such as described in U.S. patent application Ser. No. 12/919,750, filed Aug. 26, 2010 or PCT Application No. PCT/US10/40502, filed on Jun. 29, 2010, which are herein incorporated by reference in their entireties. The procedures and techniques for growing the organism to produce a fuel or other desirable chemical such as is described in incorporated U.S. patent application Ser. No. 12/720,574 which is herein incorporated by reference in its entirety.

In one embodiment, the pH of the medium is controlled at less than about pH 7.2 for at least a portion of the fermentation. In one embodiment, the pH is controlled within a range of about pH 3.0 to about 7.1 or about pH 4.5 to about 7.1, or about pH 5.0 to about 6.3, or about pH 5.5 to about 6.3, or about pH 6.0 to about 6.5, or about pH 5.5 to about 6.9 or about pH 6.2 to about 6.7. The pH can be controlled by the addition of a pH modifier. In one embodiment, a pH modifier is an acid, a base, a buffer, or a material that reacts with other materials present to serve to raise of lower the pH. In one embodiment, more than one pH modifier can be used, such as more than one acid, more than one base, one or more acid with one or more bases, one or more acids with one or more buffers, one or more bases with one or more buffers, or one or more acids with one or more bases with one or more buffers. When more than one pH modifiers are utilized, they can be added at the same time or at different times. In one embodiment, one or more acids and one or more bases can be combined, resulting in a buffer. In one embodiment, media components, such as a carbon source or a nitrogen source can also serve as a pH modifier; suitable media components include those with high or low pH or those with buffering capacity. Exemplary media components include acid- or base-hydrolyzed plant polysaccharides having with residual acid or base, AFEX treated plant material with residual ammonia, lactic acid, corn steep solids or liquor.

In one embodiment, the pH modifier can be added as a part of the medium components prior to inoculation with a genetically modified microorganism adapted for reduced vitamin dependency (e.g., a genetically modified Clostridium biocatalyst). In one embodiment, the pH modifier can also be added after inoculation with the Clostridium biocatalysts. In one embodiment, sufficient buffer capacity can be added to the seed fermentation by way of various pH modifiers and/or other medium components and/or metabolites to provide adequate pH control during the final fermentation stage. In one embodiment, a pH modifier is added only to the final fermentation stage. In one embodiment, pH modifier is added to both the seed stage and the final stage. In one embodiment, the pH is monitored throughout the fermentation and is adjusted in response to changes in the fermentation. In one embodiment, the pH modifier is added whenever the pH of the fermentation changes by a pH value of about 0.005, 0.01, 0.05, 0.1, 0.2, 0.3, 0.4, 0.5 or more at any stage of the fermentation. In one embodiment, the pH modifier is added whenever the alcohol content of the fermentation is about 0.5 g/L, 1.0 g/L, 2.0 g/L, or 5.0 g/L or more. In one embodiment, different types of pH modifiers are utilized at different stages or points in the fermentation, such as a buffer being used at the seed stage, and base and/or acid added in the final fermentation, or an acid being used at one time and a base at another time.

In one embodiment, a constant pH can be utilized throughout the fermentation. In one embodiment, the timing and/or amount of pH reduction can be related to the growth conditions of the cells, such as in relation to the cell count, the alcohol produced, the alcohol present, or the rate of alcohol production. In one embodiment, the pH reduction can be made in relation to physical or chemical properties of the fermentation, such as viscosity, medium composition, gas production, off gas composition, etc.

Non-limiting examples of suitable buffers include salts of phosphoric acid, including monobasic, dibasic, and tribasic salts, mixtures of these salts and mixtures with the acid; salts of citric acid, including the various basic forms, mixtures and mixtures with the acid; and salts of carbonate.

Suitable acids and bases that can be used as pH modifiers include any liquid or gaseous acid or base that is compatible with the organism. Examples include ammonia, ammonium hydroxide, sulfuric acid, lactic acid, citric acid, phosphoric acid, sodium hydroxide, and HCl. In some cases, the selection of the acid or base can be influenced by the compatibility of the acid or base with equipment being used for fermentation. In some cases, both an acid addition, to lower pH or consume base, and a base addition, to raise pH or consume acid, can be used in the same fermentation.

The timing and amount of pH modifier to add can be determined from a measurement of the pH of the contents of the fermentor, such as by grab sample or by a submerged pH probe, or it can be determined based on other parameters such as the time into the fermentation, gas generation, viscosity, alcohol production, titration, etc. In one embodiment, a combination of these techniques can be used.

In one embodiment, the pH of the fermentation is initiated at a neutral pH and then is reduced to an acidic pH when the production of alcohol is detected. In another embodiment, the pH of the fermentation is initiated at an acidic pH and is maintained at an acidic pH until the fermentation reaches a stationary phase of growth.

### Fatty Acid Medium Component and Acidic Culture Conditions

In another embodiment, a combination of adding a fatty acid comprising compound to the medium and fermenting at reduced pH can be used. In one embodiment, addition of a fatty acid, such as a free fatty acid fulfills both techniques: adding a fatty acid compound and lowering the pH of the fermentation. In one embodiment, different compounds can be added to accomplish each technique. For example, a vegetable oil can be added to the medium to supply the fatty acid and then a mineral acid or an organic acid can be added during the fermentation to reduce the pH to a suitable level, as described above. When the fermentation includes both operation at reduced pH and addition of fatty acid comprising compounds, the methods and techniques described herein for each type of operation separately can be used together. In one embodiment, the operation at low pH and the presence of the fatty acid comprising compounds will be at the same time. In one embodiment, the presence of fatty acid comprising compounds will precede operation at low pH, and in one embodiment, operation at low pH will precede the addition of fatty acid comprising compounds. In one embodiment, the operation at low pH and the presence of the fatty acid will be prior to inoculation with Clostridium biocatalysts. In one embodiment, the operation at low pH will be prior to inoculation with Clostridium biocatalysts and the presence of the fatty acid will occur after or during inoculation with Clostridium biocatalysts. In one embodiment, the presence of the fatty acid will be prior to inoculation with Clostridium biocatalysts and the operation at low pH will occur after or during the inoculation with Clostridium biocatalysts. In one embodiment, the operation at low pH and the presence of the fatty acid will be after inoculation with Clostridium biocatalysts. In one embodiment, the operation at low pH and the presence of the fatty acid will be at other stages of fermentation.

Genetic Modification of Clostridium Biocatalysts

In another aspect, provided herein are compositions and methods to produce a fermentation end-product, e.g., a fuel such as one or more alcohols, e.g., ethanol, by the creation and use of a genetically modified Clostridium biocatalyst. In one embodiment, regulating fermentative biochemical pathways, expression of saccharolytic enzymes, or increasing tolerance of environmental conditions during fermentation of Clostridium biocatalysts is provided. One example of methods that can be used to enhance expression of saccharolytic enzymes can be found in U.S. patent application Ser. No. 12/630,784 filed Dec. 3, 2009. In one embodiment, a Clostridium biocatalyst is transformed with heterologous polynucleotides encoding one or more genes for the pathway, enzyme, or protein of interest. In another embodiment, a Clostridium biocatalyst is transformed to produce multiple copies of one or more genes for the pathway, enzyme, or protein of interest. In one embodiment, Clostridium biocatalysts are transformed with heterologous polynucleotides encoding one or more genes encoding enzymes for the hydrolysis and/or fermentation of a hexose, wherein said genes are expressed at sufficient levels to confer upon said Clostridium biocatalyst transformant the ability to produce ethanol at increased concentrations, productivity levels or yields compared to Clostridium biocatalysts that are not transformed. In such ways, an enhanced rate of ethanol production can be achieved.

In another embodiment, a Clostridium biocatalysts is transformed with heterologous polynucleotides encoding one or more genes encoding saccharolytic enzymes for the saccharification of a polysaccharide, wherein said genes are expressed at sufficient levels to confer upon said Clostridium biocatalyst transformant the ability to saccharify a polysaccharide to mono-, di- or oligosaccharides at increased concentrations, rates of saccharification or yields of mono-, di- or oligosaccharides compared to Clostridium biocatalysts that are not transformed. The production of a saccharolytic enzyme by the host, and the subsequent release of that saccharolytic enzyme into the medium, reduces the amount of commercial enzyme necessary to degrade biomass or polysaccharides into fermentable monosaccharides and oligosaccharides. The saccharolytic DNA can be native to the host, although more often the DNA will be foreign, and heterologous. Advantageous saccharolytic genes include cellulolytic, xylanolytic, and starch-degrading enzymes such as cellulases, xylanases, and amylases. The saccharolytic enzymes can be at least partially secreted by the host, or it can be accumulated substantially intracellularly for subsequent release. Advantageously, intracellularly-accumulated enzymes which are thermostable, can be released when desired by heat-induced lysis. Combinations of enzymes can be encoded by the heterologous DNA, some of which are secreted, and some of which are accumulated.

Other modifications can be made to enhance the ethanol production of the recombinant bacteria. For example, the host can further comprise an additional heterologous DNA segment, the expression product of which is a protein involved in the transport of mono- and/or oligosaccharides into the recombinant host. Likewise, additional genes from the glycolytic pathway can be incorporated into the host to redirect the bioenergetics of the ethanolic production pathways. In such ways, an enhanced rate of ethanol production can be achieved.

In order to improve the production of biofuels (e.g. ethanol), modifications can be made in transcriptional regulators, genes for the formation of organic acids, carbohydrate transporter genes, sporulation genes, genes that influence the formation/regenerate of enzymatic cofactors, genes that further influence ethanol tolerance, genes that influence salt tolerance, genes that influence growth rate, genes that influence oxygen tolerance, genes that influence catabolite repression, genes that influence hydrogen production, genes that influence resistance to heavy metals, genes that influence resistance to acids or genes that influence resistance to aldehydes.

Those skilled in the art will appreciate that a number of modifications can be made to the methods exemplified herein. For example, a variety of promoters can be utilized to drive expression of the heterologous genes in the recombinant Clostridium sp. host. The skilled artisan, having the benefit of the instant disclosure, will be able to readily choose and utilize any one of the various promoters available for this purpose. Similarly, skilled artisans, as a matter of routine preference, can utilize a higher copy number plasmid. In another embodiment, constructs can be prepared for chromosomal integration of the desired genes. Chromosomal integration of foreign genes can offer several advantages over plasmid-based constructions, the latter having certain limitations for commercial processes. Ethanologenic genes have been integrated chromosomally in E. coli B; see Ohta et al. (1991) Appl. Environ. Microbiol. 57:893-900. In general, this is accomplished by purification of a DNA fragment containing (1) the desired genes upstream from an antibiotic resistance gene and (2) a fragment of homologous DNA from the target organism. This DNA can be ligated to form circles without replicons and used for transformation. Thus, the gene of interest can be introduced in a heterologous host such as E. coli, and short, random fragments can be isolated and ligated in Clostridium sp. to promote homologous recombination.

### Biofuel Plant and Process of Producing Biofuel:

Large Scale Ethanol Production from Biomass

Generally, there are two basic approaches to producing fuel grade ethanol from biomass on a large scale utilizing of microbial cells, especially Clostridium biocatalyst cells. In the first method, one first hydrolyzes a biomass material that includes high molecular weight carbohydrates to lower molecular weight carbohydrates, and then ferments the lower molecular weight carbohydrates utilizing of microbial cells to produce ethanol. In the second method, one ferments the biomass material itself without chemical and/or enzymatic pretreatment. In the first method, hydrolysis can be accomplished using acids, e.g., Bronsted acids (e.g., sulfuric or hydrochloric acid), bases, e.g., sodium hydroxide, hydrothermal processes, ammonia fiber explosion processes (“AFEX”), lime processes, enzymes, or combination of these. Hydrogen, and other products of the fermentation can be captured and purified if desired, or disposed of, e.g., by burning. For example, the hydrogen gas can be flared, or used as an energy source in the process, e.g., to drive a steam boiler, e.g., by burning. Hydrolysis and/or steam treatment of the biomass can, e.g., increase porosity and/or surface area of the biomass, often leaving the cellulosic materials more exposed to the biocatalyst cells, which can increase fermentation rate and yield. Removal of lignin can, e.g., provide a combustible fuel for driving a boiler, and can also, e.g., increase porosity and/or surface area of the biomass, often increasing fermentation rate and yield. Generally, in any of the below described embodiments, the initial concentration of the carbohydrates in the medium is greater than 20 mM, e.g., greater than 30 mM, 50 mM, 75 mM, 100 mM, 150 mM, 200 mM, or even greater than 500 mM.

Biomass Processing Plant and Process of Producing Products from Biomass

In one aspect, a fuel plant that includes a hydrolysis unit configured to hydrolyze a biomass material that includes a high molecular weight carbohydrate, a fermentor configured to house a medium with Clostridium biocatalyst cells or another C5/C6 hydrolyzing organism dispersed therein, and one or more product recovery system(s) to isolate a product or products and associated by-products and co-products is provided.

In another aspect, methods of making a product or products that include combining Clostridium biocatalyst cells or another C5/C6 hydrolyzing organism and a biomass feed in a medium, and fermenting the biomass material under conditions and for a time sufficient to produce a biofuel, chemical product or fermentation end-products, e.g. ethanol, propanol, hydrogen, lignin, terpenoids, and the like as described above, is provided.

In another aspect, products made by any of the processes described herein is also provided herein.

Large Scale Chemical Production from Biomass

Generally, there are two basic approaches to producing chemical products from biomass on a large scale utilizing microorganisms such as Clostridium biocatalysts or other C5/C6 hydrolyzing organisms. In all methods, depending on the type of biomass and its physical manifestation, one of the processes can comprise a milling of the carbonaceous material, via wet or dry milling, to reduce the material in size and increase the surface to volume ratio (physical modification).

In a first method, one first hydrolyzes a biomass material that includes high molecular weight carbohydrates to delignify it or to separate the carbohydrate compounds from noncarbohydrate compounds. Using any combination of heat, chemical, and/or enzymatic treatment, the hydrolyzed material can be separated to form liquid and dewatered streams, which may or may not be separately treated and kept separate or recombined, and then ferments the lower molecular weight carbohydrates utilizing Clostridium biocatalyst cells or another C5/C6 hydrolyzing biocatalyst to produce one or more chemical products. In the second method, one ferments the biomass material itself without heat, chemical, and/or enzymatic pretreatment. In the first method, hydrolysis can be accomplished using acids (e.g. sulfuric or hydrochloric acids), bases (e.g. sodium hydroxide), hydrothermal processes, ammonia fiber explosion processes (“AFEX”), lime processes, enzymes, or combination of these. Hydrolysis and/or steam treatment of the biomass can, e.g., increase porosity and/or surface area of the biomass, often leaving the cellulosic materials more exposed to any C5/C6 hydrolyzing organism, such as Clostridium biocatalysts, which can increase fermentation rate and yield. Hydrolysis and/or steam treatment of the biomass can, e.g., produce by-products or co-products which can be separated or treated to improve fermentation rate and yield, or used to produce power to run the process, or used as products with or without further processing. Removal of lignin can, e.g., provide a combustible fuel for driving a boiler. Gaseous, e.g., hydrogen and CO₂, liquid, e.g. ethanol and organic acids, and solid, e.g. lignin, products of the fermentation can be captured and purified if desired, or disposed of, e.g., by burning. For example, the hydrogen gas can be flared, or used as an energy source in the process, e.g., to drive a steam boiler, e.g., by burning. Products exiting the fermentor can be further processed, e.g. ethanol can be transferred to distillation and rectification, producing a concentrated ethanol mixture or solids can be separated for use to provide energy or as chemical products. It is understood that other methods of producing fermentation end-products or biofuels can incorporate any and all of the processes described as well as additional or substitute processes that can be developed to economically or mechanically streamline these methods, all of which are meant to be incorporated in their entirety within the scope of this disclosure.

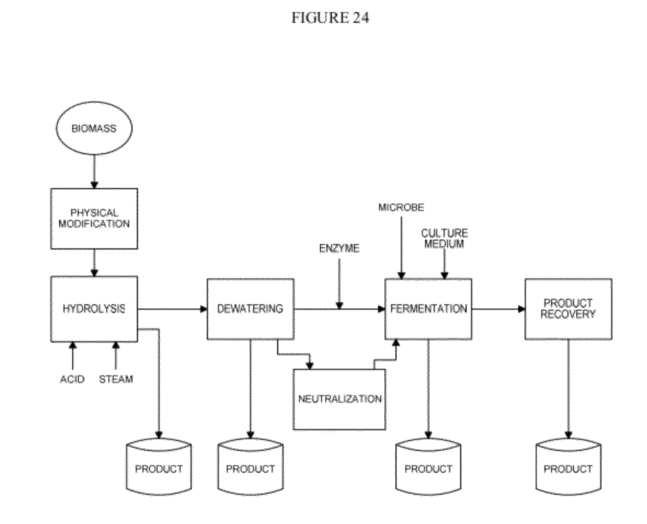

FIG. 24 is an example of a method for producing chemical products from biomass by first treating biomass with an acid at elevated temperature and pressure in a hydrolysis unit. The biomass can first be heated by addition of hot water or steam. The biomass can be acidified by bubbling gaseous sulfur dioxide through the biomass that is suspended in water, or by adding a strong acid, e.g., sulfuric, hydrochloric, or nitric acid with or without preheating/presteaming/water addition. During the acidification, the pH is maintained at a low level, e.g., below about 5. The temperature and pressure can be elevated after acid addition. In addition to the acid already in the acidification unit, optionally, a metal salt such as ferrous sulfate, ferric sulfate, ferric chloride, aluminum sulfate, aluminum chloride, magnesium sulfate, or mixtures of these can be added to aid in the hydrolysis of the biomass. The acid-impregnated biomass is fed into the hydrolysis section of the pretreatment unit. Steam is injected into the hydrolysis portion of the pretreatment unit to directly contact and heat the biomass to the desired temperature. The temperature of the biomass after steam addition is, e.g., between about 130° C. and 220° C. The hydrolysate can then be discharged into the flash tank portion of the pretreatment unit, and can be held in the tank for a period of time to further hydrolyze the biomass, e.g., into oligosaccharides and monomeric sugars or oligomers. Steam explosion can also be used to further break down biomass. Alternatively, the biomass can be subject to discharge through a pressure lock for any high-pressure pretreatment process. Hydrolysate is then discharged from the pretreatment reactor, with or without the addition of water, e.g., at solids concentrations between about 15% and 60%.

After pretreatment, the biomass can be dewatered and/or washed with a quantity of water, e.g. by squeezing or by centrifugation, or by filtration using, e.g. a countercurrent extractor, wash press, filter press, pressure filter, a screw conveyor extractor, or a vacuum belt extractor to remove acidified fluid. The acidified fluid, with or without further treatment, e.g. addition of alkali (e.g. lime) and or ammonia (e.g. ammonium phosphate), can be re-used, e.g., in the acidification portion of the pretreatment unit, or added to the fermentation, or collected for other use/treatment. Products can be derived from treatment of the acidified fluid, e.g., gypsum or ammonium phosphate. Enzymes or a mixture of enzymes can be added during pretreatment to assist, e.g. endoglucanases, exoglucanases, cellobiohydrolases (CBH), beta-glucosidases, glycoside hydrolases, glycosyltransferases, lyases, and esterases active against components of cellulose, hemicelluloses, pectin, and starch, in the hydrolysis of high molecular weight components.

The fermentor is fed with hydrolyzed biomass, any liquid fraction from biomass pretreatment, an active seed culture of Clostridium sp. cells, if desired a co-fermenting microorganism, e.g., yeast or E. coli, and, if desired, nutrients to promote growth of Clostridium sp. or other microorganisms. Alternatively, the pretreated biomass or liquid fraction can be split into multiple fermentors, each containing a different strain of Clostridium sp. and/or other microorganisms, and each operating under specific physical conditions. Fermentation is allowed to proceed for a period of time, e.g., between about 15 and 150 hours, while maintaining a temperature of, e.g., between about 25° C. and 50° C. Gas produced during the fermentation is swept from fermentor and is discharged, collected, or flared with or without additional processing, e.g. hydrogen gas can be collected and used as a power source or purified as a co-product.

After fermentation, the contents of the fermentor are transferred to product recovery. Products are extracted, e.g., ethanol is recovered through distilled and rectification.

Chemical Production from Biomass without Pretreatment

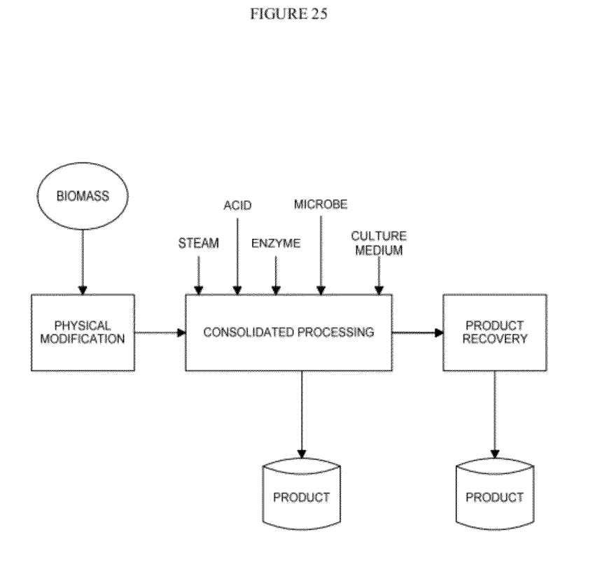

FIG. 25 depicts a method for producing chemicals from biomass by charging biomass to a fermentation vessel. The biomass can be allowed to soak for a period of time, with or without addition of heat, water, enzymes, or acid/alkali. The pressure in the processing vessel can be maintained at or above atmospheric pressure. Acid or alkali can be added at the end of the pretreatment period for neutralization. At the end of the pretreatment period, or at the same time as pretreatment begins, an active seed culture of Clostridium sp. cells or another C5/C6 hydrolyzing organism and, if desired, a co-fermenting microorganism, e.g., yeast or E. coli, and, if desired, nutrients to promote growth of Clostridium sp. or other microorganisms are added. Fermentation is allowed to proceed as described above. After fermentation, the contents of the fermentor are transferred to product recovery as described above.

Any combination of the chemical production methods and/or features can be utilized to make a hybrid production method. In any of the methods described herein, products can be removed, added, or combined at any step. Clostridium sp. can be used alone, or synergistically in combination with one or more other microorganisms (e.g. yeasts, fungi, or other bacteria). Different methods can be used within a single plant to produce different products.

In another aspect, a fuel plant that includes a hydrolysis unit configured to hydrolyze a biomass material that includes a high molecular weight carbohydrate, and a fermentor configured to house a medium and contains Clostridium cells dispersed therein, is provided.

In another aspect, methods of making a fuel or fuels that include combining Clostridium sp. cells and a lignocellulosic material (and/or other biomass material) in a medium, and fermenting the lignocellulosic material under conditions and for a time sufficient to produce a fuel or fuels, e.g., ethanol, propanol and/or hydrogen or another chemical compound is provided herein.

In one embodiment, a process for producing ethanol and hydrogen from biomass using acid hydrolysis pretreatment is provided. In one embodiment, a process for producing ethanol and hydrogen from biomass using enzymatic hydrolysis pretreatment is provided. In one embodiment, the process for producing ethanol and hydrogen from biomass is by using biomass that has not been enzymatically pretreated. Still in another embodiment, the process for producing ethanol and hydrogen from biomass is by using biomass that has not been chemically or enzymatically pretreated, but is optionally steam treated.

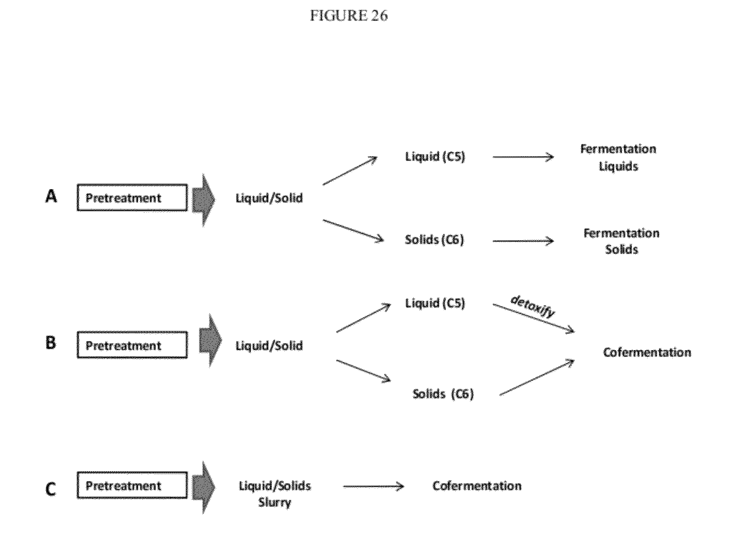

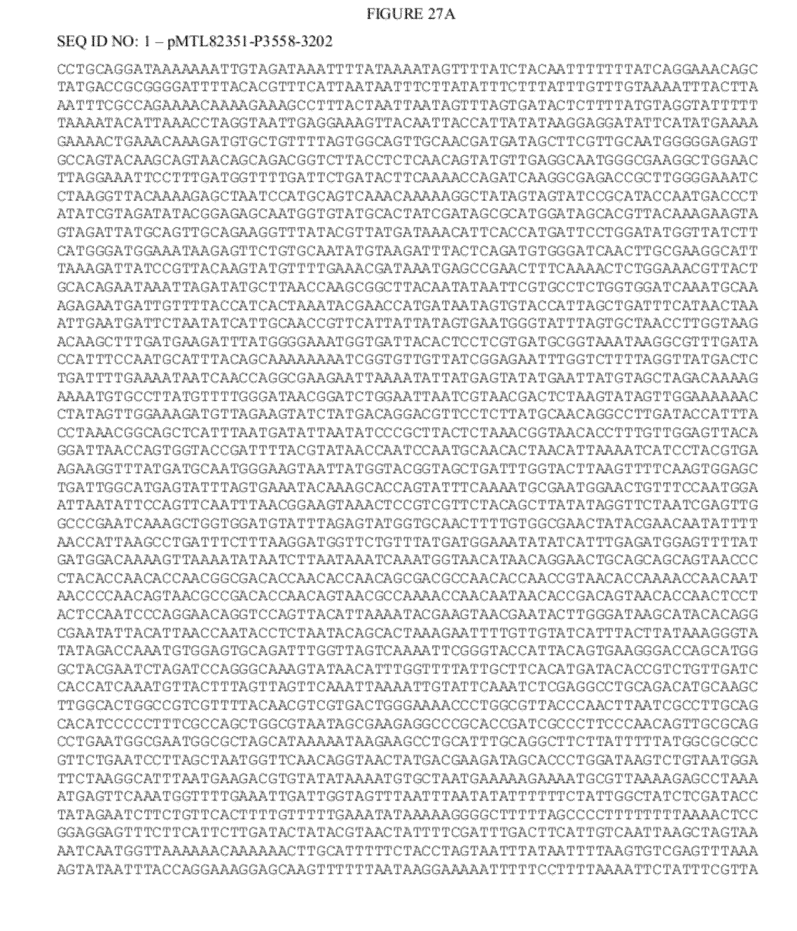

FIG. 26 discloses pretreatments that produce hexose or pentose saccharides or oligomers that are then unprocessed or processed further and either, fermented separately or together. FIG. 26A depicts a process (e.g., acid pretreatment) that produces a solids phase and a liquid phase which are then fermented separately. FIG. 26B depicts a similar pretreatment that produces a solids phase and liquids phase. The liquids phase is separated from the solids and elements that are toxic to the fermenting microorganism are removed prior to fermentation. At initiation of fermentation, the two phases are recombined and cofermented together. This is a more cost-effective process than fermenting the phases separately. The third process (FIG. 26C) is the least costly. The pretreatment results in a slurry of liquids or solids that are then cofermented. There is little loss of saccharides component and minimal equipment used.

Genetic Modification

In one embodiment, a genetically modified microorganism adapted for reduced vitamin dependency can be furthermodified to enhance enzyme activity of one or more enzymes, including but not limited to hydrolytic enzymes (such as cellulase(s), hemice/lulase(s), or pectinases etc.). In another embodiment, an enzyme can be selected from the annotated genome of C. phytofermentans, another bacterial species, such as B. subtilis, E. coli, various Clostridium species, such as C. cellulolyticum or C. sp. Q.D., or yeasts such as S. cerevisiae for utilization in products and processes described herein. Examples include enzymes such as L-butanediol dehydrogenase, acetoin reductase, 3-hydroxyacyl-CoA dehydrogenase, cis-aconitate decarboxylase or the like, to create pathways for new products from biomass.

Examples of such modifications include modifying endogenous nucleic acid regulatory elements to increase expression of one or more enzymes (e.g., operably linking a gene encoding a target enzyme to a strong promoter), introducing into a microorganism additional copies of endogenous nucleic acid molecules to provide enhanced activity of an enzyme by increasing its production, and operably linking genes encoding one or more enzymes to an inducible promoter or a combination thereof. A detailed description of methods of genetic modification of microorganisms, including Clostridia species, is disclosed in the International Application Publication WO2011008564, which is hereby incorporated by reference in its entirety.

In another embodiment a genetically modified microorganism adapted for reduced vitamin dependency can be further modified to enhance an activity of one or more cellulases, or enzymes associated with cellulose processing. The classification of cellulases is usually based on grouping enzymes together that forms a family with similar or identical activity, but not necessary the same substrate specificity. One of these classifications is the CAZy system (CAZy stands for Carbohydrate-Active enZymes), for example, where there are 115 different Glycoside Hydrolases (GH) listed, named GH1 to GH155. Each of the different protein families usually has a corresponding enzyme activity. This database includes both cellulose and hemicellulase active enzymes. Furthermore, the entire annotated genome of C. phytofermentans is available on the worldwideweb at www.ncbi.nlm.nih.gov/sites/entrez. Detailed methods for genetic modification

Several examples of cellulase enzymes whose function can be enhanced for expression endogenously or for expression heterologously in a microorganism include one or more of the genes disclosed in Table 2.

TABLE 2
Cellulase enzymes

| Cellulase Protein ID | Description (on www.ncbi.nlm.nih.gov/sites/entrez) |
|---|---|
| ABX43556 | Cellulase [Clostridium phytofermentans ISDg] |
| | gi\|160429993\|gb\|ABX43556.1\|[160429993] |
| | Cphy_3302 |
| ABX42426 | Cellulase [Clostridium phytofermentans ISDg] |
| | gi\|160428863\|gb\|ABX42426.1\|[160428863] |
| | Cphy_2058 |
| ABX41541 | Cellulase [Clostridium phytofermentans ISDg] |
| | gi\|160427978\|gb\|ABX41541.1\|[160427978] |
| | Cphy_1163 |
| ABX43720 | Cellulose 1,4-beta-cellobiosidase [Clostridium |
| | phytofermentans ISDg] |
| | gi\|160430157\|gb\|ABX43720.1\|[160430157] |
| | Cphy_3367 |
| ABX41478 | Cellulase M Cphy_1100 |
| ABX41884 | Endo-1,4-beta-xylanase Cphy_1510 |
| ABX43721 | Cellulase 1,4-beta-cellobiosidase Cphy_3368 |
| ABX42494 | Mannan endo-1,4-beta-mannosidase, Cellulase 1,4- |
| | beta-cellobiosidase Cphy_2128 |

### Directed Evolution

Various methods can be used to produce and select mutants that differ from wild-type cells. In some instances, bacterial populations are treated with a mutagenic agent, for example, nitrosoguanidine (N-methyl-N′-nitro-N-nitrosoguanidine) or the like, to increase the mutation frequency above that of spontaneous mutagenesis. This is induced mutagenesis. Techniques for inducing mutagenesis include, but are not limited to, exposure of the bacteria to a mutagenic agent, such as x-rays or chemical mutagenic agents. More sophisticated procedures involve isolating the gene of interest and making a change in the desired location, then reinserting the gene into bacterial cells. This is site-directed mutagenesis.

Directed evolution is usually performed as three steps which can be repeated more than once. First, the gene encoding a protein of interest is mutated and/or recombined at random to create a large library of gene variants. The library is then screened or selected for the presence of mutants or variants that show the desired property. Screens enable the identification and isolation of high-performing mutants by hand; selections automatically eliminate all non functional mutants. Then the variants identified in the selection or screen are replicated, enabling DNA sequencing to determine what mutations occurred. Directed evolution can be carried out in vivo or in vitro. See, for example, Often, L. G.; Quax, W. J. (2005). Biomolecular Engineering 22 (1-3): 1-9; Yuan, L., et al. (2005) Microbiol. Mol. Biol. Rev. 69 (3): 373-392.

In another aspect, the products made by any of the processes described herein is provided.

### EXAMPLES

The following examples serve to illustrate certain embodiments and aspects and are not to be construed as limiting the scope thereof

### Example 1

### Propagation and Fermentation Media for C. phytofermentans and Other Mesophilic Clostridium Species

Seed propagation media (BM) recipe:

| BM Base medium: | per L: | |
|---|---|---|
| KH₂PO₄ | 1.60 | g |
| K₂HPO₄ | 3.00 | g |
| NaCl | 1.00 | g |
| Ammonium sulfate | 2.00 | g |
| 0.1% (w/v) Resazurin solution | 0.500 | ml |
| Bacto yeast extract | 2.50 | |
| Adjust pH to 7.5 with NaOH | | |

| 10x Substrate Stock | g/L: |
|---|---|
| 20% Cellobiose | 20 |

| 100X BM Salts solution: | g/L |
|---|---|
| Tri-Sodium Citrate | 10.000 |
| CaCl2•2H₂O | 0.500 |
| MgSO₄•7H₂O | 6.000 |
| FeSO₄•7H₂O | 0.400 |
| CoSO₄•H₂O | 0.200 |
| ZnSO₄•7H₂O | 0.200 |
| NiCl₂ | 0.200 |
| MnSO₄•H₂O | 0.500 |
| CuSO₄•5H₂O | 0.040 |
| H₃BO₃ | 0.040 |
| Ammonium Molybdate tetrahydrate (H₂₄Mo₇N₆O₂₄•4H₂O) | 0.040 |
| Sodium Selenite (Na₂SeO₃) | 0.040 |

| 100X B Vitamins solution: | g/L |
|---|---|
| Pantethine | 1.000 |
| Nicotinic Acid (B₃) | 3.000 |
| Pyridoxine (B₆) | 1.000 |
| Cyanocobalamine (B₁₂) | 1.000 |
| Thiamine (B₁) | 1.000 |
| Riboflavin (B₂) | 1.000 |
| Folinic Acid | 0.030 |
| 100X Amino Acids solution: | g/L |
| L-Cysteine HCl monohydrate | 100 |
| L-Histidine | 25 |

The seed propagation medium was prepared according to the recipe above. Base media, salts and substrates were degassed with nitrogen prior to autoclave sterilization. Following sterilization, 87 ml of base media was combined with 10 ml of 10× Substrate stock and 1 ml each of 100× salts solution, 100× amino acids and 100× B-Vitamins solution to achieve final concentrations. All additions were prepared anaerobically and aseptically.

Fermentation Media: (FM Media)

Base media (g/L) was prepared with: 50 g/l NaOH pretreated corn stover, yeast extract 10, corn steep powder 5, K₂HPO₄ 3, KH₂PO₄ 1.6, TriSodium citrate 2H₂O₂ 2, Citric acid H₂O1.2, (NH₄)₂SO₄ 0.5, NaCl 1, Cysteine.HCl 1, dissolved in deionized water to achieve final volume, adjusted to pH to 6.5, degassed with nitrogen and autoclaved 121° C. for 30 min.

100× Salt Stock (g/L):

MgCl₂.6H₂O 80, CaCl₂.2H₂O 10, FeSO₄.7H₂O 0.125, TriSodium citrate.2H₂O₂ 3.0

The fermentation media was prepared according to the protocol above. Components of the Base media were combined to a single vessel and degassed with nitrogen prior to sterilization. A 100× salts stock was prepared and sterilized separately. After sterilization base media was supplemented with a 1% v/v dose of 100× salts to achieve a final concentration. All additions were prepared anaerobically and aseptically.

### Example 2

### Thiamine Supplementation

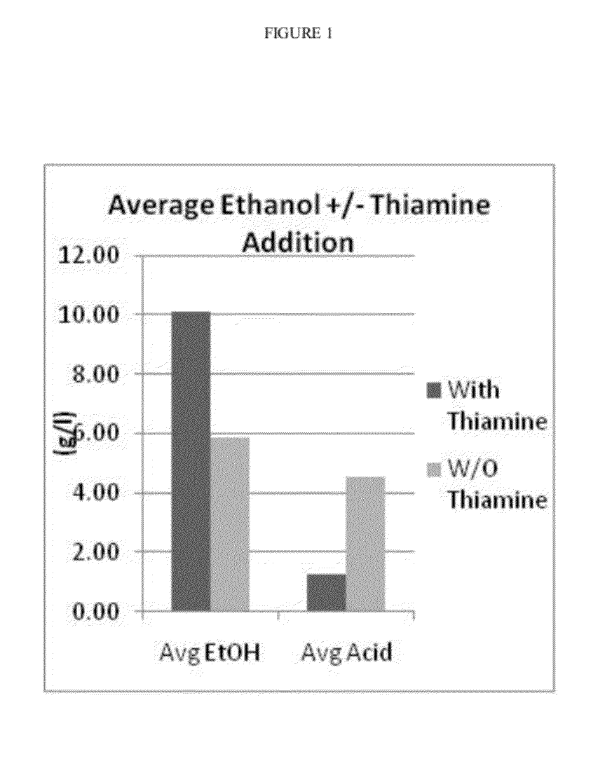

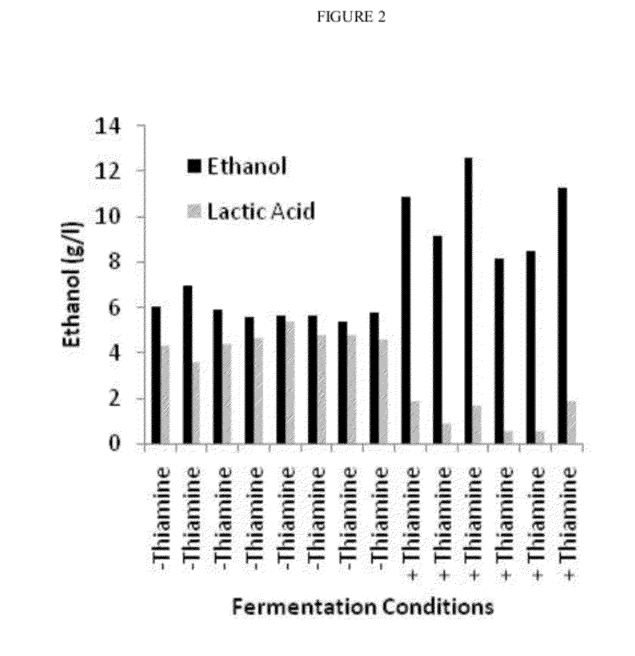

A pure culture of C. phytofermentans microorganism strain (Q.8) was propagated to mid exponential stage growth in FM medium containing yeast extract and casein hydrolysate, supplemented with vitamins except for thiamine and inorganic salts. The 0.1 L shake flask fermentations were conducted in septa sealed 0.25 L screw cap bottles under an atmosphere of nitrogen gas. The Q.8 culture was grown to mid-exponential stage prior to inoculation into the experimental shake flask series at 10% v/v. The inoculated shake flasks were incubated at 35° C. for 137 hrs. Final samples were analyzed for ethanol, lactic, acetic acid and residual sugars by high pressure liquid chromatography. The addition of thiamine at a final concentration of 5 mg/L produces a significant increase in ethanol titer and overall yield corresponding to a significant decrease in the production of lactic acid. (FIGS. 1 & 2).

### Example 3

### Microorganism Modification and Vector Construction

Plasmid Construction

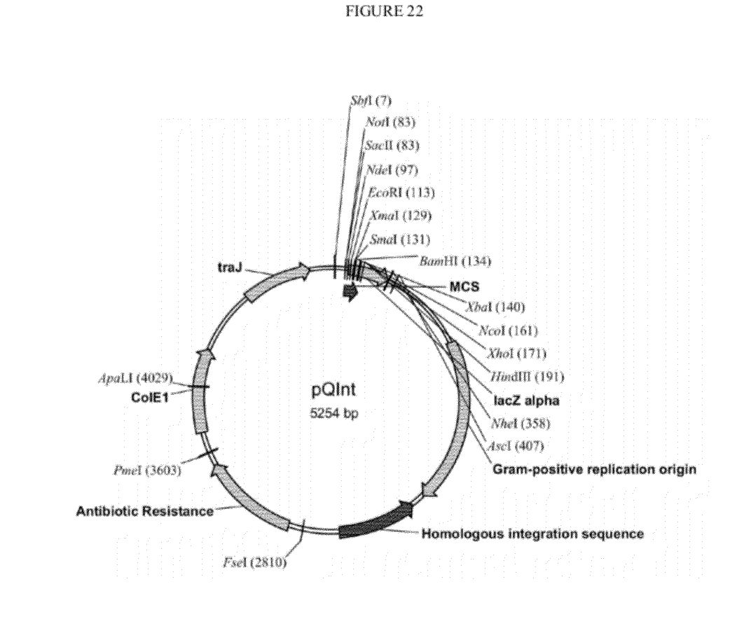

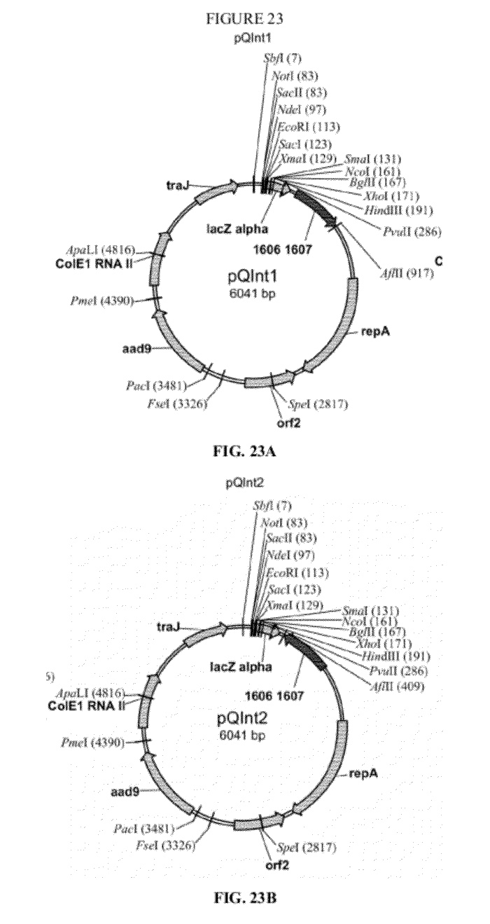

A general illustration of an integrating replicative plasmid, pQInt, is shown in FIG. 22. Identified elements include a Multi-cloning site (MCS) with a LacZ-α reporter for use in E. coli; a gram-positive replication origin; the homologous integration sequence; an antibiotic-resistance cassette; the ColE1 gram-negative replication origin and the traJ origin for conjugal transfer. Several unique restriction sites are indicated but are not meant to be limiting on any embodiment. The arrangement of the elements can be modified.

Another embodiment, depicted in FIG. 23, is a map of the plasmids pQInt1 and pQInt2. These plasmids contain gram-negative (ColE1) and gram-positive (repA/Orf2) replication origins; the bi-functional aad9 spectinomycin-resistance gene; traJ origin for conjugal transfer; LacZ-α/MCS and the 1606-1607 region of chromosomal homology. Since the 1606-1607 region of homology is cloned into a single AscI site, it can be obtained in two different orientations in a single cloning step. Plasmid pQInt2 is identical to pQInt1 except the orientation of the homology region is reversed.

These plasmids consist of five key elements. 1) A gram-negative origin of replication for propagation of the plasmid in E. coli or other gram-negative host(s). 2) A gram-positive replication origin for propagation of the plasmid in gram-positive organisms. In C. phytofermentans, this origin allows for suitable levels of replication prior to integration. 3) A selectable marker; typically a gene encoding antibiotic resistance. 4) An integration sequence; a sequence of DNA at least 400 base pairs in length and identical to a locus in the host chromosome. This represents the targeted site of integration. 5) A multi-cloning site (“MCS”) with or without a heterologous gene expression cassette cloned. An additional element for conjugal transfer of plasmid DNA is an optional element described in certain embodiments.

Plasmid Utilization

The plasmid is digested with suitable restriction enzyme(s) to allow a heterologous gene expression cassette (“insert”) to be ligated in the MCS. Examples of such restriction enzymes include but are not limited to BamHI, HindIII, SalI, Sad and NdeI. Ligation products are transformed into a suitable cloning host, typically E. coli. Antibiotic resistant transformants are screened to verify the presence of the desired insert. The plasmid is then transformed into C. phytofermentans or other suitable expression host strain. Transformants are selected based on resistance to the appropriate antibiotic. Resistant colonies are propagated in the presence of antibiotic to allow for homologous recombination integration of the plasmid. Integration is verified by a “junction PCR” protocol. This protocol uses either a preparation of host chromosomal DNA or a sample of transformed cells. The junction PCR utilizes one primer that hybridizes to the plasmid backbone flanking the MCS and a second primer that hybridizes to the chromosome flanking the site of integration. The primers can be designed so they are unique. That is, the plasmid primer cannot hybridize to chromosomal sequences and the chromosomal primer cannot hybridize to the plasmid. The ability to amplify a PCR product demonstrates integration at the correct site.

Standard gene expression systems use autonomously replicating plasmids (“episomes” or “episomal plasmids”). Such plasmids are not suitable for use in C. phytofermentans, C. sp. Q.D and most other Clostridia due to segregational instability. The use of homologous sequences to allow for integration of a replicative gene expression in C. phytofermentans is not usual for transformation.

Use of a series of plasmids each containing a different antibiotic resistance gene, allows for versatility in cases where certain antibiotics are not suitable for specific organisms. The embodiment uses an “integration sequence” which is easily cloned from the chromosome by PCR using primers with tails that encode the appropriate restriction enzyme recognition sequences. This allows for the targeted integration of the entire plasmid at a chosen locus. The inclusion of a gram-negative replication origin allows for cloning and the easy propagation of the plasmid in a host such as E. coli. The gram-positive replication origin allows for a level of replication of the plasmid in C. phytofernmentans after transformation and prior to integration. This contrasts with true suicide integration which utilizes non-replicating plasmids. In true suicide integration, the only way to obtain an antibiotic resistant transformant is to have the plasmid integrate immediately after transformation. This is a low probability event. Replication from the gram-positive origin after transformation results in a greater number of transformed cells which makes the integration event statistically more likely.

The integrated plasmid can be stable. The transformed strain can be propagated without loss of plasmid DNA. The transformant can be evaluated for heterologous gene expression under any suitable conditions. Stability of the integrated DNA can be ensured by continuous culture in the presence of the appropriate antibiotic. It is also possible to remove the antibiotic if so desired.

Constitutive Expression of Cellulases I

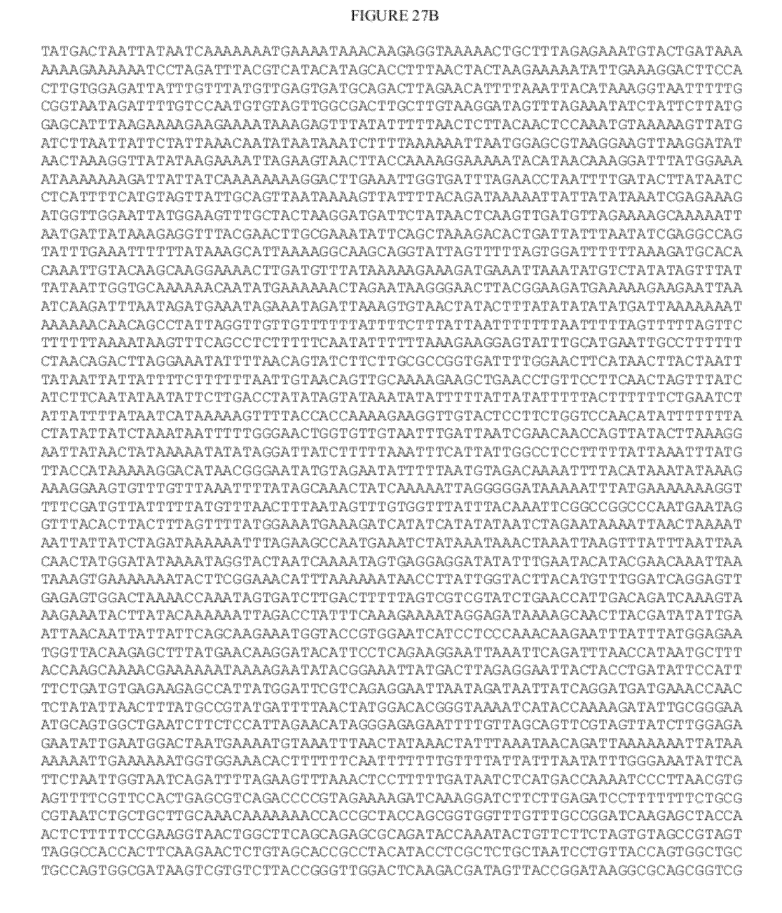

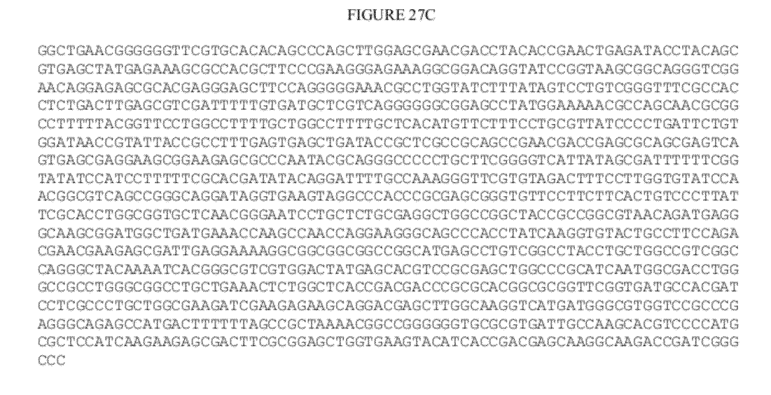

Plasmids suitable for use in Clostridium phytofermentans were constructed using pMTL82351 with the promoter from the C. phytofermentans pyruvate ferredoxin oxidase reductase gene Cphy_{—}3558 and the C. phytofermentans cellulase gene Cphy_{—}3202. The sequence of this vector (pMTL82351-P3558-3202) inserted DNA is found in FIG. 27.

The successful transfer of pMTL82351-P3558-3202 into the C. phytofermentans strain via electroporation was demonstrated by the ability to grow in the presence of 10 μg/mL erythromycin. The plasmid has been serially propagated in this transformant for over four months.

Constitutive Promoter

Several other promoters from C. phytofermentans were chosen for vector use that show high expression of their corresponding genes in all growth stages as well as on different substrates. A promoter element can be selected by selecting key genes that would necessarily be involved in constitutive pathways (e.g., ribosomal genes, or for ethanol production, alcohol dehydrogenase genes). Examples of promoters from such genes include but are not limited to:

Cphy_{—}1029: iron-containing alcohol dehydrogenase

Cphy_{—}3510: Ig domain-containing protein [S-layer protein]

Cphy_{—}3925: bifunctional acetaldehyde-CoA/alcohol dehydrogenase

Cloning of Cellulase Genes

One or more genes disclosed in Table 2, which can include each gene's own ribosome binding sites, were amplified via PCR and subsequently digested with the appropriate enzymes as described previously under Cloning of Promoter. Resulting plasmids were also treated with the corresponding restriction enzymes and the amplified genes are mobilized into plasmids through standard ligation. E. coli were transformed with the plasmids and correct inserts were verified from transformants selected on selection plates.

### Example 4

### Transconjugation

E. coli DH5a along with the helper plasmid pRK2030, were transformed with the different plasmids discussed above. E. coli colonies with both of the foregoing plasmids were selected on LB plates with 100 μg/mlampicillin and 50 μg/mlkanamycin after growing overnight at 37° C. Single colonies were obtained after re-streaking on selective plates at 37° C. Growth media for E. coli (e.g. LB or LB supplemented with 1% glucose and 1% cellobiose) was inoculated with a single colony and either grown aerobically at 37° C. or anaerobically at 35° C. overnight. Fresh growth media was inoculated 1:100 with the overnight culture and grown until mid log phase. A C. phytofermentans strain was also grown in the same media until mid log.

The two different cultures, C. phytofermentans and E. coli with pRK2030 and one of the plasmids, were then mixed in different ratios, e.g. 1:1000, 1:100, 1:10, 1:1, 10:1, 100:1, 1000:1. The mating was performed in either liquid media, on plates or on 25 mm Nucleopore Track-Etch Membrane (Whatman, Inc., 800 Centennial Avenue, Piscataway, N.J. 08854 USA) at 35° C. The time was varied between 2 h and 24 h, and the mating media was the same growth media in which the culture was grown prior to the mating. After the mating procedure, the bacteria mixture was either spread directly onto plates or first grown on liquid media for 6 h to 18 h and then plated. The plates contain 10 μg/ml erythromycin as selective agent for C. phytofermentans and 10 μg/ml Trimethoprim, 150 μg/ml Cyclosporin and 100 μg/mlNalidixic acid as counter selectable media for E. coli.

After 3 to 5 days incubation at 35° C., erythromycin-resistant colonies were picked from the plates and restreaked on fresh selective plates. Single colonies were picked and the presence of the plasmid is confirmed by PCR reaction.

Cellulase Gene Expression

The expression of the cellulase genes on the different plasmids was then tested under conditions where there is little to no expression of the corresponding genes from the chromosomal locus. Positive candidates showed constitutive expression of the cloned cellulases.

### Example 5

### Electroporation Procedure

All procedures were conducted anaerobically except centrifugation wherein the centrifuge tubes were sealed from the atmosphere.

A single colony of C. phytofermentans was inoculated into 100 ml of culture broth (BM) and grown at 37° C. overnight. The culture was then diluted 1:100 (i.e. 1 ml in 100 ml) in fresh BM medium and incubated at 37° C. for approximately 24 hr. The entire culture was transferred to two 50 mL Falcon tubes which were spun at 8,500 RPM (˜18,000 g) for 10 minutes. The supernatant was discarded and the pellet resuspended with 2.0 mL of ice-cold Electroporation Buffer (EPB: 250 mM sucrose, 5 mM sodium phosphate). The suspension was again spun at 8,500 RPM (˜18,000 g) for 10 minutes. The supernatant was discarded and the pellet resuspended with 2.0 mL ice-cold EPB wherein the sample was placed on ice.

500 μL of competent C. phytofermentans cells were transferred into a 0.4 cm electroporation cuvette (BioRad, Inc., 1000 Alfred Nobel Drive, Hercules, Calif. 94547), and the cuvettes kept on ice. 25 μL of DNA (˜1.0 μg) was added to each cuvette on ice. The solution was mixed by gently circulating the pipette tip. It was not mixed by pipetting or vortexing. The cells were incubated on ice for 4 minutes.

When ready for electroporation, the metal contacts of the electroporation cuvette were cleaned with a Kimwipe or other adsorbent material to ensure no trace of moisture was present. Electroporation was conducted using a Gene Pulser Xcell™ apparatus (BioRad, Inc.) at 1500 V to 2500 V [e.g., 2250V], 25 μF, and 600 ohms. The time constant was in the interval of 4.5 to 6.0 ms.

Immediately, the contents of the cuvette were diluted with 1 mL of prewarmed (37° C.) BM media. The entire solution was poured into a 15 mL Falcon tube containing an additional 5 ml BM and incubated anaerobically at 37° C. for 2 to 6 hours after which time the cells were spread directly on selective plates (BM agar supplemented with 250 μg/ml spectinomycin). Cells were spread in a dilution series so that isolated single-cell colonies could be obtained.

### Example 6

### Assays

The transformants from the BM plate, which contained 250 μg/ml of spectinomycin, were transfered into BM liquid medium, which contained 2% cellobiose and 20 μg/ml of erythromycin. The enzyme activities from the supernatant of overnight culture were assayed by CMC-congo red plate assay and Cellazyme T assay kit (Megazyme International Ireland, Ltd., Bray Business Park, Bray, Co., Wicklow, Ireland). The CMC-congo plate and the Cellazyme T assays indicated the transformant of another vector C. phytofermentans pCphy3510_{—}1163 showed increased activity than that of the control strain.

Using the methods above, other pQint vectors, as listed below, have been constructed and different genes electroporated into C. phytofermentans strains. Several are listed below.

TABLE 3

| Vector backbone | Promoter | Gene(s) |
|---|---|---|
| pMTL82351 | P3558 | Cpy_3202 |
| pMTL82351 | P3558 | Zymomonas PDC |
| pMTL82351 | P3558 | Zm PDC/AdhB |
| pMTL82351 | P3510 | glcP (B. subtilis glf)/Zm glk |
| pMTL82351 | P1029 | Ccel_3478-3479-3480 (NAD) |
| pMTL82351 | P1029 | Ccel_1310 (DHFR) |
| pMTL82351 | P1029 | B. sub LacA (beta-galactosidase) |
| pMTL82351 | P1029 | ermB (erythromycin-resistance) |
| pMTL82351 | P3925 | Q13_3925 (Adh) |
| pMTL82351 | None | Δpta (internal fragment) |
| pMTL82351 | None | Δpfl (double crossover) |
| pMTL82351 | None | Cpy_1163 |
| pMTL82251 | P3558 | Zm PDC |
| pMTL82251 | P3558 | Zm PDC/AdhB |
| pMTL82254 | P3668 | Himar1 (transposase) + Tn(spec) |
| pMTL82351 | P3668 | Himar1 (transposase) + Tn(catP) |
| pMTL82151 | P3558 | Zm PDC |
| pMTL82151 | P3558 | Zm PDC/AdhB |
| pMTL82151 | None | None |
| pMTL82251 | None | None |
| pMTL82351 | None | None |
| pMTL82351 | P1029 | None |

### Example 7

### Operon Construction for Thiamine Synthesis

FIG. 6 represents the molecular pathways, for example, Kegg map 00730 (www.genome.jp), that can be used to synthesize thiamine and its derivatives. Rectangles represent gene products and open small circles are other molecules, mostly products. General metabolic processes are described in round-cornered rectangles and dashed lines with arrows indicate some of the products of these processes. Solid lines are molecular interactions with arrows to illustrate the direction of the reaction. C. phytofermentans synthesizes many enzymes and molecules involved in the molecular processes necessary to form thiamine (2-[3-[(4-Amino-2-methyl-pyrimidin-5-yl)methyl]-4-methyl-thiazol-5-yl]ethanol) and only lacks a few enzymes to complete several of the molecular steps. These blocked steps are indicated with an “X”. Several of the inherent C. phytofermentans genes available for this synthesis are indicated on the diagram; for example, Cphy2301 is the equivalent of ThiI.

TABLE 4
putative function of the C. phytofermentans (Cphy) genes:

| | |
|---|---|
| Cphy_0181 | aminotransferase class V; K04487 cysteine desulfurase |
| | [EC: 2.8.1.7] [KO: K04487] [EC: 2.8.1.7] |
| Cphy_2179 | cysteine desulfurase (EC: 2.8.1.7), K04487 cysteine |
| | desulfurase [EC: 2.8.1.7] [KO: K04487] [EC: 2.8.1.7] |
| Cphy_2302 | aminotransferase class V; K04487 cysteine desulfurase |
| | [EC: 2.8.1.7] [KO: K04487] [EC: 2.8.1.7] |
| Cphy_2907 | cysteine desulfurase NifS (EC: 2.8.1.7); K04487 cysteine |
| | desulfurase [EC: 2.8.1.7] [KO: K04487] [EC: 2.8.1.7] |
| Cphy_1684 | SufS subfamily cysteine desulfurase; K11717 cysteine |
| | desulfurase/selenocysteine lyase [EC: 2.8.1.7 4.4.1.16] |
| | [KO: K11717] [EC: 4.4.1.16 2.8.1.7] |
| Cphy_2301 | thiamine biosynthesis/tRNA modification protein ThiI; |
| | K03151 thiamine biosynthesis protein ThiI [KO: K03151] |
| Cphy_2082 | thiH; thiamine biosynthesis protein ThiH [KO: K03150] |
| Cphy_0369 | RdgB/HAM1 family non-canonical purine NTP |
| | pyrophosphatase; K01516 nucleoside-triphosphatase |
| | [EC: 3.6.1.15] [KO: K01516] [EC: 3.6.1.15] |
| Cphy_2483 | thiamine pyrophosphokinase; K00949 thiamine |
| | pyrophosphokinase [EC: 2.7.6.2] [KO: K00949] |
| | [EC: 2.7.6.2] |

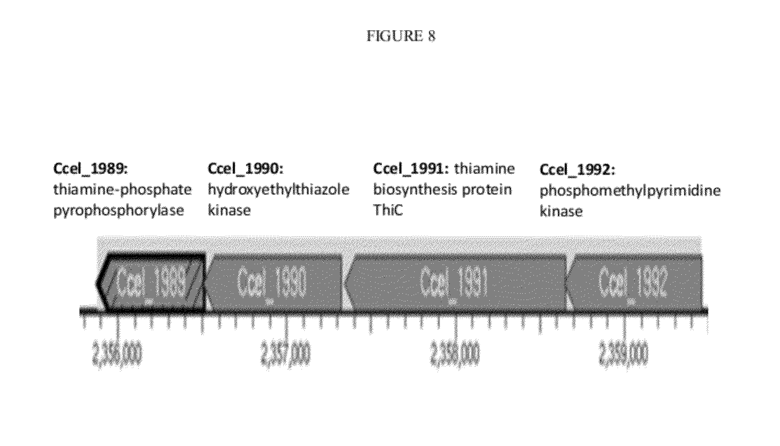

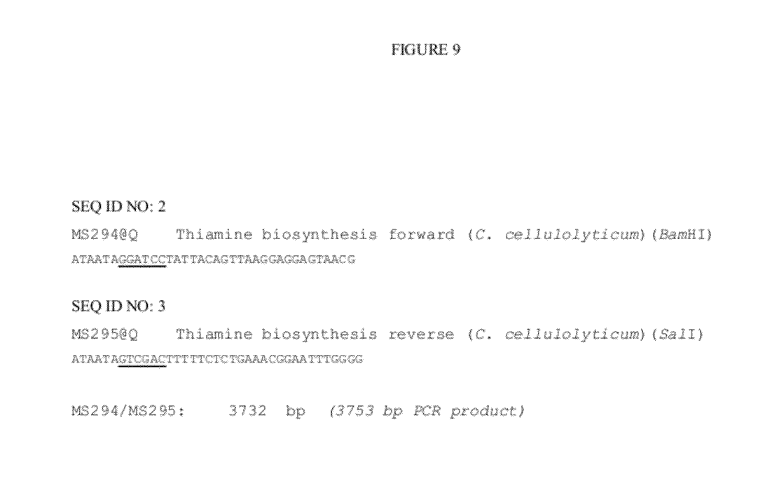

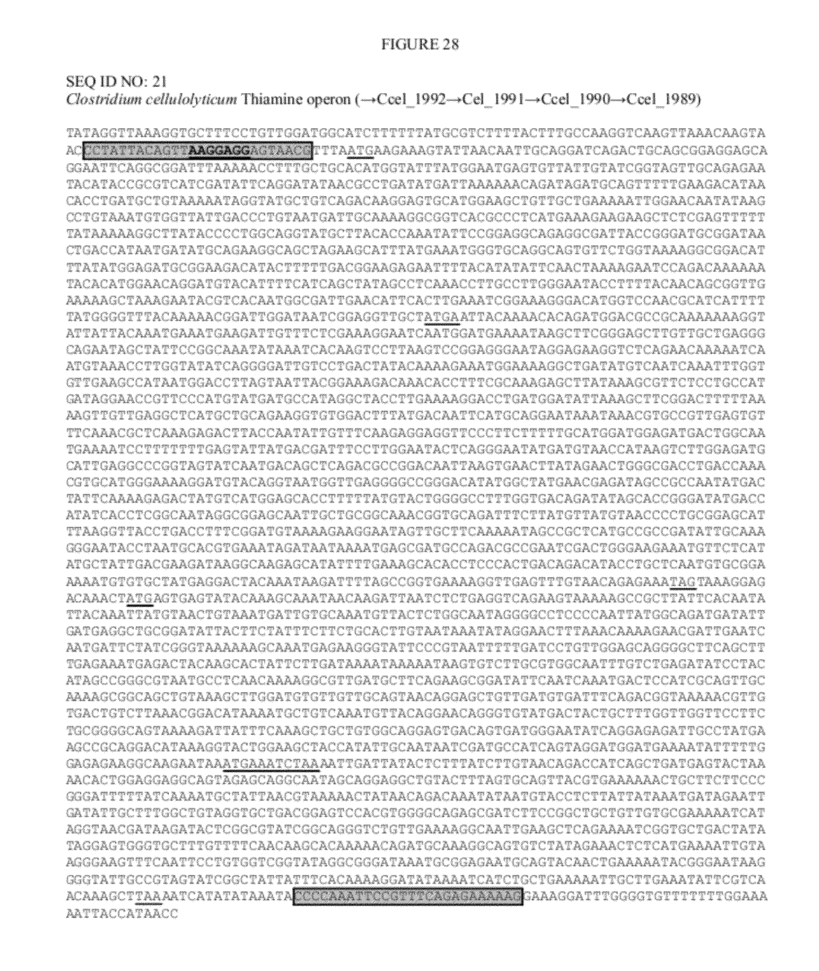

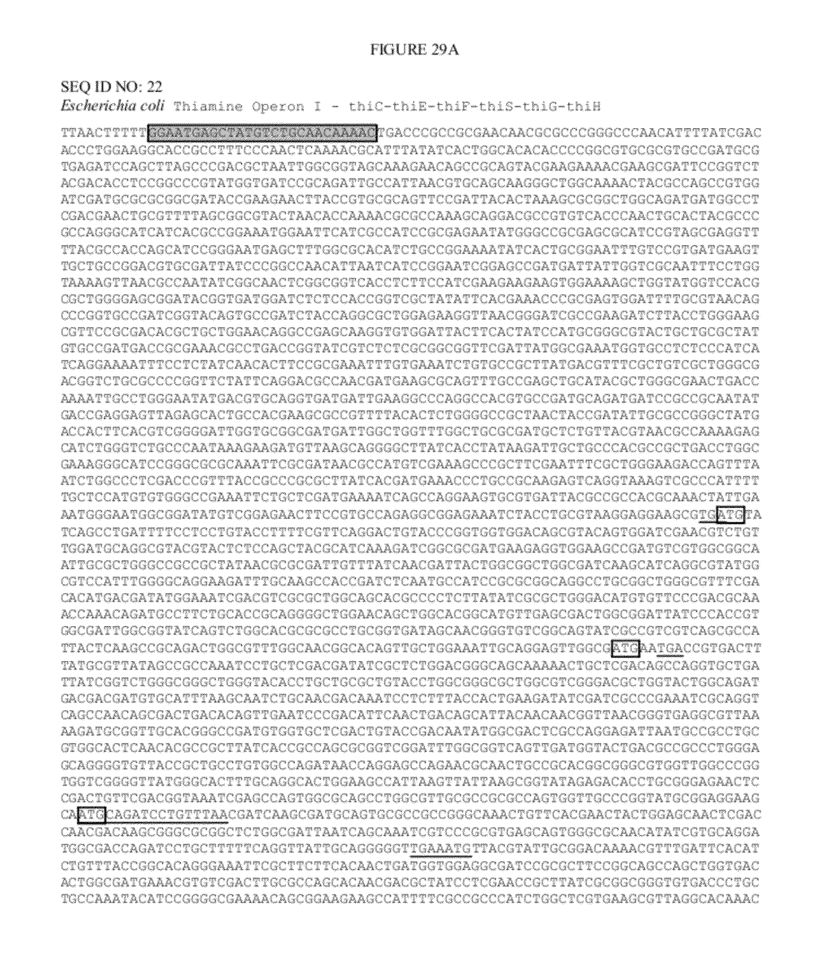

There are several methods by which a complete thiamine biosynthesis pathway can be constructed in C. phytofermentans. One cloning strategy for synthesis and possible overexpression of TPP in C. phytofermentans involves cloning four genes from another Clostridium anaerobe, Clostridium cellulolyticum. C. cellulolyticum has the essential genes to convert the side products of purine metabolism [aminoimidazole ribotide] and glycolysis [4-methyl-5-(2-hydroxyethyl)-thiazole] to thiamine phosphate. These four genes, Ccel_{—}1989, Ccel_{—}1990, Ccel_{—}1991, and Ccel_{—}1992 are used to construct an operon (FIG. 8) which is amplified via MS294@Q and MS295@Q primers (FIG. 9). After digesting the PCR product, it is inserted into plasmid pUniExp via BamHI and SalI restriction sites. The plasmid is ligated, inserted and transformed into E. coli, then isolated and the sequence verified. Finally, it is transferred into C. phytofermentans by electroporation as described supra and the resulting strains verified. The sequence of the operon, →>Ccel_{—}1992→Cel_{—}1991→Ccel_{—}1990→Ccel_{—}1989, is found in FIG. 28; polynucleotide and polypeptide sequences can be found in Table 5.

TABLE 5
C. cellulolyticum Thiamine Metabolic Pathway Sequences

| | |
|---|---|
| SEQ ID NO: 33 | atgaagaaagtattaacaattgcaggatcagactgcagcggaggagcaggaattcaggcggat |
| Nucleotide | ttaaaaacctttgctgcacatggtatttatggaatgagtgttattgtatcggtagttgcagag |
| Clostridium | aatacataccgcgtcatcgatattcaggatataacgcctgatatgattaaaaaacagatagat |
| cellulolyticum | gcagtttttgaagacataacacctgatgctgtaaaaataggtatgctgtcagacaaggagtgc |
| Ccel_1992 | atggaagctgttgctgaaaaattggaacaatataagcctgtaaatgtggttattgaccctgta |
| | atgattgcaaaaggcggtcacgccctcatgaaagaagaagctctcgagttttttataaaaagg |
| | cttatacccctggcaggtatgcttacaccaaatattccggaggcagaggcgattaccgggatg |
| | cggataactgaccataatgatatgcagaaggcagctagaagcatttatgaaatgggtgcaggc |
| | agtgttctggtaaaaggcggacatttatatggagatgcggaagacatactttttgacggaaga |
| | gaattttacatatattcaactaaaagaatccagacaaaaaatacacatggaacaggatgtaca |
| | ttttcatcagctatagcctcaaaccttgccttgggaataccttttacaacagcggttgaaaaa |
| | gctaaagaatacgtcacaatggcgattgaacattcacttgaaatcggaaagggacatggtcca |
| | acgcatcatttttatggggtttacaaaaacggattggataatcggaggttgctatga |
| SEQ ID NO: 34 | MKKVLTIAGSDCSGGAGIQADLKTFAAHGIYGMSVIVSVVAENTYRVIDIQDITPDMIKKQID |
| Peptide | AVFEDITPDAVKIGMLSDKECMEAVAEKLEQYKPVNVVIDPVMIAKGGHALMKEEALEFFIKR |
| Clostridium | LIPLAGMLTPNIPEAEAITGMRITDHNDMQKAARSIYEMGAGSVLVKGGHLYGDAEDILFDGR |
| cellulolyticum | EFYIYSTKRIQTKNTHGTGCTFSSAIASNLALGIPFTTAVEKAKEYVTMAIEHSLEIGKGHGP |
| Ccel_1992 | THHFYGVYKNGLDNRRLL |
| SEQ ID NO: 35 | atgaattacaaaacacagatggacgccgcaaaaaaaggtattattacaaatgaaatgaagatt |
| Nucleotide | gtttctcgaaaggaatcaatggatgaaaataagcttcgggagcttgttgctgagggcagaata |
| Clostridium | gctattccggcaaatataaatcacaagtccttaagtccggagggaataggagaaggtctcaga |
| cellulolyticum | acaaaaatcaatgtaaaccttggtatatcaggggattgtcctgactatacaaaagaaatggaa |
| Ccel_1991 | aaggctgatatgtcaatcaaatttggtgttgaagccataatggaccttagtaattacggaaag |
| | acaaacacctttcgcaaagagcttataaagcgttctcctgccatgataggaaccgttcccatg |
| | tatgatgccataggctaccttgaaaaggacctgatggatattaaagcttcggactttttaaaa |
| | gttgttgaggctcatgctgcagaaggtgtggactttatgacaattcatgcaggaataaataaa |
| | cgtgccgttgagtgtttcaaacgctcaaagagacttaccaatattgtttcaagaggaggttcc |
| | cttctttttgcatggatggagatgactggcaatgaaaatcctttttttgagtattatgacgat |
| | ttccttggaatactcagggaatatgatgtaaccataagtcttggagatgcattgaggcccggt |
| | agtatcaatgacagctcagacgccggacaattaagtgaacttatagaactgggcgacctgacc |
| | aaacgtgcatgggaaaaggatgtacaggtaatggttgaggggccgggacatatggctatgaac |
| | gagatagccgccaatatgactattcaaaagagactatgtcatggagcacctttttatgtactg |
| | gggcctttggtgacagatatagcaccgggatatgaccatatcacctcggcaataggcggagca |
| | attgctgcggcaaacggtgcagatttcttatgttatgtaacccctgcggagcatttaaggtta |
| | cctgacctttcggatgtaaaagaaggaatagttgcttcaaaaatagccgctcatgccgccgat |
| | attgcaaagggaatacctaatgcacgtgaaatagataataaaatgagcgatgccagacgccga |
| | atcgactgggaagaaatgttctcatatgctattgacgaagataaggcaagagcatattttgaa |
| | agcacacctcccactgacagacatacctgctcaatgtgcggaaaaatgtgtgctatgaggact |
| | acaaataagattttagccggtgaaaaggttgagtttgtaacagagaaatag |
| SEQ ID NO: 36 | MNYKTQMDAAKKGIITNEMKIVSRKESMDENKLRELVAEGRIAIPANINHKSLSPEGIGEGLR |
| Peptide | TKINVNLGISGDCPDYTKEMEKADMSIKFGVEAIMDLSNYGKTNTFRKELIKRSPAMIGTVPM |
| Clostridium | YDAIGYLEKDLMDIKASDFLKVVEAHAAEGVDFMTIHAGINKRAVECFKRSKRLTNIVSRGGS |
| cellulolyticum | LLFAWMEMTGNENPFFEYYDDFLGILREYDVTISLGDALRPGSINDSSDAGQLSELIELGDLT |
| Ccel_1991 | KRAWEKDVQVMVEGPGHMAMNEIAANMTIQKRLCHGAPFYVLGPLVTDIAPGYDHITSAIGGA |
| | IAAANGADFLCYVTPAEHLRLPDLSDVKEGIVASKIAAHAADIAKGIPNAREIDNKMSDARRR |
| | IDWEEMFSYAIDEDKARAYFESTPPTDRHTCSMCGKMCAMRTTNKILAGEKVEFVTEK |
| SEQ ID NO: 37 | atgagtgagtatacaaagcaaataacaagattaatctctgaggtcagaagtaaaaagccgctt |
| Nucleotide | attcacaatattacaaattatgtaactgtaaatgattgtgcaaatgttactctggcaataggg |
| Clostridium | gcctccccaattatggcagatgatattgatgaggctgcggatattacttctatttcttctgca |
| cellulolyticum | cttgtaataaatataggaactttaaacaaaagaacgattgaatcaatgattctatcgggtaaa |
| Ccel_1990 | aaagcaaatgagaagggtattcccgtaatttttgatcctgttggagcaggggcttcagctttg |
| | agaaatgagactacaagcactattcttgataaaataaaaataagtgtcttgcgtggcaatttg |
| | tctgagatatcctacatagccgggcgtaatgcctcaacaaaaggcgttgatgcttcagaagcg |
| | gatattcaatcaaatgactccatcgcagttgcaaaagcggcagctgtaaagcttggatgtgtt |
| | gttgcagtaacaggagctgttgatgtgatttcagacggtaaaaacgttgtgactgtcttaaac |
| | ggacataaaatgctgtcaaatgttacaggaacagggtgtatgactactgctttggttggttcc |
| | ttctgcggggcagtaaaagattatttcaaagctgctgtggcaggagtgacagtgatgggaata |
| | tcaggagagattgcctatgaagccgcaggacataaaggtactggaagctaccatattgcaata |
| | atcgatgccatcagtaggatggatgaaaatatttttggagagaaggcaagaataaatgaaatc |
| | taa |
| SEQ ID NO: 38 | MSEYTKQITRLISEVRSKKPLIHNITNYVTVNDCANVTLAIGASPIMADDIDEAADITSISSA |
| Peptide | LVINIGTLNKRTIESMILSGKKANEKGIPVIFDPVGAGASALRNETTSTILDKIKISVLRGNL |
| Clostridium | SEISYIAGRNASTKGVDASEADIQSNDSIAVAKAAAVKLGCVVAVTGAVDVISDGKNVVTVLN |
| cellulolyticum | GHKMLSNVTGTGCMTTALVGSFCGAVKDYFKAAVAGVTVMGISGEIAYEAAGHKGTGSYHIAI |
| Ccel_1990 | IDAISRMDENIFGEKARINEI |
| SEQ ID NO: 39 | atgaaatctaaaattgattatactctttatcttgtaacagaccatcagctgatgagtactaaa |
| Nucleotide | acactggaggaggcagtagagcaggcaatagcaggaggctgtactttagtgcagttacgtgaa |
| Clostridium | aaaactgcttcttcccgggatttttatcaaaatgctattaacgtaaaaactataacagacaaa |
| cellulolyticum | tataatgtacctcttattataaatgatagaattgatattgctttggctgtaggtgctgacgga |
| Ccel_1989 | gtccacgtggggcagagcgatcttccggctgctgttgtgcgaaaaatcataggtaacgataag |
| | atactcggcgtatcggcagggtctgttgaaaaggcaattgaagctcagaaaatcggtgctgac |
| | tatataggagtgggtgctttgttttcaacaagcacaaaaacagatgcaaaggcagtgtctata |
| | gaaactctcatgaaaattgtaagggaagtttcaattcctgtggtcggtataggcgggataaat |
| | gcggagaatgcagtacaactgaaaaatacgggaataaggggtattgccgtagtatcggctatt |
| | atttcacaaaaggatataaaatcatctgctgaaaaattgcttgaaatattcgtcaacaaagct |
| | taa |
| SEQ ID NO: 40 | MKSKIDYTLYLVTDHQLMSTKTLEEAVEQAIAGGCTLVQLREKTASSRDFYQNAINVKTITDK |
| Peptide | YNVPLIINDRIDIALAVGADGVHVGQSDLPAAVVRKIIGNDKILGVSAGSVEKAIEAQKIGAD |
| Clostridium | YIGVGALFSTSTKTDAKAVSIETLMKIVREVSIPVVGIGGINAENAVQLKNTGIRGIAVVSAI |
| cellulolyticum | ISQKDIKSSAEKLLEIFVNKA |
| Ccel_1989 | |

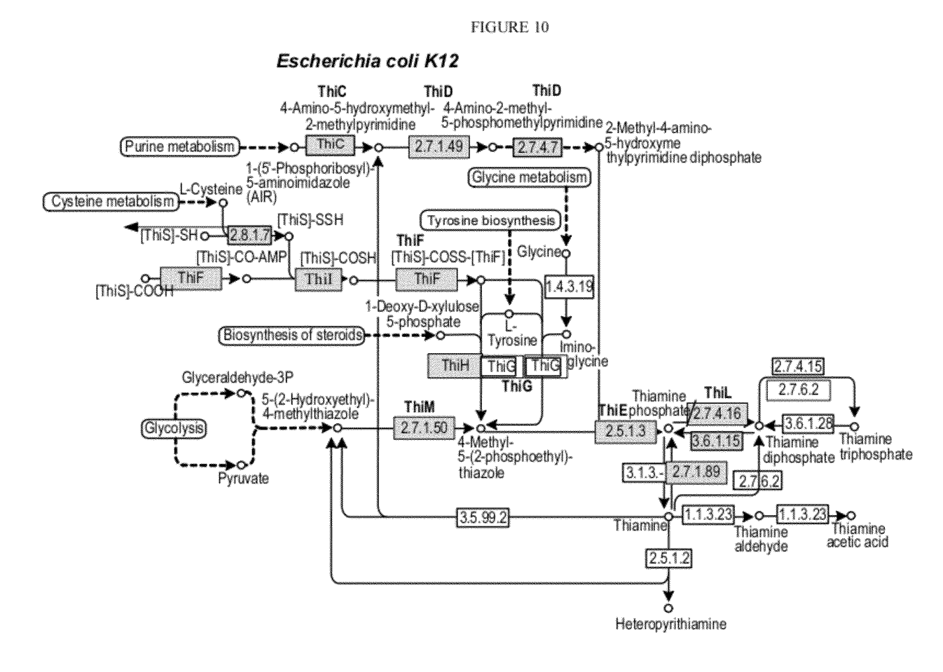

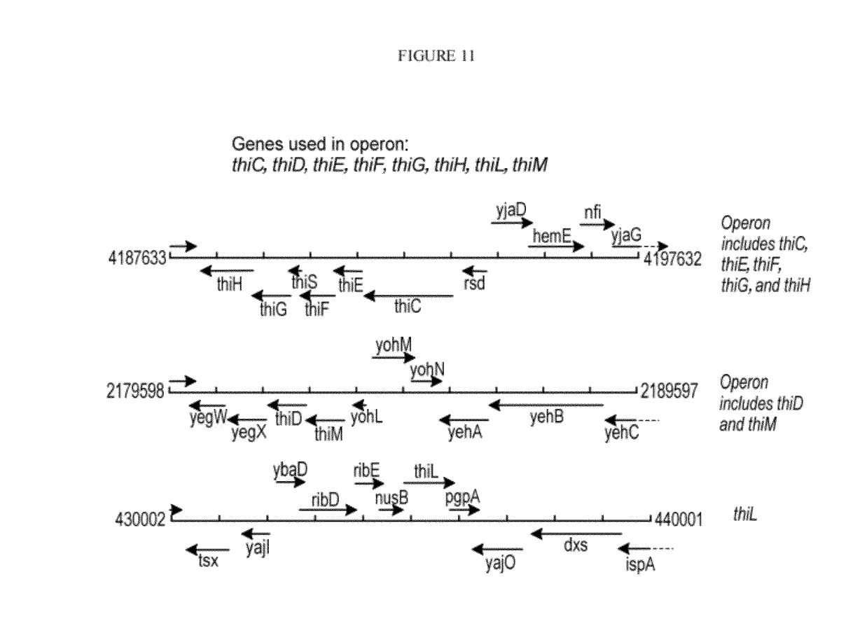

Alternatively, Escherichia. coli synthesizes all the essential genes to convert the side products of purine metabolism [aminoimidazole ribotide] and cysteine metabolism to thiamine diphosphate (FIG. 10). These genes, thiC, thiD, thiE, thiF, thiG, thiH, thiL, thiM are present in three operons (FIG. 11), and are incorporated into the C. phytofermentans genome. Cloning of the complete operon includes: thiC-thiE-thiF-thiS-thiG-thill-thiM-thiD-thiL.

Two different approaches are considered. In the first approach, the protein sequences from E. coli are reverse translated using the codon usage table of C. phytofermentans. Then an operon is synthesized using C. phytofermentans codons, incorporated with a promoter, such as Cphy3558 (pyruvate ferredoxin oxidoreductase), into a plasmid, such as the plasmids described supra and integrated into the C. phytofermentans genome. The promoter can be constitutive and moderately expressed.

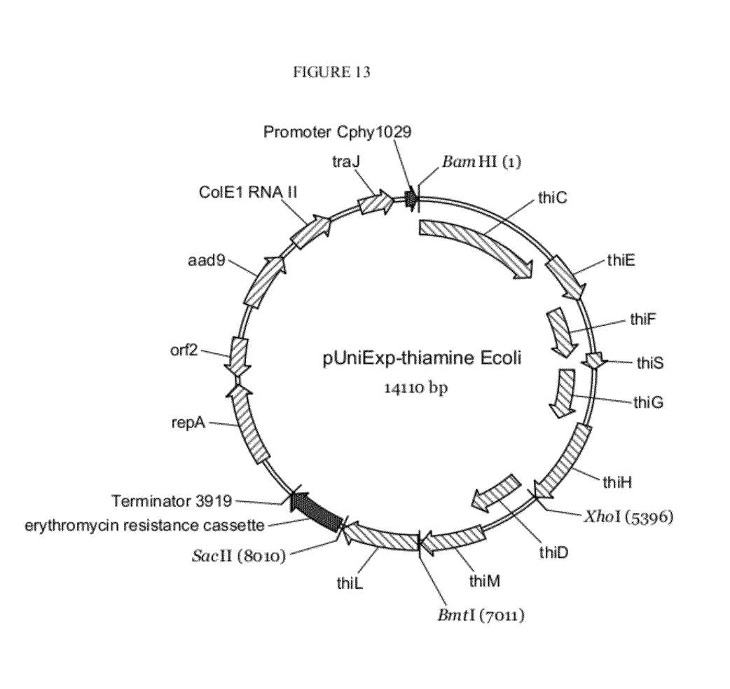

The alternative approach amplifys the three operons (below) from Escherichia coli chromosomal DNA and inserts them into pUniExp (FIG. 13) in whole or in part to collect these genes for integration. The promoter Cphy_{—}1029 can be used for the expression of the genes oriented as an operon.

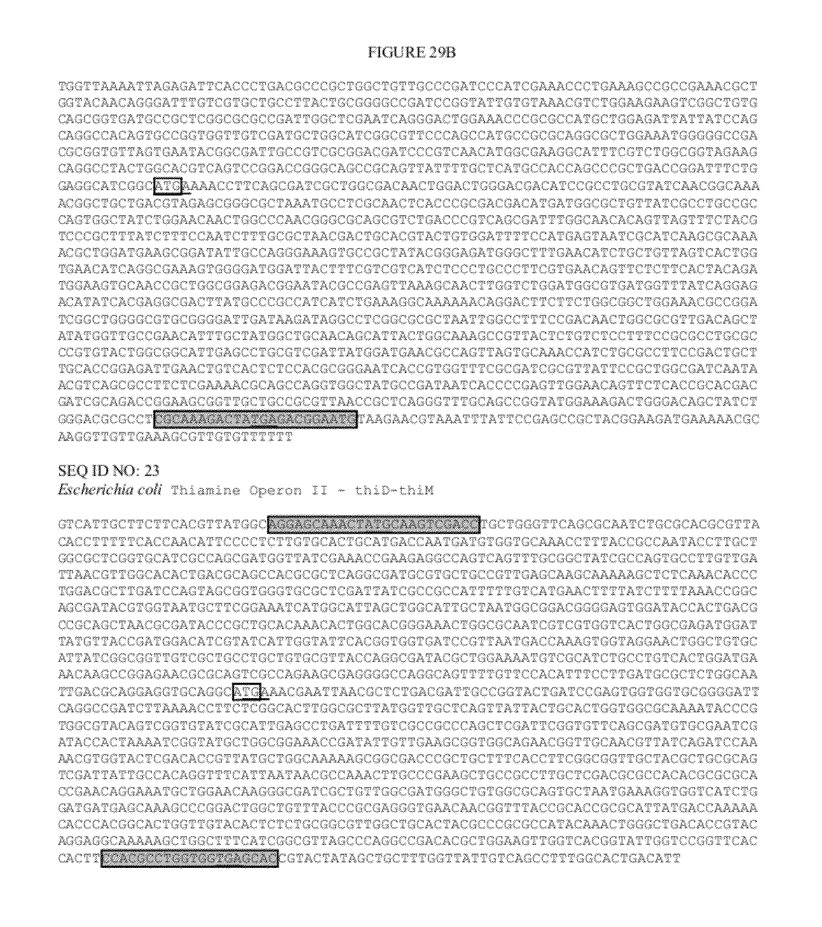

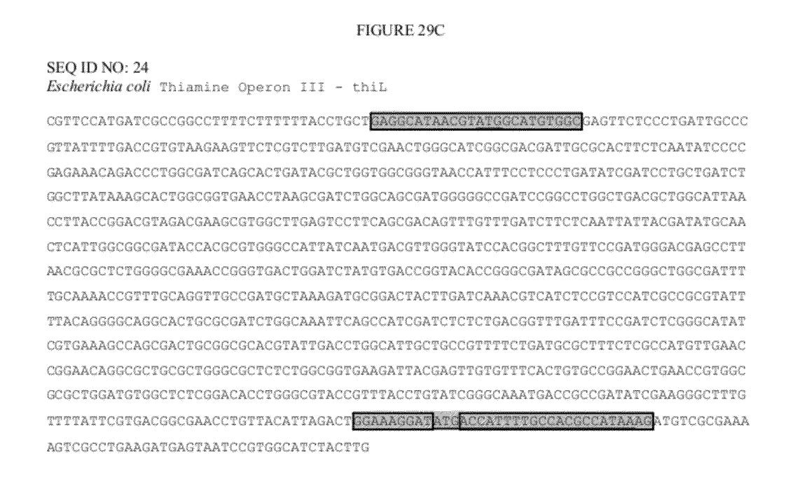

Operons for amplification via PCR can be found in FIG. 29; polynucleotide and polypeptide sequences relating to E. coli thiamine metabolic pathway genes can be found in Table 6.

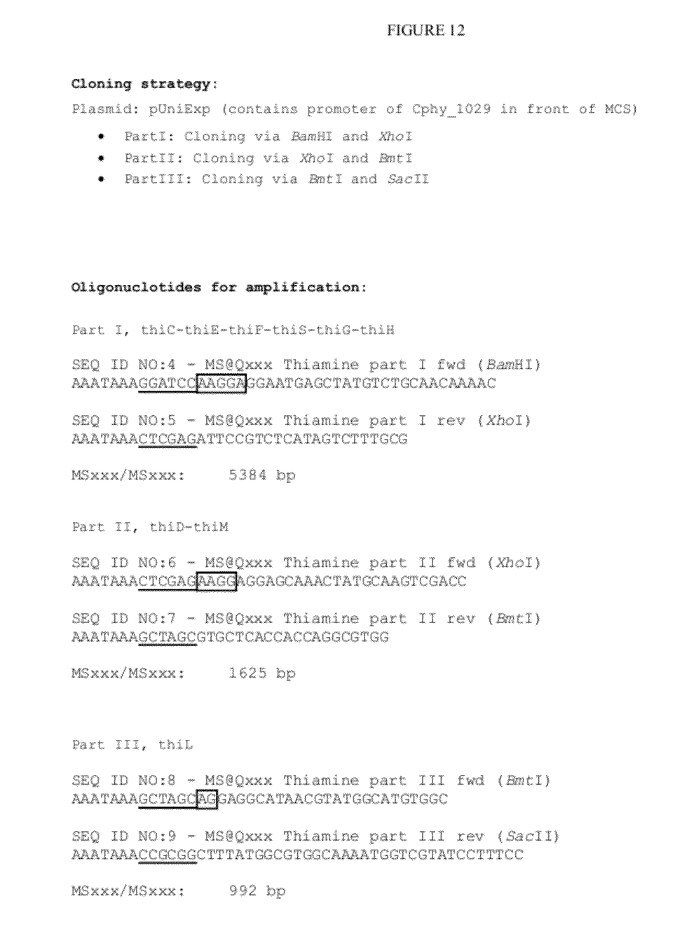

For example, restriction enzymes at sitesl and 2 are used to cleave thiH, thiG, thiS, thiF, thi E and thiC from E. coli operon 1; at sites 3 and 4, thiD and thiM are similarly selected out of E. coli operon 2; and at sites 5 and 6, thiL is selected from E. coli operon 3. The cloning strategy and amplification primers are described in FIG. 12.

In the sequence of the E. coli gene thiL there is an ATG triplet (boxed) that is not in the same frame as the major open reading frame. In addition, there is a potential ribosome-binding sequence (Shine-Delgarno sequence) upstream of this ATG. These two elements could lead to inappropriate translation initiation near the end of the thiL gene (underlined sequence). Since this ATG is not in the same frame as the TAA stop codon, translation would continue into the 3′-untranslated region of the mRNA. The ATG overlaps an in-frame TAT codon. Changing this codon to TAC preserves the identity of the coded amino acid (tyrosine) while eliminating the out-of-frame ATG (becomes ACG). This change in the coding sequence ensures proper translation of the thiL mRNA while preventing inadvertent translation initiation near the 3′ end of the gene.

TABLE 6
E. coli Thiamine Metabolic Pathway Sequences

| | |
|---|---|
| SEQ ID NO: 41 | atgtctgcaa caaaactgac ccgccgcgaa caacgcgccc gggcccaaca |
| Nucleotide | ttttatcgac accctggaag gcaccgcctt tcccaactca aaacgcattt |
| Escherichia | atatcactgg cacacacccc ggcgtgcgcg tgccgatgcg tgagatccag |
| coli | cttagcccga cgctaattgg cggtagcaaa gaacagccgc agtacgaaga |
| ThiC | aaacgaagcg attccggtct acgacacctc cggcccgtat ggtgatccgc |
| | agattgccat taacgtgcag caagggctgg caaaactacg ccagccgtgg |
| | atcgatgcgc gcggcgatac cgaagaactt accgtgcgca gttccgatta |
| | cactaaagcg cggctggcag atgatggcct cgacgaactg cgttttagcg |
| | gcgtactaac accaaaacgc gccaaagcag gacgccgtgt cacccaactg |
| | cactacgccc gccagggcat catcacgccg gaaatggaat tcatcgccat |
| | ccgcgagaat atgggccgcg agcgcatccg tagcgaggtt ttacgccacc |
| | agcatccggg aatgagcttt ggcgcacatc tgccggaaaa tatcactgcg |
| | gaatttgtcc gtgatgaagt tgctgccgga cgtgcgatta tcccggccaa |
| | cattaatcat ccggaatcgg agccgatgat tattggtcgc aatttcctgg |
| | taaaagttaa cgccaatatc ggcaactcgg cggtcacctc ttccatcgaa |
| | gaagaagtgg aaaagctggt atggtccacg cgctggggag cggatacggt |
| | gatggatctc tccaccggtc gctatattca cgaaacccgc gagtggattt |
| | tgcgtaacag cccggtgccg atcggtacag tgccgatcta ccaggcgctg |
| | gagaaggtta acgggatcgc cgaagatctt acctgggaag cgttccgcga |
| | cacgctgctg gaacaggccg agcaaggtgt ggattacttc actatccatg |
| | cgggcgtact gctgcgctat gtgccgatga ccgcgaaacg cctgaccggt |
| | atcgtctctc gcggcggttc gattatggcg aaatggtgcc tctcccatca |
| | tcaggaaaat ttcctctatc aacacttccg cgaaatttgt gaaatctgtg |
| | ccgcttatga cgtttcgctg tcgctgggcg acggtctgcg ccccggttct |
| | attcaggacg ccaacgatga agcgcagttt gccgagctgc atacgctggg |
| | cgaactgacc aaaattgcct gggaatatga cgtgcaggtg atgattgaag |
| | gcccaggcca cgtgccgatg cagatgatcc gccgcaatat gaccgaggag |
| | ttagagcact gccacgaagc gccgttttac actctggggc cgctaactac |
| | cgatattgcg ccgggctatg accacttcac gtcggggatt ggtgcggcga |
| | tgattggctg gtttggctgc gcgatgctct gttacgtaac gccaaaagag |
| | catctgggtc tgcccaataa agaagatgtt aagcaggggc ttatcaccta |
| | taagattgct gcccacgccg ctgacctggc gaaagggcat ccgggcgcgc |
| | aaattcgcga taacgccatg tcgaaagccc gcttcgaatt tcgctgggaa |
| | gaccagttta atctggccct cgacccgttt accgcccgcg cttaCcacga |
| | tgaaaccctg ccgcaagagt caggtaaagt cgcccattct tgctccatgt |
| | gtgggccgaa attctgctcg atgaaaatca gccaggaagt gcgtgattac |
| | gccgccacgc aaactattga aatgggaatg gcggatatgt cggagaactt |
| | ccgcgccaga ggcgggagaa atctacctgc gtaa |
| SEQ ID NO: 42 | MSATKLTRREQRARAQHFIDTLEGTAFPNSKRIYITGTHPGVRVPMREIQLSPTLIG |
| Peptide | GSKEQPQYEENEAIPVYDTSGPYGDPQIAINVQQGLAKLRQPWIDARGDTEELTVRS |
| Escherichia | SDYTKARLADDGLDELRFSGVLTPKRAKAGRRVTQLHYARQGIITPEMEFIAIRENM |
| coli | GRERIRSEVLRHQHPGMSFGAHLPENITAEFVRDEVAAGRAIIPANINHPESEPMII |
| ThiC | GRNFLVKVNANIGNSAVTSSIEEEVEKLVWSTRWGADTVMDLSTGRYIHETREWILR |
| | NSPVPIGTVPIYQALEKVNGIAEDLTWEAFRDTLLEQAEQGVDYFTIHAGVLLRYVP |
| | MTAKRLTGIVSRGGSIMAKWCLSHHQENFLYQHFREICEICAAYDVSLSLGDGLRPG |
| | SIQDANDEAQFAELHTLGELTKIAWEYDVQVMIEGPGHVPMQMIRRNMTEELEHCHE |
| | APFYTLGPLTTDIAPGYDHFTSGIGAAMIGWFGCAMLCYVTPKEHLGLPNKEDVKQG |
| | LITYKIAAHAADLAKGHPGAQIRDNAMSKARFEFRWEDQFNLALDPFTARAYHDETL |
| | PQESGKVAHFCSMCGPKFCSMKISQEVRDYAATQTIEMGMADMSENFRARGGRNLPA |
| SEQ ID NO: 43 | atgtatcagc ctgattttcc tcctgtacct tttcgttcag gactgtaccc |
| Nucleotide | ggtggtggac agcgtacagt ggatcgaacg tctgttggat gcaggcgtac |
| Escherichia | gtactctcca gctacgcatc aaagatcggc gcgatgaaga ggtggaagcc |
| coli | gatgtcgtgg cggcaattgc gctgggccgc cgctataacg cgcgattgtt |
| ThiE | tatcaacgat tactggcggc tggcgatcaa gcatcaggcg tatggcgtcc |
| | atttggggca ggaagatttg caagccaccg atctcaatgc catccgcgcg |
| | gcaggcctgc ggctgggcgt ttcgacacat gacgatatgg aaatcgacgt |
| | cgcgctggca gcacgcccct cttatatcgc gctgggacat gtgttcccga |
| | cgcaaaccaa acagatgcct tctgcaccgc aggggctgga acagctggca |
| | cggcatgttg agcgactggc ggattatccc accgtggcga ttggcggtat |
| | cagtctggca cgcgcgcctg cggtgatagc aacgggtgtc ggcagtatcg |
| | ccgtcgtcag cgccattact caagccgcag actggcgttt ggcaacggca |
| | cagttgctgg aaattgcagg agttggcgat gaatga |
| SEQ ID NO: 44 | MYQPDFPPVPFRSGLYPVVDSVQWIERLLDAGVRTLQLRIKDRRDEEVEADVVAAIA |
| Peptide | LGRRYNARLFINDYWRLAIKHQAYGVHLGQEDLQATDLNAIRAAGLRLGVSTHDDME |
| Escherichia | IDVALAARPSYIALGHVFPTQTKQMPSAPQGLEQLARHVERLADYPTVAIGGISLAR |
| coli | APAVIATGVGSIAVVSAITQAADWRLATAQLLEIAGVGDE |
| ThiE | |
| SEQ ID NO: 45 | atgaatgacc gtgactttat gcgttatagc cgccaaatcc tgctcgacga |
| Nucleotide | tatcgctctg gacgggcagc aaaaactgct cgacagccag gtgctgatta |
| Escherichia | tcggtctggg cgggctgggt acacctgctg cgctgtacct ggcgggcgct |
| coli | ggcgtcggga cgctggtact ggcagatgac gacgatgtgc atttaagcaa |
| ThiF | tctgcaacga caaatcctct ttaccactga agatatcgat cgcccgaaat |
| | cgcaggtcag ccaacagcga ctgacacagt tgaatcccga cattcaactg |
| | acagcattac aacaacggtt aacgggtgag gcgttaaaag atgcggttgc |
| | acgggccgat gtggtgctcg actgtaccga caatatggcg actcgccagg |
| | agattaatgc cgcctgcgtg gcactcaaca cgccgcttat caccgccagc |
| | gcggtcggat ttggcggtca gttgatggta ctgacgccgc cctgggagca |
| | ggggtgttac cgctgcctgt ggccagataa ccaggagcca gaacgcaact |
| | gccgcacggc gggcgtggtt ggcccggtgg tcggggttat gggcactttg |
| | caggcactgg aagccattaa gttattaagc ggtatagaga cacctgcggg |
| | agaactccga ctgttcgacg gtaaatcgag ccagtgcagc ctggcgttgc |
| | gccgcgccag tggttgcccg gtatgcggag gaagcaatgc agatcctgtt |
| | taa |
| SEQ ID NO: 46 | MNDRDFMRYSRQILLDDIALDGQQKLLDSQVLIIGLGGLGTPAALYLAGAGVGTLVL |
| Peptide | ADDDDVHLSNLQRQILFTTEDIDRPKSQVSQQRLTQLNPDIQLTALQQRLTGEALKD |
| Escherichia | AVARADVVLDCTDNMATRQEINAACVALNTPLITASAVGFGGQLMVLTPPWEQGCYR |
| coli | CLWPDNQEPERNCRTAGVVGPVVGVMGTLQALEAIKLLSGIETPAGELRLFDGKSSQ |
| ThiF | CSLALRRASGCPVCGGSNADPV |
| SEQ ID NO: 47 | atgcagatcc tgtttaacga tcaagcgatg cagtgcgccg ccgggcaaac |
| Nucleotide | tgttcacgaa ctactggagc aactcgacca acgacaagcg ggcgcggccc |
| Escherichia | tggcgattaa tcagcaaatc gtcccgcgtg agcagtgggc gcaacatatc |
| coli | gtgcaggatg gcgaccagat cctgcttttt caggttattg cagggggttg a |
| ThiS | |
| SEQ ID NO: 48 | MQILFNDQAMQCAAGQTVHELLEQLDQRQAGAALAINQQIVPREQWAQHIVQDGDQI |
| Peptide | LLFQVIAGG |
| Escherichia | |
| coli | |
| ThiS | |
| SEQ ID NO: 49 | atgttacgta ttgcggacaa aacgtttgat tcacatctgt ttaccggcac |
| Nucleotide | agggaaattc gcttcttcac aactgatggt ggaggcgatc cgcgcttccg |
| Escherichia | gcagccagct ggtgacactg gcgatgaaac gtgtcgactt gcgccagcac |
| coli | aacgacgcta tcctcgaacc gcttatcgcg gcgggtgtga ccctgctgcc |
| ThiG | aaatacatcc ggggcgaaaa cagcggaaga agccattttc gccgcccatc |
| | tggctcgtga agcgttaggc acaaactggt taaaattaga gattcaccct |
| | gacgcccgct ggctgttgcc cgatcccatc gaaaccctga aagccgccga |
| | aacgctggta caacagggat ttgtcgtgct gccttactgc ggggccgatc |
| | cggtattgtg taaacgtctg gaagaagtcg gctgtgcagc ggtgatgccg |
| | ctcggcgcgc cgattggctc gaatcaggga ctggaaaccc gcgccatgct |
| | ggagattatt atccagcagg ccacagtgcc ggtggttgtc gatgctggca |
| | tcggcgttcc cagccatgcc gcgcaggcgc tggaaatggg ggccgacgcg |
| | gtgttagtga atacggcgat tgccgtcgcg gacgatcccg tcaacatggc |
| | gaaggcattt cgtctggcgg tagaagcagg cctactggca cgtcagtccg |
| | gaccgggcag ccgcagttat tttgctcatg ccaccagccc gctgaccgga |
| | tttctggagg catcggcatg a |
| SEQ ID NO: 50 | MLRIADKTFDSHLFTGTGKFASSQLMVEAIRASGSQLVTLAMKRVDLRQHNDAILEP |
| Peptide | LIAAGVTLLPNTSGAKTAEEAIFAAHLAREALGTNWLKLEIHPDARWLLPDPIETLK |
| Escherichia | AAETLVQQGFVVLPYCGADPVLCKRLEEVGCAAVMPLGAPIGSNQGLETRAMLEIII |
| coli | QQATVPVVVDAGIGVPSHAAQALEMGADAVLVNTAIAVADDPVNMAKAFRLAVEAGL |
| ThiG | LARQSGPGSRSYFAHATSPLTGFLEASA |
| SEQ ID NO: 51 | atgaaaacct tcagcgatcg ctggcgacaa ctggactggg acgacatccg |
| Nucleotide | cctgcgtatc aacggcaaaa cggctgctga cgtagagcgg gcgctaaatg |
| Escherichia | cctcgcaact cacccgcgac gacatgatgg cgctgttatc gcctgccgcc |
| coli | agtggctatc tggaacaact ggcccaacgg gcgcagcgtc tgacccgtca |
| ThiH | gcgatttggc aacacagtta gtttctacgt cccgctttat ctttccaatc |
| | tttgcgctaa cgactgcacg tactgtggat tttccatgag taatcgcatc |
| | aagcgcaaaa cgctggatga agcggatatt gccagggaaa gtgccgctat |
| | acgggagatg ggctttgaac atctgctgtt agtcactggt gaacatcagg |
| | cgaaagtggg gatggattac tttcgtcgtc atctccctgc ccttcgtgaa |
| | cagttctctt cactacagat ggaagtgcaa ccgctggcgg agacggaata |
| | cgccgagtta aagcaacttg gtctggatgg cgtgatggtt tatcaggaga |
| | catatcacga ggcgacttat gcccgccatc atctgaaagg caaaaaacag |
| | gacttcttct ggcggctgga aaccccggat cggctggggc gtgcggggat |
| | tgataagata ggcctcggcg cgctaattgg cctttccgac aactggcgcg |
| | ttgacagcta tatggttgcc gaacatttgc tatggctgca acagcattac |
| | tggcaaagcc gttactctgt ctcctttccg cgcctgcgcc cgtgtactgg |
| | cggcattgag cctgcgtcga ttatggatga acgccagtta gtgcaaacca |
| | tctgcgcctt ccgactgctt gcaccggaga ttgaactgtc actctccacg |
| | cgggaatcac cgtggtttcg cgatcgcgtt attccgctgg cgatcaataa |
| | cgtcagcgcc ttctcgaaaa cgcagccagg tggctatgcc gataatcacc |
| | ccgagttgga acagttctca ccgcacgacg atcgcagacc ggaagcggtt |
| | gctgccgcgt taaccgctca gggtttgcag ccggtatgga aagactggga |
| | cagctatctg ggacgcgcct cgcaaagact atga |
| SEQ ID NO: 52 | MKTFSDRWRQLDWDDIRLRINGKTAADVERALNASQLTRDDMMALLSPAASGYLEQL |
| Peptide | AQRAQRLTRQRFGNTVSFYVPLYLSNLCANDCTYCGFSMSNRIKRKTLDEADIARES |
| Escherichia | AAIREMGFEHLLLVTGEHQAKVGMDYFRRHLPALREQESSLQMEVQPLAETEYAELK |
| coli | QLGLDGVMVYQETYHEATYARHHLKGKKQDFFWRLETPDRLGRAGIDKIGLGALIGL |
| ThiH | SDNWRVDSYMVAEHLLWLQQHYWQSRYSVSFPRLRPCTGGIEPASIMDERQLVQTIC |
| | AFRLLAPEIELSLSTRESPWFRDRVIPLAINNVSAFSKTQPGGYADNHPELEQFSPH |
| | DDRRPEAVAAALTAQGLQPVWKDWDSYLGRASQRL |
| SEQ ID NO: 53 | atgaaacgaa ttaacgctct gacgattgcc ggtactgatc cgagtggtgg |
| Nucleotide | tgcggggatt caggccgatc ttaaaacctt ctcggcactt ggcgcttatg |
| Escherichia | gttgctcagt tattactgca ctggtagcgc aaaatacccg tggcgtacag |
| coli | tcggtgtatc gcattgagcc tgattttgtc gccgcccagc tcgattcggt |
| ThiD | gttcagcgat gtgcgaatcg acaccactaa aatcggtatg ttggcggaaa |
| | ccgatattgt tgaagcggtg gcagaacggt tgcaacgtta tcagatccaa |
| | aacgtggtac tcgacaccgt tatgctggca aaaagcggcg acccgctgct |
| | ttcaccttcg gcggttgtta cgctgcgcag tcgattattg ccacaggttt |
| | cattaataac gccaaacttg cccgaagctg ccgccttgct cgacgcgcca |
| | cacgcgcgca ccgaacagga aatgctggaa caagggcgat cgctgttggc |
| | gatgggatgt ggcgcagtgc taatgaaagg tggtcatctg gatgatgagc |
| | aaagcccgga ctggctgttt acccgcgagg gtgaacaacg gtttaccgca |
| | ccgcgcatta tgaccaaaaa cacccacggc actggttgta cactctctgc |
| | ggcgttggct gcactacgcc cgcgccatac aaactgggct gacaccgtac |
| | aggaggcaaa aagctggctt tcatcggcgt tagcccaggc cgacacgctg |
| | gaagttggtc acggtattgg tccggttcac cacttccacg cctggtggtg a |
| SEQ ID NO: 54 | MKRINALTIAGTDPSGGAGIQADLKTFSALGAYGCSVITALVAQNTRGVQSVYRIEP |
| Peptide | DFVAAQLDSVFSDVRIDTTKIGMLAETDIVEAVAERLQRYQIQNVVLDTVMLAKSGD |
| Escherichia | PLLSPSAVVTLRSRLLPQVSLITPNLPEAAALLDAPHARTEQEMLEQGRSLLAMGCG |
| coli | AVLMKGGHLDDEQSPDWLFTREGEQRFTAPRIMTKNTHGTGCTLSAALAALRPRHTN |
| ThiD | WADTVQEAKSWLSSALAQADTLEVGHGIGPVHHFHAWW |
| SEQ ID NO: 55 | tcatgcctgc acctcctgcg tcaattgcca gagcgcatca aggaaatgtg |
| Nucleotide | gaacaaaact gcctggcccc tcgcttctgg cgactgcgcg ttctccggct |
| Escherichia | tgtttcatcc agtgacaggc agatgcgaca ttttccagcg tatcgcctgg |
| coli | taacgcacag caggcagcga caaccgccga taatgcacag ccagttccta |
| ThiM | ccactttggt cattaacgga tcgccaccgt gaatgccaac ggtgcgacga |
| | ccatcggtaa cataatccat ctcgccagtg accacgacga ttgcgccagt |
| | ttcccgtgcc agtgtttgtg cagcgggtat cgcattagct gccgcgtcag |
| | tggtatccac tccccgtccg ccagtagcaa cgccagctaa tgccatgatt |
| | tccgaagcat taccacgtat cgctgccggt ttaaaagata aaagttcatg |
| | acaaaaatgg cgacgataat cgagcgcacc aaccgctact ggatcaagcg |
| | tccagggggt ttgagagctt tttgcttgct caacggcggc acgcatcgcc |
| | tgtgcgcgtg gctgcgtcag cgtgccaacg ttaatcaaca aggcactggc |
| | gatagccgca aactgactgg cctcttcggt ttcgataacc atcgctggcg |
| | atgcaccgat cgccagcaag gtattggcag taaaggtttg caccacatcg |
| | ttggtcatgc aatgcacaag aggggaatgt tggtgaaaaa ggtgtaacgc |
| | gtgcgcagat tgcgcggaac tcagcaggtc gacttgcat |
| SEQ ID NO: 56 | MQVDLLSSAQSAHALHLFHQHSPLVHCMTNDVVQTFTANTLLAIGASPAMVIETEEA |
| Peptide | SQFAAIASALLINVGTLTQPRAQAMRAAVEQAKSSQTPWTLDPVAVGALDYRRHFCH |
| Escherichia | ELLSFKPAAIRGNASEIMALAGVATGGRGVDTTDAAANAIPAAQTLARETGAIVVVT |
| coli | GEMDYVTDGRRTVGIHGGDPLMTKVVGTGCALSAVVAACCALPGDTLENVASACHWM |
| ThiM | KQAGERAVARSEGPGSFVPHFLDALWQLTQEVQA |
| SEQ ID NO: 57 | atggcatgtg gcgagttctc cctgattgcc cgttattttg accgtgtaag |
| Nucleotide | aagttctcgt cttgatgtcg aactgggcat cggcgacgat tgcgcacttc |
| Escherichia | tcaatatccc cgagaaacag accctggcga tcagcactga tacgctggtg |
| coli | gcgggtaacc atttcctccc tgatatcgat cctgctgatc tggcttataa |
| ThiL | agcactggcg gtgaacctaa gcgatctggc agcgatgggg gccgatccgg |
| | cctggctgac gctggcatta accttaccgg acgtagacga agcgtggctt |
| | gagtccttca gcgacagttt gtttgatctt ctcaattatt acgatatgca |
| | actcattggc ggcgatacca cgcgtgggcc attatcaatg acgttgggta |
| | tccacggctt tgttccgatg ggacgagcct taacgcgctc tggggcgaaa |
| | ccgggtgact ggatctatgt gaccggtaca ccgggcgata gcgccgccgg |
| | gctggcgatt ttgcaaaacc gtttgcaggt tgccgatgct aaagatgcgg |
| | actacttgat caaacgtcat ctccgtccat cgccgcgtat tttacagggg |
| | caggcactgc gcgatctggc aaattcagcc atcgatctct ctgacggttt |
| | gatttccgat ctcgggcata tcgtgaaagc cagcgactgc ggcgcacgta |
| | ttgacctggc attgctgccg ttttctgatg cgctttctcg ccatgttgaa |
| | ccggaacagg cgctgcgctg ggcgctctct ggcggtgaag attacgagtt |
| | gtgtttcact gtgccggaac tgaaccgtgg cgcgctggat gtggctctcg |
| | gacacctggg cgtaccgttt acctgtatcg ggcaaatgac cgccgatatc |
| | gaagggcttt gttttattcg tgacggcgaa cctgttacat tagactggaa |
| | aggatatgac cattttgcca cgccataa |
| SEQ ID NO: 58 | MACGEFSLIARYFDRVRSSRLDVELGIGDDCALLNIPEKQTLAISTDTLVAGNHFLP |
| Peptide | DIDPADLAYKALAVNLSDLAAMGADPAWLTLALTLPDVDEAWLESFSDSLFDLLNYY |
| Escherichia | DMQLIGGDTTRGPLSMTLGIHGFVPMGRALTRSGAKPGDWIYVTGTPGDSAAGLAIL |
| coli | QNRLQVADAKDADYLIKRHLRPSPRILQGQALRDLANSAIDLSDGLISDLGHIVKAS |
| ThiL | DCGARIDLALLPFSDALSRHVEPEQALRWALSGGEDYELCFTVPELNRGALDVALGH |
| | LGVPFTCIGQMTADIEGLCFIRDGEPVTLDWKGYDHFATP |

### Example 8

### Examination of the Effect of Nicotinic Acid on the Growth and Ethanol Titer of Clostridium phytofermentans

Supplementation of fermentation media with nicotinic acid at 30 mg/L increased the yield of ethanol (FIG. 14). Fermentations were performed at 35° C. and pH 6.5 in a FM medium containing yeast extract (YE) supplemented with vitamins except for nicotinic acid. Pretreated corn stover was the carbohydrate source. YE naturally contains some levels of nicotinate or nicotinamide; however, the levels are limiting.

To examine the effect of individual nutrients (e.g., vitamins or amino acids) on the growth and/or ethanol production of C. phytofermentans, complex nutrients such as yeast extract were reduced to a minimal concentration that will permit sub-optimal growth. Each specific nutrient is added in a titration of increasing amount until a “saturation” of effect is observed.

For examination of nicotinic acid, basal growth medium (BM) was prepared containing Corn Stover (113 g/L) as the carbohydrate source at 10% w/v. Corn Stover, Mono and Dibasic Potassium Phosphate and Sodium Chloride were sterilized by autoclaving and the remaining components were added aseptically to each fermentation flask. DBY and CSP were separately prepared each as 100 g/L stocks and autoclaved.

Nicotinic acid was prepared in water (1 g/L) filter sterilized and added to autoclaved flasks containing basal growth medium at (mg/L): 0, 5, 10, and 30 final concentrations. Final concentrations of 15, 20, 40, 50, 90 mg/L can also be used.

All solutions and materials were degassed prior to use. Prior to inoculation, all flasks receive a sterile addition of commercial cellulases (from Novozymes) consisting of (per 100 mL medium): 0.25 mL Cat# NS50013 and 0.83 mL of diluted (1/10 in water) Cat# NS22002. Each flask was inoculated with 10 mL (10% v/v) of C. phytofermentans, grown in modified version of the basal medium described above (yeast extract at 5 g/L replaces DBY and CSP and cellobiose at 20 g/L replaced Corn Stover as carbohydrate) to mid-log phase O.D.₆₆₀ equal to about 0.4.

Following inoculation, flasks were placed in shaking fermenters at 35° C. with agitation at 175 rpm and sampled daily. Sterile NaOH (4M) is used to adjust pH to about 6.8 as needed. Samples were analyzed for sugars (cellobiose, xylose, arabinose, and glucose) and fermentation products (formic, lactic, and acetic acids and ethanol) using HPLC.

FIG. 15 discloses ethanol yields in g/L (Y-axis) verses time in hours (X-axis) for fermentations performed with as described above with basal growth medium supplemented with 0, 5, 10, and 30 mg/L of nicotinic acid. Increasing amounts of nicotinic acid supplementation resulted in increased ethanol yields.

### Example 9

### Expression of the Clostridium cellulolyticum NAD Biosynthesis Pathway in Clostridium phytofermentans

Clostridium phytofermentans does not grow in the absence of Nicotinate or Nicotinamide in the media, suggesting that one or more genes involved in Nicotinate and Nicotinamide metabolism are missing. FIG. 16 discloses the KEGG (Kyoto Encyclopedia of Genes and Genomes) pathway map illustrating nicotinate and nicotinamide metabolism. In FIG. 16, genes are identified by their Enzyme Commission number (EC number), which identifies enzymes based upon their activity, not species of origin. Three genes involved in the biosynthesis of nicotinate D-ribonucleotides are highlighted: EC 1.4.3.16 (gene names: L-aspartate oxidase, NadB, Laspo, and AO; solid outline, FIG. 16), NadA (EC 2.5.1.72; gene names: quinolinate synthetase, QS, quinolinate synthase, and quinolinate synthetase complex subunit A; dashed outline, FIG. 16), and EC 2.4.2.19 (gene names: nicotinate-nucleotide pyrophosphorylase, nicotinate-nucleotide diphosphorylase, quinolinate phosphoribosyltransferase, quinolinic acid phosphoribosyltransferase, QAPRTase, NAD+ pyrophosphorylase, nicotinate mononucleotide pyrophosphorylase, and quinolinic phosphoribosyltransferase; dashed outline, FIG. 16). L-aspartate oxidase catalyzes the first step in the de novo synthesis of NAD+ from alanine, aspartate and glutamate metabolism; it catalyzes the conversion of L-aspartate to iminoaspartate. Quinolinate synthetase (EC 2.5.1.72) catalyzes the second step in the de novo synthesis of NAD+ from aspartate; it catalyzes the conversion of iminoaspartate to quinolinate. Nicotinate-nucleotide pyrophosphorylase is involved in the de novo synthesis of NAD+ from products of tryptophan metabolism and alanine, aspartate and glutamate metabolism; it catalyzes the conversion of quinolinate to nicotinate D-ribonucleotide. Clostridium phytofermentans contains an endogenous gene encoding for an L-aspartate oxidase (solid outline, FIG. 16); however, Clostridium phytofermentans does not contain a gene that encodes an NadA or EC 2.4.2.19 enzyme (dashed outline, FIG. 16).

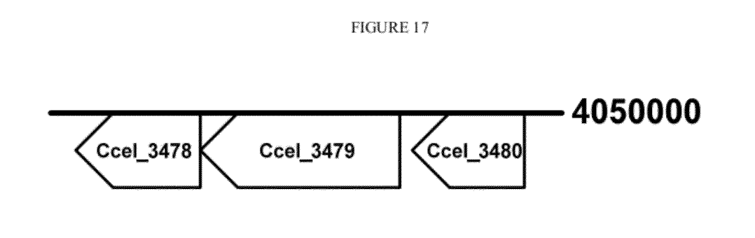

The genome of Clostridium cellulolyticum contains an operon with three genes coding for proteins involved in the biosynthesis of NAD+(FIG. 17). These gene products catalyze the reactions from Quinolinate (part of the tryptophan metabolism) and L-Aspartate to Nicotinate-D-ribonucleotides. Only one of the genes is present in the Clostridium phytofermentans while the other two are missing (FIG. 16). Polynucleotides and polypeptides relating to C. cellulolyticum genes in the nicotinate and nicotinamide metabolic pathway are found in Table 7. To establish the biosynthesis of Nicotinate-D-ribonucleotides, the NAD biosynthesis operon from C. cellulolyticum will be cloned into a shuttle plasmid and transferred into the Clostridium phytofermentans.

TABLE 7
C. cellulolyticum Nicotinate and Nicotinamide Metabolic Pathway Sequences

| | |
|---|---|
| SEQ ID NO: 59 | atggataaag atttgttgat tagtaacatt aaaaaaatga agaaagagca |
| Nucleotide | gaacgcagtt attgttgctc acagttatca ggttgatgag gtgcaggaga |
| Clostridium | ttgctgacgt tacaggagat tcattagctc taagtcaatt ttgtgcctcc |
| cellulolyticum | agtcaggcgg atactatagt tttttgcggg gtacacttta tggcagaaag |
| Ccel_3480 | tgcgaagctt ctatcgcctg aaaaaacggt tctgttgcct gaaataaatg |
| | caggttgccc aatggcagat atggttacgg ctgaggctct gaaagaggct |
| | aagaaaaagt atcctcacgc agctgttgta tgttatataa actcaagtgc |
| | tgaggttaag gccgagtgtg atatctgctg tacatcttca aatgcggaga |
| | aagtaatcag atctatcgat aaaaaggata ttatatttgc tccagataaa |
| | aatcttggca gttatgtagc aaaaaaggtt cctgaaaaaa acattatttt |
| | ttgggaaggc tactgcatta cacatcataa gattaagaaa gatgctgtca |
| | tagagtcaaa gagacttcat cctgatgcta ttttgctggt acatccggag |
| | tgccgacccg aaatacagga gcttgctgat tttgtgggaa gcacaaagca |
| | gattatagat tatgcaagaa attccgagca tgacaaattt attattggaa |
| | ctgagatggg tgttctttac cagttaaaaa aggagaaccc aaacaagact |
| | ttttatatga tgtcaacagg gctgatttgt ccgaatatga agaaaacatc |
| | attacagagt gttcatgatg ccttagctaa gaggcaatac gaaattacat |
| | tggatagcgg tattatagaa cgtgcatccg gtagtttgaa tagaatgctg |
| | gcagtaggga aatag |
| SEQ ID NO: 60 | MDKDLLISNIKKMKKEQNAVIVAHSYQVDEVQEIADVTGDSLALSQFCASSQADTIVFCGVHF |
| Peptide | MAESAKLLSPEKTVLLPEINAGCPMADMVTAEALKEAKKKYPHAAVVCYINSSAEVKAECDIC |
| Clostridium | CTSSNAEKVIRSIDKKDIIFAPDKNLGSYVAKKVPEKNIIFWEGYCITHHKIKKDAVIESKRL |
| cellulolyticum | HPDAILLVHPECRPEIQELADFVGSTKQIIDYARNSEHDKFIIGTEMGVLYQLKKENPNKTFY |
| Ccel_3480 | MMSTGLICPNMKKTSLQSVHDALAKRQYEITLDSGIIERASGSLNRMLAVGK |
| SEQ ID NO: 61 | atggaagagg atagtaataa ggttgatgtt gaggtcatac acaaggatgt |
| Nucleotide | cgtcatcatt ggtagcggaa tagccggagt atatactgca ttggaaatac |
| Clostridium | ccgacagttt ccagataggg ataattacca aagagacact ggacataagc |
| cellulolyticum | aattcagttc tcgcacaagg gggaatagca gtatctcttg atgagaagaa |
| Ccel_3479 | tgattctcca caactacatt tcaaagatac tctttttgca ggtgcaggat |
| | taaatgatca aaagagcgta tgggttctgg tagaagaggc tgctgaaaat |
| | attagaattt tgtgcagcct aggggtaaac tttgataaaa gcggacaaca |
| | tctatccctt actagagaag gggcccatag tgtaaataga attattcact |
| | caggagatac gactggtaag gaagtctgtg acaagcttat tgaggttgcc |
| | cggagaaaga agaacatatc gatttttgag agtcactttg cagtcgatct |
| | tgtgatcgaa gagggcaaat gcaaaggtgt aatagtttat gacgaaattg |
| | aagataaaat taagatattt aaatccggct cggtagttgt tgcaactggg |
| | ggttttggac agatttatgc acatactact aatcctgagg tcgcaactgg |
| | tgacggagtc ggaatgtgtt tgagagcggg tgcccaggcc atggatatgg |
| | agtttataca gttccatcct acagtactat accacccaaa agacaagagt |
| | ttcctaatat ctgaggcggt tagaggagag ggtgctcaac ttaaaaatag |
| | caacggtgag ccttttatga agaaatatca cgagttgggt gaactggcac |
| | ccagagacat tgtttcaaga gcgattttta aagaaatgta tcttactgat |
| | tccaaaaatg tatttctgga tataacattt aaaggtaggg aatatatcga |
| | aagtaggttt cctaatatct ttaaaacatg tctggattac ggtattgata |
| | tttctaagga ttttattccg gttgctcccg cagagcatta ttgtatgggg |
| | ggagtaaaaa cagatgttga cgggcagaca aatattccgg gtctgtatgc |
| | atgtggagag gtagcttgta cagggattca cggtgcaaac aggctcgcaa |
| | gcaattcttt gctagaagga ctggttttcg gcaggaaaat cgcaaagaag |
| | atcgagtccg aaggaagacc ttgtaataat tcagccgtca attcaaggct |
| | ctgttatgta tccaataaag aaaatgatgc ggctcttaaa tctatgaagg |
| | aagagataca ggctgcaatg acaaagtatg taggtataat cagaagtcaa |
| | caaggtcttg aaaaggctgc ccaaattatt aaagatattt ataagaagta |
| | cacggatttt acaggattca gtcttgtaaa gctggaagtg ttgaatatgc |
| | ttacagtagc ggggcttgtt atagaatcag ctcttgaaag aaaagagagc |
| | agaggtgctc attatagaac agactacgac aaaactgatg atacgaactg |
| | gagaaaaaat atagttaagg aattggagaa gggaaaaatg gcttcaccat tttaa |
| SEQ ID NO: 62 | MEEDSNKVDVEVIHKDVVIIGSGIAGVYTALEIPDSFQIGIITKETLDISNSVLAQGGIAVSL |
| Peptide | DEKNDSPQLHFKDTLFAGAGLNDQKSVWVLVEEAAENIRILCSLGVNFDKSGQHLSLTREGAH |
| Clostridium | SVNRIIHSGDTTGKEVCDKLIEVARRKKNISIFESHFAVDLVIEEGKCKGVIVYDEIEDKIKI |
| cellulolyticum | FKSGSVVVATGGFGQIYAHTTNPEVATGDGVGMCLRAGAQAMDMEFIQFHPTVLYHPKDKSFL |
| Ccel_3479 | ISEAVRGEGAQLKNSNGEPFMKKYHELGELAPRDIVSRAIFKEMYLTDSKNVFLDITFKGREY |
| | IESRFPNIFKTCLDYGIDISKDFIPVAPAEHYCMGGVKTDVDGQTNIPGLYACGEVACTGIHG |
| | ANRLASNSLLEGLVFGRKIAKKIESEGRPCNNSAVNSRLCYVSNKENDAALKSMKEEIQAAMT |
| | KYVGIIRSQQGLEKAAQIIKDIYKKYTDFTGFSLVKLEVLNMLTVAGLVIESALERKESRGAH |
| | YRTDYDKTDDTNWRKNIVKELEKGKMASPF |
| SEQ ID NO: 63 | atgaaactca gtaatcttta tatccatgaa atagttatga atgcattaaa |
| Nucleotide | agaggatatg ccactaggtg atattacaac agacaatatt ctttcagaag |
| Clostridium | gagattcatc cagagccgaa tttatggcaa agcaggatgc ggttattgca |
| cellulolyticum | gggctcgatg ttgcgaagta tgtttttgag gtactggata gcggcatatg |
| Ccel_3478 | ttttaaggcc tttgtaaaag atggagacaa ggtttcgaaa ggtgatatta |
| | tagccgaggt aagcggttcg acaagagctt tgttaaaagg tgaaaggact |
| | gcattgaact ttatgcaaag gttatctgca attgctacta tgactaacag |
| | atatgttagt aaagttcagg ggttacctgt aaaggtaact gatacaagaa |
| | aaactactcc cggtctgaga cttctggaga aatatgcagt aagtgcagga |
| | ggaggagcca atcacagatt ttcgctttct gacggtgttc tcataaagga |
| | taaccacatt gctgctgccg gaggaataac aaatgcggtt caacgtgtaa |
| | gaaacagtat tcctcatact gtaaagatcg aagtagaagt agagtccatg |
| | gaagaggttc gtgaggctct cgaatgcaag gcagatataa ttatgcttga |
| | taatatgtca aatgaacaga tggctgaggc tgtcaagctt ataaataaaa |
| | gagctcttgc ggaggcctcg gggaatataa gtgaagaaac tatatataat |
| | gtagcgttaa caggagttga tattatatct ataggtaaac ttactcactc |
| | tgcaaattct attgatataa gtatgaatat agaatag |
| SEQ ID NO: 64 | MKLSNLYIHEIVMNALKEDMPLGDITTDNILSEGDSSRAEFMAKQDAVIAGLDVAKYVFEVLD |
| Peptide | SGICFKAFVKDGDKVSKGDIIAEVSGSTRALLKGERTALNFMQRLSAIATMTNRYVSKVQGLP |
| Clostridium | VKVTDTRKTTPGLRLLEKYAVSAGGGANHRFSLSDGVLIKDNHIAAAGGITNAVQRVRNSIPH |
| cellulolyticum | TVKIEVEVESMEEVREALECKADIIMLDNMSNEQMAEAVKLINKRALAEASGNISEETIYNVA |
| Ccel_3478 | LTGVDIISIGKLTHSANSIDISMNIE |

Strains and Media

Chemically competent cells of Escherichia coli Top10 (Invitrogen) are used for cloning and plasmid amplification steps. E. coli are grown in LB media supplemented with 100 μg/mlampicillin at 37° C. Agar is used as needed.

Clostridium phytofermentans strains are grown in basal media (BM). Agar is used as needed. Cultures are incubated at 35° C. under anaerobic conditions. Spectinomycin and/or erythromycin are added to the medium where indicated at 150 μg/ml and 35 μg/ml, respectively.

Cloning of the C. cellulolyticum NAD Operon

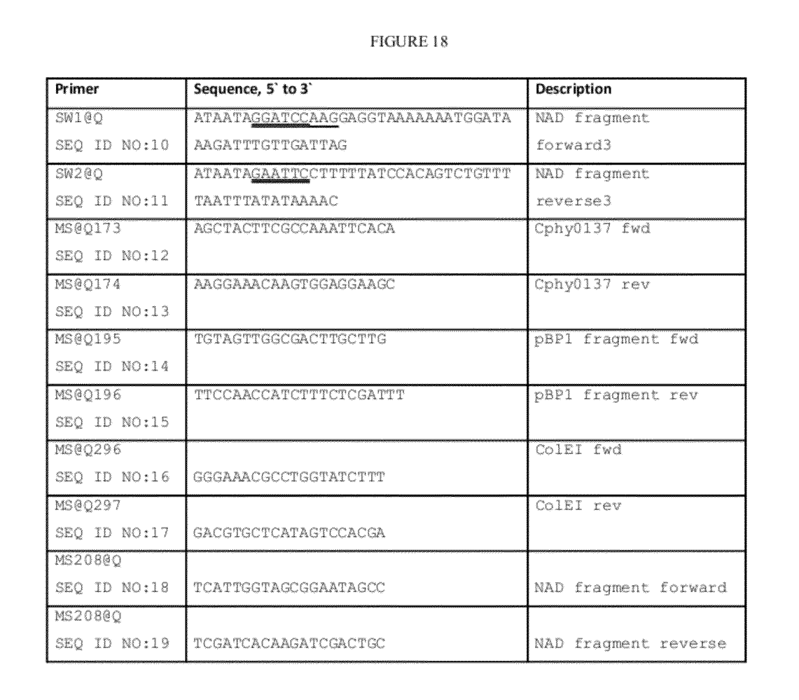

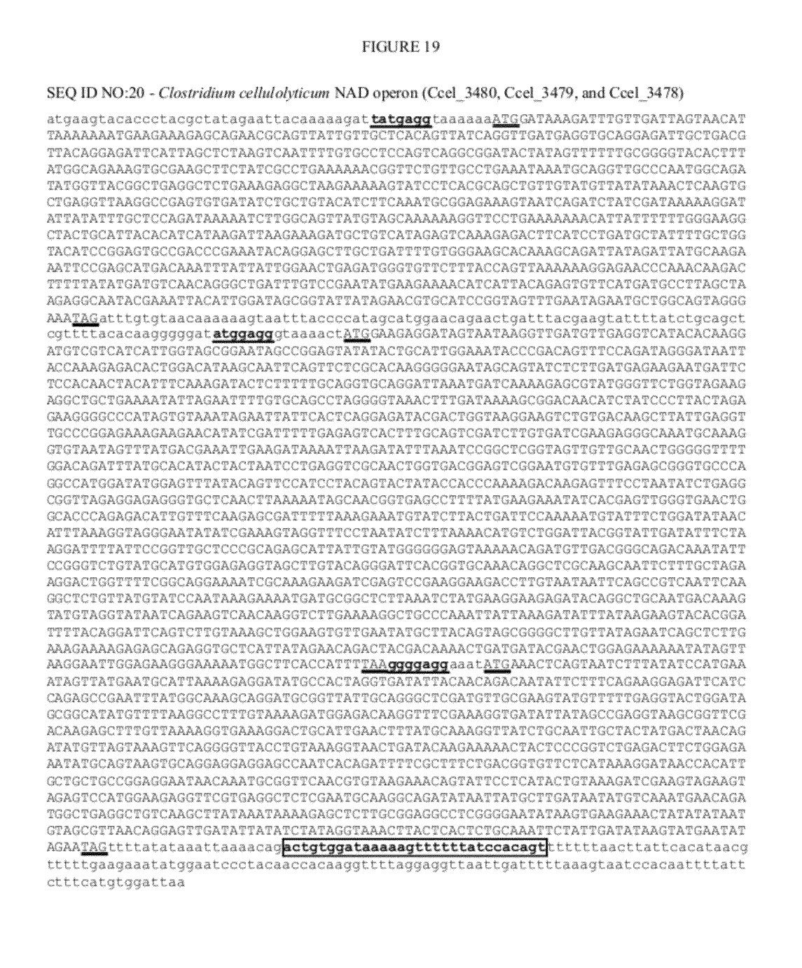

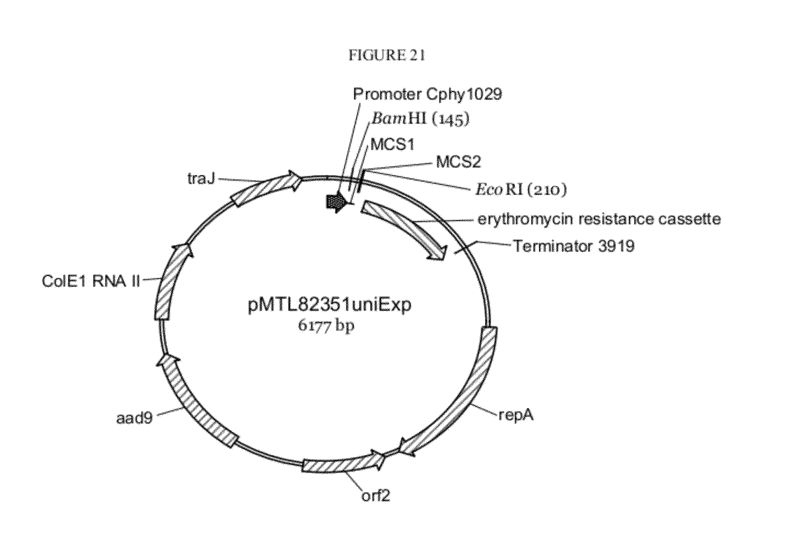

Chromosomal DNA isolated from Clostridium cellulolyticum (“FastPrep”, MP Biomedicals, LLC, Solon, Ohio) was used as a template for PCR amplification of target genes. Template DNA (100 ng chromosomal DNA), nucleotides (250 μM each nucleotide, dNTP) and primers SW1 @Q and SW2@Q (0.25 μM each; FIG. 18) and 1 μL Herculase II Fusion DNA Polymerase (Agilent Technologies) were combined in a 50 μL reaction volume as per manufacturer's recommendations and subjected to PCR as follows (initial denaturation at 95° C., 2 min; 20 sec at 95° C., 20 sec at 55° C., 2 min at 72° C. for 30 cycles; one final extension at 72° C. for 3 min). The resulting PCR product was 3549 by and constitutes three separate genes of the C. cellulolyticum NAD biosynthesis operon (FIG. 17) including an optimized ribosome-binding cassette for Ccel_{—}3480 but not the predicted terminator following Ccel_{—}3478 (FIG. 19). Plasmid pMTL82351UniExp (FIG. 21) was used as the vector used to clone and express the heterologous 3549 by fragment (NAD operon, FIG. 19) in Clostridium phytofermentans. FIG. 19 discloses the sequence of the NAD operon containing: gene coding regions (in order: Ccel_{—}3480, Ccel_{—}3479, and Ccel_{—}3478) are capitalized, putative ribosome binding sites are underlined, start and stop codons are double-underlined, and the predicted terminator is boxed.

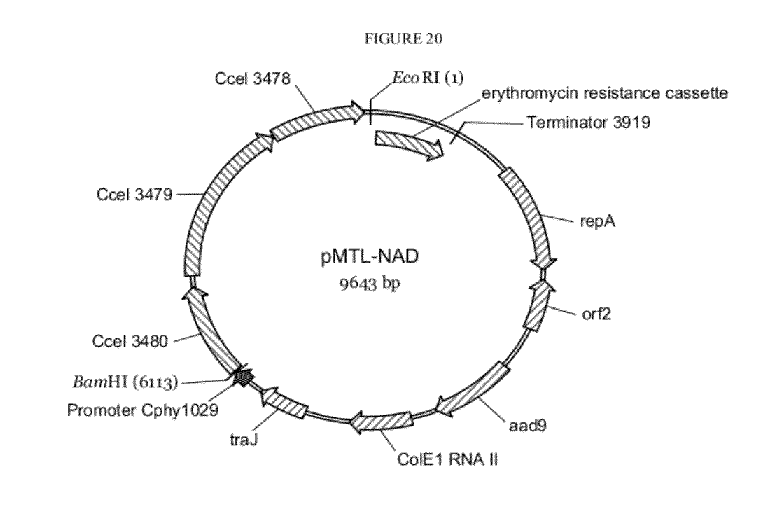

The NAD operon and pMTL82351UniExp were digested with BamHI-HF and EcoRI-HF (New England Biolabs) according to the manufacturer's recommendation. The restriction digestions were purified using a PCR purification kit (Qiagen) as recommended by manufacturer. Ligation reactions were prepared with 50 ng of the purified pMTL82351UniExp and 5-fold molar excess of restricted NAD operon in a total volume of 10 μL. The reaction was incubated for 3 h with T4-DNA Ligase (New England Biolabs) at 16° C. The ligation reaction was transformed into E. coli Top10 competent cells according to the manufacturer's recommendation (Invitrogen) and then plated on LB agar containing ampicillin (LB-Amp). Agar plated transformants were incubated at 37° C. for 18-24 h, and antibiotic-resistant colonies were picked and re-streaked on fresh LB-Amp agar plates. Re-streaked colonies were then transferred to LB-Amp broth, incubated (37° C. overnight), then plasmids were isolated from stationary phase cultures. The correct orientation of the NAD operon in pMTL82351UniExp was verified by PCR using the corresponding cloning primers, followed by digestion with BamHI and EcoRI. The newly constructed plasmid was designated as pMTL-NAD (FIG. 20).

Electroporation of Clostridium phytofermentans Strains

All procedures involving C. phytofermentans are performed under anaerobic conditions. Only centrifugation of cultures is performed outside an anaerobic chamber in closed 50 ml falcon tubes sealed with tape-sealed caps.

Electrocompetent Clostridium phytofermentans are prepared following growth in 100 ml BM broth until the culture reached an optical density of ˜0.5. The bacterial culture is cooled on ice for 10 min and then centrifuged at 4° C. in 2×50 ml falcon tubes (10 min at 5000 rpm). Supernatant is discarded and cells are washed by resuspension in 20 ml electroporation buffer (270 mM Sucrose, 7 mM Sodium Phosphate, 1 mM MgSO₄). Washed cells are re-centrifuged as before. Supernatant is discarded and cells resuspended in 2 ml electroporation buffer and kept on ice until used. Electrocompetent cells (500 μL) are transferred to a 0.4 cm electroporation cuvette (BioRad) and mixed with 1 μg of purified pMTL-NAD plasmid (in 25 μL volume). Electrocompetent cells (500 μL) plus 25 μL water are used as a negative control reaction. The cells/plasmid suspension are kept on ice for 4 min. Cells are then electroporated using BioRad Gene Pulser XCell set at 2250 Volts, 25 μF and 600 Ohms. Pre-warmed BM broth (1 mL) is added to the cuvette following pulse discharge and the cell suspension is transferred to a total volume of 5 ml BM broth and incubated at 35° C. for 4 hours for recovery. Following recovery, 250 μL aliquots of culture are plated on BM agar plates containing spectinomycin (BM-Spec) and incubated at 35° C. until transformant colonies were visible. Clostridium phytofermentans transformants obtained on BM-Spec plates are streaked to isolate single colonies on new BM-Spec agar plates. These single colonies are then tested to verify the presence of the pMTL-NAD plasmid by Colony PCR.

Colony PCR of Clostridium phytofermentans Transformants

Single colonies from a BM-Spec plate are resuspended in 20 μL water and vortexed until a homogenous suspension is reached. A 1 μL aliquot of this cell suspension is used as template in a PCR reaction using GoTaq Green Master mix (Promega) in a total solution volume of 20 μL. The PCR reaction contains 10 pmol of each primer. Three different primer pairs are used to:
1) Verify the culture as C. phytofermentans by amplification of a species specific chromosomal locus (primers MS@Q173, MS@Q174);
2) Verify the presence of the gram-positive origin of replication (primers MS@Q195, MS@Q196);
3) Verify the presence of the gram-negative origin of replication (primers MS@Q296, MS@Q297).
   Primer sequences are listed in FIG. 18. The colony PCR protocol is: 10 min at 95° C., 25×(45 seconds at 95° C., 30 seconds at 55° C., and 60 seconds at 72° C.), and 1-3 min at 72° C. For transformants that test positive for both origins in the plasmid, the presence of the NAD operon is then verified by an additional PCR reaction with primers specific for the Ccel_{—}3479 gene (MS@Q208 and MS@Q209, FIG. 18).

### Example 10

### Cloning and Evaluation of Pyridoxal, NAD and DHF

Construction of Plasmids

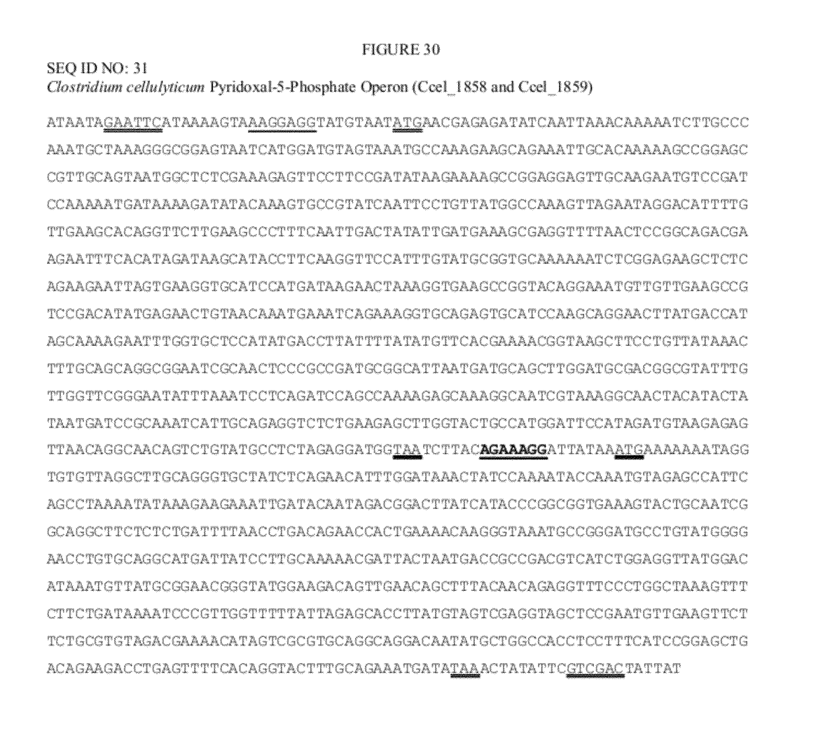

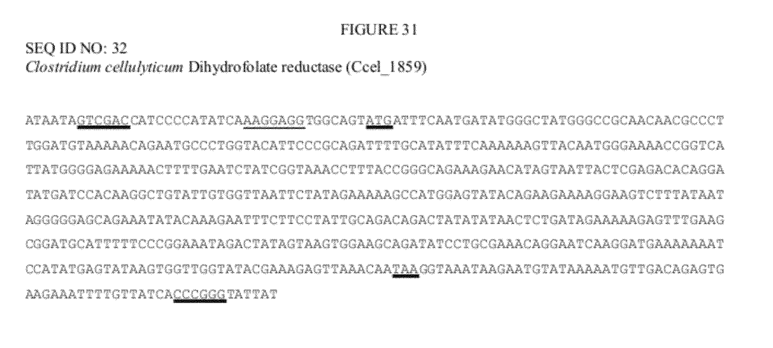

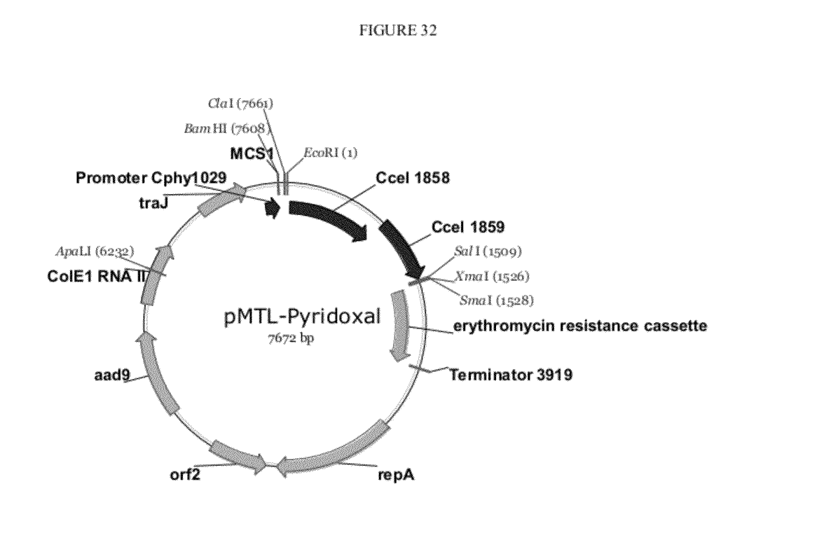

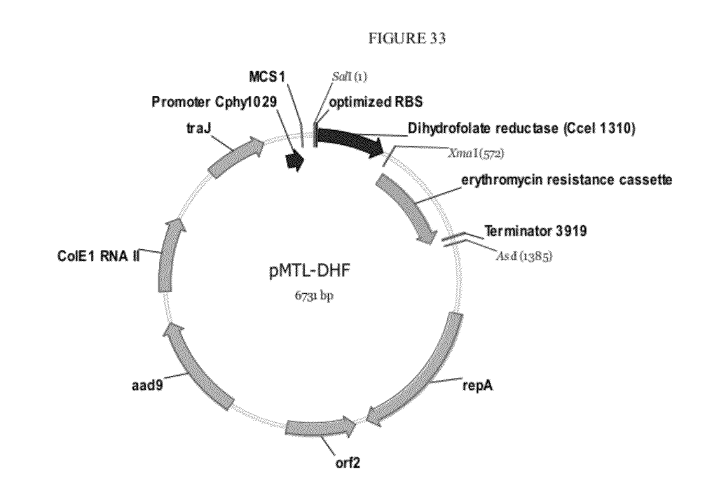

Plasmids containing genes for the biosynthesis of Pyridoxal-5-Phosphate, Nicotinate D-ribonucleotide, Tetrahydrofolate, or combinations thereof were constructed. All plasmids bearing genes for one or more of the biosynthesis operons are based on the plasmid pMTL82351uniExp (FIG. 21). For the expression of genes leading to the generation of Pyridoxal-5-Phosphate, genes Ccel_{—}1858 (YaaD in FIGS. 41 & 42) and Ccel_{—}1859 (YaaE in FIGS. 41 & 42) from Clostridium cellulolyticum were cloned. For the expression of Dihydrofolate reductase, Gene Ccel_{—}1310 (1.5.1.3 in FIG. 43) from Clostridium cellulolyticum was cloned. Cloning of genes for the NAD biosynthesis operon of Clostridium cellulolyticum is described in Example 9. The genes were amplified via PCR according to the manufacturers recommendation using Clostridium cellulolyticum chromosomal DNA as template (see Example 8). The primers for NAD are shown in FIG. 18, the primers for Pyridoxal genes and DHF are shown in Table 10. The sequences for the NAD biosynthesis operon are shown in FIG. 19; the amplified sequences for the Pyridoxal genes and DHF reductase gene are shown in FIG. 30 and FIG. 31, respectively. Polypeptide and polynucleotide sequences relating the C. cellulolyticum vitamin B6 metabolic pathway genes can be found in Table 8. Polypeptide and polynucleotide sequences relating to C. cellulolyticum one carbon pool by folate metabolic pathway genes can be found in Table 9. The 5′-Primer sequences of all genes contain optimized ribosome-binding sequences to enhance translation of the cloned genes. Restriction digestion, ligation, selection and verification were carried out as described in Example 8. Restriction sites used were EcoRI/SalI and SalI/XmaI for the pyridoxal and the DHF genes, respectively. The newly constructed plasmids were designated pMTL-NAD (FIG. 20), pMTL-Pyridoxal (FIG. 32) and pMTL-DHF (FIG. 33).

TABLE 8
C. cellulolyticum Vitamin B₆ Metabolic Pathway Sequences

| | |
|---|---|
| SEQ ID NO: 65 | atgaacgaga gatatcaatt aaacaaaaat cttgcccaaa tgctaaaggg |
| Nucleotide | cggagtaatc atggatgtag taaatgccaa agaagcagaa attgcacaaa |
| Clostridium | aagccggagc cgttgcagta atggctctcg aaagagttcc ttccgatata |
| cellulolyticum | agaaaagccg gaggagttgc aagaatgtcc gatccaaaaa tgataaaaga |
| Ccel_1858 | tatacaaagt gccgtatcaa ttcctgttat ggccaaagtt agaataggac |
| | attttgttga agcacaggtt cttgaagccc tttcaattga ctatattgat |
| | gaaagcgagg ttttaactcc ggcagacgaa gaatttcaca tagataagca |
| | taccttcaag gttccatttg tatgcggtgc aaaaaatctc ggagaagctc |
| | tcagaagaat tagtgaaggt gcatccatga taagaactaa aggtgaagcc |
| | ggtacaggaa atgttgttga agccgtccga catatgagaa ctgtaacaaa |
| | tgaaatcaga aaggtgcaga gtgcatccaa gcaggaactt atgaccatag |
| | caaaagaatt tggtgctcca tatgacctta ttttatatgt tcacgaaaac |
| | ggtaagcttc ctgttataaa ctttgcagca ggcggaatcg caactcccgc |
| | cgatgcggca ttaatgatgc agcttggatg cgacggcgta tttgttggtt |
| | cgggaatatt taaatcctca gatccagcca aaagagcaaa ggcaatcgta |
| | aaggcaacta catactataa tgatccgcaa atcattgcag aggtctctga |
| | agagcttggt actgccatgg attccataga tgtaagagag ttaacaggca |
| | acagtctgta tgcctctaga ggatggtaa |
| SEQ ID NO: 66 | MNERYQLNKNLAQMLKGGVIMDVVNAKEAEIAQKAGAVAVMALERVPSDIRKAGGVARMSDPK |
| Peptide | MIKDIQSAVSIPVMAKVRIGHFVEAQVLEALSIDYIDESEVLTPADEEFHIDKHTFKVPFVCG |
| Clostridium | AKNLGEALRRISEGASMIRTKGEAGTGNVVEAVRHMRTVTNEIRKVQSASKQELMTIAKEFGA |
| cellulolyticum | PYDLILYVHENGKLPVINFAAGGIATPADAALMMQLGCDGVFVGSGIFKSSDPAKRAKAIVKA |
| Ccel_1858 | TTYYNDPQIIAEVSEELGTAMDSIDVRELTGNSLYASRGW |
| SEQ ID NO: 67 | atgaaaaaaa taggtgtgtt aggcttgcag ggtgctatct cagaacattt |
| Nucleotide | ggataaacta tccaaaatac caaatgtaga gccattcagc ctaaaatata |
| Clostridium | aagaagaaat tgatacaata gacggactta tcatacccgg cggtgaaagt |
| cellulolyticum | actgcaatcg gcaggcttct ctctgatttt aacctgacag aaccactgaa |
| Ccel_1859 | aacaagggta aatgccggga tgcctgtatg gggaacctgt gcaggcatga |
| | ttatccttgc aaaaacgatt actaatgacc gccgacgtca tctggaggtt |
| | atggacataa atgttatgcg gaacgggtat ggaagacagt tgaacagctt |
| | tacaacagag gtttccctgg ctaaagtttc ttctgataaa atcccgttgg |
| | tttttattag agcaccttat gtagtcgagg tagctccgaa tgttgaagtt |
| | cttctgcgtg tagacgaaaa catagtcgcg tgcaggcagg acaatatgct |
| | ggccacctcc tttcatccgg agctgacaga agacctgagt tttcacaggt |
| | actttgcaga aatgatataa |
| SEQ ID NO: 68 | MKKIGVLGLQGAISEHLDKLSKIPNVEPFSLKYKEEIDTIDGLIIPGGESTAIGRLLSDFNLT |
| Peptide | EPLKTRVNAGMPVWGTCAGMIILAKTITNDRRRHLEVMDINVMRNGYGRQLNSFTTEVSLAKV |
| Clostridium | SSDKIPLVFIRAPYVVEVAPNVEVLLRVDENIVACRQDNMLATSFHPELTEDLSFHRYFAEMI |
| cellulolyticum | |
| Ccel_1859 | |

TABLE 9
C. cellulolyticum One Carbon Pool by Folate Metabolic Pathway Sequences

| | |
|---|---|
| SEQ ID NO: 65 | atgatttcaa tgatatgggc tatgggccgc aacaacgccc ttggatgtaa |
| Nucleotide | aaacagaatg ccctggtaca ttcccgcaga ttttgcatat ttcaaaaaag |
| Clostridium | ttacaatggg aaaaccggtc attatgggga gaaaaacttt tgaatctatc |
| cellulolyticum | ggtaaacctt taccgggcag aaagaacata gtaattactc gagacacagg |
| Ccel_1310 | atatgatcca caaggctgta ttgtggttaa ttctatagaa aaagccatgg |
| | agtatacaga agaaaaggaa gtctttataa tagggggagc agaaatatac |
| | aaagaatttc ttcctattgc agacagacta tatataactc tgatagaaaa |
| | agagtttgaa gcggatgcat ttttcccgga aatagactat agtaagtgga |
| | agcagatatc ctgcgaaaca ggaatcaagg atgaaaaaaa tccatatgag |
| | tataagtggt tggtatacga aagagttaaa caataa |
| SEQ ID NO: 66 | MISMIWAMGRNNALGCKNRMPWYIPADFAYFKKVTMGKPVIMGRKTFESIGKPLPGRKNIVIT |
| Peptide | RDTGYDPQGCIVVNSIEKAMEYTEEKEVFIIGGAEIYKEFLPIADRLYITLIEKEFEADAFFP |
| Clostridium | EIDYSKWKQISCETGIKDEKNPYEYKWLVYERVKQ |
| cellulolyticum | |
| Ccel_1310 | |

TABLE 10
Primers for cloning Pyridoxal and DHF Genes

| Primer | Sequence, 5′ to 3′ | Description |
|---|---|---|
| SW3@Q | ATAATAGAATTCATAAAAGTAAAGGAGGTAT | Pyridoxal Forward |
| SEQ ID NO: 25 | GTAATATGAACGAGAGATATCAATTAAAC | |
| SW4@Q | ATAATAGTCGACGAATATAGTTTATATCATT | Pyridoxal Reverse |
| SEQ ID NO: 26 | TCTGCAAAGTACCTGTG | |
| SW5@ | ATAATAGTCGACCATCCCCATATCAAAGGAG | DHF Forward |
| SEQ ID NO: 27 | GTGGCAGTATGATTTC | |
| SW6@ | ATAATACCCGGGTGATAACAAAATTTCTTCA | DHF Reverse |
| SEQ ID NO: 28 | CTCTGTC | |
| MS@Q277 | ATAATAGGCGCGCCTTAAGCCCGATAAATGG | Cphy1606-1607 fwd2 |
| SEQ ID NO: 29 | GATGGAG | |
| MS@Q278 | ATAATAGGCGCGCCGCTAGCAGTAGCTTTCC | Cphy1606-1607 rev2 |
| SEQ ID NO: 30 | TGAATGGA | |

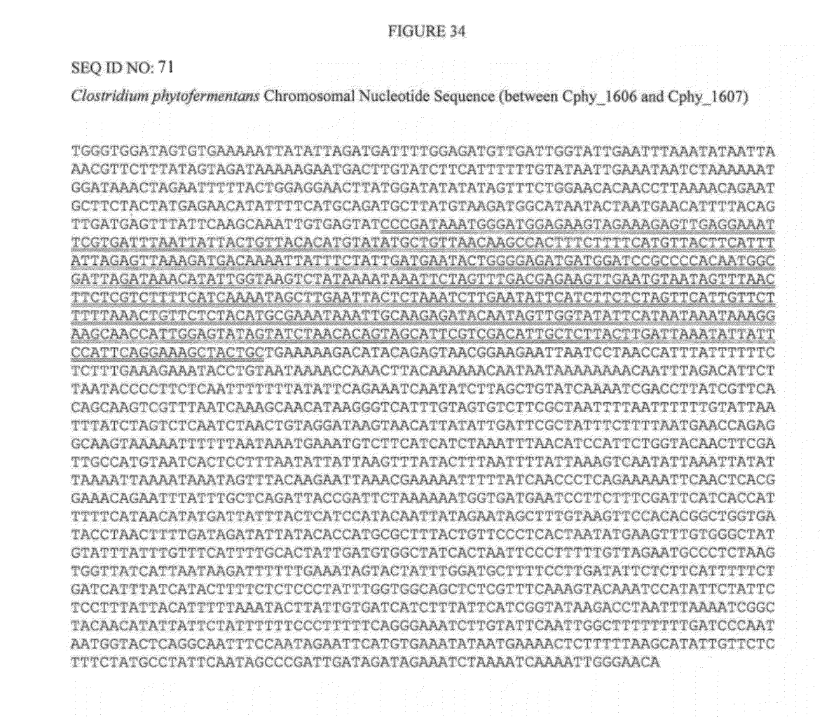

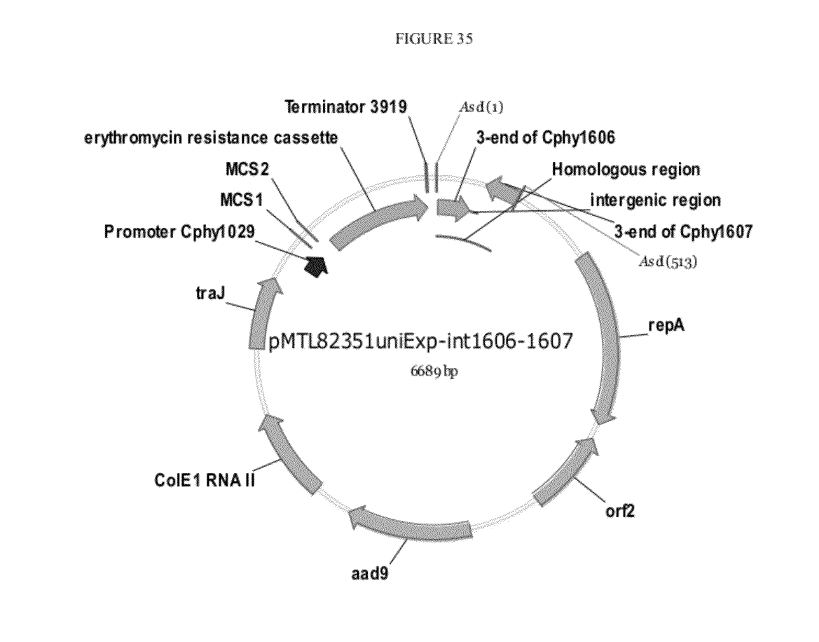

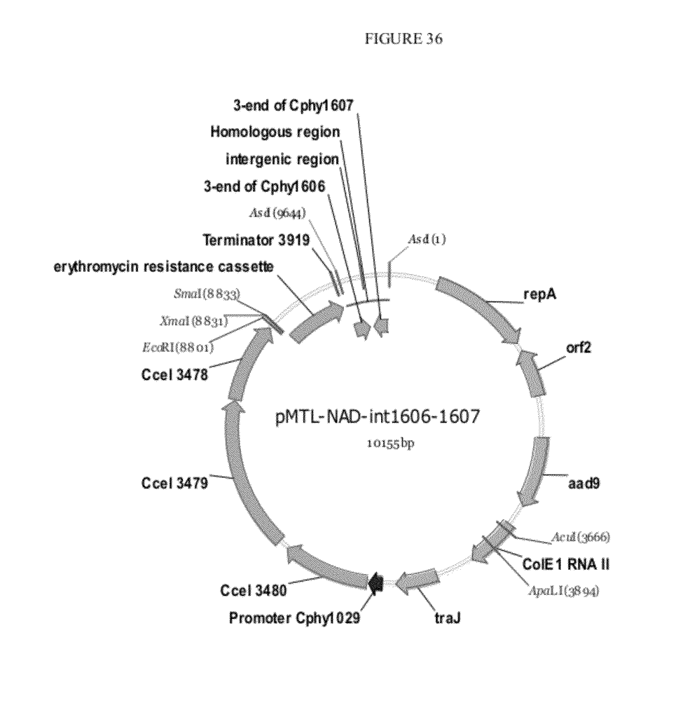

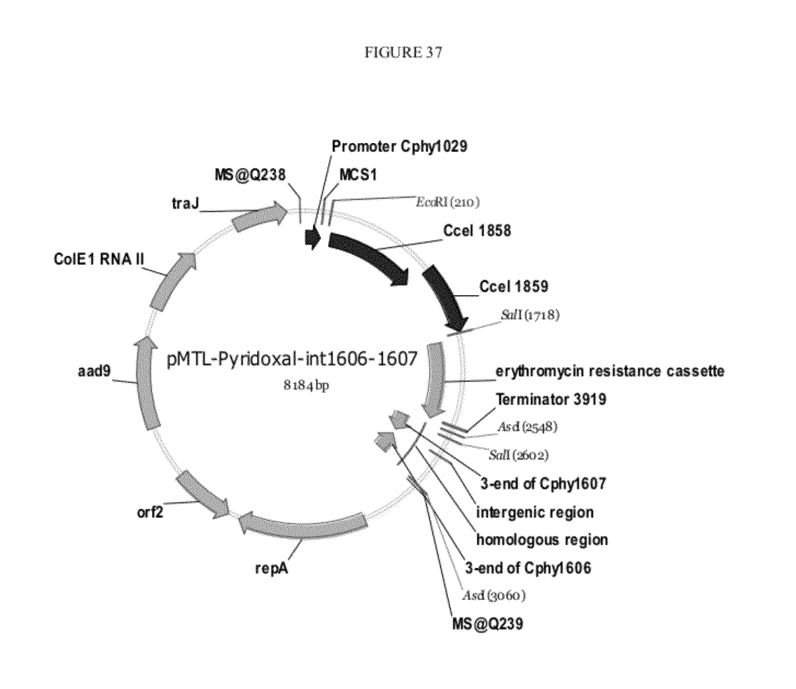

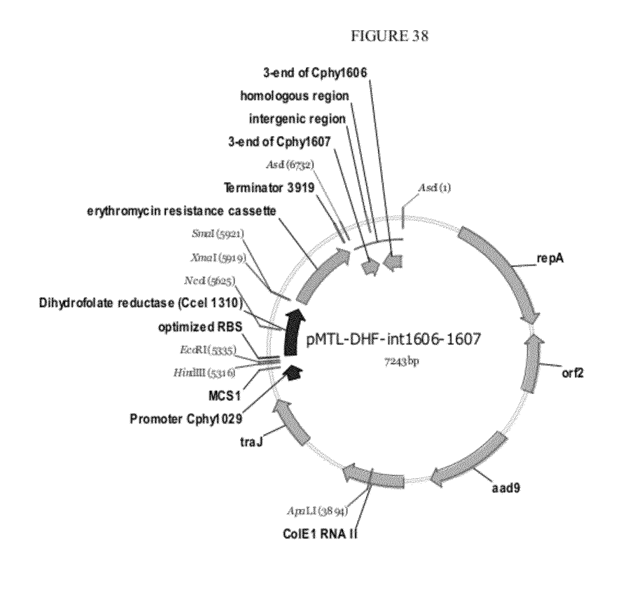

To enable the integration of the vitamin plasmids into the Q chromosome, a 499 by homologous integration site was cloned into the plasmids. The same integration between the Q genes Cphy_{—}1606 and Cphy_{—}1607 was used as in the plasmid pQint (FIG. 22). The chromosomal region was amplified using the primers MS@Q277 and MS@Q278 (primer sequences shown in Table 10) using standard PCR techniques described supra. The genetic sequences between Cphy_{—}1606 and Cphy_{—}1607 is shown in FIG. 34, the region amplified and therefore homologous to the region present in the plasmids is underlined. The PCR product and the fours plasmids pMTL82351uniExp, pMTL-NAD, pMTL-Pyridoxal and pMTL-DHF were digested with the restriction enzymes Ascl (New England Biolabs, Ipswich, Mass. 01938-2723) according to the manufacturer's recommendation. The restriction digestions were purified using a PCR purification kit (Qiagen or Invitrogen PureLink Kit; Qiagen, Inc., Valencia, Calif. 91355). Four ligation reactions were prepared with 50 ng of the digested plasmids and 5-fold molar excess of the digested PCR product (Cphy_{—}1606/Cphy_{—}1607 region). The reaction was incubated for 2 h with T4-DNA Ligase (New England Biolabs) at 16° C. The ligation reaction was transformed into E. coli Top10 competent cells according to the manufacturer's recommendation (Invitrogen, Inc., Carlsbad, Calif. 92008) and then plated on LB agar containing spectinomycin (LB-spec). Antibiotic-resistant colonies were picked and re-streaked on fresh LB-spec plates. Plasmids were isolated from liquid cultures (inoculated with single colonies) using Qiagen's MiniPrep Kit and verified by digestion. The plasmids were designated pMTL82351uniExp-int1606-1607 (see FIG. 35), pMTL-NAD-int1606-1607 (see FIG. 36), pMTL-Pyridoxal-int1606-1607 (see FIG. 37) and pMTL-DHF-int1606-1607 (see FIG. 38).

Transfer to C. phytofermentans

The four plasmids (pMTL82351uniExp-int1606-1607, pMTL-NAD-int1606-1607, pMTL-Pyridoxal-int1606-1607 and pMTL-DHF-int1606-1607) were transferred to Clostridium phytofermentans by electroporation (see Example 5). Clostridium phytofermentans strains obtained on BM-spec plates were restreaked and the presence of the plasmid confirmed by Colony PCR as described in Example 9. The resulting strains were designated QX36 (carrying plasmid pMTL-NAD-int1606-1607), QX37 (carrying plasmid pMTL-DHF-int1606-1607), QX45 (carrying plasmid pMTL-Pyridoxal-int1606-1607) and QX46 (carrying plasmid pMTL82351uniExp-int1606-1607).

Growth on Minimal Media

A single colony of each strain (QX36, QX37, QX45 and QX46) was streaked on Basal Media (BM) agar plates containing 150 ng/mL spectinomycin and incubated a 35° C. A single colony from each strain on these BM-spec plates was then streaked on minimal media (Table 11) plates containing 5 g/L casamino acids. The MM (minimal media) agar plates were then incubated at 35° C. until visible colony growth occurred. A single colony of each of the four strains grown on these MM agar plates was then streaked onto MM plates and minimal media agar plates lacking one of the following components: Nicotinic Acid (B₃), Pyridoxine (B₆) and Folinic Acid. Growth was observed for all strains on the minimal media plates. Absent or poor growth was observed on the plates missing either Nicotinic Acid (B₃) or Folinic Acid. On the agar plates missing Pyridoxine (B₆), only the strain QX45 carrying the genes for the pyridoxal biosynthesis genes grew, indicating that the pyridoxal biosynthesis genes were functionally expressed in Clostridium phytofermentans. Single colonies of QX45 from the minimal media plate lacking Pyridoxine were restreaked on new minimal media agar plates without pyridoxine. Robust growth on these plates was confirmed and a single colony of QX45 was used to inoculated liquid minimal media with and without pyridoxine. The parental Clostridium phytofermentans strain was used as a control. The liquid minimal media bottles were incubated for 5 days at 35° C. Both strains grew in the minimal media but only QX45 grew in the minimal media lacking Pyridoxine.

Fermentation

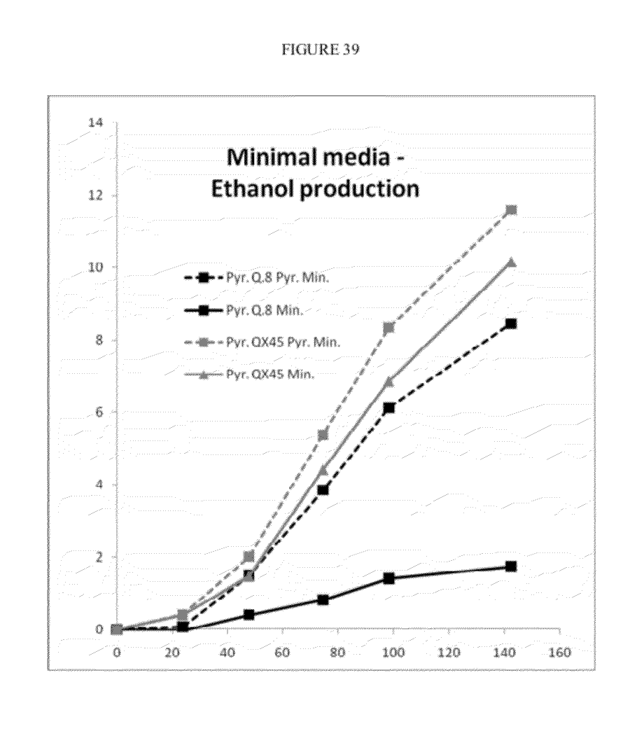

Clostridium phytofermentans strains QX45 and its parental strain C. phytofermentans Q.8 were tested in fermentation. Single colonies from minimal media plates were used to inoculate 100 mL liquid minimal media and incubated at 35° C. until an optical density of 0.3 or higher was reached. These cultures were used as inoculums for shake flasks containing minimal media with or without pyridoxine. A 10% inoculum was used and the fermentation of cellobiose carried out in duplicates. The concentration of cellobiose, ethanol, acetic acid, lactic acid and formic acid were determined daily. The mean ethanol production curves for the four tested conditions are shown in FIG. 39. As shown in the previous experiment, the Clostridium phytofermentans strain Q.8 did not grow in minimal media without pyridoxine (black solid line). The initial ethanol production under these conditions can be a caused by the carry-over of pyridoxine from the inoculation culture. When pyridoxine is added, strain Q.8 grows and produced up to 9 g/L ethanol under the tested conditions (black dotted line). QX45 did grow better than its parental strain and produced up to 11 g/L ethanol under the same conditions even in the absence of pyridoxine in the medium (grey solid line). In the cultures containing pyridoxine, QX45 did grow even better and produced slightly more ethanol, 12 g/L under these conditions (grey dotted line).

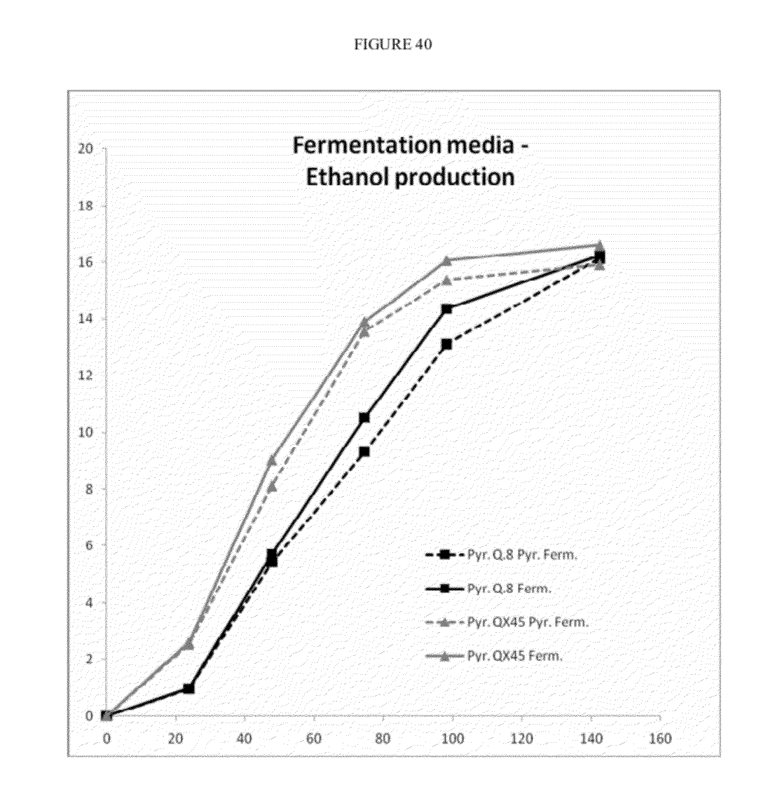

The Clostridium strains QX45 and Q.8 were also tested in complex media containing 5 g/L yeast extract. Under these test conditions QX45 and Q.8 grew comparably and produced the same levels of ethanol (FIG. 40). The expression of the pyridoxal biosynthesis genes in QX45 results in faster growth and ethanol production as judged by the corresponding fermentation curves (black vs. grey lines, both solid and dotted).Minimal media (MM) was prepared from minimal media base by adding cellobiose, minimal media salts, minimal media vitamins, CAA and Purines and Pyrimidines after autoclaving. Minimal media agar plates were prepared by adding 17 g/L agar to the minimal media base prior to autoclaving. Base media, salts and substrates were degassed with nitrogen prior to autoclave sterilization. Following sterilization, 87 mL of base media was combined with 10 ml of 10× Substrate stock and 1 ml each of 100× salts solution, 100× amino acids and 100× B-Vitamins solution to achieve final concentrations. All additions were prepared anaerobically and aseptically.

TABLE 11
Minimal media composition

| Minimal medium base: | per L: | |
|---|---|---|
| KH₂PO₄ | 1.60 | g |
| K₂HPO₄ | 3.00 | g |
| NaCl | 1.00 | g |
| Ammonium sulfate | 2.00 | g |
| 0.1% (w/v) Resazurin solution | 0.500 | mL |
| Adjust pH to 7.5 with NaOH | | |

| 10x Substrate Stock | g/L: |
|---|---|
| 20% Cellobiose | 20 |

| 100X Minimal Media Salts solution: | g/L |
|---|---|
| Tri-Sodium Citrate | 10.000 |
| CaCl2•2H₂O | 0.500 |
| MgSO₄•7H₂O | 6.000 |
| FeSO₄•7H₂O | 0.400 |
| CoSO₄•H₂O | 0.200 |
| ZnSO₄•7H₂O | 0.200 |
| NiCl₂ | 0.200 |
| MnSO₄•H₂O | 0.500 |
| CuSO₄•5H₂O | 0.040 |
| H₃BO₃ | 0.040 |
| Ammonium Molybdate tetrahydrate (H₂₄Mo₇N₆O₂₄•4H₂O) | 0.040 |
| Sodium Selenite (Na₂SeO₃) | 0.040 |

| 100X Minimal Media Vitamin solution: | g/L |
|---|---|
| Pantethine | 1.000 |
| Nicotinic Acid (B₃) | 3.000 |
| Pyridoxine (B₆) | 1.000 |
| Cyanocobalamine (B₁₂) | 1.000 |
| Thiamine (B₁) | 1.000 |
| Biotin | 1.000 |
| Folinic Acid | 0.030 |
| Para aminobenzoic acid | 1.000 |
| Riboflavin (B₂) | 1.000 |
| Folic Acid | 0.030 |

| 20X Casamino acids solution (CAA): | g/L |
|---|---|
| Casamino acids | 100 |

| 100X Minimal Media Purines and Pyrimidines solution: | g/L |
|---|---|
| Xanthine | 1.000 |
| Thymine | 1.000 |

### Example 11

### Fermentation with Genetically Modified Microorganisms

Genetically modified microorganisms that are capable of growth in a medium lacking one or more vitamins required for growth of an unmodified microorganisms of the same species are tested in fermentation. The unmodified parent microorganism is also tested under identical conditions.

Minimal Media Fermentations with and without Supplementation by Vitamins.

Single colonies from minimal media plates are used to inoculate an appropriate volume (e.g., 100 mL) of liquid minimal media and incubated at an appropriate temperature (e.g., 35° C.) until an optical density of 0.3 or higher is reached. These cultures are used as inoculums for shake flasks containing minimal media with or without vitamin supplementation (e.g., supplementation with one or more of thiamine, nicotinic acid, folinic acid, pyridoxine, etc.). A 10% inoculum is used and the fermentation of a biomass (e.g., cellobiose, pretreated corn stover, etc.) carried out in duplicates. The concentration of cellobiose and fermentation end-products such as ethanol, acetic acid, lactic acid and formic acid are determined daily. The unmodified microorganism is not expected to grow in minimal media without the vitamins; however, a low level of initial fermentation end-product production can occur under these conditions, which is likely to be a caused by the carry-over of the vitamins from the inoculation culture. In conditions with supplementation by the vitamins, the unmodified microorganism is expected to grow and produce the fermentation end-products. The genetically modified microorganism is expected to produce at least as much (and in many cases more) fermentation end-product without vitamin supplementation as the unmodified microorganism produces with vitamin supplementation. Production of fermentation end-products by genetically modified microorganism is expected to be further increased under minimal media fermentations with vitamin supplementation.

The isolated strains disclosed herein have been deposited in the Agricultural Research Service culture Collection (NRRL), an International Depositary Authority, National Center for Agricultural Utilization Research, Agricultural Research Service, U.S. Department of Agriculture, 1815 North University Street, Peoria, Ill. 61604 U.S.A. in accordance with and under the provisions of the Budapest Treaty for the Deposit of Microorganisms. The strains were tested by the NRRL and determined to be viable. The depositor acknowledges the duty to replace the deposits should the depository be unable to furnish a sample when requested, due to the condition of the deposits. All restrictions on the availability to the public of the subject culture deposits will be irrevocably removed upon the granting of a patent disclosing them. The deposits are available as required by foreign patent laws in countries wherein counterparts of the subject application, or its progeny, are filed. However, it should be understood that the availability of a deposit does not constitute a license to practice the subject matter disclosed herein in derogation of patent rights granted by governmental action.

The term “comprising” as used herein is synonymous with “including,” “containing,” or “characterized by,” and is inclusive or open-ended and does not exclude additional, unrecited elements or method steps.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure herein. It should be understood that various alternatives to the embodiments of the disclosure described herein can be employed in practicing the described subject matter. It is intended that the following claims define the scope of the disclosure and that methods and structures within the scope of these claims and their equivalents be covered thereby.

What is claimed is:

1. A genetically modified microorganism adapted for decreased vitamin dependency that ferments a biomass to produce one or more fermentation end-products, wherein said microorganism comprises a genetic modification that decreases vitamin dependency.

2. The microorganism of claim 1, wherein said genetic modification comprises one or more heterologous polynucleotides that encode for enzymes in one or more metabolic pathways, wherein said metabolic pathways comprise a thiamine metabolic pathway, a nicotinate and nicotinamide metabolic pathway, a vitamin B₆ metabolic pathway, a one carbon pool by folate pathway, or a combination thereof.

3. The microorganism of claim 2, wherein at least one of said polynucleotides has at least about 60% identity to SEQ ID NO:20, SEQ ID NO:21, SEQ ID NO:22, SEQ ID NO:23, SEQ ID NO:24, SEQ ID NO:31, SEQ ID NO:32, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or a combination thereof.

4. The microorganism of claim 1, wherein said microorganism hydrolyzes and ferments hemicellulosic or lignocellulosic material.

5. The microorganism of claim 1, wherein said microorganism is a genetically modified Clostridium species.

6. The microorganism of claim 1, wherein said microorganism is a genetically modified Clostridium phytofermentans or Clostridium sp Q.D.

7. The microorganism of claim 1, wherein said one or more fermentation end-products comprise one or more alcohols.

8. A method of producing one or more fermentation end-products comprising:
a. providing a biomass in a medium;
b. contacting said medium with a genetically modified microorganism adapted for decreased vitamin dependency, wherein said microorganism comprises a genetic modification that decreases vitamin dependency; and;
c. allowing sufficient time for said microorganism to produce said fermentation end-products from said biomass.

9. The method of claim 8, wherein said genetic modification comprises one or more heterologous polynucleotides that encode for enzymes in one or more metabolic pathways, wherein said metabolic pathways comprise a thiamine metabolic pathway, a nicotinate and nicotinamide metabolic pathway, a vitamin B₆ metabolic pathway, a one carbon pool by folate pathway, or a combination thereof.

10. The method of claim 9, wherein at least one of said polynucleotides has at least about 60% identity to SEQ ID NO:20, SEQ ID NO:21, SEQ ID NO:22, SEQ ID NO:23, SEQ ID NO:24, SEQ ID NO:31, SEQ ID NO:32, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or a combination thereof.

11. The method of claim 8, wherein said microorganism hydrolyzes and ferments hemicellulosic or lignocellulosic material.

12. The method of claim 8, wherein said microorganism is a genetically modified Clostridium species.

13. The method of claim 8, wherein said medium is supplemented with one or more vitamins, wherein at least one of said vitamins is at a concentration below the minimal nutritional requirements for growth of an unmodified microorganism of the same species.

14. The method of claim 8, further comprising a second microorganism, wherein said second microorganism produces at least one vitamin that is used by said genetically modified microorganism.

15. The method of claim 8, wherein said one or more fermentation end-products comprise one or more alcohols.

16. A system for the production of one or more fermentation end-products comprising:
a. a media comprising a biomass;
b. a genetically modified microorganism adapted for decreased vitamin dependency, wherein said microorganism comprises a genetic modification that decreases vitamin dependency; and,
c. a fermenter configured to house said media and said microorganism.

17. The system of claim 16, wherein said genetic modification comprises one or more heterologous polynucleotides that encode for enzymes in one or more metabolic pathways, wherein said metabolic pathways comprise a thiamine metabolic pathway, a nicotinate and nicotinamide metabolic pathway, a vitamin B₆ metabolic pathway, a one carbon pool by folate pathway, or a combination thereof.

18. The system of claim 17, wherein at least one of said polynucleotides has at least about 60% identity to SEQ ID NO:20, SEQ ID NO:21, SEQ ID NO:22, SEQ ID NO:23, SEQ ID NO:24, SEQ ID NO:31, SEQ ID NO:32, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or a combination thereof.

19. The system of claim 16, wherein said microorganism hydrolyzes and ferments hemicellulosic or lignocellulosic material.

20. The system of claim 16, wherein said microorganism is a genetically modified Clostridium species.


SEQUENCE LISTING (71 sequences)

SEQ ID NO: 1 - DNA, 7887 bp, Artificial Sequence
  Description of Artificial Sequence Synthetic polynucleotide
cctgcaggataaaaaaattgtagataaattttataaaatagttttatctacaatttttttatcaggaaacagctatgaccgcggggattttacacgtttcattaataatttcttatatttctttatttgtttgtaaaatttacttaaatttcgccagaaaacaaaagaaagcctttactaattaatagtttagtgatactcttttatgtaggtattttttaaaatacattaaacctaggtaattgaggaaagttacaattaccattatataaggaggatattcatatgaaaagaaaactgaaacaaagatgtgctgttttagtggcagttgcaacgatgatagcttcgttgcaatgggggagagtgccagtacaagcagtaacagcagacggtcttacctctcaacagtatgttgaggcaatgggcgaaggctggaacttaggaaattcctttgatggttttgattctgatacttcaaaaccagatcaaggcgagaccgcttggggaaatcctaaggttacaaaagagctaatccatgcagtcaaacaaaaaggctatagtagtatccgcataccaatgaccctatatcgtagatatacggagagcaatggtgtatgcactatcgatagcgcatggatagcacgttacaaagaagtagtagattatgcagttgcagaaggtttatacgttatgataaacattcaccatgattcctggatatggttatcttcatgggatggaaataagagttctgtgcaatatgtaagatttactcagatgtgggatcaacttgcgaaggcatttaaagattatccgttacaagtatgttttgaaacgataaatgagccgaactttcaaaactctggaaacgttactgcacagaataaattagatatgcttaaccaagcggcttacaatataattcgtgcctctggtggatcaaatgcaaagagaatgattgttttaccatcactaaatacgaaccatgataatagtgtaccattagctgatttcataactaaattgaatgattctaatatcattgcaaccgttcattattatagtgaatgggtatttagtgctaaccttggtaagacaagctttgatgaagatttatggggaaatggtgattacactcctcgtgatgcggtaaataaggcgtttgataccatttccaatgcatttacagcaaaaaaaatcggtgttgttatcggagaatttggtcttttaggttatgactctgattttgaaaataatcaaccaggcgaagaattaaaatattatgagtatatgaattatgtagctagacaaaagaaaatgtgccttatgttttgggataacggatctggaattaatcgtaacgactctaagtatagttggaaaaaacctatagttggaaagatgttagaagtatctatgacaggacgttcctcttatgcaacaggccttgataccatttacctaaacggcagctcatttaatgatattaatatcccgcttactctaaacggtaacacctttgttggagttacaggattaaccagtggtaccgattttacgtataaccaatccaatgcaacactaacattaaaatcatcctacgtgaagaaggtttatgatgcaatgggaagtaattatggtacggtagctgatttggtacttaagttttcaagtggagctgattggcatgagtatttagtgaaatacaaagcaccagtatttcaaaatgcgaatggaactgtttccaatggaattaatattccagttcaatttaacggaagtaaactccgtcgttctacagcttatataggttctaatcgagttggcccgaatcaaagctggtggatgtatttagagtatggtgcaacttttgtggcgaactatacgaacaatattttaaccattaagcctgatttctttaaggatggttctgtttatgatggaaatatatcatttgagatggagttttatgatggacaaaagttaaaatataatcttaataaatcaaatggtaacataacaggaactgcagcagcagtaacccctacaccaacaccaacggcgacaccaacaccaacagcgacgccaacaccaaccgtaacaccaaaaccaacaataaccccaacagtaacgccgacaccaacagtaacgccaaaaccaacaataacaccgacagtaacaccaactcctactccaatcccaggaacaggtccagttacattaaaatacgaagtaacgaatacttgggataagcatacacaggcgaatattacattaaccaatacctctaatacagcactaaagaattttgttgtatcatttacttataaagggtatatagaccaaatgtggagtgcagatttggttagtcaaaattcgggtaccattacagtgaagggaccagcatgggctacgaatctagatccagggcaaagtataacatttggttttattgcttcacatgatacaccgtctgttgatccaccatcaaatgttactttagttagttcaaattaaaattgtattcaaatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgttctgaatccttagctaatggttcaacaggtaactatgacgaagatagcaccctggataagtctgtaatggattctaaggcatttaatgaagacgtgtatataaaatgtgctaatgaaaaagaaaatgcgttaaaagagcctaaaatgagttcaaatggttttgaaattgattggtagtttaatttaatatattttttctattggctatctcgatacctatagaatcttctgttcacttttgtttttgaaatataaaaaggggctttttagccccttttttttaaaactccggaggagtttcttcattcttgatactatacgtaactattttcgatttgacttcattgtcaattaagctagtaaaatcaatggttaaaaaacaaaaaacttgcatttttctacctagtaatttataattttaagtgtcgagtttaaaagtataatttaccaggaaaggagcaagttttttaataaggaaaaatttttccttttaaaattctatttcgttatatgactaattataatcaaaaaaatgaaaataaacaagaggtaaaaactgctttagagaaatgtactgataaaaaaagaaaaaatcctagatttacgtcatacatagcacctttaactactaagaaaaatattgaaaggacttccacttgtggagattatttgtttatgttgagtgatgcagacttagaacattttaaattacataaaggtaatttttgcggtaatagattttgtccaatgtgtagttggcgacttgcttgtaaggatagtttagaaatatctattcttatggagcatttaagaaaagaagaaaataaagagtttatatttttaactcttacaactccaaatgtaaaaagttatgatcttaattattctattaaacaatataataaatcttttaaaaaattaatggagcgtaaggaagttaaggatataactaaaggttatataagaaaattagaagtaacttaccaaaaggaaaaatacataacaaaggatttatggaaaataaaaaaagattattatcaaaaaaaaggacttgaaattggtgatttagaacctaattttgatacttataatcctcattttcatgtagttattgcagttaataaaagttattttacagataaaaattattatataaatcgagaaagatggttggaattatggaagtttgctactaaggatgattctataactcaagttgatgttagaaaagcaaaaattaatgattataaagaggtttacgaacttgcgaaatattcagctaaagacactgattatttaatatcgaggccagtatttgaaattttttataaagcattaaaaggcaagcaggtattagtttttagtggattttttaaagatgcacacaaattgtacaagcaaggaaaacttgatgtttataaaaagaaagatgaaattaaatatgtctatatagtttattataattggtgcaaaaaacaatatgaaaaaactagaataagggaacttacggaagatgaaaaagaagaattaaatcaagatttaatagatgaaatagaaatagattaaagtgtaactatactttatatatatatgattaaaaaaataaaaaacaacagcctattaggttgttgttttttattttctttattaatttttttaatttttagtttttagttcttttttaaaataagtttcagcctctttttcaatattttttaaagaaggagtatttgcatgaattgccttttttctaacagacttaggaaatattttaacagtatcttcttgcgccggtgattttggaacttcataacttactaatttataattattattttcttttttaattgtaacagttgcaaaagaagctgaacctgttccttcaactagtttatcatcttcaatataatattcttgacctatatagtataaatatatttttattatatttttacttttttctgaatctattattttataatcataaaaagttttaccaccaaaagaaggttgtactccttctggtccaacatatttttttactatattatctaaataatttttgggaactggtgttgtaatttgattaatcgaacaaccagttatacttaaaggaattataactataaaaatatataggattatctttttaaatttcattattggcctcctttttattaaatttatgttaccataaaaaggacataacgggaatatgtagaatatttttaatgtagacaaaattttacataaatataaagaaaggaagtgtttgtttaaattttatagcaaactatcaaaaattagggggataaaaatttatgaaaaaaaggttttcgatgttatttttatgtttaactttaatagtttgtggtttatttacaaattcggccggcccaatgaataggtttacacttactttagttttatggaaatgaaagatcatatcatatataatctagaataaaattaactaaaataattattatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaagaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccc

SEQ ID NO: 2 - DNA, 36 bp, Artificial Sequence
  Description of Artificial Sequence Synthetic primer
ataataggatcctattacagttaaggaggagtaacg

SEQ ID NO: 3 - DNA, 37 bp, Artificial Sequence
  Description of Artificial Sequence Synthetic primer
ataatagtcgactttttctctgaaacggaatttgggg

SEQ ID NO: 4 - DNA, 44 bp, Artificial Sequence
  Description of Artificial Sequence Synthetic primer
aaataaaggatccaaggaggaatgagctatgtctgcaacaaaac

SEQ ID NO: 5 - DNA, 35 bp, Artificial Sequence
  Description of Artificial Sequence Synthetic primer
aaataaactcgagattccgtctcatagtctttgcg

SEQ ID NO: 6 - DNA, 41 bp, Artificial Sequence
  Description of Artificial Sequence Synthetic primer
aaataaactcgagaaggaggagcaaactatgcaagtcgacc

SEQ ID NO: 7 - DNA, 33 bp, Artificial Sequence
  Description of Artificial Sequence Synthetic primer
aaataaagctagcgtgctcaccaccaggcgtgg

SEQ ID NO: 8 - DNA, 39 bp, Artificial Sequence
  Description of Artificial Sequence Synthetic primer
aaataaagctagcaggaggcataacgtatggcatgtggc

SEQ ID NO: 9 - DNA, 47 bp, Artificial Sequence
  Description of Artificial Sequence Synthetic primer
aaataaaccgcggctttatggcgtggcaaaatggtcgtatcctttcc

SEQ ID NO: 10 - DNA, 49 bp, Artificial Sequence
  Description of Artificial Sequence Synthetic primer
ataataggatccaaggaggtaaaaaaatggataaagatttgttgattag

SEQ ID NO: 11 - DNA, 48 bp, Artificial Sequence
  Description of Artificial Sequence Synthetic primer
ataatagaattcctttttatccacagtctgttttaatttatataaaac

SEQ ID NO: 12 - DNA, 21 bp, Artificial Sequence
  Description of Artificial Sequence Synthetic primer
agctacttcgccaaattcaca

SEQ ID NO: 13 - DNA, 21 bp, Artificial Sequence
  Description of Artificial Sequence Synthetic primer
aaggaaacaagtggaggaagc

SEQ ID NO: 14 - DNA, 20 bp, Artificial Sequence
  Description of Artificial Sequence Synthetic primer
tgtagttggcgacttgcttg

SEQ ID NO: 15 - DNA, 22 bp, Artificial Sequence
  Description of Artificial Sequence Synthetic primer
ttccaaccatctttctcgattt

SEQ ID NO: 16 - DNA, 20 bp, Artificial Sequence
  Description of Artificial Sequence Synthetic primer
gggaaacgcctggtatcttt

SEQ ID NO: 17 - DNA, 20 bp, Artificial Sequence
  Description of Artificial Sequence Synthetic primer
gacgtgctcatagtccacga

SEQ ID NO: 18 - DNA, 20 bp, Artificial Sequence
  Description of Artificial Sequence Synthetic primer
tcattggtagcggaatagcc

SEQ ID NO: 19 - DNA, 20 bp, Artificial Sequence
  Description of Artificial Sequence Synthetic primer
tcgatcacaagatcgactgc

SEQ ID NO: 20 - DNA, 3696 bp, Clostridium cellulolyticum
atgaagtacaccctacgctatagaattacaaaaagattatgaggtaaaaaaatggataaagatttgttgattagtaacattaaaaaaatgaagaaagagcagaacgcagttattgttgctcacagttatcaggttgatgaggtgcaggagattgctgacgttacaggagattcattagctctaagtcaattttgtgcctccagtcaggcggatactatagttttttgcggggtacactttatggcagaaagtgcgaagcttctatcgcctgaaaaaacggttctgttgcctgaaataaatgcaggttgcccaatggcagatatggttacggctgaggctctgaaagaggctaagaaaaagtatcctcacgcagctgttgtatgttatataaactcaagtgctgaggttaaggccgagtgtgatatctgctgtacatcttcaaatgcggagaaagtaatcagatctatcgataaaaaggatattatatttgctccagataaaaatcttggcagttatgtagcaaaaaaggttcctgaaaaaaacattattttttgggaaggctactgcattacacatcataagattaagaaagatgctgtcatagagtcaaagagacttcatcctgatgctattttgctggtacatccggagtgccgacccgaaatacaggagcttgctgattttgtgggaagcacaaagcagattatagattatgcaagaaattccgagcatgacaaatttattattggaactgagatgggtgttctttaccagttaaaaaaggagaacccaaacaagactttttatatgatgtcaacagggctgatttgtccgaatatgaagaaaacatcattacagagtgttcatgatgccttagctaagaggcaatacgaaattacattggatagcggtattatagaacgtgcatccggtagtttgaatagaatgctggcagtagggaaatagatttgtgtaacaaaaaagtaatttaccccatagcatggaacagaactgatttacgaagtattttatctgcagctcgttttacacaagggggatatggagggtaaaactatggaagaggatagtaataaggttgatgttgaggtcatacacaaggatgtcgtcatcattggtagcggaatagccggagtatatactgcattggaaatacccgacagtttccagatagggataattaccaaagagacactggacataagcaattcagttctcgcacaagggggaatagcagtatctcttgatgagaagaatgattctccacaactacatttcaaagatactctttttgcaggtgcaggattaaatgatcaaaagagcgtatgggttctggtagaagaggctgctgaaaatattagaattttgtgcagcctaggggtaaactttgataaaagcggacaacatctatcccttactagagaaggggcccatagtgtaaatagaattattcactcaggagatacgactggtaaggaagtctgtgacaagcttattgaggttgcccggagaaagaagaacatatcgatttttgagagtcactttgcagtcgatcttgtgatcgaagagggcaaatgcaaaggtgtaatagtttatgacgaaattgaagataaaattaagatatttaaatccggctcggtagttgttgcaactgggggttttggacagatttatgcacatactactaatcctgaggtcgcaactggtgacggagtcggaatgtgtttgagagcgggtgcccaggccatggatatggagtttatacagttccatcctacagtactataccacccaaaagacaagagtttcctaatatctgaggcggttagaggagagggtgctcaacttaaaaatagcaacggtgagccttttatgaagaaatatcacgagttgggtgaactggcacccagagacattgtttcaagagcgatttttaaagaaatgtatcttactgattccaaaaatgtatttctggatataacatttaaaggtagggaatatatcgaaagtaggtttcctaatatctttaaaacatgtctggattacggtattgatatttctaaggattttattccggttgctcccgcagagcattattgtatggggggagtaaaaacagatgttgacgggcagacaaatattccgggtctgtatgcatgtggagaggtagcttgtacagggattcacggtgcaaacaggctcgcaagcaattctttgctagaaggactggttttcggcaggaaaatcgcaaagaagatcgagtccgaaggaagaccttgtaataattcagccgtcaattcaaggctctgttatgtatccaataaagaaaatgatgcggctcttaaatctatgaaggaagagatacaggctgcaatgacaaagtatgtaggtataatcagaagtcaacaaggtcttgaaaaggctgcccaaattattaaagatatttataagaagtacacggattttacaggattcagtcttgtaaagctggaagtgttgaatatgcttacagtagcggggcttgttatagaatcagctcttgaaagaaaagagagcagaggtgctcattatagaacagactacgacaaaactgatgatacgaactggagaaaaaatatagttaaggaattggagaagggaaaaatggcttcaccattttaaggggaggaaatatgaaactcagtaatctttatatccatgaaatagttatgaatgcattaaaagaggatatgccactaggtgatattacaacagacaatattctttcagaaggagattcatccagagccgaatttatggcaaagcaggatgcggttattgcagggctcgatgttgcgaagtatgtttttgaggtactggatagcggcatatgttttaaggcctttgtaaaagatggagacaaggtttcgaaaggtgatattatagccgaggtaagcggttcgacaagagctttgttaaaaggtgaaaggactgcattgaactttatgcaaaggttatctgcaattgctactatgactaacagatatgttagtaaagttcaggggttacctgtaaaggtaactgatacaagaaaaactactcccggtctgagacttctggagaaatatgcagtaagtgcaggaggaggagccaatcacagattttcgctttctgacggtgttctcataaaggataaccacattgctgctgccggaggaataacaaatgcggttcaacgtgtaagaaacagtattcctcatactgtaaagatcgaagtagaagtagagtccatggaagaggttcgtgaggctctcgaatgcaaggcagatataattatgcttgataatatgtcaaatgaacagatggctgaggctgtcaagcttataaataaaagagctcttgcggaggcctcggggaatataagtgaagaaactatatataatgtagcgttaacaggagttgatattatatctataggtaaacttactcactctgcaaattctattgatataagtatgaatatagaatagttttatataaattaaaacagactgtggataaaaagttttttatccacagtttttttaacttattcacataacgtttttgaagaaatatggaatccctacaaccacaaggttttaggaggttaattgatttttaaagtaatccacaattttattctttcatgtggattaa

SEQ ID NO: 21 - DNA, 3773 bp, Clostridium cellulolyticum
tataggttaaaggtgctttcctgttggatggcatcttttttatgcgtcttttactttgccaaggtcaagttaaacaagtaaccctattacagttaaggaggagtaacgtttaatgaagaaagtattaacaattgcaggatcagactgcagcggaggagcaggaattcaggcggatttaaaaacctttgctgcacatggtatttatggaatgagtgttattgtatcggtagttgcagagaatacataccgcgtcatcgatattcaggatataacgcctgatatgattaaaaaacagatagatgcagtttttgaagacataacacctgatgctgtaaaaataggtatgctgtcagacaaggagtgcatggaagctgttgctgaaaaattggaacaatataagcctgtaaatgtggttattgaccctgtaatgattgcaaaaggcggtcacgccctcatgaaagaagaagctctcgagttttttataaaaaggcttatacccctggcaggtatgcttacaccaaatattccggaggcagaggcgattaccgggatgcggataactgaccataatgatatgcagaaggcagctagaagcatttatgaaatgggtgcaggcagtgttctggtaaaaggcggacatttatatggagatgcggaagacatactttttgacggaagagaattttacatatattcaactaaaagaatccagacaaaaaatacacatggaacaggatgtacattttcatcagctatagcctcaaaccttgccttgggaataccttttacaacagcggttgaaaaagctaaagaatacgtcacaatggcgattgaacattcacttgaaatcggaaagggacatggtccaacgcatcatttttatggggtttacaaaaacggattggataatcggaggttgctatgaattacaaaacacagatggacgccgcaaaaaaaggtattattacaaatgaaatgaagattgtttctcgaaaggaatcaatggatgaaaataagcttcgggagcttgttgctgagggcagaatagctattccggcaaatataaatcacaagtccttaagtccggagggaataggagaaggtctcagaacaaaaatcaatgtaaaccttggtatatcaggggattgtcctgactatacaaaagaaatggaaaaggctgatatgtcaatcaaatttggtgttgaagccataatggaccttagtaattacggaaagacaaacacctttcgcaaagagcttataaagcgttctcctgccatgataggaaccgttcccatgtatgatgccataggctaccttgaaaaggacctgatggatattaaagcttcggactttttaaaagttgttgaggctcatgctgcagaaggtgtggactttatgacaattcatgcaggaataaataaacgtgccgttgagtgtttcaaacgctcaaagagacttaccaatattgtttcaagaggaggttcccttctttttgcatggatggagatgactggcaatgaaaatcctttttttgagtattatgacgatttccttggaatactcagggaatatgatgtaaccataagtcttggagatgcattgaggcccggtagtatcaatgacagctcagacgccggacaattaagtgaacttatagaactgggcgacctgaccaaacgtgcatgggaaaaggatgtacaggtaatggttgaggggccgggacatatggctatgaacgagatagccgccaatatgactattcaaaagagactatgtcatggagcacctttttatgtactggggcctttggtgacagatatagcaccgggatatgaccatatcacctcggcaataggcggagcaattgctgcggcaaacggtgcagatttcttatgttatgtaacccctgcggagcatttaaggttacctgacctttcggatgtaaaagaaggaatagttgcttcaaaaatagccgctcatgccgccgatattgcaaagggaatacctaatgcacgtgaaatagataataaaatgagcgatgccagacgccgaatcgactgggaagaaatgttctcatatgctattgacgaagataaggcaagagcatattttgaaagcacacctcccactgacagacatacctgctcaatgtgcggaaaaatgtgtgctatgaggactacaaataagattttagccggtgaaaaggttgagtttgtaacagagaaatagtaaaggagacaaactatgagtgagtatacaaagcaaataacaagattaatctctgaggtcagaagtaaaaagccgcttattcacaatattacaaattatgtaactgtaaatgattgtgcaaatgttactctggcaataggggcctccccaattatggcagatgatattgatgaggctgcggatattacttctatttcttctgcacttgtaataaatataggaactttaaacaaaagaacgattgaatcaatgattctatcgggtaaaaaagcaaatgagaagggtattcccgtaatttttgatcctgttggagcaggggcttcagctttgagaaatgagactacaagcactattcttgataaaataaaaataagtgtcttgcgtggcaatttgtctgagatatcctacatagccgggcgtaatgcctcaacaaaaggcgttgatgcttcagaagcggatattcaatcaaatgactccatcgcagttgcaaaagcggcagctgtaaagcttggatgtgttgttgcagtaacaggagctgttgatgtgatttcagacggtaaaaacgttgtgactgtcttaaacggacataaaatgctgtcaaatgttacaggaacagggtgtatgactactgctttggttggttccttctgcggggcagtaaaagattatttcaaagctgctgtggcaggagtgacagtgatgggaatatcaggagagattgcctatgaagccgcaggacataaaggtactggaagctaccatattgcaataatcgatgccatcagtaggatggatgaaaatatttttggagagaaggcaagaataaatgaaatctaaaattgattatactctttatcttgtaacagaccatcagctgatgagtactaaaacactggaggaggcagtagagcaggcaatagcaggaggctgtactttagtgcagttacgtgaaaaaactgcttcttcccgggatttttatcaaaatgctattaacgtaaaaactataacagacaaatataatgtacctcttattataaatgatagaattgatattgctttggctgtaggtgctgacggagtccacgtggggcagagcgatcttccggctgctgttgtgcgaaaaatcataggtaacgataagatactcggcgtatcggcagggtctgttgaaaaggcaattgaagctcagaaaatcggtgctgactatataggagtgggtgctttgttttcaacaagcacaaaaacagatgcaaaggcagtgtctatagaaactctcatgaaaattgtaagggaagtttcaattcctgtggtcggtataggcgggataaatgcggagaatgcagtacaactgaaaaatacgggaataaggggtattgccgtagtatcggctattatttcacaaaaggatataaaatcatctgctgaaaaattgcttgaaatattcgtcaacaaagcttaaaatcatatataaataccccaaattccgtttcagagaaaaaggaaaggatttggggtgtttttttggaaaaattaccataacc

SEQ ID NO: 22 - DNA, 5467 bp, Escherichia coli
ttaactttttggaatgagctatgtctgcaacaaaactgacccgccgcgaacaacgcgcccgggcccaacattttatcgacaccctggaaggcaccgcctttcccaactcaaaacgcatttatatcactggcacacaccccggcgtgcgcgtgccgatgcgtgagatccagcttagcccgacgctaattggcggtagcaaagaacagccgcagtacgaagaaaacgaagcgattccggtctacgacacctccggcccgtatggtgatccgcagattgccattaacgtgcagcaagggctggcaaaactacgccagccgtggatcgatgcgcgcggcgataccgaagaacttaccgtgcgcagttccgattacactaaagcgcggctggcagatgatggcctcgacgaactgcgttttagcggcgtactaacaccaaaacgcgccaaagcaggacgccgtgtcacccaactgcactacgcccgccagggcatcatcacgccggaaatggaattcatcgccatccgcgagaatatgggccgcgagcgcatccgtagcgaggttttacgccaccagcatccgggaatgagctttggcgcacatctgccggaaaatatcactgcggaatttgtccgtgatgaagttgctgccggacgtgcgattatcccggccaacattaatcatccggaatcggagccgatgattattggtcgcaatttcctggtaaaagttaacgccaatatcggcaactcggcggtcacctcttccatcgaagaagaagtggaaaagctggtatggtccacgcgctggggagcggatacggtgatggatctctccaccggtcgctatattcacgaaacccgcgagtggattttgcgtaacagcccggtgccgatcggtacagtgccgatctaccaggcgctggagaaggttaacgggatcgccgaagatcttacctgggaagcgttccgcgacacgctgctggaacaggccgagcaaggtgtggattacttcactatccatgcgggcgtactgctgcgctatgtgccgatgaccgcgaaacgcctgaccggtatcgtctctcgcggcggttcgattatggcgaaatggtgcctctcccatcatcaggaaaatttcctctatcaacacttccgcgaaatttgtgaaatctgtgccgcttatgacgtttcgctgtcgctgggcgacggtctgcgccccggttctattcaggacgccaacgatgaagcgcagtttgccgagctgcatacgctgggcgaactgaccaaaattgcctgggaatatgacgtgcaggtgatgattgaaggcccaggccacgtgccgatgcagatgatccgccgcaatatgaccgaggagttagagcactgccacgaagcgccgttttacactctggggccgctaactaccgatattgcgccgggctatgaccacttcacgtcggggattggtgcggcgatgattggctggtttggctgcgcgatgctctgttacgtaacgccaaaagagcatctgggtctgcccaataaagaagatgttaagcaggggcttatcacctataagattgctgcccacgccgctgacctggcgaaagggcatccgggcgcgcaaattcgcgataacgccatgtcgaaagcccgcttcgaatttcgctgggaagaccagtttaatctggccctcgacccgtttaccgcccgcgcttatcacgatgaaaccctgccgcaagagtcaggtaaagtcgcccatttttgctccatgtgtgggccgaaattctgctcgatgaaaatcagccaggaagtgcgtgattacgccgccacgcaaactattgaaatgggaatggcggatatgtcggagaacttccgtgccagaggcggagaaatctacctgcgtaaggaggaagcgtgatgtatcagcctgattttcctcctgtaccttttcgttcaggactgtacccggtggtggacagcgtacagtggatcgaacgtctgttggatgcaggcgtacgtactctccagctacgcatcaaagatcggcgcgatgaagaggtggaagccgatgtcgtggcggcaattgcgctgggccgccgctataacgcgcgattgtttatcaacgattactggcggctggcgatcaagcatcaggcgtatggcgtccatttggggcaggaagatttgcaagccaccgatctcaatgccatccgcgcggcaggcctgcggctgggcgtttcgacacatgacgatatggaaatcgacgtcgcgctggcagcacgcccctcttatatcgcgctgggacatgtgttcccgacgcaaaccaaacagatgccttctgcaccgcaggggctggaacagctggcacggcatgttgagcgactggcggattatcccaccgtggcgattggcggtatcagtctggcacgcgcgcctgcggtgatagcaacgggtgtcggcagtatcgccgtcgtcagcgccattactcaagccgcagactggcgtttggcaacggcacagttgctggaaattgcaggagttggcgatgaatgaccgtgactttatgcgttatagccgccaaatcctgctcgacgatatcgctctggacgggcagcaaaaactgctcgacagccaggtgctgattatcggtctgggcgggctgggtacacctgctgcgctgtacctggcgggcgctggcgtcgggacgctggtactggcagatgacgacgatgtgcatttaagcaatctgcaacgacaaatcctctttaccactgaagatatcgatcgcccgaaatcgcaggtcagccaacagcgactgacacagttgaatcccgacattcaactgacagcattacaacaacggttaacgggtgaggcgttaaaagatgcggttgcacgggccgatgtggtgctcgactgtaccgacaatatggcgactcgccaggagattaatgccgcctgcgtggcactcaacacgccgcttatcaccgccagcgcggtcggatttggcggtcagttgatggtactgacgccgccctgggagcaggggtgttaccgctgcctgtggccagataaccaggagccagaacgcaactgccgcacggcgggcgtggttggcccggtggtcggggttatgggcactttgcaggcactggaagccattaagttattaagcggtatagagacacctgcgggagaactccgactgttcgacggtaaatcgagccagtggcgcagcctggcgttgcgccgcgccagtggttgcccggtatgcggaggaagcaatgcagatcctgtttaacgatcaagcgatgcagtgcgccgccgggcaaactgttcacgaactactggagcaactcgaccaacgacaagcgggcgcggctctggcgattaatcagcaaatcgtcccgcgtgagcagtgggcgcaacatatcgtgcaggatggcgaccagatcctgctttttcaggttattgcagggggttgaaatgttacgtattgcggacaaaacgtttgattcacatctgtttaccggcacagggaaattcgcttcttcacaactgatggtggaggcgatccgcgcttccggcagccagctggtgacactggcgatgaaacgtgtcgacttgcgccagcacaacgacgctatcctcgaaccgcttatcgcggcgggtgtgaccctgctgccaaatacatccggggcgaaaacagcggaagaagccattttcgccgcccatctggctcgtgaagcgttaggcacaaactggttaaaattagagattcaccctgacgcccgctggctgttgcccgatcccatcgaaaccctgaaagccgccgaaacgctggtacaacagggatttgtcgtgctgccttactgcggggccgatccggtattgtgtaaacgtctggaagaagtcggctgtgcagcggtgatgccgctcggcgcgccgattggctcgaatcagggactggaaacccgcgccatgctggagattattatccagcaggccacagtgccggtggttgtcgatgctggcatcggcgttcccagccatgccgcgcaggcgctggaaatgggggccgacgcggtgttagtgaatacggcgattgccgtcgcggacgatcccgtcaacatggcgaaggcatttcgtctggcggtagaagcaggcctactggcacgtcagtccggaccgggcagccgcagttattttgctcatgccaccagcccgctgaccggatttctggaggcatcggcatgaaaaccttcagcgatcgctggcgacaactggactgggacgacatccgcctgcgtatcaacggcaaaacggctgctgacgtagagcgggcgctaaatgcctcgcaactcacccgcgacgacatgatggcgctgttatcgcctgccgccagtggctatctggaacaactggcccaacgggcgcagcgtctgacccgtcagcgatttggcaacacagttagtttctacgtcccgctttatctttccaatctttgcgctaacgactgcacgtactgtggattttccatgagtaatcgcatcaagcgcaaaacgctggatgaagcggatattgccagggaaagtgccgctatacgggagatgggctttgaacatctgctgttagtcactggtgaacatcaggcgaaagtggggatggattactttcgtcgtcatctccctgcccttcgtgaacagttctcttcactacagatggaagtgcaaccgctggcggagacggaatacgccgagttaaagcaacttggtctggatggcgtgatggtttatcaggagacatatcacgaggcgacttatgcccgccatcatctgaaaggcaaaaaacaggacttcttctggcggctggaaacgccggatcggctggggcgtgcggggattgataagataggcctcggcgcgctaattggcctttccgacaactggcgcgttgacagctatatggttgccgaacatttgctatggctgcaacagcattactggcaaagccgttactctgtctcctttccgcgcctgcgcccgtgtactggcggcattgagcctgcgtcgattatggatgaacgccagttagtgcaaaccatctgcgccttccgactgcttgcaccggagattgaactgtcactctccacgcgggaatcaccgtggtttcgcgatcgcgttattccgctggcgatcaataacgtcagcgccttctcgaaaacgcagccaggtggctatgccgataatcaccccgagttggaacagttctcaccgcacgacgatcgcagaccggaagcggttgctgccgcgttaaccgctcagggtttgcagccggtatggaaagactgggacagctatctgggacgcgcctcgcaaagactatgagacggaatgtaagaacgtaaatttattccgagccgctacggaagatgaaaaacgcaaggttgttgaaagcgttgtgtttttt

SEQ ID NO: 23 - DNA, 1671 bp, Escherichia coli
gtcattgcttcttcacgttatggcaggagcaaactatgcaagtcgacctgctgggttcagcgcaatctgcgcacgcgttacacctttttcaccaacattcccctcttgtgcactgcatgaccaatgatgtggtgcaaacctttaccgccaataccttgctggcgctcggtgcatcgccagcgatggttatcgaaaccgaagaggccagtcagtttgcggctatcgccagtgccttgttgattaacgttggcacactgacgcagccacgcgctcaggcgatgcgtgctgccgttgagcaagcaaaaagctctcaaacaccctggacgcttgatccagtagcggtgggtgcgctcgattatcgccgccatttttgtcatgaacttttatcttttaaaccggcagcgatacgtggtaatgcttcggaaatcatggcattagctggcattgctaatggcggacggggagtggataccactgacgccgcagctaacgcgatacccgctgcacaaacactggcacgggaaactggcgcaatcgtcgtggtcactggcgagatggattatgttaccgatggacatcgtatcattggtattcacggtggtgatccgttaatgaccaaagtggtaggaactggctgtgcattatcggcggttgtcgctgcctgctgtgcgttaccaggcgatacgctggaaaatgtcgcatctgcctgtcactggatgaaacaagccggagaacgcgcagtcgccagaagcgaggggccaggcagttttgttccacatttccttgatgcgctctggcaattgacgcaggaggtgcaggcatgaaacgaattaacgctctgacgattgccggtactgatccgagtggtggtgcggggattcaggccgatcttaaaaccttctcggcacttggcgcttatggttgctcagttattactgcactggtggcgcaaaatacccgtggcgtacagtcggtgtatcgcattgagcctgattttgtcgccgcccagctcgattcggtgttcagcgatgtgcgaatcgataccactaaaatcggtatgctggcggaaaccgatattgttgaagcggtggcagaacggttgcaacgttatcagatccaaaacgtggtactcgacaccgttatgctggcaaaaagcggcgacccgctgctttcaccttcggcggttgctacgctgcgcagtcgattattgccacaggtttcattaataacgccaaacttgcccgaagctgccgccttgctcgacgcgccacacgcgcgcaccgaacaggaaatgctggaacaagggcgatcgctgttggcgatgggctgtggcgcagtgctaatgaaaggtggtcatctggatgatgagcaaagcccggactggctgtttacccgcgagggtgaacaacggtttaccgcaccgcgcattatgaccaaaaacacccacggcactggttgtacactctctgcggcgttggctgcactacgcccgcgccatacaaactgggctgacaccgtacaggaggcaaaaagctggctttcatcggcgttagcccaggccgacacgctggaagttggtcacggtattggtccggttcaccacttccacgcctggtggtgagcaccgtactatagctgctttggttattgtcagcctttggcactgacatt

SEQ ID NO: 24 - DNA, 1077 bp, Escherichia coli
cgttccatgatcgccggccttttcttttttacctgctgaggcataacgtatggcatgtggcgagttctccctgattgcccgttattttgaccgtgtaagaagttctcgtcttgatgtcgaactgggcatcggcgacgattgcgcacttctcaatatccccgagaaacagaccctggcgatcagcactgatacgctggtggcgggtaaccatttcctccctgatatcgatcctgctgatctggcttataaagcactggcggtgaacctaagcgatctggcagcgatgggggccgatccggcctggctgacgctggcattaaccttaccggacgtagacgaagcgtggcttgagtccttcagcgacagtttgtttgatcttctcaattattacgatatgcaactcattggcggcgataccacgcgtgggccattatcaatgacgttgggtatccacggctttgttccgatgggacgagccttaacgcgctctggggcgaaaccgggtgactggatctatgtgaccggtacaccgggcgatagcgccgccgggctggcgattttgcaaaaccgtttgcaggttgccgatgctaaagatgcggactacttgatcaaacgtcatctccgtccatcgccgcgtattttacaggggcaggcactgcgcgatctggcaaattcagccatcgatctctctgacggtttgatttccgatctcgggcatatcgtgaaagccagcgactgcggcgcacgtattgacctggcattgctgccgttttctgatgcgctttctcgccatgttgaaccggaacaggcgctgcgctgggcgctctctggcggtgaagattacgagttgtgtttcactgtgccggaactgaaccgtggcgcgctggatgtggctctcggacacctgggcgtaccgtttacctgtatcgggcaaatgaccgccgatatcgaagggctttgttttattcgtgacggcgaacctgttacattagactggaaaggatatgaccattttgccacgccataaagatgtcgcgaaaagtcgcctgaagatgagtaatccgtggcatctacttg

SEQ ID NO: 25 - DNA, 60 bp, Artificial Sequence
  Description of Artificial Sequence Synthetic primer
ataatagaattcataaaagtaaaggaggtatgtaatatgaacgagagatatcaattaaac

SEQ ID NO: 26 - DNA, 48 bp, Artificial Sequence
  Description of Artificial Sequence Synthetic primer
ataatagtcgacgaatatagtttatatcatttctgcaaagtacctgtg

SEQ ID NO: 27 - DNA, 47 bp, Artificial Sequence
  Description of Artificial Sequence Synthetic primer
ataatagtcgaccatccccatatcaaaggaggtggcagtatgatttc

SEQ ID NO: 28 - DNA, 38 bp, Artificial Sequence
  Description of Artificial Sequence Synthetic primer
ataatacccgggtgataacaaaatttcttcactctgtc

SEQ ID NO: 29 - DNA, 38 bp, Artificial Sequence
  Description of Artificial Sequence Synthetic primer
ataataggcgcgccttaagcccgataaatgggatggag

SEQ ID NO: 30 - DNA, 39 bp, Artificial Sequence
  Description of Artificial Sequence Synthetic primer
ataataggcgcgccgctagcagtagctttcctgaatgga

SEQ ID NO: 31 - DNA, 1526 bp, Clostridium cellulyticum
ataatagaattcataaaagtaaaggaggtatgtaatatgaacgagagatatcaattaaacaaaaatcttgcccaaatgctaaagggcggagtaatcatggatgtagtaaatgccaaagaagcagaaattgcacaaaaagccggagccgttgcagtaatggctctcgaaagagttccttccgatataagaaaagccggaggagttgcaagaatgtccgatccaaaaatgataaaagatatacaaagtgccgtatcaattcctgttatggccaaagttagaataggacattttgttgaagcacaggttcttgaagccctttcaattgactatattgatgaaagcgaggttttaactccggcagacgaagaatttcacatagataagcataccttcaaggttccatttgtatgcggtgcaaaaaatctcggagaagctctcagaagaattagtgaaggtgcatccatgataagaactaaaggtgaagccggtacaggaaatgttgttgaagccgtccgacatatgagaactgtaacaaatgaaatcagaaaggtgcagagtgcatccaagcaggaacttatgaccatagcaaaagaatttggtgctccatatgaccttattttatatgttcacgaaaacggtaagcttcctgttataaactttgcagcaggcggaatcgcaactcccgccgatgcggcattaatgatgcagcttggatgcgacggcgtatttgttggttcgggaatatttaaatcctcagatccagccaaaagagcaaaggcaatcgtaaaggcaactacatactataatgatccgcaaatcattgcagaggtctctgaagagcttggtactgccatggattccatagatgtaagagagttaacaggcaacagtctgtatgcctctagaggatggtaatcttacagaaaggattataaatgaaaaaaataggtgtgttaggcttgcagggtgctatctcagaacatttggataaactatccaaaataccaaatgtagagccattcagcctaaaatataaagaagaaattgatacaatagacggacttatcatacccggcggtgaaagtactgcaatcggcaggcttctctctgattttaacctgacagaaccactgaaaacaagggtaaatgccgggatgcctgtatggggaacctgtgcaggcatgattatccttgcaaaaacgattactaatgaccgccgacgtcatctggaggttatggacataaatgttatgcggaacgggtatggaagacagttgaacagctttacaacagaggtttccctggctaaagtttcttctgataaaatcccgttggtttttattagagcaccttatgtagtcgaggtagctccgaatgttgaagttcttctgcgtgtagacgaaaacatagtcgcgtgcaggcaggacaatatgctggccacctcctttcatccggagctgacagaagacctgagttttcacaggtactttgcagaaatgatataaactatattcgtcgactattat

SEQ ID NO: 32 - DNA, 589 bp, Clostridium cellulyticum
ataatagtcgaccatccccatatcaaaggaggtggcagtatgatttcaatgatatgggctatgggccgcaacaacgcccttggatgtaaaaacagaatgccctggtacattcccgcagattttgcatatttcaaaaaagttacaatgggaaaaccggtcattatggggagaaaaacttttgaatctatcggtaaacctttaccgggcagaaagaacatagtaattactcgagacacaggatatgatccacaaggctgtattgtggttaattctatagaaaaagccatggagtatacagaagaaaaggaagtctttataatagggggagcagaaatatacaaagaatttcttcctattgcagacagactatatataactctgatagaaaaagagtttgaagcggatgcatttttcccggaaatagactatagtaagtggaagcagatatcctgcgaaacaggaatcaaggatgaaaaaaatccatatgagtataagtggttggtatacgaaagagttaaacaataaggtaaataagaatgtataaaaatgttgacagagtgaagaaattttgttatcacccgggtattat

SEQ ID NO: 33 - DNA, 813 bp, Clostridium cellulolyticum
atgaagaaagtattaacaattgcaggatcagactgcagcggaggagcaggaattcaggcggatttaaaaacctttgctgcacatggtatttatggaatgagtgttattgtatcggtagttgcagagaatacataccgcgtcatcgatattcaggatataacgcctgatatgattaaaaaacagatagatgcagtttttgaagacataacacctgatgctgtaaaaataggtatgctgtcagacaaggagtgcatggaagctgttgctgaaaaattggaacaatataagcctgtaaatgtggttattgaccctgtaatgattgcaaaaggcggtcacgccctcatgaaagaagaagctctcgagttttttataaaaaggcttatacccctggcaggtatgcttacaccaaatattccggaggcagaggcgattaccgggatgcggataactgaccataatgatatgcagaaggcagctagaagcatttatgaaatgggtgcaggcagtgttctggtaaaaggcggacatttatatggagatgcggaagacatactttttgacggaagagaattttacatatattcaactaaaagaatccagacaaaaaatacacatggaacaggatgtacattttcatcagctatagcctcaaaccttgccttgggaataccttttacaacagcggttgaaaaagctaaagaatacgtcacaatggcgattgaacattcacttgaaatcggaaagggacatggtccaacgcatcatttttatggggtttacaaaaacggattggataatcggaggttgctatga

SEQ ID NO: 34 - protein, 270 aa, Clostridium cellulolyticum
MKKVLTIAGSDCSGGAGIQADLKTFAAHGIYGMSVIVSVVAENTYRVIDIQDITPDMIKKQIDAVFEDITPDAVKIGMLSDKECMEAVAEKLEQYKPVNVVIDPVMIAKGGHALMKEEALEFFIKRLIPLAGMLTPNIPEAEAITGMRITDHNDMQKAARSIYEMGAGSVLVKGGHLYGDAEDILFDGREFYIYSTKRIQTKNTHGTGCTFSSAIASNLALGIPFTTAVEKAKEYVTMAIEHSLEIGKGHGPTHHFYGVYKNGLDNRRLL

SEQ ID NO: 35 - DNA, 1311 bp, Clostridium cellulolyticum
atgaattacaaaacacagatggacgccgcaaaaaaaggtattattacaaatgaaatgaagattgtttctcgaaaggaatcaatggatgaaaataagcttcgggagcttgttgctgagggcagaatagctattccggcaaatataaatcacaagtccttaagtccggagggaataggagaaggtctcagaacaaaaatcaatgtaaaccttggtatatcaggggattgtcctgactatacaaaagaaatggaaaaggctgatatgtcaatcaaatttggtgttgaagccataatggaccttagtaattacggaaagacaaacacctttcgcaaagagcttataaagcgttctcctgccatgataggaaccgttcccatgtatgatgccataggctaccttgaaaaggacctgatggatattaaagcttcggactttttaaaagttgttgaggctcatgctgcagaaggtgtggactttatgacaattcatgcaggaataaataaacgtgccgttgagtgtttcaaacgctcaaagagacttaccaatattgtttcaagaggaggttcccttctttttgcatggatggagatgactggcaatgaaaatcctttttttgagtattatgacgatttccttggaatactcagggaatatgatgtaaccataagtcttggagatgcattgaggcccggtagtatcaatgacagctcagacgccggacaattaagtgaacttatagaactgggcgacctgaccaaacgtgcatgggaaaaggatgtacaggtaatggttgaggggccgggacatatggctatgaacgagatagccgccaatatgactattcaaaagagactatgtcatggagcacctttttatgtactggggcctttggtgacagatatagcaccgggatatgaccatatcacctcggcaataggcggagcaattgctgcggcaaacggtgcagatttcttatgttatgtaacccctgcggagcatttaaggttacctgacctttcggatgtaaaagaaggaatagttgcttcaaaaatagccgctcatgccgccgatattgcaaagggaatacctaatgcacgtgaaatagataataaaatgagcgatgccagacgccgaatcgactgggaagaaatgttctcatatgctattgacgaagataaggcaagagcatattttgaaagcacacctcccactgacagacatacctgctcaatgtgcggaaaaatgtgtgctatgaggactacaaataagattttagccggtgaaaaggttgagtttgtaacagagaaatag

SEQ ID NO: 36 - protein, 436 aa, Clostridium cellulolyticum
MNYKTQMDAAKKGIITNEMKIVSRKESMDENKLRELVAEGRIAIPANINHKSLSPEGIGEGLRTKINVNLGISGDCPDYTKEMEKADMSIKFGVEAIMDLSNYGKTNTFRKELIKRSPAMIGTVPMYDAIGYLEKDLMDIKASDFLKVVEAHAAEGVDFMTIHAGINKRAVECFKRSKRLTNIVSRGGSLLFAWMEMTGNENPFFEYYDDFLGILREYDVTISLGDALRPGSINDSSDAGQLSELIELGDLTKRAWEKDVQVMVEGPGHMAMNEIAANMTIQKRLCHGAPFYVLGPLVTDIAPGYDHITSAIGGAIAAANGADFLCYVTPAEHLRLPDLSDVKEGIVASKIAAHAADIAKGIPNAREIDNKMSDARRRIDWEEMFSYAIDEDKARAYFESTPPTDRHTCSMCGKMCAMRTTNKILAGEKVEFVTEK

SEQ ID NO: 37 - DNA, 822 bp, Clostridium cellulolyticum
atgagtgagtatacaaagcaaataacaagattaatctctgaggtcagaagtaaaaagccgcttattcacaatattacaaattatgtaactgtaaatgattgtgcaaatgttactctggcaataggggcctccccaattatggcagatgatattgatgaggctgcggatattacttctatttcttctgcacttgtaataaatataggaactttaaacaaaagaacgattgaatcaatgattctatcgggtaaaaaagcaaatgagaagggtattcccgtaatttttgatcctgttggagcaggggcttcagctttgagaaatgagactacaagcactattcttgataaaataaaaataagtgtcttgcgtggcaatttgtctgagatatcctacatagccgggcgtaatgcctcaacaaaaggcgttgatgcttcagaagcggatattcaatcaaatgactccatcgcagttgcaaaagcggcagctgtaaagcttggatgtgttgttgcagtaacaggagctgttgatgtgatttcagacggtaaaaacgttgtgactgtcttaaacggacataaaatgctgtcaaatgttacaggaacagggtgtatgactactgctttggttggttccttctgcggggcagtaaaagattatttcaaagctgctgtggcaggagtgacagtgatgggaatatcaggagagattgcctatgaagccgcaggacataaaggtactggaagctaccatattgcaataatcgatgccatcagtaggatggatgaaaatatttttggagagaaggcaagaataaatgaaatctaa

SEQ ID NO: 38 - protein, 273 aa, Clostridium cellulolyticum
MSEYTKQITRLISEVRSKKPLIHNITNYVTVNDCANVTLAIGASPIMADDIDEAADITSISSALVINIGTLNKRTIESMILSGKKANEKGIPVIFDPVGAGASALRNETTSTILDKIKISVLRGNLSEISYIAGRNASTKGVDASEADIQSNDSIAVAKAAAVKLGCVVAVTGAVDVISDGKNVVTVLNGHKMLSNVTGTGCMTTALVGSFCGAVKDYFKAAVAGVTVMGISGEIAYEAAGHKGTGSYHIAIIDAISRMDENIFGEKARINEI

SEQ ID NO: 39 - DNA, 633 bp, Clostridium cellulolyticum
atgaaatctaaaattgattatactctttatcttgtaacagaccatcagctgatgagtactaaaacactggaggaggcagtagagcaggcaatagcaggaggctgtactttagtgcagttacgtgaaaaaactgcttcttcccgggatttttatcaaaatgctattaacgtaaaaactataacagacaaatataatgtacctcttattataaatgatagaattgatattgctttggctgtaggtgctgacggagtccacgtggggcagagcgatcttccggctgctgttgtgcgaaaaatcataggtaacgataagatactcggcgtatcggcagggtctgttgaaaaggcaattgaagctcagaaaatcggtgctgactatataggagtgggtgctttgttttcaacaagcacaaaaacagatgcaaaggcagtgtctatagaaactctcatgaaaattgtaagggaagtttcaattcctgtggtcggtataggcgggataaatgcggagaatgcagtacaactgaaaaatacgggaataaggggtattgccgtagtatcggctattatttcacaaaaggatataaaatcatctgctgaaaaattgcttgaaatattcgtcaacaaagcttaa

SEQ ID NO: 40 - protein, 210 aa, Clostridium cellulolyticum
MKSKIDYTLYLVTDHQLMSTKTLEEAVEQAIAGGCTLVQLREKTASSRDFYQNAINVKTITDKYNVPLIINDRIDIALAVGADGVHVGQSDLPAAVVRKIIGNDKILGVSAGSVEKAIEAQKIGADYIGVGALFSTSTKTDAKAVSIETLMKIVREVSIPVVGIGGINAENAVQLKNTGIRGIAVVSAIISQKDIKSSAEKLLEIFVNKA

SEQ ID NO: 41 - DNA, 1884 bp, Escherichia coli
atgtctgcaacaaaactgacccgccgcgaacaacgcgcccgggcccaacattttatcgacaccctggaaggcaccgcctttcccaactcaaaacgcatttatatcactggcacacaccccggcgtgcgcgtgccgatgcgtgagatccagcttagcccgacgctaattggcggtagcaaagaacagccgcagtacgaagaaaacgaagcgattccggtctacgacacctccggcccgtatggtgatccgcagattgccattaacgtgcagcaagggctggcaaaactacgccagccgtggatcgatgcgcgcggcgataccgaagaacttaccgtgcgcagttccgattacactaaagcgcggctggcagatgatggcctcgacgaactgcgttttagcggcgtactaacaccaaaacgcgccaaagcaggacgccgtgtcacccaactgcactacgcccgccagggcatcatcacgccggaaatggaattcatcgccatccgcgagaatatgggccgcgagcgcatccgtagcgaggttttacgccaccagcatccgggaatgagctttggcgcacatctgccggaaaatatcactgcggaatttgtccgtgatgaagttgctgccggacgtgcgattatcccggccaacattaatcatccggaatcggagccgatgattattggtcgcaatttcctggtaaaagttaacgccaatatcggcaactcggcggtcacctcttccatcgaagaagaagtggaaaagctggtatggtccacgcgctggggagcggatacggtgatggatctctccaccggtcgctatattcacgaaacccgcgagtggattttgcgtaacagcccggtgccgatcggtacagtgccgatctaccaggcgctggagaaggttaacgggatcgccgaagatcttacctgggaagcgttccgcgacacgctgctggaacaggccgagcaaggtgtggattacttcactatccatgcgggcgtactgctgcgctatgtgccgatgaccgcgaaacgcctgaccggtatcgtctctcgcggcggttcgattatggcgaaatggtgcctctcccatcatcaggaaaatttcctctatcaacacttccgcgaaatttgtgaaatctgtgccgcttatgacgtttcgctgtcgctgggcgacggtctgcgccccggttctattcaggacgccaacgatgaagcgcagtttgccgagctgcatacgctgggcgaactgaccaaaattgcctgggaatatgacgtgcaggtgatgattgaaggcccaggccacgtgccgatgcagatgatccgccgcaatatgaccgaggagttagagcactgccacgaagcgccgttttacactctggggccgctaactaccgatattgcgccgggctatgaccacttcacgtcggggattggtgcggcgatgattggctggtttggctgcgcgatgctctgttacgtaacgccaaaagagcatctgggtctgcccaataaagaagatgttaagcaggggcttatcacctataagattgctgcccacgccgctgacctggcgaaagggcatccgggcgcgcaaattcgcgataacgccatgtcgaaagcccgcttcgaatttcgctgggaagaccagtttaatctggccctcgacccgtttaccgcccgcgcttatcacgatgaaaccctgccgcaagagtcaggtaaagtcgcccatttttgctccatgtgtgggccgaaattctgctcgatgaaaatcagccaggaagtgcgtgattacgccgccacgcaaactattgaaatgggaatggcggatatgtcggagaacttccgtgccagaggcgggagaaatctacctgcgtaa

SEQ ID NO: 42 - protein, 627 aa, Escherichia coli
MSATKLTRREQRARAQHFIDTLEGTAFPNSKRIYITGTHPGVRVPMREIQLSPTLIGGSKEQPQYEENEAIPVYDTSGPYGDPQIAINVQQGLAKLRQPWIDARGDTEELTVRSSDYTKARLADDGLDELRFSGVLTPKRAKAGRRVTQLHYARQGIITPEMEFIAIRENMGRERIRSEVLRHQHPGMSFGAHLPENITAEFVRDEVAAGRAIIPANINHPESEPMIIGRNFLVKVNANIGNSAVTSSIEEEVEKLVWSTRWGADTVMDLSTGRYIHETREWILRNSPVPIGTVPIYQALEKVNGIAEDLTWEAFRDTLLEQAEQGVDYFTIHAGVLLRYVPMTAKRLTGIVSRGGSIMAKWCLSHHQENFLYQHFREICEICAAYDVSLSLGDGLRPGSIQDANDEAQFAELHTLGELTKIAWEYDVQVMIEGPGHVPMQMIRRNMTEELEHCHEAPFYTLGPLTTDIAPGYDHFTSGIGAAMIGWFGCAMLCYVTPKEHLGLPNKEDVKQGLITYKIAAHAADLAKGHPGAQIRDNAMSKARFEFRWEDQFNLALDPFTARAYHDETLPQESGKVAHFCSMCGPKFCSMKISQEVRDYAATQTIEMGMADMSENFRARGGRNLPA

SEQ ID NO: 43 - DNA, 636 bp, Escherichia coli
atgtatcagcctgattttcctcctgtaccttttcgttcaggactgtacccggtggtggacagcgtacagtggatcgaacgtctgttggatgcaggcgtacgtactctccagctacgcatcaaagatcggcgcgatgaagaggtggaagccgatgtcgtggcggcaattgcgctgggccgccgctataacgcgcgattgtttatcaacgattactggcggctggcgatcaagcatcaggcgtatggcgtccatttggggcaggaagatttgcaagccaccgatctcaatgccatccgcgcggcaggcctgcggctgggcgtttcgacacatgacgatatggaaatcgacgtcgcgctggcagcacgcccctcttatatcgcgctgggacatgtgttcccgacgcaaaccaaacagatgccttctgcaccgcaggggctggaacagctggcacggcatgttgagcgactggcggattatcccaccgtggcgattggcggtatcagtctggcacgcgcgcctgcggtgatagcaacgggtgtcggcagtatcgccgtcgtcagcgccattactcaagccgcagactggcgtttggcaacggcacagttgctggaaattgcaggagttggcgatgaatga

SEQ ID NO: 44 - protein, 211 aa, Escherichia coli
MYQPDFPPVPFRSGLYPVVDSVQWIERLLDAGVRTLQLRIKDRRDEEVEADVVAAIALGRRYNARLFINDYWRLAIKHQAYGVHLGQEDLQATDLNAIRAAGLRLGVSTHDDMEIDVALAARPSYIALGHVFPTQTKQMPSAPQGLEQLARHVERLADYPTVAIGGISLARAPAVIATGVGSIAVVSAITQAADWRLATAQLLEIAGVGDE

SEQ ID NO: 45 - DNA, 753 bp, Escherichia coli
atgaatgaccgtgactttatgcgttatagccgccaaatcctgctcgacgatatcgctctggacgggcagcaaaaactgctcgacagccaggtgctgattatcggtctgggcgggctgggtacacctgctgcgctgtacctggcgggcgctggcgtcgggacgctggtactggcagatgacgacgatgtgcatttaagcaatctgcaacgacaaatcctctttaccactgaagatatcgatcgcccgaaatcgcaggtcagccaacagcgactgacacagttgaatcccgacattcaactgacagcattacaacaacggttaacgggtgaggcgttaaaagatgcggttgcacgggccgatgtggtgctcgactgtaccgacaatatggcgactcgccaggagattaatgccgcctgcgtggcactcaacacgccgcttatcaccgccagcgcggtcggatttggcggtcagttgatggtactgacgccgccctgggagcaggggtgttaccgctgcctgtggccagataaccaggagccagaacgcaactgccgcacggcgggcgtggttggcccggtggtcggggttatgggcactttgcaggcactggaagccattaagttattaagcggtatagagacacctgcgggagaactccgactgttcgacggtaaatcgagccagtgcagcctggcgttgcgccgcgccagtggttgcccggtatgcggaggaagcaatgcagatcctgtttaa

SEQ ID NO: 46 - protein, 250 aa, Escherichia coli
MNDRDFMRYSRQILLDDIALDGQQKLLDSQVLIIGLGGLGTPAALYLAGAGVGTLVLADDDDVHLSNLQRQILFTTEDIDRPKSQVSQQRLTQLNPDIQLTALQQRLTGEALKDAVARADVVLDCTDNMATRQEINAACVALNTPLITASAVGFGGQLMVLTPPWEQGCYRCLWPDNQEPERNCRTAGVVGPVVGVMGTLQALEAIKLLSGIETPAGELRLFDGKSSQCSLALRRASGCPVCGGSNADPV

SEQ ID NO: 47 - DNA, 201 bp, Escherichia coli
atgcagatcctgtttaacgatcaagcgatgcagtgcgccgccgggcaaactgttcacgaactactggagcaactcgaccaacgacaagcgggcgcggccctggcgattaatcagcaaatcgtcccgcgtgagcagtgggcgcaacatatcgtgcaggatggcgaccagatcctgctttttcaggttattgcagggggttga

SEQ ID NO: 48 - protein, 66 aa, Escherichia coli
MQILFNDQAMQCAAGQTVHELLEQLDQRQAGAALAINQQIVPREQWAQHIVQDGDQILLFQVIAGG

SEQ ID NO: 49 - DNA, 771 bp, Escherichia coli
atgttacgtattgcggacaaaacgtttgattcacatctgtttaccggcacagggaaattcgcttcttcacaactgatggtggaggcgatccgcgcttccggcagccagctggtgacactggcgatgaaacgtgtcgacttgcgccagcacaacgacgctatcctcgaaccgcttatcgcggcgggtgtgaccctgctgccaaatacatccggggcgaaaacagcggaagaagccattttcgccgcccatctggctcgtgaagcgttaggcacaaactggttaaaattagagattcaccctgacgcccgctggctgttgcccgatcccatcgaaaccctgaaagccgccgaaacgctggtacaacagggatttgtcgtgctgccttactgcggggccgatccggtattgtgtaaacgtctggaagaagtcggctgtgcagcggtgatgccgctcggcgcgccgattggctcgaatcagggactggaaacccgcgccatgctggagattattatccagcaggccacagtgccggtggttgtcgatgctggcatcggcgttcccagccatgccgcgcaggcgctggaaatgggggccgacgcggtgttagtgaatacggcgattgccgtcgcggacgatcccgtcaacatggcgaaggcatttcgtctggcggtagaagcaggcctactggcacgtcagtccggaccgggcagccgcagttattttgctcatgccaccagcccgctgaccggatttctggaggcatcggcatga

SEQ ID NO: 50 - protein, 256 aa, Escherichia coli
MLRIADKTFDSHLFTGTGKFASSQLMVEAIRASGSQLVTLAMKRVDLRQHNDAILEPLIAAGVTLLPNTSGAKTAEEAIFAAHLAREALGTNWLKLEIHPDARWLLPDPIETLKAAETLVQQGFVVLPYCGADPVLCKRLEEVGCAAVMPLGAPIGSNQGLETRAMLEIIIQQATVPVVVDAGIGVPSHAAQALEMGADAVLVNTAIAVADDPVNMAKAFRLAVEAGLLARQSGPGSRSYFAHATSPLTGFLEASA

SEQ ID NO: 51 - DNA, 1134 bp, Escherichia coli
atgaaaaccttcagcgatcgctggcgacaactggactgggacgacatccgcctgcgtatcaacggcaaaacggctgctgacgtagagcgggcgctaaatgcctcgcaactcacccgcgacgacatgatggcgctgttatcgcctgccgccagtggctatctggaacaactggcccaacgggcgcagcgtctgacccgtcagcgatttggcaacacagttagtttctacgtcccgctttatctttccaatctttgcgctaacgactgcacgtactgtggattttccatgagtaatcgcatcaagcgcaaaacgctggatgaagcggatattgccagggaaagtgccgctatacgggagatgggctttgaacatctgctgttagtcactggtgaacatcaggcgaaagtggggatggattactttcgtcgtcatctccctgcccttcgtgaacagttctcttcactacagatggaagtgcaaccgctggcggagacggaatacgccgagttaaagcaacttggtctggatggcgtgatggtttatcaggagacatatcacgaggcgacttatgcccgccatcatctgaaaggcaaaaaacaggacttcttctggcggctggaaaccccggatcggctggggcgtgcggggattgataagataggcctcggcgcgctaattggcctttccgacaactggcgcgttgacagctatatggttgccgaacatttgctatggctgcaacagcattactggcaaagccgttactctgtctcctttccgcgcctgcgcccgtgtactggcggcattgagcctgcgtcgattatggatgaacgccagttagtgcaaaccatctgcgccttccgactgcttgcaccggagattgaactgtcactctccacgcgggaatcaccgtggtttcgcgatcgcgttattccgctggcgatcaataacgtcagcgccttctcgaaaacgcagccaggtggctatgccgataatcaccccgagttggaacagttctcaccgcacgacgatcgcagaccggaagcggttgctgccgcgttaaccgctcagggtttgcagccggtatggaaagactgggacagctatctgggacgcgcctcgcaaagactatga

SEQ ID NO: 52 - protein, 377 aa, Escherichia coli
MKTFSDRWRQLDWDDIRLRINGKTAADVERALNASQLTRDDMMALLSPAASGYLEQLAQRAQRLTRQRFGNTVSFYVPLYLSNLCANDCTYCGFSMSNRIKRKTLDEADIARESAAIREMGFEHLLLVTGEHQAKVGMDYFRRHLPALREQFSSLQMEVQPLAETEYAELKQLGLDGVMVYQETYHEATYARHHLKGKKQDFFWRLETPDRLGRAGIDKIGLGALIGLSDNWRVDSYMVAEHLLWLQQHYWQSRYSVSFPRLRPCTGGIEPASIMDERQLVQTICAFRLLAPEIELSLSTRESPWFRDRVIPLAINNVSAFSKTQPGGYADNHPELEQFSPHDDRRPEAVAAALTAQGLQPVWKDWDSYLGRASQRL

SEQ ID NO: 53 - DNA, 801 bp, Escherichia coli
atgaaacgaattaacgctctgacgattgccggtactgatccgagtggtggtgcggggattcaggccgatcttaaaaccttctcggcacttggcgcttatggttgctcagttattactgcactggtagcgcaaaatacccgtggcgtacagtcggtgtatcgcattgagcctgattttgtcgccgcccagctcgattcggtgttcagcgatgtgcgaatcgacaccactaaaatcggtatgttggcggaaaccgatattgttgaagcggtggcagaacggttgcaacgttatcagatccaaaacgtggtactcgacaccgttatgctggcaaaaagcggcgacccgctgctttcaccttcggcggttgttacgctgcgcagtcgattattgccacaggtttcattaataacgccaaacttgcccgaagctgccgccttgctcgacgcgccacacgcgcgcaccgaacaggaaatgctggaacaagggcgatcgctgttggcgatgggatgtggcgcagtgctaatgaaaggtggtcatctggatgatgagcaaagcccggactggctgtttacccgcgagggtgaacaacggtttaccgcaccgcgcattatgaccaaaaacacccacggcactggttgtacactctctgcggcgttggctgcactacgcccgcgccatacaaactgggctgacaccgtacaggaggcaaaaagctggctttcatcggcgttagcccaggccgacacgctggaagttggtcacggtattggtccggttcaccacttccacgcctggtggtga

SEQ ID NO: 54 - protein, 266 aa, Escherichia coli
MKRINALTIAGTDPSGGAGIQADLKTFSALGAYGCSVITALVAQNTRGVQSVYRIEPDFVAAQLDSVFSDVRIDTTKIGMLAETDIVEAVAERLQRYQIQNVVLDTVMLAKSGDPLLSPSAVVTLRSRLLPQVSLITPNLPEAAALLDAPHARTEQEMLEQGRSLLAMGCGAVLMKGGHLDDEQSPDWLFTREGEQRFTAPRIMTKNTHGTGCTLSAALAALRPRHTNWADTVQEAKSWLSSALAQADTLEVGHGIGPVHHFHAWW

SEQ ID NO: 55 - DNA, 789 bp, Escherichia coli
tcatgcctgcacctcctgcgtcaattgccagagcgcatcaaggaaatgtggaacaaaactgcctggcccctcgcttctggcgactgcgcgttctccggcttgtttcatccagtgacaggcagatgcgacattttccagcgtatcgcctggtaacgcacagcaggcagcgacaaccgccgataatgcacagccagttcctaccactttggtcattaacggatcgccaccgtgaatgccaacggtgcgacgaccatcggtaacataatccatctcgccagtgaccacgacgattgcgccagtttcccgtgccagtgtttgtgcagcgggtatcgcattagctgccgcgtcagtggtatccactccccgtccgccagtagcaacgccagctaatgccatgatttccgaagcattaccacgtatcgctgccggtttaaaagataaaagttcatgacaaaaatggcgacgataatcgagcgcaccaaccgctactggatcaagcgtccagggggtttgagagctttttgcttgctcaacggcggcacgcatcgcctgtgcgcgtggctgcgtcagcgtgccaacgttaatcaacaaggcactggcgatagccgcaaactgactggcctcttcggtttcgataaccatcgctggcgatgcaccgatcgccagcaaggtattggcagtaaaggtttgcaccacatcgttggtcatgcaatgcacaagaggggaatgttggtgaaaaaggtgtaacgcgtgcgcagattgcgcggaactcagcaggtcgacttgcat

SEQ ID NO: 56 - protein, 262 aa, Escherichia coli
MQVDLLSSAQSAHALHLFHQHSPLVHCMTNDVVQTFTANTLLAIGASPAMVIETEEASQFAAIASALLINVGTLTQPRAQAMRAAVEQAKSSQTPWTLDPVAVGALDYRRHFCHELLSFKPAAIRGNASEIMALAGVATGGRGVDTTDAAANAIPAAQTLARETGAIVVVTGEMDYVTDGRRTVGIHGGDPLMTKVVGTGCALSAVVAACCALPGDTLENVASACHWMKQAGERAVARSEGPGSFVPHFLDALWQLTQEVQA

SEQ ID NO: 57 - DNA, 978 bp, Escherichia coli
atggcatgtggcgagttctccctgattgcccgttattttgaccgtgtaagaagttctcgtcttgatgtcgaactgggcatcggcgacgattgcgcacttctcaatatccccgagaaacagaccctggcgatcagcactgatacgctggtggcgggtaaccatttcctccctgatatcgatcctgctgatctggcttataaagcactggcggtgaacctaagcgatctggcagcgatgggggccgatccggcctggctgacgctggcattaaccttaccggacgtagacgaagcgtggcttgagtccttcagcgacagtttgtttgatcttctcaattattacgatatgcaactcattggcggcgataccacgcgtgggccattatcaatgacgttgggtatccacggctttgttccgatgggacgagccttaacgcgctctggggcgaaaccgggtgactggatctatgtgaccggtacaccgggcgatagcgccgccgggctggcgattttgcaaaaccgtttgcaggttgccgatgctaaagatgcggactacttgatcaaacgtcatctccgtccatcgccgcgtattttacaggggcaggcactgcgcgatctggcaaattcagccatcgatctctctgacggtttgatttccgatctcgggcatatcgtgaaagccagcgactgcggcgcacgtattgacctggcattgctgccgttttctgatgcgctttctcgccatgttgaaccggaacaggcgctgcgctgggcgctctctggcggtgaagattacgagttgtgtttcactgtgccggaactgaaccgtggcgcgctggatgtggctctcggacacctgggcgtaccgtttacctgtatcgggcaaatgaccgccgatatcgaagggctttgttttattcgtgacggcgaacctgttacattagactggaaaggatatgaccattttgccacgccataa

SEQ ID NO: 58 - protein, 325 aa, Escherichia coli
MACGEFSLIARYFDRVRSSRLDVELGIGDDCALLNIPEKQTLAISTDTLVAGNHFLPDIDPADLAYKALAVNLSDLAAMGADPAWLTLALTLPDVDEAWLESFSDSLFDLLNYYDMQLIGGDTTRGPLSMTLGIHGFVPMGRALTRSGAKPGDWIYVTGTPGDSAAGLAILQNRLQVADAKDADYLIKRHLRPSPRILQGQALRDLANSAIDLSDGLISDLGHIVKASDCGARIDLALLPFSDALSRHVEPEQALRWALSGGEDYELCFTVPELNRGALDVALGHLGVPFTCIGQMTADIEGLCFIRDGEPVTLDWKGYDHFATP

SEQ ID NO: 59 - DNA, 915 bp, Clostridium cellulolyticum
atggataaagatttgttgattagtaacattaaaaaaatgaagaaagagcagaacgcagttattgttgctcacagttatcaggttgatgaggtgcaggagattgctgacgttacaggagattcattagctctaagtcaattttgtgcctccagtcaggcggatactatagttttttgcggggtacactttatggcagaaagtgcgaagcttctatcgcctgaaaaaacggttctgttgcctgaaataaatgcaggttgcccaatggcagatatggttacggctgaggctctgaaagaggctaagaaaaagtatcctcacgcagctgttgtatgttatataaactcaagtgctgaggttaaggccgagtgtgatatctgctgtacatcttcaaatgcggagaaagtaatcagatctatcgataaaaaggatattatatttgctccagataaaaatcttggcagttatgtagcaaaaaaggttcctgaaaaaaacattattttttgggaaggctactgcattacacatcataagattaagaaagatgctgtcatagagtcaaagagacttcatcctgatgctattttgctggtacatccggagtgccgacccgaaatacaggagcttgctgattttgtgggaagcacaaagcagattatagattatgcaagaaattccgagcatgacaaatttattattggaactgagatgggtgttctttaccagttaaaaaaggagaacccaaacaagactttttatatgatgtcaacagggctgatttgtccgaatatgaagaaaacatcattacagagtgttcatgatgccttagctaagaggcaatacgaaattacattggatagcggtattatagaacgtgcatccggtagtttgaatagaatgctggcagtagggaaatag

SEQ ID NO: 60 - protein, 304 aa, Clostridium cellulolyticum
MDKDLLISNIKKMKKEQNAVIVAHSYQVDEVQEIADVTGDSLALSQFCASSQADTIVFCGVHFMAESAKLLSPEKTVLLPEINAGCPMADMVTAEALKEAKKKYPHAAVVCYINSSAEVKAECDICCTSSNAEKVIRSIDKKDIIFAPDKNLGSYVAKKVPEKNIIFWEGYCITHHKIKKDAVIESKRLHPDAILLVHPECRPEIQELADFVGSTKQIIDYARNSEHDKFIIGTEMGVLYQLKKENPNKTFYMMSTGLICPNMKKTSLQSVHDALAKRQYEITLDSGIIERASGSLNRMLAVGK

SEQ ID NO: 61 - DNA, 1605 bp, Clostridium cellulolyticum
atggaagaggatagtaataaggttgatgttgaggtcatacacaaggatgtcgtcatcattggtagcggaatagccggagtatatactgcattggaaatacccgacagtttccagatagggataattaccaaagagacactggacataagcaattcagttctcgcacaagggggaatagcagtatctcttgatgagaagaatgattctccacaactacatttcaaagatactctttttgcaggtgcaggattaaatgatcaaaagagcgtatgggttctggtagaagaggctgctgaaaatattagaattttgtgcagcctaggggtaaactttgataaaagcggacaacatctatcccttactagagaaggggcccatagtgtaaatagaattattcactcaggagatacgactggtaaggaagtctgtgacaagcttattgaggttgcccggagaaagaagaacatatcgatttttgagagtcactttgcagtcgatcttgtgatcgaagagggcaaatgcaaaggtgtaatagtttatgacgaaattgaagataaaattaagatatttaaatccggctcggtagttgttgcaactgggggttttggacagatttatgcacatactactaatcctgaggtcgcaactggtgacggagtcggaatgtgtttgagagcgggtgcccaggccatggatatggagtttatacagttccatcctacagtactataccacccaaaagacaagagtttcctaatatctgaggcggttagaggagagggtgctcaacttaaaaatagcaacggtgagccttttatgaagaaatatcacgagttgggtgaactggcacccagagacattgtttcaagagcgatttttaaagaaatgtatcttactgattccaaaaatgtatttctggatataacatttaaaggtagggaatatatcgaaagtaggtttcctaatatctttaaaacatgtctggattacggtattgatatttctaaggattttattccggttgctcccgcagagcattattgtatggggggagtaaaaacagatgttgacgggcagacaaatattccgggtctgtatgcatgtggagaggtagcttgtacagggattcacggtgcaaacaggctcgcaagcaattctttgctagaaggactggttttcggcaggaaaatcgcaaagaagatcgagtccgaaggaagaccttgtaataattcagccgtcaattcaaggctctgttatgtatccaataaagaaaatgatgcggctcttaaatctatgaaggaagagatacaggctgcaatgacaaagtatgtaggtataatcagaagtcaacaaggtcttgaaaaggctgcccaaattattaaagatatttataagaagtacacggattttacaggattcagtcttgtaaagctggaagtgttgaatatgcttacagtagcggggcttgttatagaatcagctcttgaaagaaaagagagcagaggtgctcattatagaacagactacgacaaaactgatgatacgaactggagaaaaaatatagttaaggaattggagaagggaaaaatggcttcaccattttaa

SEQ ID NO: 62 - protein, 534 aa, Clostridium cellulolyticum
MEEDSNKVDVEVIHKDVVIIGSGIAGVYTALEIPDSFQIGIITKETLDISNSVLAQGGIAVSLDEKNDSPQLHFKDTLFAGAGLNDQKSVWVLVEEAAENIRILCSLGVNFDKSGQHLSLTREGAHSVNRIIHSGDTTGKEVCDKLIEVARRKKNISIFESHFAVDLVIEEGKCKGVIVYDEIEDKIKIFKSGSVVVATGGFGQIYAHTTNPEVATGDGVGMCLRAGAQAMDMEFIQFHPTVLYHPKDKSFLISEAVRGEGAQLKNSNGEPFMKKYHELGELAPRDIVSRAIFKEMYLTDSKNVFLDITFKGREYIESRFPNIFKTCLDYGIDISKDFIPVAPAEHYCMGGVKTDVDGQTNIPGLYACGEVACTGIHGANRLASNSLLEGLVFGRKIAKKIESEGRPCNNSAVNSRLCYVSNKENDAALKSMKEEIQAAMTKYVGIIRSQQGLEKAAQIIKDIYKKYTDFTGFSLVKLEVLNMLTVAGLVIESALERKESRGAHYRTDYDKTDDTNWRKNIVKELEKGKMASPF

SEQ ID NO: 63 - DNA, 837 bp, Clostridium cellulolyticum
atgaaactcagtaatctttatatccatgaaatagttatgaatgcattaaaagaggatatgccactaggtgatattacaacagacaatattctttcagaaggagattcatccagagccgaatttatggcaaagcaggatgcggttattgcagggctcgatgttgcgaagtatgtttttgaggtactggatagcggcatatgttttaaggcctttgtaaaagatggagacaaggtttcgaaaggtgatattatagccgaggtaagcggttcgacaagagctttgttaaaaggtgaaaggactgcattgaactttatgcaaaggttatctgcaattgctactatgactaacagatatgttagtaaagttcaggggttacctgtaaaggtaactgatacaagaaaaactactcccggtctgagacttctggagaaatatgcagtaagtgcaggaggaggagccaatcacagattttcgctttctgacggtgttctcataaaggataaccacattgctgctgccggaggaataacaaatgcggttcaacgtgtaagaaacagtattcctcatactgtaaagatcgaagtagaagtagagtccatggaagaggttcgtgaggctctcgaatgcaaggcagatataattatgcttgataatatgtcaaatgaacagatggctgaggctgtcaagcttataaataaaagagctcttgcggaggcctcggggaatataagtgaagaaactatatataatgtagcgttaacaggagttgatattatatctataggtaaacttactcactctgcaaattctattgatataagtatgaatatagaatag

SEQ ID NO: 64 - protein, 278 aa, Clostridium cellulolyticum
MKLSNLYIHEIVMNALKEDMPLGDITTDNILSEGDSSRAEFMAKQDAVIAGLDVAKYVFEVLDSGICFKAFVKDGDKVSKGDIIAEVSGSTRALLKGERTALNFMQRLSAIATMTNRYVSKVQGLPVKVTDTRKTTPGLRLLEKYAVSAGGGANHRFSLSDGVLIKDNHIAAAGGITNAVQRVRNSIPHTVKIEVEVESMEEVREALECKADIIMLDNMSNEQMAEAVKLINKRALAEASGNISEETIYNVALTGVDIISIGKLTHSANSIDISMNIE

SEQ ID NO: 65 - DNA, 879 bp, Clostridium cellulolyticum
atgaacgagagatatcaattaaacaaaaatcttgcccaaatgctaaagggcggagtaatcatggatgtagtaaatgccaaagaagcagaaattgcacaaaaagccggagccgttgcagtaatggctctcgaaagagttccttccgatataagaaaagccggaggagttgcaagaatgtccgatccaaaaatgataaaagatatacaaagtgccgtatcaattcctgttatggccaaagttagaataggacattttgttgaagcacaggttcttgaagccctttcaattgactatattgatgaaagcgaggttttaactccggcagacgaagaatttcacatagataagcataccttcaaggttccatttgtatgcggtgcaaaaaatctcggagaagctctcagaagaattagtgaaggtgcatccatgataagaactaaaggtgaagccggtacaggaaatgttgttgaagccgtccgacatatgagaactgtaacaaatgaaatcagaaaggtgcagagtgcatccaagcaggaacttatgaccatagcaaaagaatttggtgctccatatgaccttattttatatgttcacgaaaacggtaagcttcctgttataaactttgcagcaggcggaatcgcaactcccgccgatgcggcattaatgatgcagcttggatgcgacggcgtatttgttggttcgggaatatttaaatcctcagatccagccaaaagagcaaaggcaatcgtaaaggcaactacatactataatgatccgcaaatcattgcagaggtctctgaagagcttggtactgccatggattccatagatgtaagagagttaacaggcaacagtctgtatgcctctagaggatggtaa

SEQ ID NO: 66 - protein, 292 aa, Clostridium cellulolyticum
MNERYQLNKNLAQMLKGGVIMDVVNAKEAEIAQKAGAVAVMALERVPSDIRKAGGVARMSDPKMIKDIQSAVSIPVMAKVRIGHFVEAQVLEALSIDYIDESEVLTPADEEFHIDKHTFKVPFVCGAKNLGEALRRISEGASMIRTKGEAGTGNVVEAVRHMRTVTNEIRKVQSASKQELMTIAKEFGAPYDLILYVHENGKLPVINFAAGGIATPADAALMMQLGCDGVFVGSGIFKSSDPAKRAKAIVKATTYYNDPQIIAEVSEELGTAMDSIDVRELTGNSLYASRGW

SEQ ID NO: 67 - DNA, 570 bp, Clostridium cellulolyticum
atgaaaaaaataggtgtgttaggcttgcagggtgctatctcagaacatttggataaactatccaaaataccaaatgtagagccattcagcctaaaatataaagaagaaattgatacaatagacggacttatcatacccggcggtgaaagtactgcaatcggcaggcttctctctgattttaacctgacagaaccactgaaaacaagggtaaatgccgggatgcctgtatggggaacctgtgcaggcatgattatccttgcaaaaacgattactaatgaccgccgacgtcatctggaggttatggacataaatgttatgcggaacgggtatggaagacagttgaacagctttacaacagaggtttccctggctaaagtttcttctgataaaatcccgttggtttttattagagcaccttatgtagtcgaggtagctccgaatgttgaagttcttctgcgtgtagacgaaaacatagtcgcgtgcaggcaggacaatatgctggccacctcctttcatccggagctgacagaagacctgagttttcacaggtactttgcagaaatgatataa

SEQ ID NO: 68 - protein, 189 aa, Clostridium cellulolyticum
MKKIGVLGLQGAISEHLDKLSKIPNVEPFSLKYKEEIDTIDGLIIPGGESTAIGRLLSDFNLTEPLKTRVNAGMPVWGTCAGMIILAKTITNDRRRHLEVMDINVMRNGYGRQLNSFTTEVSLAKVSSDKIPLVFIRAPYVVEVAPNVEVLLRVDENIVACRQDNMLATSFHPELTEDLSFHRYFAEMI

SEQ ID NO: 69 - DNA, 486 bp, Clostridium cellulolyticum
atgatttcaatgatatgggctatgggccgcaacaacgcccttggatgtaaaaacagaatgccctggtacattcccgcagattttgcatatttcaaaaaagttacaatgggaaaaccggtcattatggggagaaaaacttttgaatctatcggtaaacctttaccgggcagaaagaacatagtaattactcgagacacaggatatgatccacaaggctgtattgtggttaattctatagaaaaagccatggagtatacagaagaaaaggaagtctttataatagggggagcagaaatatacaaagaatttcttcctattgcagacagactatatataactctgatagaaaaagagtttgaagcggatgcatttttcccggaaatagactatagtaagtggaagcagatatcctgcgaaacaggaatcaaggatgaaaaaaatccatatgagtataagtggttggtatacgaaagagttaaacaataa

SEQ ID NO: 70 - protein, 161 aa, Clostridium cellulolyticum
MISMIWAMGRNNALGCKNRMPWYIPADFAYFKKVTMGKPVIMGRKTFESIGKPLPGRKNIVITRDTGYDPQGCIVVNSIEKAMEYTEEKEVFIIGGAEIYKEFLPIADRLYITLIEKEFEADAFFPEIDYSKWKQISCETGIKDEKNPYEYKWLVYERVKQ

SEQ ID NO: 71 - DNA, 2105 bp, Clostridium phytofermentans
tgggtggatagtgtgaaaaattatattagatgattttggagatgttgattggtattgaatttaaatataattaaacgttctttatagtagataaaaagaatgacttgtatcttcattttttgtataattgaaataatctaaaaaatggataaactagaatttttactggaggaacttatggatatatatagtttctggaacacaaccttaaaacagaatgcttctactatgagaacatattttcatgcagatgcttatgtaagatggcataatactaatgaacattttacagttgatgagtttattcaagcaaattgtgagtatcccgataaatgggatggagaagtagaaagagttgaggaaattcgtgatttaattattactgttacacatgtatatgctgttaacaagccactttcttttcatgttacttcatttattagagttaaagatgacaaaattatttctattgatgaatactggggagatgatggatccgccccacaatggcgattagataaacatattggtaagtctataaaataaattctagtttgacgagaagttgaatgtaatagtttaacttctcgtcttttcatcaaaatagcttgaattactctaaatcttgaatattcatcttctctagttcattgttctttttaaactgttctctacatgcgaaataaattgcaagagatacaatagttggtatattcataataaataaaggaagcaaccattggagtatagtatctaacacagtagcattcgtcgacattgctcttacttgattaaatattattccattcaggaaagctactgctgaaaaagacatacagagtaacggaagaattaatcctaaccatttattttttctctttgaaagaaatacctgtaataaaaccaaacttacaaaaaacaataataaaaaaaacaatttagacattcttaataccccttctcaatttttttatattcagaaatcaatatcttagctgtatcaaaatcgaccttatcgttcacagcaagtcgtttaatcaaagcaacataagggtcatttgtagtgtcttcgctaattttaattttttgtattaatttatctagtctcaatctaactgtaggataagtaacattatattgattcgctatttcttttaatgaaccagaggcaagtaaaaattttttaataaatgaaatgtcttcatcatctaaatttaacatccattctggtacaacttcgattgccatgtaatcactcctttaatattattaagtttatactttaattttattaaagtcaatattaaattatattaaaattaaaataaatagtttacaagaattaaacgaaaaatttttatcaaccctcagaaaaattcaactcacggaaacagaatttatttgctcagattaccgattctaaaaaatggtgatgaatccttctttcgattcatcaccatttttcataacatatgattatttactcatccatacaattatagaatagctttgtaagttccacacggctggtgatacctaacttttgatagatattatacaccatgcgctttactgttccctcactaatatgaagtttgtgggctatgtatttatttgtttcattttgcactattgatgtggctatcactaattccctttttgttagaatgccctctaagtggttatcattaataagattttttgaaatagtactatttggatgcttttccttgatattctcttcatttttctgatcatttatcatacttttctctccctatttggtggcagctctcgtttcaaagtacaaatccatattctattctcctttattacatttttaaatacttattgtgatcatctttattcatcggtataagacctaatttaaaatcggctacaacatattattctattttttccctttttcagggaaatcttgtattcaattggcttttttttgatcccaataatggtactcaggcaatttccaatagaattcatgtgaaatataatgaaaactctttttaagcatattgttctctttctatgcctattcaatagcccgattgatagatagaaatctaaaatcaaaattgggaaca